SEQUENCE LISTING (540 sequences)

SEQ ID NO: 1 - protein, 16 aa, Homo sapiens
PFAYYSDLCGVNGVDY

SEQ ID NO: 2 - protein, 16 aa, Lama Glama
PFSYYSHLCGVNGYDY

SEQ ID NO: 3 - protein, 16 aa, Lama Glama
PFSYYSSLCGVNEYDY

SEQ ID NO: 4 - protein, 15 aa, Lama Glama
PWDSWYCGIGNDYDY

SEQ ID NO: 5 - protein, 16 aa, Lama Glama
PFIHYSDLCGVNGYDY

SEQ ID NO: 6 - protein, 22 aa, Lama Glama
DPIHNCYSGSSYYYSPEAVYDY

SEQ ID NO: 7 - protein, 16 aa, Lama Glama
PFAHYSDLCGVNGYDY

SEQ ID NO: 8 - protein, 15 aa, Lama Glama
SGSYYYPTDVHEYDY

SEQ ID NO: 9 - protein, 16 aa, Lama Glama
PFEYYSDLCGVNGYDY

SEQ ID NO: 10 - protein, 21 aa, Lama Glama
DPIHNCYSGRYYYSPEAVYEY

SEQ ID NO: 11 - protein, 16 aa, Lama Glama
PFSHYSDLCGVNAIDY

SEQ ID NO: 12 - protein, 16 aa, Lama Glama
PFEYYSDLCGVNGYDY

SEQ ID NO: 13 - protein, 15 aa, Lama Glama
SGSYYYPTDVFEYDY

SEQ ID NO: 14 - protein, 21 aa, Lama Glama
HPLQNCCGGSAYASPEAVYEY

SEQ ID NO: 15 - protein, 16 aa, Lama Glama
PFAYYSNLCGVNGYDY

SEQ ID NO: 16 - protein, 21 aa, Lama Glama
DPIHNCYSGNYYASPEAVYDY

SEQ ID NO: 17 - protein, 15 aa, Lama Glama
DPLVCGYNDPRLADY

SEQ ID NO: 18 - protein, 16 aa, Lama Glama
PFAHYSDLCGVNGYDY

SEQ ID NO: 19 - protein, 16 aa, Lama Glama
PFTHYSDLCGVNEYDY

SEQ ID NO: 20 - protein, 11 aa, Lama Glama
PGIAACRGIHY

SEQ ID NO: 21 - protein, 11 aa, Lama Glama
PGIAACRGIHY

SEQ ID NO: 22 - protein, 14 aa, Lama Glama
PRGWGPTGPHEYGY

SEQ ID NO: 23 - protein, 15 aa, Lama Glama
SFQSGAAPGANFYDY

SEQ ID NO: 24 - protein, 12 aa, Lama Glama
PAPGSSGYEYDY

SEQ ID NO: 25 - protein, 12 aa, Lama Glama
PSPGSSGYEYDY

SEQ ID NO: 26 - protein, 14 aa, Lama Glama
SLRGWDTTRIDYEY

SEQ ID NO: 27 - protein, 11 aa, Lama Glama
PGIAACRGIHY

SEQ ID NO: 28 - protein, 14 aa, Lama Glama
PRGWGPTGPHEYGY

SEQ ID NO: 29 - protein, 12 aa, Lama Glama
PAPGSSGYEYDY

SEQ ID NO: 30 - protein, 12 aa, Lama Glama
PSPGSSGYEYDY

SEQ ID NO: 31 - protein, 14 aa, Lama Glama
RAADTRLGPYEYDY

SEQ ID NO: 32 - protein, 14 aa, Lama Glama
PRGWGPTGPHEYGY

SEQ ID NO: 33 - protein, 11 aa, Lama Glama
PGIAACRGIHY

SEQ ID NO: 34 - protein, 13 aa, Lama Glama
GRGFYHDYSSYEY

SEQ ID NO: 35 - protein, 9 aa, Lama Glama
GPDCSSYDY

SEQ ID NO: 36 - protein, 14 aa, Lama Glama
SLRGWDTTRIDYEY

SEQ ID NO: 37 - protein, 13 aa, Lama Glama
ARGDIDVYTLSDS

SEQ ID NO: 38 - protein, 13 aa, Lama Glama
ARGDIDVYTLSDS

SEQ ID NO: 39 - protein, 14 aa, Lama Glama
PRGWGPTGPHEYGY

SEQ ID NO: 40 - protein, 20 aa, Lama Glama
RLFSGGCAVVAGTSWADFGS

SEQ ID NO: 41 - protein, 20 aa, Lama Glama
RLFKGGCAVVAGTSWADFGS

SEQ ID NO: 42 - protein, 12 aa, Lama Glama
LATTVTPSWVNY

SEQ ID NO: 43 - protein, 14 aa, Lama Glama
PRGWGPTGPHEYDY

SEQ ID NO: 44 - protein, 14 aa, Lama Glama
SLRGWDTTWIDYEY

SEQ ID NO: 45 - protein, 13 aa, Lama Glama
DSRRGGVGNFFRS

SEQ ID NO: 46 - protein, 14 aa, Lama Glama
PRGWGPTGPIEYAY

SEQ ID NO: 47 - protein, 8 aa, Lama Glama
RRNFLSNY

SEQ ID NO: 48 - protein, 11 aa, Lama Glama
PGIAACRGIHY

SEQ ID NO: 49 - protein, 14 aa, Lama Glama
AWCDSSWYRSFVGY

SEQ ID NO: 50 - protein, 20 aa, Lama Glama
RLFSGGCAVVARTSWADFGS

SEQ ID NO: 51 - protein, 11 aa, Lama Glama
PGIAACRGIHY

SEQ ID NO: 52 - protein, 14 aa, Lama Glama
PRGWGPTGPHEYGY

SEQ ID NO: 53 - protein, 11 aa, Lama Glama
PVKVAGLEYAY

SEQ ID NO: 54 - protein, 13 aa, Lama Glama
DVQHSAWLKPLTY

SEQ ID NO: 55 - protein, 3 aa, Lama Glama
DIY

SEQ ID NO: 56 - protein, 15 aa, Lama Glama
PYYSDFEGTTTEYDY

SEQ ID NO: 57 - protein, 16 aa, Lama Glama
TTRGRYSALSASAYDY

SEQ ID NO: 58 - protein, 19 aa, Lama Glama
DRYIRARQGDYWGAYEYDY

SEQ ID NO: 59 - protein, 21 aa, Lama Glama
DPIHNCYSGRYYASPDAVYDY

SEQ ID NO: 60 - protein, 15 aa, Lama Glama
DPLVCGYNDPRLADY

SEQ ID NO: 61 - protein, 16 aa, Lama Glama
PFNHYSDLCGVNAIDY

SEQ ID NO: 62 - protein, 16 aa, Lama Glama
PFSYYSSLCGVNEYDY

SEQ ID NO: 63 - protein, 16 aa, Lama Glama
PFSYYNNLCGVNGVDY

SEQ ID NO: 64 - protein, 20 aa, Lama Glama
RLFSGGCAVVAGTSWADFGS

SEQ ID NO: 65 - protein, 16 aa, Lama Glama
PFAHYSDLCGVNGYDY

SEQ ID NO: 66 - protein, 16 aa, Lama Glama
PFAYYSDLCGVNEYDY

SEQ ID NO: 67 - protein, 15 aa, Lama Glama
PHSDYDEEAPSDFGS

SEQ ID NO: 68 - protein, 20 aa, Lama Glama
RLFSGGCAVVVGTSWADFGS

SEQ ID NO: 69 - protein, 11 aa, Lama Glama
SLGSSWCAYDY

SEQ ID NO: 70 - protein, 15 aa, Lama Glama
DPLVCGYNDPRLADY

SEQ ID NO: 71 - protein, 20 aa, Lama Glama
RLFSGGCAVVAGTSWADFGS

SEQ ID NO: 72 - protein, 15 aa, Lama Glama
SGSYYYPTDVHEYAY

SEQ ID NO: 73 - protein, 16 aa, Lama Glama
PFEYYSAYCGVNRYDY

SEQ ID NO: 74 - protein, 18 aa, Lama Glama
AHDNYWFTDDSLGRGLKY

SEQ ID NO: 75 - protein, 11 aa, Lama Glama
KTFGSNWYDDY

SEQ ID NO: 76 - protein, 21 aa, Lama Glama
DPIHNCYSGRYYASPEAVYDY

SEQ ID NO: 77 - protein, 21 aa, Lama Glama
DPFHNCYSGSHYSSPEAVYEY

SEQ ID NO: 78 - protein, 16 aa, Lama Glama
PFEYYSDLCGVNGYDY

SEQ ID NO: 79 - protein, 16 aa, Lama Glama
PFNYYSDLCGVNGVDY

SEQ ID NO: 80 - protein, 15 aa, Lama Glama
SGSYYYPTDVHEYAY

SEQ ID NO: 81 - protein, 16 aa, Lama Glama
PFAHYSDLCGVNGYDY

SEQ ID NO: 82 - protein, 8 aa, Lama Glama
EMDGSRYV

SEQ ID NO: 83 - protein, 9 aa, Lama Glama
VHPSTGFGS

SEQ ID NO: 84 - protein, 15 aa, Lama Glama
PHSDYDEEAPSDFGS

SEQ ID NO: 85 - protein, 20 aa, Lama Glama
RLFSGGCAVVASTSWADFGS

SEQ ID NO: 86 - protein, 20 aa, Lama Glama
RLFRGGCAVVAGTSWADFGS

SEQ ID NO: 87 - protein, 15 aa, Lama Glama
DPLVCGYNDPRLADY

SEQ ID NO: 88 - protein, 21 aa, Lama Glama
DPIHNCYSGRYYASPDAVYEY

SEQ ID NO: 89 - protein, 21 aa, Lama Glama
DPIHNCYSGNGYDSPEAVYDY

SEQ ID NO: 90 - protein, 21 aa, Lama Glama
DPFHNCYSGSAYSSPEAVYEY

SEQ ID NO: 91 - protein, 11 aa, Lama Glama
PVKVAGLEYDY

SEQ ID NO: 92 - protein, 11 aa, Lama Glama
SVGSSWCAYDY

SEQ ID NO: 93 - protein, 11 aa, Lama Glama
SLGSSWCAYDY

SEQ ID NO: 94 - protein, 14 aa, Lama Glama
DSLPCYYDKMVYDY

SEQ ID NO: 95 - protein, 14 aa, Lama Glama
DSFACDYGKMIYDY

SEQ ID NO: 96 - protein, 11 aa, Lama Glama
SLGSSWCAYDY

SEQ ID NO: 97 - protein, 15 aa, Lama Glama
DPLVCGYNDPRLADY

SEQ ID NO: 98 - protein, 15 aa, Lama Glama
SGSYYYPTDVHEYDY

SEQ ID NO: 99 - protein, 16 aa, Lama Glama
PFNYYSNLCGVNGVDY

SEQ ID NO: 100 - protein, 21 aa, Lama Glama
DPIHNCYSGSHYYSPEAVYEY

SEQ ID NO: 101 - protein, 21 aa, Lama Glama
DPIHNCYSGSDYASPEAVYEY

SEQ ID NO: 102 - protein, 16 aa, Lama Glama
PFIHYSDLCGVNGNDY

SEQ ID NO: 103 - protein, 15 aa, Lama Glama
SGSYYYPTDVHEYDY

SEQ ID NO: 104 - protein, 14 aa, Lama Glama
APIFECPSGEIYDY

SEQ ID NO: 105 - protein, 21 aa, Lama Glama
DPIHNCYSGTYYASPEAVYEY

SEQ ID NO: 106 - protein, 21 aa, Lama Glama
DPIHNCYSGSYYASPEAVYDY

SEQ ID NO: 107 - protein, 15 aa, Lama Glama
SGSYYYPTDVHEYAY

SEQ ID NO: 108 - protein, 15 aa, Lama Glama
SGSYYYPTDVHEYAY

SEQ ID NO: 109 - protein, 16 aa, Lama Glama
PFAYYSDLCGVNEYDY

SEQ ID NO: 110 - protein, 16 aa, Lama Glama
PFNHYSFLCGVNEYDY

SEQ ID NO: 111 - protein, 15 aa, Lama Glama
SGSYYYPTEVYEYDY

SEQ ID NO: 112 - protein, 15 aa, Lama Glama
SGSYYYPTDVHEYDY

SEQ ID NO: 113 - protein, 16 aa, Lama Glama
PFEYYSDLCGVNGYDY

SEQ ID NO: 114 - protein, 21 aa, Lama Glama
DPIHNCYGGSYYASPEAVYEY

SEQ ID NO: 115 - protein, 12 aa, Lama Glama
AGASSWCFPPGY

SEQ ID NO: 116 - protein, 21 aa, Lama Glama
DPIHNCYSGIYYASPEAVYDY

SEQ ID NO: 117 - protein, 15 aa, Lama Glama
SGSYYYPTDVHEYDY

SEQ ID NO: 118 - protein, 21 aa, Lama Glama
DPIHNCYSGRYYASPDAVYDY

SEQ ID NO: 119 - protein, 16 aa, Lama Glama
PFSYYSDLCGVNGYDY

SEQ ID NO: 120 - protein, 15 aa, Lama Glama
SGSYYYPSDVHEYDY

SEQ ID NO: 121 - protein, 16 aa, Lama Glama
PFSYYSGLCGVNGVDY

SEQ ID NO: 122 - protein, 11 aa, Lama Glama
SLGSSWCAYDY

SEQ ID NO: 123 - protein, 21 aa, Lama Glama
DPVHNCYSGRYYASPDAVYEY

SEQ ID NO: 124 - protein, 15 aa, Lama Glama
SGSYYYPTDVHEYAY

SEQ ID NO: 125 - protein, 16 aa, Lama Glama
PFSHYNDLCGVNAIDY

SEQ ID NO: 126 - protein, 21 aa, Lama Glama
DPIHNCYSGNGYDSPEAVYDY

SEQ ID NO: 127 - protein, 16 aa, Lama Glama
PFEYYSDLCGVNGYDY

SEQ ID NO: 128 - protein, 16 aa, Lama Glama
PFEYYSNLCGVNGYDY

SEQ ID NO: 129 - protein, 21 aa, Lama Glama
DPLHNCYSGRGYYSPEAVYEY

SEQ ID NO: 130 - protein, 21 aa, Lama Glama
DPIHNCYSGSYYASPEAVYEY

SEQ ID NO: 131 - protein, 16 aa, Lama Glama
PFEYYSNLCGVNGYDY

SEQ ID NO: 132 - protein, 11 aa, Lama Glama
PGIAACRGIHY

SEQ ID NO: 133 - protein, 11 aa, Lama Glama
PGIAACRGIHY

SEQ ID NO: 134 - protein, 11 aa, Lama Glama
PGIAACRGIHY

SEQ ID NO: 135 - protein, 11 aa, Lama Glama
PGIAACRGIHY

SEQ ID NO: 136 - protein, 11 aa, Lama Glama
PGIAACRGIHY

SEQ ID NO: 137 - protein, 17 aa, Lama Glama
KPNLKYGSYWPPRGYDY

SEQ ID NO: 138 - protein, 17 aa, Lama Glama
KPNLKYGSTWPPRGYDY

SEQ ID NO: 139 - protein, 17 aa, Lama Glama
KPNLKYGSYWPPRGYDY

SEQ ID NO: 140 - protein, 17 aa, Lama Glama
KPNLKYGSDWPPRGYDY

SEQ ID NO: 141 - protein, 8 aa, Lama Glama
RTSRSPRP

SEQ ID NO: 142 - protein, 15 aa, Lama Glama
SNYYSVYDDRPVMDY

SEQ ID NO: 143 - protein, 7 aa, Lama Glama
GSGSWGV

SEQ ID NO: 144 - protein, 19 aa, Lama Glama
NEGYCSGYGCYEDSGQYDY

SEQ ID NO: 145 - protein, 19 aa, Lama Glama
NEGYCSGYGCYEDSGQYDY

SEQ ID NO: 146 - protein, 12 aa, Lama Glama
GGRSFLPFVPAY

SEQ ID NO: 147 - protein, 19 aa, Lama Glama
NEGYCSGYGCYEDSGQYDY

SEQ ID NO: 148 - protein, 19 aa, Lama Glama
NGGYCSGYGCYEDSGQYDY

SEQ ID NO: 149 - protein, 11 aa, Lama Glama
PGIAACRGIHY

SEQ ID NO: 150 - protein, 11 aa, Lama Glama
PGIAACRGIHY

SEQ ID NO: 151 - protein, 11 aa, Lama Glama
PGIAACRGIHY

SEQ ID NO: 152 - protein, 14 aa, Lama Glama
PRGWGPTGPIEYAY

SEQ ID NO: 153 - protein, 14 aa, Lama Glama
PRGWGPTGPIEYGY

SEQ ID NO: 154 - protein, 14 aa, Lama Glama
PRGWGPTGPHEYGY

SEQ ID NO: 155 - protein, 14 aa, Lama Glama
PRGWGPTGPHEYAY

SEQ ID NO: 156 - protein, 12 aa, Lama Glama
PAPGSSGYEYDY

SEQ ID NO: 157 - protein, 11 aa, Lama Glama
PGIAACRGIHY

SEQ ID NO: 158 - protein, 14 aa, Lama Glama
PRGWGPTGPHEYAY

SEQ ID NO: 159 - protein, 14 aa, Lama Glama
PRGWGPTGPLEYGY

SEQ ID NO: 160 - protein, 14 aa, Lama Glama
PRGWGPTGPHEYGY

SEQ ID NO: 161 - protein, 14 aa, Lama Glama
PRGWGPTGPHEYGY

SEQ ID NO: 162 - protein, 14 aa, Lama Glama
PRGWGPTGPHEYGY

SEQ ID NO: 163 - protein, 14 aa, Lama Glama
PRGWGPTGPHEYGY

SEQ ID NO: 164 - protein, 14 aa, Lama Glama
PRGWGPTGPHEYAY

SEQ ID NO: 165 - protein, 14 aa, Lama Glama
PRGWGPTGPHEYGY

SEQ ID NO: 166 - protein, 14 aa, Lama Glama
PRGWGPTGPHEYAY

SEQ ID NO: 167 - protein, 125 aa, Lama Glama
EVQLVESGGGLVQPGGSLRLSCAASGFTLDTYNIGWFRQAPGKEREWVSCISSSDGSTNYADSVKGRFTISRDNAKNTVYLQMNNLKPEDTAVYYCAAPFAYYSDLCGVNGVDYWGQGTQVTVSS

SEQ ID NO: 168 - protein, 125 aa, Lama Glama
EVQLVESGGGLVQPGGSLRLSCAASGFTLDYYNIGWFRQAPGKEREWVSCINSSDGSTYYTDSVKGRFTISRDNAKNTVYLQMNSLKPEDTAIYYCAAPFSYYSHLCGVNGYDYWGQGTQVTVSS

SEQ ID NO: 169 - protein, 125 aa, Lama Glama
EVQLVESGGGLVQPGGSLRLSCAASGFTLDYYNIGWFRQAPGKEREWVSCISSSDGSTAYADSVKGRFTISRDNAKNTVYLQMNSLKPEDTAVYYCAAPFSYYSSLCGVNEYDYWGQGTQVTVSS

SEQ ID NO: 170 - protein, 124 aa, Lama Glama
EVQLVESGGGLVQPGGSLRLSCAISGFTLDLHVIGWLRQAPGKEREWVSCISSSDGSTYYADSVKGRFTISRDNAKNTVYLQMNSLKPEDTAVYYCAAPWDSWYCGIGNDYDYWGQGTQVTVSS

SEQ ID NO: 171 - protein, 125 aa, Lama Glama
EVQLVESGGGLVQPGGSLRLSCAASGFTLDYYNIGWFRQAPGKEREWVSCIRGSNGSTGYTDSVKGRFTISRDNAKNTVYLQMNSLKPEDTAVYYCAAPFIHYSDLCGVNGYDYWGQGTQVTVSS

SEQ ID NO: 172 - protein, 131 aa, Lama Glama
EVQLVESGGGLVQAGGSLRLSCAASGFTLDKYAIGWFRQAPGKEREGVSCISSRGGSTYYVDSVKGRFTISRDNAKNTVYLQMNSLKPEDTAVYYCAADPIHNCYSGSSYYYSPEAVYDYWGQGTQVTVSS

SEQ ID NO: 173 - protein, 125 aa, Lama Glama
EVQLVESGGGLVQPGGSLRLSCAASGFTLDYYNIGWFRQAPGKEREWVSCITSSNGSTYYTDSVKGRFTISRDNAKNTVYLQMNSLKPEDTAVYYCAAPFAHYSDLCGVNGYDYWGQGTQVTVSS

SEQ ID NO: 174 - protein, 123 aa, Lama Glama
EVQLVESEGGLVQAGGSLRLSCAASGSTFSSYAMGWYRQAPGKQRELVAVISNGGITNYPNSVKGRFTISRDNAKNTVYLQMNSLKPEDTAVYYCFYSGSYYYPTDVHEYDYWGQGTQVTVSS

SEQ ID NO: 175 - protein, 125 aa, Lama Glama
EVQLVESGGGLVQPGGSLRLSCAASGFTLDYYNIGWFRQAPGKEREWVSCINSSDGSTYYADSVKGRFTISRDNAKNTVYLQMNSLKPEDTAVYYCAAPFEYYSDLCGVNGYDYWGQGTQVTVSS

SEQ ID NO: 176 - protein, 130 aa, Lama Glama
EVQLVESGGGLVQAGGSLRLSCAASGFTLDYYAIGWFRQAPGKEREGISCISSRGGSTFYVDSVKGRFTISRDNAKNTVYLQMNSLKPEDTAVYYCAADPIHNCYSGRYYYSPEAVYEYWGQGTQVTVSS

SEQ ID NO: 177 - protein, 125 aa, Lama Glama
EVQLVESGGGLVQPGGSLRLSCAASGFTLDYHNIGWFRQAPGKEREWVSCISSSGGSTAYADSVKGRFTISRDNAKNTVYLQMNSLKPEDTAVYYCAAPFSHYSDLCGVNAIDYWGQGTQVTVSS

SEQ ID NO: 178 - protein, 125 aa, Lama Glama
EVQLVESGGGLVQPGGSLRLSCAASGFTLDYYNIGWFRQAPGKEREWVSCINSSDGSTYYADSVKGRFTVSRDNAKNTVYLQMNSLKPEDTAVYYCAAPFEYYSDLCGVNGYDYWGQGTQVTVSS

SEQ ID NO: 179 - protein, 123 aa, Lama Glama
EVQLVESGGGLVQAGGSLRLSCAASGSTFNSYAMGWYRQAPGKQREWVAAFSTGGSTNYADSVKGRFTISRDNAKNTVYLQMNSLKPEDTAVYYCFYSGSYYYPTDVFEYDYWGQGTQVTVSS

SEQ ID NO: 180 - protein, 130 aa, Lama Glama
EVQLVESGGGLVQAGGSLRLSCAASGFALDYYAVGWFRQAPGKEREGVSCISSRGGSTFYADSVKGRFTTSRNNAKNTVYLQMNSLKPEDTAVYYCAAHPLQNCCGGSAYASPEAVYEYWGQGTQVTVSS

SEQ ID NO: 181 - protein, 125 aa, Lama Glama
EVQLVESGGGLVQPGGSLRLSCAASGFTLDYYNIGWFRQAPGKEREWVSCINSSDGSTYYADSVKGRFTISRDNAKNTVYLQMNSLKPEDTAVYYCAAPFAYYSNLCGVNGYDYWGQGTQVTVSS

SEQ ID NO: 182 - protein, 130 aa, Lama Glama
EVQLVESGGGLVQAGGSLRLSCAASGFALDYYAVGWFRQAPGKEREGVSCISSRGGSTYYVDSVKGRFTTSRDNAKNTVYLQMNSLKPEDTAVYYCAADPIHNCYSGNYYASPEAVYDYWGQGTQVTVSS

SEQ ID NO: 183 - protein, 124 aa, Lama Glama
EVQLVESGGGLVQAGGSLRLSCAASGFTFDDYAIGWFRQAPGKEREGVSCISSHDRTTYYADSVKGRFTISSDNAKNTVYLQMNSLKPEDTAVYYCAADPLVCGYNDPRLADYWGQGTQVTVSS

SEQ ID NO: 184 - protein, 125 aa, Lama Glama
EVQLVESGGGLVQPGGSLRLSCAASGFTLDYYNIGWFRQAPGKEREWVSCITSSYGSTYYTDSVKGRFTISRDNAKNTVYLQMNSLKPEDTAVYYCAAPFAHYSDLCGVNGYDYWGQGTQVTVSS

SEQ ID NO: 185 - protein, 125 aa, Lama Glama
EVQLVESGGGLVQPGGSLRLSCAASGFTLDYHNIGWFRQAPGKEREWVSCISSSDGRTAYADSVKGRFTISRDNAKNTVYLQMNSLKPEDTAVYYCAAPFTHYSDLCGVNEYDYWGQGTQVTVSS

SEQ ID NO: 186 - protein, 120 aa, Lama Glama
EVQLVESGGGLVQPGGSLRLSCAASGFTFDDYAIGWFRQAPGKEPEGIGCISSSGGSTYYADSVKGRFTISRDNAKNTVYLQMNSLKPEDTAVYYCATPGIAACRGIHYWGQGTQVTVSS

SEQ ID NO: 187 - protein, 120 aa, Lama Glama
KVQLVESGGGLVQPGGSLRLSCAASGFTFDDYAIGWFRQAPGKEPEGISCISSSGGITYYADSVKGRFTISRDNAKNTVYLQMNSLKPEDTAVYYCATPGIAACRGIHYTGQGTQVTVSS

SEQ ID NO: 188 - protein, 123 aa, Lama Glama
EVQLVESGGGLVQPGGSLRLSCAASGFTFGNYDMSWVRQAPGKGPEWVSAINSGGGTTYYADSVKGRFTISRDNAKNTLYLQMNSLKPEDTAVYYCATPRGWGPTGPHEYGYWGQGTQVTVSS

SEQ ID NO: 189 - protein, 124 aa, Lama Glama
EVQLVESGGGLVQAGGSLRLSCAASGRTFSNYAMGWFRQAPGKEREFVAAISWSGGDTYYADSVKGRFTISRDNAKNTVCLQMNSLKPEDTAVYYCAASFQSGAAPGANFYDYWGQGTQVTVSS

SEQ ID NO: 190 - protein, 121 aa, Lama Glama
EVQLVESEGGSVQAGGSLRLSCAASGRTFSSYAMGWFRQAPGKEREFVAAINWSGGYTYYADSVRGRFTISRDNAKNTVYLQMNSLKPEDTAVYYCAAPAPGSSGYEYDYWGQGTQVTVSS

SEQ ID NO: 191 - protein, 121 aa, Lama Glama
EVQLVESGGGLVQAGGSLRLSCAASGRTFSSYAMGWFRQAPGKEREFVAAIFWSGGSTYYADSVRGRFTISRDIAKNTVYLQMNSLKPEDTAVYYCAAPSPGSSGYEYDYWGQGTQVTVSS

SEQ ID NO: 192 - protein, 123 aa, Lama Glama
EVQLVESGGGLVQTGDSLRLSCAASGSTFSNYRMGWFRQGPGKEREFVAAIGRNGQNTYYTDSVKGRFTVTRDNAKNMMYLQMNSLKPEDSAVYTCAASLRGWDTTRIDYEYWGQGTQVTVSS

SEQ ID NO: 193 - protein, 120 aa, Lama Glama
EVQLVESGGGLVQPGGSLRLSCTASGFTFDVYAIGWFRQAPGKEPEGISCISSSGSITYYADSVKGRFTTSRDSAKNTVYLQMNSLKPEDTAVYYCATPGIAACRGIHYWGQGTQVTVSS

SEQ ID NO: 194 - protein, 123 aa, Lama Glama
EVQLVESGGGLVQPGGSLRLSCAASGFTFGNYDMSWVRQAPGKGPEWVSAINSGGDTTYYADSVKGRFTISRDNAKNTLYLQMNSLKPEDTAVYYCATPRGWGPTGPHEYGYWGQGTQVTVSS

SEQ ID NO: 195 - protein, 121 aa, Lama Glama
EVQLVESRGGLVQAGGSLRLSCAASGRTFNSYAMGWFRQAPGKEREFVATINWSGGSTYYADSVKGRFTISRDNAKNTAYLQMNSLKPEDTAVYYCAAPAPGSSGYEYDYWGQGTQVTVSS

SEQ ID NO: 196 - protein, 121 aa, Lama Glama
EVQLVESGGGSVQAGGSLRLSCAASGRTFSSYAMGWFRQAPGKEREFVAAVYWSGGSTYYADSVRGRFTISRDNAKNTVYLQMNSLKPEDTAVYYCAAPSPGSSGYEYDYWGQGTQVTVSS

SEQ ID NO: 197 - protein, 123 aa, Lama Glama
EVQLVESGGGLVQAGGSLRLSCAASGRTFSSYAMAWFRQAPGKEREFVAAIRWSGGTAYYADSVQGRFTISRDNAKNTVYLQMNSLKPEDTAVYYCANRAADTRLGPYEYDYWGQGTQVTVSS

SEQ ID NO: 198 - protein, 123 aa, Lama Glama
EVQLVESGGGLVQPGGSLRLSCAASGFTFGNYDMSWVRQAPGKGPEWVSAINSGGGITYYADFVKGRFTISRDNAKNTLYLQMSSLKPEDTAVYYCATPRGWGPTGPHEYGYWGQGTQVTVSS

SEQ ID NO: 199 - protein, 120 aa, Lama Glama
EVQLVESGGGLVQPGGSLRLSCAASGFTFDDYAIGWFRQAPGKEPEGISCISSSGGITYYTDSVKGRFTISRDNAKNTVYLQMNSLKPEDTAVYYCATPGIAACRGIHYTGQGTQVTVSS

SEQ ID NO: 200 - protein, 122 aa, Lama Glama
EVQLVESGGGSAQAGGSLRLSCAASGRTSSTYAMGWFRQAPGKEHEFVSAIGRGTGATSYGDSVKGRFTISRDNAKNTVYLQMNSLQLEDTGDYYCVAGRGFYHDYSSYEYRGQGTQVTVSS

SEQ ID NO: 201 - protein, 118 aa, Lama Glama
EVQLVESGGGLVQPGGSLRLSCAASGFTLGYYTIVWFRQAPGKERKGVSCISSRDGSRYYADSVKGRFTISRDNAKNTVYLRMNSLKPEDTAVYYCAAGPDCSSYDYWGQGTQVTVSS

SEQ ID NO: 202 - protein, 123 aa, Lama Glama
EVQLVESGGGLVQTGDSLRLSCAASGSTFSNYRMGWFRQGPGKEREFVAAIGRNGQNTYYTDSVKGRFTVTRDNAKNMVYLQMNSLKPEDSAVYTCAASLRGWDTTRIDYEYWGQGTQVTVSS

SEQ ID NO: 203 - protein, 121 aa, Lama Glama
EVQLMESGGGLVQPGGSLRLSCAASGFTFSSYAMSWVRQAPGKGLEWVSRITSGGRTTYRDSVKGRFTISRDNSKNTLYLQMNSLKPEDTALYYCAKARGDIDVYTLSDSRGQGTQVTVSS

SEQ ID NO: 204 - protein, 121 aa, Lama Glama
EVQLVESGGGLVQPGGSLRLSCAASGFTFSSYAMSWVRQAPGKGLEWVSRITSGGRATYRDSVKGRFTISRDNSKNTLYLQMNSLKPEDTALYYCAKARGDIDVYTLSDSRGQGTQVTVSS

SEQ ID NO: 205 - protein, 123 aa, Lama Glama
EVQLVESGGGLVQPGGSLRLSCAASGFTFGNYDMSWVRRPPGKGPEWVSAINSGGGSTYYADSVKGRFTISRDNAKNTLYLQMNSLKPEDTAVYYCATPRGWGPTGPHEYGYWGQGTQVTVSS

SEQ ID NO: 206 - protein, 129 aa, Lama Glama
EVQLVESGGGLVQAGGSLRLSCAASGFTFDDYAIGWFRQAPGKEREGVSCISSSDGSTYYADSVKGRFTVSSNNADDTVYLQMNSLKPEDTAVYYCAVRLFSGGCAVVAGTSWADFGSSGQGTQVTVSS

SEQ ID NO: 207 - protein, 129 aa, Lama Glama
AVQLVESGGGLVQAGGSLRLSCAASGFTFDDYAIGWFRQAPGKEREGVSCISSSDGSTHYADSVKGRFTISSDKVKNTVYLQMNSLKPEDTAVYYCAVRLFKGGCAVVAGTSWADFGSTGQGTQVTVSS

SEQ ID NO: 208 - protein, 119 aa, Lama Glama
EVQLVESGGGLVQAGGSLRLSCAASGDIPRIAAMGWYRQAPGKQRELVATVSNAATTRYADSAKGRFTISRDNAKTVSLQMDNLKPEDTGVYYCYSLATTVTPSWVNYWGQGTQVTVSS

SEQ ID NO: 209 - protein, 123 aa, Lama Glama
EVQLVESGGGLVQPGGSLRLSCAASGFAFGYYDMSWVRQAPGKGPEWVSAINSGGGSTYYADSVKGRFTISRDNAKNMLYLQMNSLKPEDTAVYYCATPRGWGPTGPHEYDYWGQGTQVTVSS

SEQ ID NO: 210 - protein, 123 aa, Lama Glama
EVQLVESGGGLVQAGGSLRLSCDASGRGFSYYRMGWFRQAPGKEREFVAAIGKSGRNTYYGDYVKGRFTVSRDNAKNTVYLQMTSLKPEDTAVYTCAASLRGWDTTWIDYEYWGQGTQVTVSS

SEQ ID NO: 211 - protein, 121 aa, Lama Glama
EVQLVESGGGSVQAGGSLRLSCAASGSISRIDVMAWYRQAPGKERELVASISSGGSTNYADSVKGRFTISREYFKNMMYLQMNSLKFEDTAVYYCNADSRRGGVGNFFRSWGQGTQVTVSS

SEQ ID NO: 212 - protein, 123 aa, Lama Glama
EVQLVESRGGLVQPGGSLRLSCAASGFTFGSYDMSWVRQAPGKGPEWVSAINSGGGSTYYADSVKGRFTISRDNAKSTLYLQMNSLKPEDTAVYYCAIPRGWGPTGPIEYAYWGQGTQVTVSS

SEQ ID NO: 213 - protein, 116 aa, Lama Glama
EVQLVESGGGLVQAGGSLRLSCAAAGSTFSSYVMGWYRQAPGKQRELVAHISTRGITYYADSVKGRFTISRDNAKNTMYLQMNSLKPEDTAVYYCNTRRNFLSNYWGQGTQVTVSS

SEQ ID NO: 214 - protein, 120 aa, Lama Glama
EVQLVESGGGLVQPGGSLRLSCAASGFTFDDYAIGWFRQAPGKEPEGISCISSSGGITYYADSVKGRFTISRDNAKSTVYLQMNSLKPEDTAVYYCATPGIAACRGIHYTGQGTQVTVSS

SEQ ID NO: 215 - protein, 123 aa, Lama Glama
EVQLVESGGGLVQAGGSLRLSCAASGFTFDDYAIGWFRQAPGKEREGVSCISSSDGSTYYADSVKGRFTISSDNAKNTVYLQMNSLKPEDTAVYYCATAWCDSSWYRSFVGYWGQGTQVTVSS

SEQ ID NO: 216 - protein, 129 aa, Lama Glama
EVQLVESGGGLVQAGGSLRLSCAASGFTFDDYAIGWFRQAPGKEREGVSCISSSDDSTYYADSVKGRFTISSNNAKNTAYLQMNSLKPEDTAVYYCAVRLFSGGCAVVARTSWADFGSSGQGTQVTVSS

SEQ ID NO: 217 - protein, 120 aa, Lama Glama
EVQLVESGGGFVQPGGSLRLSCAASGFTFDDYAIGWFRQAPGKEPEEISCISSSGGITYYADSVKGRFTISRDNAKNTVYLQMNSLKPEDTAVYYCATPGIAACRGIHYWGQGTQVTVSS

SEQ ID NO: 218 - protein, 123 aa, Lama Glama
EVQLVESGGGLVQSGGSLRLSCAASGFTFGSYDMSWVRQAPGKGPEWVSAINSGGGITYYADSVNGRFTISRDNTKNTLYLQMNSLKPEDTAVYYCATPRGWGPTGPHEYGYWGQGTQVTVSS

SEQ ID NO: 219 - protein, 119 aa, Lama Glama
EVQLVESGGGLVQAGGSLRLSCAASGSTFSNYAMGWYRQAPGKQRELVVGISSDGSTHYADSAKGRFTISRDDAKNTVYLQMNSLKTEDTAVYYCYVPVKVAGLEYAYWGQGTQVTVSS

SEQ ID NO: 220 - protein, 121 aa, Lama Glama
EVQLVESGGGLVQPGGSLRLSCEVSGSIGSVSDMRWYRQAPGLQYELVARITSGSITDYSDSVKGRFTISRDNAKNTVYLQMNSLKPEDTAVYYCNADVQHSAWLKPLTYWGQGTQVTVSS

SEQ ID NO: 221 - protein, 111 aa, Lama Glama
EVQLVESGGGLVQPGGSLRLSCAASGIRFSINGMGWYRQAPGKQREAVATITRGGIRDYTDSVKGRFTISRDIARNTVYLQMNNLKPEDSAVYYCNIDIYWGRGTQVTVSS

SEQ ID NO: 222 - protein, 121 aa, Lama Glama
EVQLVESGGGLVQAGGSLRLSCAAFGRTPYGMGWFRQAPGKEREFVAAITSDGSTNYADSVKGRFTISRDNAKNAVSLQMNSLKPEDTAVYYCTAPYYSDFEGTTTEYDYWGQGTQVTVSS

SEQ ID NO: 223 - protein, 128 aa, Lama Glama
EVQLVESGGGLVQAGGSLRLSCAASGRTVRSYATGWFRQAPGKEREFVAALRWSIGSIASVYYDDSVKGRFTISGDNAENTVYLQMNALKPEDTAIYYCASTTRGRYSALSASAYDYWGQGTQVTVSS

SEQ ID NO: 224 - protein, 128 aa, Lama Glama
EVQLVESGGGLVQPGGSLRLSCAASGFTFGSYDMSWVRRSPGKGPEWVSSINSGGGSTYYADFVKGRFTISRDNAKNTLYLQMNSLKPEDTAVYYCAADRYIRARQGDYWGAYEYDYWGQGTQVTVSS

SEQ ID NO: 225 - protein, 130 aa, Lama Glama
EVQLVESGGGLVQAGGSLRLSCAASGFTLDYYAIGWFRQAPGKEREGVSCISSRGGSTYYEDSVKGRFTISRDNAKNTVYLQMNSLKPEDTAVYYCAADPIHNCYSGRYYASPDAVYDYWGQGTQVTVSS

SEQ ID NO: 226 - protein, 124 aa, Lama Glama
KVQLVESGGGLVQAGGSLRLSCAASGFTFDDYAIGWFRQAPGKEREGVSCISSHDGTTYYADSVKGRFTISSDNAKNTVYLQMNSLKPEDTAVYYCAADPLVCGYNDPRLADYWGQGTQVTVSS

SEQ ID NO: 227 - protein, 125 aa, Lama Glama
EVQLVESGGGLVQPGGSLRLSCAASGFTLDYHNIGWFRQAPGKEREWVSCISSSGGSTAYADSVKGRFTISRDNAKNTVYLQMNSLKPEDTAVYYCAAPFNHYSDLCGVNAIDYWGQGTQVTVSS

SEQ ID NO: 228 - protein, 125 aa, Lama Glama
EVQLVESGGGLVQPGGSLRLSCAASGFTLDYYNIGWFRQAPGKEREWVSCISSSDGSTAYADSVKGRFTISRDNGKNTVYLQMNSLKPEDTAVYYCAAPFSYYSSLCGVNEYDYWGQGTQVTVSS

SEQ ID NO: 229 - protein, 125 aa, Lama Glama
EVQLVESGGGLVQPGGSLRLSCAASGFTLDYYNIGWFRQAPGKEREWVSCISSTNGNTYYADSVKGRFSISRDNARNTVYLQMNSLKPEDTAVYYCAAPFSYYNNLCGVNGVDYWGQGTQVTVSS

SEQ ID NO: 230 - protein, 129 aa, Lama Glama
EVQLVESGGGLVQAGGSLRLSCAASGFTFDDYAIGWFRQAPGKEREGVSCISSSDDSTYYADSVKGRFTISSNNAKNTVYLQMNSLKPEDTAVYYCAVRLFSGGCAVVAGTSWADFGSSGQGTQVTVSS

SEQ ID NO: 231 - protein, 125 aa, Lama Glama
EVQLVESGGGLVQPGGSLRLSCAASGFTLDYYNIGWFRQAPGKEREGVSCITSSNGSTYYTDSVKGRFTISRDNAKNTVYLQMNSLKPEDTAVYYCAAPFAHYSDLCGVNGYDYWGQGTQVTVSS

SEQ ID NO: 232 - protein, 125 aa, Lama Glama
EVQLVESGGGLVQPGGSLRLSCAASGFALDYYNIGWFRQAPGKEREWVSCISSSDGSTGYADSVKGRFTISRDNAKNTVYLQMNSLKPEDTAVYYCAAPFAYYSDLCGVNEYDYWGQGTQVTVSS

SEQ ID NO: 233 - protein, 123 aa, Lama Glama
EVQLVESGGGLVQAGGSLRLSCAASGSTFTSYAMGWYRQAPGKQRELVAAISSDDSTYYADCVKGRFTISRDYAKNTVYLQMNSLKPEDTAVYYCNAPHSDYDEEAPSDFGSWGQGTQVTVSS

SEQ ID NO: 234 - protein, 129 aa, Lama Glama
EVQLVESGGGLVQAGGSLRLSCAASGFTFDDYAIGWFRQAPGKEREGVSCISSSDDSTYYADSVKGRFTISRNNAKNTVYLQMNSLKPEDTAVYYCAVRLFSGGCAVVVGTSWADFGSSGQGTQVTVSS

SEQ ID NO: 235 - protein, 120 aa, Lama Glama
EVQLVESGGGLVQPGGSLRLSCAASGFTFENYALGWFRQAPGKEREWVSCISSSDGTTYYADSVKGRFTISRDNVKNTVYLQMNRLKPEDTAIYYCALSLGSSWCAYDYWGQGTQVTVSS

SEQ ID NO: 236 - protein, 124 aa, Lama Glama
EVQLMESGGGLVQAGGSLRLSCAASGFTFDDYAIGWFRQAPGKEREGVSCISSYDGTTYYADSVKGRFTISSDNAKNTVYLQMNSLKPEDTAVYYCAADPLVCGYNDPRLADYWGQGTQVTVSS

SEQ ID NO: 237 - protein, 129 aa, Lama Glama
EVQLVESGGGLVQAGGSLRLSCAASGFTFDDYPIGWLRQAPGKEREGVSCISSSDDSTYYADSVKGRFTISSNNAKNTVYLQMNSLKPEDTAVYYCAVRLFSGGCAVVAGTSWADFGSSGQGTQVTVSS

SEQ ID NO: 238 - protein, 123 aa, Lama Glama
EVQLVESGGGLVQAGGSLRLSCAASGSTFSSYAMGWYRQAPGKQRELVAVISSGDRTNYLDSVKGRFTISRDNAKNTVYLQMNSLKPEDTAVYYCFYSGSYYYPTDVHEYAYWGQGTQVTVSS

SEQ ID NO: 239 - protein, 125 aa, Lama Glama
EVQLVESGGGLVQPGGSLRLSCAASGFTLDYYNIGWFRQAPGKEREWVSCISSGDGSTYYADSVKGRFTISRDNAKNTVYLQMNSLKPEDTAVYYCAAPFEYYSAYCGVNRYDYWGQGTQVTVSS

SEQ ID NO: 240 - protein, 127 aa, Lama Glama
EVQLVESGGGLVQAGGSLRLSCASSGRTLLNYAMGWFRQAPGKEREFVSGINWSGGSTYYADSVKGRFTISRDNAENTVYLHMNSLKPEDTAVYYCAAAHDNYWFTDDSLGRGLKYWGQGTQVTVSS

SEQ ID NO: 241 - protein, 120 aa, Lama Glama
EVQLVESGGGLVQAGGSLRLSCAASGSTFSSYAMGWYRHQAPGKQRELVAAISSDGSTHYADSVKGRFTISRDNAKNTMYLQMNSLKPEDTAVYYCNTKTFGSNWYDDYWGQGTQVTVSS

SEQ ID NO: 242 - protein, 130 aa, Lama Glama
EVQLVESGGGLVQAGGSLRLSCAASGFTLDYYAIGWFRQAPGKEREGVSCISSRGGSTYYTDSVKGRFTISRDNAKNTVYLQMNSLKPEDTGVYYCAADPIHNCYSGRYYASPEAVYDYWGQGTQVTVSS

SEQ ID NO: 243 - protein, 130 aa, Lama Glama
EVQLVESGGGLVQAGGSLRLSCAASAFTLDYYAVGWFRQAPGKEREGVSCISSSGGSTYYEDSVKGRFTISRDNAKNTVYLQMNNLKPEDTAVYYCAADPFHNCYSGSHYSSPEAVYEYWGQGTQVTVSS

SEQ ID NO: 244 - protein, 125 aa, Lama Glama
EVQLVESGGGLVQPGGSLRLSCAASGFTLDYYNIGWFRQAPGKGREWVSCINSSDGSTYYADSVKGRFTISRDNAKNTVYLQMNSLKPEDTAVYYCAAPFEYYSDLCGVNGYDYWGQGTQVTVSS

SEQ ID NO: 245 - protein, 125 aa, Lama Glama
EVQLVESGGGLVQPGGSLRLSCAASGFTLDYYNIGWFRQAPGKEREWVSCISSSDGRTNYVDSVKGRFTMSRDNAKNTVYLQMNSLKPEDTAVYYCAAPFNYYSDLCGVNGVDYWGQGTQVTVSS

SEQ ID NO: 246 - protein, 123 aa, Lama Glama
EVQLVESGGGLVQAGGSLRLSCAASGSTFSSYAMGWYRQAPGNQRELVAVISSGGSTNYADSVKGRFTISRDNAKNTVYLQMNSLKPEDTAVYCCFYSGSYYYPTDVHEYAYWGQGTQVTVSS

SEQ ID NO: 247 - protein, 125 aa, Lama Glama
EVQLVESGGGLVQPGGSLRLSCAASGFTLDNYNIGWFRQAPGKEREWVSCITSSNGSTYYTDSVKGRFTISRDNAKNTVYLQMNSLKPEDTAVYYCAAPFAHYSDLCGVNGYDYWGQGTQVTVSS

SEQ ID NO: 248 - protein, 117 aa, Lama Glama
EVQLVESGGGLVQAGDSLRLSCAASGRTVGSYDMSWVRQGPGKEREWVSSINSGVGKTYYADSVKGRFTIFRDNAKNMVYLQMNNLKPEDTAVYYCATEMDGSRYVEGQGTQVTVSS

SEQ ID NO: 249 - protein, 117 aa, Lama Glama
EVQLVESGGGLVQAEGSLRLSCAASGSTFSTYAMAWYRQAPGKQRELVAGISFDGSTHYAESVKGRFTISRDDAKNTVSLQMNSLKPEDAAVYYCYSVHPSTGFGSWGQGTQVTVSS

SEQ ID NO: 250 - protein, 123 aa, Lama Glama
EVQLVESGGGLVQAGGSLRLSCTASGSTFTSYAMGWYRQAPGKQRELVAAISSDDSTYYADCVKGRFTISRDNAKNTVYLQMNSLKPEDTAVYYCNAPHSDYDEEAPSDFGSWGQGTQVTVSS

SEQ ID NO: 251 - protein, 129 aa, Lama Glama
EVQLVESGGGLVQAGGSLRLSCAASGFTFDDYAIGWFRQAPGKEREGVSCISSSDDSTYYADSVKGRFTISSNNAKNTVYLTMNSLKPEDTAVYYCAVRLFSGGCAVVASTSWADFGSSGQGTQVTVSS

SEQ ID NO: 252 - protein, 129 aa, Lama Glama
EVQLVESGGGLVQAGGSLRLSCAASGFTFDDYAIGWFRQAPGKEREGVSCISSSDGSTYYADSVKGRFTISSNNAKNRAYLQMNSLKPEDTAVYYCAVRLFRGGCAVVAGTSWADFGSSGQGTQVTVSS

SEQ ID NO: 253 - protein, 124 aa, Lama Glama
EVQLVESGGGLVQAGGSLRLSCAASGFTFDDYAIGWFRQAPGKEREGVSCISSYDGTTYYADSVKGRFTISSDNAKNTVYLQMNSLKPEDTAVYYCAADPLVCGYNDPRLADYWGQGTQVTVSS

SEQ ID NO: 254 - protein, 130 aa, Lama Glama
EVQLVESGGGLVQAGGSLRLSCAASGFTLDYYAIGWFRQAPGKEREGVSCISSRGGSTYYEDSVKGRFTISRDNAKNTVYLQMNSLKPEDTAVYYCAADPIHNCYSGRYYASPDAVYEYWGQGTQVTVSS

SEQ ID NO: 255 - protein, 130 aa, Lama Glama
EVQLVESGGGLVQAGGSLRLSCAASGFTLDYYAIGWFRQAPGKEREGVSCISSRGSSTYYADSVKGRFTISRDNAKNTVYLQMNSLKPEDTAVYYCAADPIHNCYSGNGYDSPEAVYDYWGQGTQVTVSS

SEQ ID NO: 256 - protein, 130 aa, Lama Glama
EVQLVESGGGLVQAGGSLRLSCTASGFTLDYYAIGWFRQAPGKEREGVSCISSSGGSTYYADSVKGRFTISRDNAKNTVYLQMNSLKPEDTAVYYCAADPFHNCYSGSAYSSPEAVYEYWGQGTQVTVSS

SEQ ID NO: 257 - protein, 119 aa, Lama Glama
EVQLVESGGGLVQAGGSLRLSCAASGSTFSTYAMGWYRQDPGNQRELVAAISSDGSTHYADSVKGRFTISRDNAKNTVYLQMNSLKPEDTAVYYCYAPVKVAGLEYDYWGQGTQVTVSS

SEQ ID NO: 258 - protein, 120 aa, Lama Glama
EVQLVESGGGLVQPGGSLRLSCAASGFTLDNYAIGWFRQAPGKEREWVSCISGFDGSTYYADSVKGRFTISRDNAKNTVYLQMNSLKPEDTAVYYCAASVGSSWCAYDYWGQGTQVTVSS

SEQ ID NO: 259 - protein, 120 aa, Lama Glama
EVQLVESGGGLVQPGGSLRLSCAASGFTFENYALGWFRQAPGKEREWVSCISSSDGTTYYADSVRGRFTISRDNAKNTVYLQMNRLKPEDTAIYYCALSLGSSWCAYDYWGQGTQVTVSS

SEQ ID NO: 260 - protein, 123 aa, Lama Glama
EVQLVESGGGLVQAGGSLRLSCAASGFTLDNYVIGWFRQAPGKEREEVSCISSSGGSTDYLDSVKGRFTISRDNAKNTVYLQMNSLKPEDTAVYYCAADSLPCYYDKMVYDYWGQGTQVTVSS

SEQ ID NO: 261 - protein, 123 aa, Lama Glama
EVQLVESGGGLVQAGGSLRLSCTASGFKLDYYVIGWFRQAPGKEREGVSCTSSSGGSTYYADSVKGRFTISRDNAKNTVYLQMHSLKPEDTAVYYCAADSFACDYGKMIYDYWGQGTQVTVSS

SEQ ID NO: 262 - protein, 120 aa, Lama Glama
EVQLVESGGGLVQPGGSLRLSCAASGFGFDNYAMGWFRQAPGKEREWVSCISGSDGSTYYADSVKGRFTISRDNAKNTVYLQMHSLKPEDTAVYYCAASLGSSWCAYDYWGQGTQVTVSS

SEQ ID NO: 263 - protein, 124 aa, Lama Glama
EVQLVESGGGLVQAGGSLRLSCSASGFTFDDYAIGWFRQAPGKEREGVSCISSHDGTTYYADSVKGRFTISSDNAKNTVYLQMNSLKPEDTAVYYCAADPLVCGYNDPRLADYWGQGTQVTVSS

SEQ ID NO: 264 - protein, 123 aa, Lama Glama
EVQLVESGGGLVQAGGSLRLSCAASGSTFSSYAMGWYRQAPGKQRELVAAISNGGSTNYVDSVKGRFTISRDNAKNTVYLQMNSLKPEDTAVYYCFYSGSYYYPTDVHEYDYWGQGTQVTVSS

SEQ ID NO: 265 - protein, 125 aa, Lama Glama
EVQLVESGGGLVQPGGSLRLSCAASGFTLDYYNIGWFRQAPGKEREWVSCISSSDGRTNYVDSVKGRFTMSRDNAKNTVYLQMNSLKPEDTAVYYCAAPFNYYSNLCGVNGVDYWGQGTQVTVSS

SEQ ID NO: 266 - protein, 130 aa, Lama Glama
EVQLVESGGGLVQAGGSLRLSCAASGFSLDYYAIGWFRQAPGKEREGISCISGRGGSTYYIDSVKGRFTISRDNAKNTVYLQMNSLKPEDTAVYYCAADPIHNCYSGSHYYSPEAVYEYWGQGTQVTVSS

SEQ ID NO: 267 - protein, 130 aa, Lama Glama
EVQLVESGGGLVQAGDSLRLACAASGFALDYYAVGWFRQAPGKEREGVSCISSRGGSTFYADSLKGRFTTSRDNAKNTVYLQMNSLKPEDTAVYYCAADPIHNCYSGSDYASPEAVYEYWGQGTQVTVSS

SEQ ID NO: 268 - protein, 125 aa, Lama Glama
EVQLVESGGGLVQPGDSLRLSCAASGFTLDYYNIGWFRQAPGKEREWVACIRSSDGSTYYTDSVKGRFTISRNNAKNTVYLQMNSLKPEDTAVYYCAAPFIHYSDLCGVNGNDYWGQGTQVTVSS

SEQ ID NO: 269 - protein, 123 aa, Lama Glama
EVQLVESGGGLVQAGGSLRLSCAASGSTFNSYAMGWYRQAPGKQRELVAVISSGSVTNYADSVKGRFTISRDNAKNTVSLQMNSLKPEDTAVYYCFYSGSYYYPTDVHEYDYWGQGTQVTVSS

SEQ ID NO: 270 - protein, 123 aa, Lama Glama
EVQLVESGGGLVQPGGSLRLSCAASGFRLDYYAIGWFRQAPGKEREWVSCMGSSVRSTYYADSVKGRFTISRDNAKNTVYLQMNSLKPEDTAVYYCAAAPIFECPSGEIYDYWGQGTQVTVSS

SEQ ID NO: 271 - protein, 130 aa, Lama Glama
EVQLVESGGGLVQAGGSLRLSCAASGFTLDYYAIGWFRQAPGKEREGVSCISSRGGSTYYTDSVKGRFTISRDNAKNTVYLQMNSLKPEDTGVYYCAADPIHNCYSGTYYASPEAVYEYWGQGTQVTVSS

SEQ ID NO: 272 - protein, 130 aa, Lama Glama
EVQLVESGGGLVQAGGSLRLSCAASGFALDYYAVGWFRQAPGKEREGVSCISSRGGSTYYVDSVKGRFTTSRDNAKNTVYLEMNSLKPEDTAVYYCAADPIHNCYSGSYYASPEAVYDYWGQGTQVTVSS

SEQ ID NO: 273 - protein, 123 aa, Lama Glama
EVQLVESGGGLVQAGGSLRLSCAASGSTFSSYAMGWYRQAPGKQRELVAVISSGDSTNYSDSVKGRFTISRDNAKNTVYLQMNSLKPEDTAVYYCFYSGSYYYPTDVHEYAYWGQGTQVTVSS

SEQ ID NO: 274 - protein, 123 aa, Lama Glama
EVQLVESGGGLVQAGGSLRLSCAASGSSFSSYAMGWYRQAPGKQRELVAVISSGDRTNYLDSVKGRFTISRDNAKNTVYLQMNSLKPEDTAVYYCFYSGSYYYPTDVHEYAYWGQGTQVTVSS

SEQ ID NO: 275 - protein, 125 aa, Lama Glama
EVQLVESGGGLVQPGGSLRLSCAASGFALDYYNIGWFRQAPGKEREWVSCISGSDGSTGYADSVKGRFTISRDNAKNTVYLQMNSLKPEDTAVYYCAAPFAYYSDLCGVNEYDYWGQGTQVTVSS

SEQ ID NO: 276 - protein, 125 aa, Lama Glama
EVQLVESGGGLVQPGGSLRLSCAASGFALDGHNIGWFRQAPGKEREWVSCINSGDGSTGYADSVKGRFTISRDNAKNTVYLQMNRLKPEDTAVYYCAAPFNHYSFLCGVNEYDYWGQGTQVTVSS

SEQ ID NO: 277 - protein, 123 aa, Lama Glama
EVQLVESGGGLVQAGGSLRLSCAASGSTFSSYAMGWYRQAPGKQRELAAVISTGDNTNYADSVKGRFTISRDNAKNTVYLQMNSLKPEDTAVYHCFYSGSYYYPTEVYEYDYWGQGTQVTVSS

SEQ ID NO: 278 - protein, 123 aa, Lama Glama
EVQLVESGGGLVQAGGSLRLSCAASGSTFRSYAMGWYRQVPGNQRELVAVISSGDSANYADSVKGRFTISRDNAKNTVYLQMNSLKPEDTAVYYCFYSGSYYYPTDVHEYDYWGQGTQVTVSS

SEQ ID NO: 279 - protein, 125 aa, Lama Glama
EVQLVESGGGLVQPGGSLRLSCAASGFTLDYYNIGWFRQAPGKEREWVSCINSSDGTTYYADSVKGRFTISRDNAKNTVYLQMNSLKPEDTAVYYCAAPFEYYSDLCGVNGYDYWGQGTQVTVSS

SEQ ID NO: 280 - protein, 130 aa, Lama Glama
EVQLVESGGGLVQAGGSLRLSCGASGFSLDYYAIGWFRQAPGKEREGVSCISGRGSNTYYLDSVKGRFTISRDNAKNTVYLQMNSLKPEDTAVYYCAADPIHNCYGGSYYASPEAVYEYWGQGTQVTVSS

SEQ ID NO: 281 - protein, 120 aa, Lama Glama
EVQLVESGGGLVQPGGSLRLSCAASGFTFDDYAIGWFRQAPGKEREGVSCISSSGSTYYADSVKGRFTISRDNAKNTVYLQMNSLKPEDTAVYYCAIAGASSWCFPPGYWGQGTQVTVSS

SEQ ID NO: 282 - protein, 130 aa, Lama Glama
EVQLVESGGGLVQAGGSLRLSCAASGFALDYYAVGWFRQAPGKEREGVSCISSRGGSTYYADSVKGRFTTSRDNAKNTVYLQMNSLKPEDTAVYYCAADPIHNCYSGIYYASPEAVYDYWGQGTQVTVSS

SEQ ID NO: 283 - protein, 123 aa, Lama Glama
EVQLVESGGGLVQAGGSLRLSCAASGSTFSSYAMGWYRQAPVKQRELVAVISNGGSTNYADSVKGRFTISRDNAKNTVYLQMNSLKPEDTAVYYCFYSGSYYYPTDVHEYDYWGQGTQVTVSS

SEQ ID NO: 284 - protein, 130 aa, Lama Glama
EVQLVESGGGLVQPGGSLRLSCAASGFTLDYYAIGWFRQAPGKEREGVSCISSRGGSTYYEDSVKGRFTISRDNAKNTVYLQMNSLKPEDTAVYYCAADPIHNCYSGRYYASPDAVYDYWGQGTQVTVSS

SEQ ID NO: 285 - protein, 125 aa, Lama Glama
EVQLVESGGGLVQPGGSLRLSCAASEFTLDHYNIGWFRQAPGKEREWVSCISSSDGSTGYADSVKGRFTISRDKAKNTVYLQMNSLKPEDTAVYYCAAPFSYYSDLCGVNGYDYWGQGTQVTVSS

SEQ ID NO: 286 - protein, 123 aa, Lama Glama
EVQLVESGGGLVQAGGSLRLSCAASGSTFSSYAMGWYRQAPGKQRELVAVISSGDSTNYADSVKGRFTMSRDNAKNTVYLQMNSLRPEDTAVYYCFYSGSYYYPSDVHEYDYWGQGTQVTVSS

SEQ ID NO: 287 - protein, 125 aa, Lama Glama
EVQLVESGGGLVQPGGSLRLSCAASGFTLDYYNIGWFRQAPGKEREWVSCISSSDGSTDYADSVKGRFTISRDNAKNTVYLQMNSLKPEDTAVYYCAAPFSYYSGLCGVNGVDYWGQGTQVTVSS

SEQ ID NO: 288 - protein, 120 aa, Lama Glama
EVQLVESGGGLVQPGGSLRLSCAASGFTLGVYATGWFRQAPGKEREWVSCISGSDGSTWYADSVKGRFTISRDNAKNTVYLQMNSPKSEDTAVYYCALSLGSSWCAYDYWGQGTQVTVSS

SEQ ID NO: 289 - protein, 130 aa, Lama Glama
EVQLVESGGGLVQAGGSLRLSCAASGFTLDYYAIGWFRQAPGKEREGVSCISGRGGSTYYTDSVKGRFTISRDNAKNTVYLQMNSLKPEDTGVYYCAADPVHNCYSGRYYASPDAVYEYWGQGTQVTVSS

SEQ ID NO: 290 - protein, 123 aa, Lama Glama
EVQLVESGGGLVQPGGSLRLSCAASGSTFSSYAMGWYRQAPGKQRELVAVISNGGSTNYADSVKGRFTISRDNAKNTVYLQMNSLKPEDTAVYICFYSGSYYYPTDVHEYAYWGQGTQVTVSS

SEQ ID NO: 291 - protein, 125 aa, Lama Glama
EVQLVESGGGLVQPGGSLRLSCAASGFTLDYHNIGWFRQAPGKEREWVSCISSSGGSTAYADSVKGRFTISRDNAKNTVYLQMNSLKPEDTAVYYCAAPFSHYNDLCGVNAIDYWGQGTQVTVSS

SEQ ID NO: 292 - protein, 130 aa, Lama Glama
EVQLVESGGGLVQAGGSLRLSCAASGFTLDYYAIGWFRQAPGKEREGVSCISSRGASTYYADSVKGRFTISRDNAKNTVYLQMNSLKPEDTAVYYCAADPIHNCYSGNGYDSPEAVYDYWGQGTQVTVSS

SEQ ID NO: 293 - protein, 125 aa, Lama Glama
EVQLVESGGGLVQPGGSLRLSCAASGFTLHYYNIGWFRQAPGKEREWVSCINSSDGSTHYADSVKGRFTISRDNAKNTVYLQMNSLKPEDTAVYYCAAPFEYYSDLCGVNGYDYWGQGTQVTVSS

SEQ ID NO: 294 - protein, 125 aa, Lama Glama
KVQLVESGGGLVQPGGSLRLSCAASGFTLDYYNIGWFRQAPGKEREWVSCINSSDGSTYYADSVKGRFTISRDNAKNTVYLQMNSLKPEDTAVYYCAAPFEYYSNLCGVNGYDYWGQGTQVTVSS

SEQ ID NO: 295 - protein, 130 aa, Lama Glama
EVQLVESGGGLVQAGGSLRLSCAASGFTLDKYSIGWFRQAPGKEREGVSCISSSGGSTYYVDSVKGRFTISRDNAKNTVYLQMNNLKPEDTAVYYCAADPLHNCYSGRGYYSPEAVYEYWGQGTQVTVSS

SEQ ID NO: 296 - protein, 130 aa, Lama Glama
EVQLVESGGGLVQAGGSLRLSCAASGFTLDYYAIGWFRQAPGKEREGVSCISSRGGSTYYTDSVKGRFTISRDNAKNTVYLQMNSLKPEDTGVYYCAADPIHNCYSGSYYASPEAVYEYWGQGTQVTVSS

SEQ ID NO: 297 - protein, 125 aa, Lama Glama
EVQLVESGGGLVQPGGSLRLSCAASGFTLDYYNIGWFRQAPGKEREWVSCINSSDGSTYYADSVKGRFTISRDNAKNTVYLQMNSLKPEDTAVYYCAAPFEYYSNLCGVNGYDYWGQGTQVTVSS

SEQ ID NO: 298 - protein, 120 aa, Lama Glama
EVQLVESGGGLVQPGGSLRLSCTASGFTFDDYAIGWFRQAPGKEPEGISCISSSGSITYDADSVKGRFTISRDNAKNTVYLQMNSLKPEDTAVYYCATPGIAACRGIHYWGQGTQVTVSS

SEQ ID NO: 299 - protein, 120 aa, Lama Glama
EVQLVESGGGLVQPGGSLRLSCAASGFTFDDYAIGWFRQAPGKEPEGISCISSSGGITYYADSVKGRFTISRDNAKNTVYLQMNSLKPEDTAVYYCATPGIAACRGIHYTGQGTQVTVSS

SEQ ID NO: 300 - protein, 120 aa, Lama Glama
EVQLVESGGGLVQPGGSLRLSCAASGFTFDDYAIGWFRQAPGKEPEGISCISSSGGITYYADSVKGRFTTSRDNAKNTVYLQMNSLKPEDTAVYYCATPGIAACRGIHYTGQGTQVTVSS

SEQ ID NO: 301 - protein, 120 aa, Lama Glama; identical to SEQ ID NO: 193

SEQ ID NO: 302 - protein, 120 aa, Lama Glama
EVQLVESGGGLVQPGGSLRLSCAASGFTFDDYAIGWFRQAPGKEPEEISCISSSGGITYYADSVKGRFTISRDNAKNTVYLQMNSLKPEDTAVYYCATPGIAACRGIHYTGQGTQVTVSS

SEQ ID NO: 303 - protein, 126 aa, Lama Glama
EVQLVESGGGLVQAGGSLRLSCTTSERTVSRYSMGWFRQAPGKEREAVATISWSGDSTYYADSVKGRFTISRDNTKNTLYLQIDSLKPEDTAVYYCVAKPNLKYGSYWPPRGYDYWGQGTQVTVSS

SEQ ID NO: 304 - protein, 126 aa, Lama Glama
EVQLVESGGGLVQAGGSLRLSCTTSERTVSRYSMGWFRQAPGKEREAVATISWSGDSTYYADSVKGRFTISRDNTKNMLYLQMNSLKPEDTAVYYCVAKPNLKYGSTWPPRGYDYWGQGTQVTVSS

SEQ ID NO: 305 - protein, 126 aa, Lama Glama
EVQLVESGGGLVQAGGSLRLSCTTSERAVSRYTMGWLRQAPGKEREAVATISWSGDSTYYADSVKGRFTISRDNTKNTLYLQMNSLKPEDTADYYCAAKPNLKYGSYWPPRGYDYWGQGTQVTVSS

SEQ ID NO: 306 - protein, 126 aa, Lama Glama
EVQLVKSGGGLVQAGGSLRLSCTTSERTVSRYGMGWFRQAPGKEREAVATISWSGDSTYYADSVKGRFTISRDNTKNTLYLQMNSLKPEDTAVYYCAAKPNLKYGSDWPPRGYDYWGQGTQVTVSS

SEQ ID NO: 307 - protein, 117 aa, Lama Glama
EVQLVESGGGLVQPGGSLRLSCTASGFTFSSYAMSWVRQAPGKGLEWVSFINKDGSDTGYADSVKGRFTISRDNAKNTMYLQMNSLKPEDTAVYFCETRTSRSPRPRGQGTQVTVSS

SEQ ID NO: 308 - protein, 124 aa, Lama Glama
EVQLVESGGGLVQAGGSLRLSCAASGRTFSRYAMGWFRQAPGKEREFVAAINWSGGSTYYADSVKGRFTISRDNAKNTVYLQMNSLKPEDTAVYDCAASNYYSVYDDRPVMDYWGQGTQVTVSS

SEQ ID NO: 309 - protein, 116 aa, Lama Glama
EVQLMESGGGLVQPGGSLRLSCVAAGFTFSNYYMSWVRQAPGKGLEWVSVISPDGSNTYYADTVKGRFTISRGNAKNTLFLQMTGLKSEDAAVYYCARGSGSWGVHGQGTQVTVSS

SEQ ID NO: 310 - protein, 128 aa, Lama Glama
EVQLVESGGGLVQAGGSLRLSCAASGRTFSNYIMGWFRQAPGKEREFVAGISRYGDYTAYADSVKGRFTISRDNVKNTVYLRMNSLKPDDTAVYYCAANEGYCSGYGCYEDSGQYDYWGQGTQVTVSS

SEQ ID NO: 311 - protein, 128 aa, Lama Glama
EVQLVESGGGLVQAGGSLRLSCAASGRTFSNYIMGWFRQAPGKEREFVAGISRYGDYTYYADSVKGRFTISRDNVKNTVYLRMNSLKPDDTAVYYCAANEGYCSGYGCYEDSGQYDYWGQGTQVTVSS

SEQ ID NO: 312 - protein, 121 aa, Lama Glama
EVQLVESGGGLVQAGGSLTLSCAASGGTFTTYAMGWFRQAPGKEREFVAAVSRFGVSWDYADSVKGRFTISRDNTANTLKLRMNSLKADDTAVYYCAAGGRSFLPFVPAYWGQGTQVTVSS

SEQ ID NO: 313 - protein, 128 aa, Lama Glama
EVQLVESGGGLVQAGGSLRLSCAASGRTFSNYIMGWFRQAPGKEREFVAGISRYADYTGYADSVKGRFTISRDNVKNTVYLRMNSLKPDDTAVYYCAANEGYCSGYGCYEDSGQYDYWGQGTQVTVSS

SEQ ID NO: 314 - protein, 126 aa, Lama Glama
EVQLVESGGGLVKPGGSLRLSCAASGRTLYIMGWYRQAPGKEREFVAGISRYGDITYAADSVKGRFTISRDSVKNTVYLRMNSLKPDDTAVYYCAANGGYCSGYGCYEDSGQYDYWGQGTQVTVSS

SEQ ID NO: 315 - protein, 120 aa, Lama Glama
EVQLVESGGGLVQAGGSLRLSCAASGFTFDDYAIGWFRQAPGKEPEGISCISSSGGITYYADSVKGRFTISRDNAKNTVYLQMNSLKPEDTAVYYCATPGIAACRGIHYTGQGTQVTVSS

SEQ ID NO: 316 - protein, 120 aa, Lama Glama; identical to SEQ ID NO: 302

SEQ ID NO: 317 - protein, 120 aa, Lama Glama
EVQLVESGGGLVQAGGSLRLSCAVSGFSFDDYAIGWFRQAPGKEPEGISCISSSGGITYYADSVKGRFTISRDNAKNTVYLQMNSLKPEDTAVYYCATPGIAACRGIHYTGQGTQVTVSS

SEQ ID NO: 318 - protein, 123 aa, Lama Glama
EVQLVESGGGLVQPGGSLRLSCAASGFTFGSYDMSWVRQAPGKGPEWVSAINSGGGSTYYADSVKGRFTISRDNAKNTLYLQMNSLKPEDTAVYYCAIPRGWGPTGPIEYAYWGQGTQVTVSS

SEQ ID NO: 319 - protein, 123 aa, Lama Glama
EVQLVESGGGLVQPGGSLRLSCAASGFTFGSYDMSWVRQAPGKGPEWVSAINSGGGSTYYADSVKGRFTISRDNAKNTLYLQMNSLKPEDTAVYYCAIPRGWGPTGPIEYGYWGQGTQVTVSS

SEQ ID NO: 320 - protein, 123 aa, Lama Glama
EVQLVESGGGLVQPGGSLRLSCAASGFTFGSYDMSWVRQAPGKGPEWVSAINSGGGSTYYADSVKGRFTISRDNAKNTLYLQMNSLKPEDTGVYSCAIPRGWGPTGPHEYGYWGQGTQVTVSS

SEQ ID NO: 321 - protein, 123 aa, Lama Glama
EVQLVESGGGLVQSGGSLRLSCAASGFTFGSYDMSWVRQAPGKGPEWVSAINSGGGSTYYADSVKGRFTISRDNAKNTLYLQMNSLKPEDTAVYYCAIPRGWGPTGPHEYAYWGQGTQVTVSS

SEQ ID NO: 322 - protein, 121 aa, Lama Glama
EVQLVESGGGSVQAGGSLRLSCAASGRTFSSYAMGWFRQAPGKEREFVAAINWSGGYTYYADSVRGRFTISRDNAKNTVYLQMNSLKPEDTAVYYCAAPAPGSSGYEYDYWGQGTQVTVSS

SEQ ID NO: 323 - protein, 120 aa, Lama Glama
EVQLVESGGGLVQPGGSLRLSCTASGFTFDVYAIGWFRQAPGKEPEGISCISSSGSITYYADSVKGRFTISRDSAKNTVYLQMNSLKPEDTAVYYCATPGIAACRGIHYWGQGTQVTVSS

SEQ ID NO: 324 - protein, 123 aa, Lama Glama
EVQLVESGGGLVQPGGSLRLSCAASGFTFGNYDMSWVRHAPGKGPEWVSAINSGGGSTYYTDSVKGRFTISRDNAKNTLYLQMNSLKPEDTAVYYCAIPRGWGPTGPHEYAYWGQGTQVTVSS

SEQ ID NO: 325 - protein, 123 aa, Lama Glama
EVQLVESGGGLVQPGGSLRLSCAASGFTFGSYDMSWVRQAPGKGPEWVSAINSGGGTTYYADSVKGRFTISRDNAKNTLFLQMNSLKPEDTAVYYCAIPRGWGPTGPLEYGYWGQGTQVTVSS

SEQ ID NO: 326 - protein, 123 aa, Lama Glama
EVQLVESGGGLVQPGGSLRLSCAASGFTFGNYDMSWVRQAPGKGPEWVSAINSGGGDTYYADSVKGRFTISRDNAKNTLYLQMNSLKPEDTAVYYCATPRGWGPTGPHEYGYWGQGTQVTVSS

SEQ ID NO: 327 - protein, 123 aa, Lama Glama
EVQLVESGGGLVQPGGSLRLSCAASGFTFGNYDMSWVRQAPGKGPEWVSAINSGGGITYYADSVKGRFAISRDNAKTTLYLQMNNLQPEDTAVYYCATPRGWGPTGPHEYGYWGQGTQVTVSS

SEQ ID NO: 328 - protein, 123 aa, Lama Glama
EVQLVESGGGLVQAGGSLRLSCAASGFTFGSYDMSWVRQAPGKGPEWVSAINSGGGITYYADLVKGRFTISRDNAKNTLYLQMNSLKPEDTAVYYCAIPRGWGPTGPHEYGYWGQGTQVTVSS

SEQ ID NO: 329 - protein, 123 aa, Lama Glama
EVQLVESGGGLVQPGGSLRLSCAASGFTFGNYDMSWVRQAPGKGPEWVSAINSGGGITYYADSVKGRFTISRDNAKNTLYLQMNSLKPEDTAVYYCATPRGWGPTGPHEYGYWGQGTQVTVSS

SEQ ID NO: 330 - protein, 123 aa, Lama Glama
EVQLVESGGGLVQPGGSLRLSCAASGFTFGSYDMSWVRQAPGKGPEWVSAINSGGGSTYYADSVKGRFTISRDNAKNTLYLQMNSLTPEDTAVYYCAIPRGWGPTGPHEYAYWGQGTQVTVSS

SEQ ID NO: 331 - protein, 123 aa, Lama Glama
EVQLVESGGGLVQPGGSLRLSCTASGFTFGNYDMSWVRRPPGKGPEWVSAINSGGGSTYYADSVKGRFTISRDNAKNTLYLQMNSLKPEDTAVYYCATPRGWGPTGPHEYGYWGQGTQVTVSS

SEQ ID NO: 332 - protein, 123 aa, Lama Glama
EVQLVESGGGLVQPGGSLRLSCAASGFTFGSYDMSWVRQAPGKGPEWVSAINSGGGSTYYADSVKGRFTISRDNAKNTLYLQMNNLKPEDTAVYSCAIPRGWGPTGPHEYAYWGQGTQVTVSS

SEQ ID NO: 333 - protein, 14 aa, Artificial
  Mutated Lama sequence
RAPDTRLRPYLYDY

SEQ ID NO: 334 - protein, 14 aa, Artificial
  Mutated Lama sequence
RAPDTRLEPYEYDH

SEQ ID NO: 335 - protein, 14 aa, Artificial
  Mutated Lama sequence
RAPDTRLAPYEYDH

SEQ ID NO: 336 - protein, 14 aa, Artificial
  Mutated Lama sequence
RAPDTRLEPYLYDH

SEQ ID NO: 337 - protein, 14 aa, Artificial
  Mutated Lama sequence
RAPDTRLEPYLYDY

SEQ ID NO: 338 - protein, 14 aa, Artificial
  Mutated Lama sequence
RAPDTRLAPYLYDY

SEQ ID NO: 339 - protein, 14 aa, Artificial
  Mutated Lama sequence
RAPDTRLAPYEYDH

SEQ ID NO: 340 - protein, 14 aa, Artificial
  Mutated Lama sequence
RAPDTRLRPYLYDY

SEQ ID NO: 341 - protein, 14 aa, Artificial
  Mutated Lama sequence
RAPDTRLEPYLYDH

SEQ ID NO: 342 - protein, 14 aa, Artificial
  Mutated Lama sequence
RAPDTRLEPYEYDH

SEQ ID NO: 343 - protein, 14 aa, Artificial
  Mutated Lama sequence
RAPDTRLRPYEYDY

SEQ ID NO: 344 - protein, 14 aa, Artificial
  Mutated Lama sequence
RAPDTRLEPYLYDH

SEQ ID NO: 345 - protein, 14 aa, Artificial
  Mutated Lama sequence
RAPDTRLGPYLYDY

SEQ ID NO: 346 - protein, 14 aa, Artificial
  Mutated Lama sequence
RAPDTRLEPYEYDY

SEQ ID NO: 347 - protein, 14 aa, Artificial
  Mutated Lama sequence
RAPDTRLGPYLYDH

SEQ ID NO: 348 - protein, 14 aa, Artificial
  Mutated Lama sequence
RAPDTRLAPYLYDH

SEQ ID NO: 349 - protein, 14 aa, Artificial
  Mutated Lama sequence
RAPDTRLAPYLYDY

SEQ ID NO: 350 - protein, 14 aa, Artificial
  Mutated Lama sequence
RAPDTRLRPYLYDH

SEQ ID NO: 351 - protein, 14 aa, Artificial
  Mutated Lama sequence
RAPDTRLEPYLYDH

SEQ ID NO: 352 - protein, 14 aa, Artificial
  Mutated Lama sequence
RAPDTRLEPYEYDH

SEQ ID NO: 353 - protein, 14 aa, Artificial
  Mutated Lama sequence
RAPDTRLAPYEYDY

SEQ ID NO: 354 - protein, 123 aa, Artificial
  Mutated Lama sequence
EVQLVESGGGLVQAGGSLRLSCAASGRTFSSYAMAWFRQAPGKEREYVAAIRWSGGTAYYADSVQGRFTISRDNAKNTVYLQMNSLRPEDTAVYYCANRAPDTRLRPYLYDYWGQGTQVTVSS

SEQ ID NO: 355 - protein, 123 aa, Artificial
  Mutated Lama sequence
EVQLVESGGGLVQAGGSLSLSCAASGRTFSSYAMAWYRQAPGKEREYVAAIRWSGGTAYYADSVQGRFTISRDNAKNTVYLQMNSLKPEDTAVYYCANRAPDTRLEPYEYDHWGQGTQVTVSS

SEQ ID NO: 356 - protein, 123 aa, Artificial
  Mutated Lama sequence
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLAPYEYDHWGQGTQVTVSS

SEQ ID NO: 357 - protein, 123 aa, Artificial
  Mutated Lama sequence
EVQLVESGGGLVQAGGSLRLSCAASGRTFSSYAMAWFRQAPGKEREYVAAIRWSGGTAYYADSVQSRFTITRDNAKNTVYLQMNSLKPEDTAVYYCANRAPDTRLEPYLYDHWGQGTQVTVSS

SEQ ID NO: 358 - protein, 123 aa, Artificial
  Mutated Lama sequence
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLEPYLYDYWGQGTQVTVSS

SEQ ID NO: 359 - protein, 123 aa, Artificial
  Mutated Lama sequence
EVQLVESGGGLVQAGGSLRLSCAASGRTFSSYAMAWFRQAPGKDREYVAAIRWSGGTAYYADSVQGRFTISRDNAKNTVYLQMNSLKPEDTAVYYCANRAPDTRLAPYLYDYWGQGTQVTVSS

SEQ ID NO: 360 - protein, 123 aa, Artificial
  Mutated Lama sequence
EVQLVESGGGLVQAGGSLRLSCSASGRTFSSYAMAWYRQAPGKEREYVAAIRWSGGTAYYPDSVQGRFTISRDNAKNTVYLQMNSLKPEDTAVYYCANRAPDTRLAPYEYDHWGQGTQVTVSS

SEQ ID NO: 361 - protein, 123 aa, Artificial
  Mutated Lama sequence
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLRPYLYDYWGQGTQVTVSS

SEQ ID NO: 362 - protein, 123 aa, Artificial
  Mutated Lama sequence
EVQLVESGGGLVQAGGSLRLSCAASGRTFSSYAMAWYRQAPGKEREYVAAIRWSGETAYYADSVQGRFTISRDNAKNTVYLQMNSLKPEDTAVYYCANRAPDTRLEPYLYDHWGQGTQVTVSS

SEQ ID NO: 363 - protein, 123 aa, Artificial
  Mutated Lama sequence
EVQLVESGGGLVQAGGSLRLSCAASGRTFSSYAMAWYRQAPGKEREYVAAIRWSGGTAYYADSVQSRFTISRDNAKNTVYLQMNSLKPEDTAVYYCANRAPDTRLEPYEYDHWGQGTQVTVSS

SEQ ID NO: 364 - protein, 123 aa, Artificial
  Mutated Lama sequence
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLRPYEYDYWGQGTQVTVSS

SEQ ID NO: 365 - protein, 123 aa, Artificial
  Mutated Lama sequence
EVQLVESRGGLVQAGGSLRLSCAASGRTFSSYAMAWFRQAPGKEREYVAAIRWSGGTAYYADSVQGRFTISRDNAKNTVYLQMNSLKPEDTAVYYCANRAPDTRLEPYLYDHWGQGTQVTVSS

SEQ ID NO: 366 - protein, 123 aa, Artificial
  Mutated Lama sequence
EVQLVESGGGLVQAGGSLRLSCAASGRTFSSYAMAWYRLAPGKEREYVAAIRWSGGTAYYADSVQGRFTISRDNAKNTVYLQMNSLKPEDTAVYYCANRAPDTRLGPYLYDYWGQGTQVTVSS

SEQ ID NO: 367 - protein, 123 aa, Artificial
  Mutated Lama sequence
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLEPYEYDYWGQGTQVTVSS

SEQ ID NO: 368 - protein, 123 aa, Artificial
  Mutated Lama sequence
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLGPYLYDHWGQGTQVTVSS

SEQ ID NO: 369 - protein, 123 aa, Artificial
  Mutated Lama sequence
EVQLVESGGGLVQAGGSLRLSCAASGRTFSSYAMAWFRQAPGKEREYVAAIRWSGGTAYYADSVQGRFTISRDNAKNTVYLQMYSLKPEDTAVYYCANRAPDTRLAPYLYDHWGQGTQVTVSS

SEQ ID NO: 370 - protein, 123 aa, Artificial
  Mutated Lama sequence
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLAPYLYDYWGQGTQVTVSS

SEQ ID NO: 371 - protein, 123 aa, Artificial
  Mutated Lama sequence
EVQLVESGGGLVQAGGSLRLSCAASGRTFSSYAMAWFRQAPGKEREYVAAIRWSGGTAYYADSVQGRFTISRDNAKNTVYLQMNSLKPEDTAVYYCANRAPDTRLRPYLYDHWGQGTQVTVSS

SEQ ID NO: 372 - protein, 123 aa, Artificial
  Mutated Lama sequence
EVQLVESGGGLVQAGGSLRLSCSASGRTFSSYAMAWYRQAPGKEREYVAAIRWSGETAYYADSVQGRFTISRDNAKNTVYLQMNSLKPEDTAVYYCANRAPDTRLEPYLYDHWGQGTQVTVSS

SEQ ID NO: 373 - protein, 123 aa, Artificial
  Mutated Lama sequence
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLEPYEYDHWGQGTQVTVSS

SEQ ID NO: 374 - protein, 123 aa, Artificial
  Mutated Lama sequence
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLAPYEYDYWGQGTQVTVSS

SEQ ID NO: 375 - protein, 19 aa, Artificial
  Mutated Lama sequence
DRYIWARQGEYWGAYQYDY

SEQ ID NO: 376 - protein, 19 aa, Artificial
  Mutated Lama sequence
DRYIWARQGEYWGAYAYDY

SEQ ID NO: 377 - protein, 19 aa, Artificial
  Mutated Lama sequence
DRYIWARQGEYWGAYVYDY

SEQ ID NO: 378 - protein, 19 aa, Artificial
  Mutated Lama sequence
DRYIWARQGEYWGAYQYDY

SEQ ID NO: 379 - protein, 19 aa, Artificial
  Mutated Lama sequence
DRYIWARQGEYWGAYVYDY

SEQ ID NO: 380 - protein, 19 aa, Artificial
  Mutated Lama sequence
DRYIWARQGEYWGAYQYDY

SEQ ID NO: 381 - protein, 19 aa, Artificial
  Mutated Lama sequence
DRYIWARQGEYWGAYAYDY

SEQ ID NO: 382 - protein, 19 aa, Artificial
  Mutated Lama sequence
DRYIWARQGEYWGAYAYDY

SEQ ID NO: 383 - protein, 19 aa, Artificial
  Mutated Lama sequence
DRYIWARQGEYWGAYAYDY

SEQ ID NO: 384 - protein, 19 aa, Artificial
  Mutated Lama sequence
DRYIWARQGEYWGAYAYDY

SEQ ID NO: 385 - protein, 19 aa, Artificial
  Mutated Lama sequence
DRYIWARQGEYWGAYQYDY

SEQ ID NO: 386 - protein, 19 aa, Artificial
  Mutated Lama sequence
DRYIWARQGEYWGAYEYDY

SEQ ID NO: 387 - protein, 19 aa, Artificial
  Mutated Lama sequence
DRYIWARQGEYWGAYAYDY

SEQ ID NO: 388 - protein, 19 aa, Artificial
  Mutated Lama sequence
DRYIWARQGDYWGAYVYDY

SEQ ID NO: 389 - protein, 19 aa, Artificial
  Mutated Lama sequence
DRYIWARQGDYWGAYAYDY

SEQ ID NO: 390 - protein, 19 aa, Artificial
  Mutated Lama sequence
DRYIRARQGEYWGAYAYDY

SEQ ID NO: 391 - protein, 19 aa, Artificial
  Mutated Lama sequence
DRYIWARQGEYWGAYAYDY

SEQ ID NO: 392 - protein, 19 aa, Artificial
  Mutated Lama sequence
DRYIWARQGEYWGAYEYDY

SEQ ID NO: 393 - protein, 19 aa, Artificial
  Mutated Lama sequence
DRYIWARQGEYWGAYAYDY

SEQ ID NO: 394 - protein, 19 aa, Artificial
  Mutated Lama sequence
DRYIWARQGEYWGAYQYDY

SEQ ID NO: 395 - protein, 19 aa, Artificial
  Mutated Lama sequence
DRYIWARQGEYWGAYAYDY

SEQ ID NO: 396 - protein, 128 aa, Artificial
  Mutated Lama sequence
EVQLVESGGGLVQPGGSLRLSCAASGFTFGSYDMSWVRRSPGKGPEWVSAINSGGGSTFYTDYVKGRFTISRDNAKNTLYLQMNSLKPEDTAVYYCAADRYIWARQGEYWGAYQYDYWGQGTQVTVSS

SEQ ID NO: 397 - protein, 128 aa, Artificial
  Mutated Lama sequence
EVQLVESGGGLVQPGGSLRLSCAASGFTFGSYDMSWVRRSPGKGPEWVSAINSGGGSTYYADYVKGRFTISRDNAKNTLYLQMNSLKPEDTAVYYCAADRYIWARQGEYWGAYAYDYWGQGTQVTVSS

SEQ ID NO: 398 - protein, 128 aa, Artificial
  Mutated Lama sequence
EVQLVESGGGLVQPGGSLRLSCAASGFTFGSYDMSWVRRSPGKGPEWVSAINSGGGSTYYTDYVKGRFTISRDNAKNTLYLQMNSLKPEDTAVYYCAADRYIWARQGEYWGAYVYDYWGQGTQVTVSS

SEQ ID NO: 399 - protein, 128 aa, Artificial
  Mutated Lama sequence
EVQLVESGGGLVQPGGSLRLSCAASGFTIGSYDMSWVRRSPGKGPEWVSAINSGGGSTYYADYVKGRFTISRDNAKNTLYLQMNSLKPEDTAVYYCAADRYIWARQGEYWGAYQYDYWGQGTQVTVSS

SEQ ID NO: 400 - protein, 128 aa, Artificial
  Mutated Lama sequence
EVQLVESGGGLVQPGGSLRLSCAASGFTIGSYDMSWVRRSPGKGPEWVSAINSGGGSTYYTDYVKGRFTISRDNAKNTLYLQMNSLKPEDTAVYYCAADRYIWARQGEYWGAYVYDYWGQGTQVTVSS

SEQ ID NO: 401 - protein, 128 aa, Artificial
  Mutated Lama sequence
EVQLVESGGGLVQPGGSLRLSCAASGFTFGSYDMSWVRRSPGKGPEWVSAINSGGGSTYYTDYVKGRFTISRDNAKNTLYLQMNSLKPEDTAVYYCAADRYIWARQGEYWGAYQYDYWGQGTQVTVSS

SEQ ID NO: 402 - protein, 128 aa, Artificial
  Mutated Lama sequence
EVQLVESGGGLVQPGGSLRLSCAASGFTFGSYDMSWVRRSPGKGPEWVSAINSGGGSTYYTDYVKGRFTISRDNAKNTLYLQMNSLKPEDTAVYYCAADRYIWARQGEYWGAYAYDYWGQGTQVTVSS

SEQ ID NO: 403 - protein, 128 aa, Artificial
  Mutated Lama sequence
EVQLVESGGGLVQPGGSLRLSCAASGFTIGSYDMSWVRRSPGKGPEWVSAINSGGGSTYYTDYVKGRFTISRDNAKNTLYLQMNSLKPEDTAVYYCAADRYIWARQGEYWGAYAYDYWGQGTQVTVSS

SEQ ID NO: 404 - protein, 128 aa, Artificial
  Mutated Lama sequence
EVQLVESGGGLVQPGGSLRLSCAASGFTIGSYDMSWVRRSPGKGPEWVSAINSGGGSTYYTDFVKGRFTISRDNAKNTLYLQMNSLKPEDTAVYYCAADRYIWARQGEYWGAYAYDYWGQGTQVTVSS

SEQ ID NO: 405 - protein, 128 aa, Artificial
  Mutated Lama sequence
EVQLVESGGGLVQPGGSLRLSCAASGFTIGSYDMSWVRRSPGKGPEWVSSINSGGGSTYYTDYVKGRFTISRDNAKNTLYLQMNSLKPEDTAVYYCAADRYIWARQGEYWGAYAYDYWGQGTQVTVSS

SEQ ID NO: 406 - protein, 128 aa, Artificial; identical to SEQ ID NO: 399
  Mutated Lama sequence

SEQ ID NO: 407 - protein, 128 aa, Artificial
  Mutated Lama sequence
EVQLVESGGGLVQPGGSLRLSCAASGFTFGSYDMSWVRRSPGKGPEWVSSINSGGGSTYYADYVKGRFTISRDNAKNTLYLQMNSLKPEDTAVYYCAADRYIWARQGEYWGAYEYDYWGQGTQVTVSS

SEQ ID NO: 408 - protein, 128 aa, Artificial
  Mutated Lama sequence
EVQLVESGGGLVQPGGSLRLSCAASGFTIGSYDMSWVRRSPGKGPEWVSAINSGGDSTFYADYVKGRFTISRDNAKNTLYLQMNSLKPEDTAVYYCAADRYIWARQGEYWGAYAYDYWGQGTQVTVSS

SEQ ID NO: 409 - protein, 128 aa, Artificial
  Mutated Lama sequence
EVQLVESGGGLVQPGGSLRLSCAASGFTFGSYDMSWLRRSPGKGPEWVSAINSGGGSTYYADYVKGRFTISRDNAKNTLYLQMNSLKPEDTAVYYCAADRYIWARQGDYWGAYVYDYWGQGTQVTVSS

SEQ ID NO: 410 - protein, 128 aa, Artificial
  Mutated Lama sequence
EVQLVESGGGLVQPGGSLRLSCAASGFTFGSYDMSWVRRSPGKGPEWVSAINSGGGSTYYTDYVKGRFTISRDNAKNTLYLQMNSLKPEDTAVYYCAADRYIWARQGDYWGAYAYDYWGQGTQVTVSS

SEQ ID NO: 411 - protein, 128 aa, Artificial
  Mutated Lama sequence
EVQLVESGGGLVQPGGSLRLSCAASGFTFGSYDMSWVRRSPGKGPEWVSAINSGGGSTYYTDYVKGRFTISRDNAKNTLYLQMNSLKPEDTAVYYCAADRYIRARQGEYWGAYAYDYWGQGTQVTVSS

SEQ ID NO: 412 - protein, 128 aa, Artificial
  Mutated Lama sequence
EVQLVESGGGLVQPGGSLRLSCAASGFTIGSYDMSWVRRSPGKGPEWVSSINSGGGSTYYTDFVKGRFTISRDNAKNTLYLQMNSLKPEDTAVYYCAADRYIWARQGEYWGAYAYDYWGQGTQVTVSS

SEQ ID NO: 413 - protein, 128 aa, Artificial
  Mutated Lama sequence
EVQLVESGGGLVQPGGSLRLSCAASGFTIGSYDMSWVRRSPGKGPEWVSAINSGGGSTYYTDYVKGRFTISRDNAKNTLYLQMNSLKPEDTAVYYCAADRYIWARQGEYWGAYEYDYWGQGTQVTVSS

SEQ ID NO: 414 - protein, 128 aa, Artificial
  Mutated Lama sequence
EVQLVESGGGLVQPGGSLRLSCAASGFTIGSYDMSWVRRSPGKGPEWVSSINSGGGSTFYTDFVKGRFTISRDNAKNTLYLQMNSLKPEDTAVYYCAADRYIWARQGEYWGAYAYDYWGQGTQVTVSS

SEQ ID NO: 415 - protein, 128 aa, Artificial
  Mutated Lama sequence
EVQLVESGGGLVQPGGSLRLSCAASGFTFGSYDMSWVRRSPGKGPEWVSAINSGGGSTYYTDYVKGRFTISRNNAKNTLYLQMNSLKPEDTAVYYCAADRYIWARQGEYWGAYQYDYWGQGTQVTVSS

SEQ ID NO: 416 - protein, 128 aa, Artificial
  Mutated Lama sequence
EVQLVESGGGLVQPGGSLRLSCAASGFTIGSYDMSWVRRSPGKGPEWVSAINSGGGSTYYADYVKGRFTISRDNAKNTLYLQMNSLKPEDTAVYYCAADRYIWARQGEYWGAYAYDYWGQGTQVTVSS

SEQ ID NO: 417 - protein, 687 aa, Homo sapiens
HMMAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 418 - protein, 707 aa, Macaca mulatta
MACACAMLATTARHESSMNKEYMAAASWSASGWALLLLVALWQQRAAGSGVFQLQLQEFVNERGVLASGRPCEPGCRTFFRVCLKHFQAVVSPGPCTFGSVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGASYACECPPNFTGSNCEKKVDRCTSNPCANGGQCLNRGPSRMCRCRPGFTGTYCERHVSDCARNPCAHGGTCHDLESGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 419 - protein, 472 aa, Homo sapiens
EVQLVESGGGLVQPGGSLRLSCAASGFTFTDNWISWVRQAPGKGLEWVGYISPNSGFTYYADSVKGRFTISADTSKNTAYLQMNSLRAEDTAVYYCARDNFGGYFDYWGQGTLVTVSSGEWILCAWAQLCPTPRSHGTTSLAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 420 - protein, 115 aa, Homo sapiens
DIQMTQSPSSLSASVGDRVTITCRASQDVSTAVAWYQQKPGKAPKLLIYSASFLYSGVPSRFSGSGSGTDFTLTISSLQPEDFATTYYCQQSYTGTVTFGQGTKVEIKREWIHLG

SEQ ID NO: 421 - DNA, 21 bp, Artificial
  Primer
gcgaacagagccagattgagg

SEQ ID NO: 422 - DNA, 21 bp, Artificial
  Primer
ggatgtccaggtaggctcctg

SEQ ID NO: 423 - DNA, 20 bp, Artificial
  Primer
gagcgacatccctaacaagc

SEQ ID NO: 424 - DNA, 20 bp, Artificial
  Primer
cctcaactctgttcccttgg

SEQ ID NO: 425 - DNA, 21 bp, Artificial
  Primer
gcgaacagagccagattcagg

SEQ ID NO: 426 - DNA, 20 bp, Artificial
  Primer
ccagacagacacccaaaggt

SEQ ID NO: 427 - DNA, 45 bp, Artificial
  Primer
gaggtgcaattggtggagtctgggggtggtctggttcaggctggt

SEQ ID NO: 428 - DNA, 45 bp, Artificial
  Primer
tcctgcgcagcttctggtcgtaccttctccagctacgcgatggct

SEQ ID NO: 429 - DNA, 45 bp, Artificial
  Primer
ccaggcaaagaacgcgagtwcgtagccgcaatccgttggagcggt

SEQ ID NO: 430 - DNA, 44 bp, Artificial
  Primer
ctgattccgttcagggtcgtttcaccatctctcgtgacaacgcg

SEQ ID NO: 431 - DNA, 45 bp, Artificial
  Primer
ctgcagatgaactctctgaaaccggaagatacggcagtctactac

SEQ ID NO: 432 - DNA, 46 bp, Artificial
  Primer
gacactcgtctgcgtccgtacctgtacgacyattggggtcagggta

SEQ ID NO: 433 - DNA, 46 bp, Artificial
  Primer
gacactcgtctggvaccgtacctgtacgacyattggggtcagggta

SEQ ID NO: 434 - DNA, 46 bp, Artificial
  Primer
gacactcgtctgcgtccgtacgagtacgacyattggggtcagggta

SEQ ID NO: 435 - DNA, 46 bp, Artificial
  Primer
gacactcgtctggvaccgtacgagtacgacyattggggtcagggta

SEQ ID NO: 436 - DNA, 45 bp, Artificial
  Primer
cagacgagtgtccggcgcacggtttgcacagtagtagactgccgt

SEQ ID NO: 437 - DNA, 45 bp, Artificial
  Primer
cagacgagtgtctrccgcacggtttgcacagtagtagactgccgt

SEQ ID NO: 438 - DNA, 45 bp, Artificial
  Primer
agagttcatctgcagatagacggtgtttttcgcgttgtcacgaga

SEQ ID NO: 439 - DNA, 45 bp, Artificial
  Primer
ctgaacggaatcagsgtaatacgcagttycaccgctccaacggat

SEQ ID NO: 440 - DNA, 45 bp, Artificial
  Primer
gcgttctttgcctggagcctgacgawaccaagccatcgcgtagct

SEQ ID NO: 441 - DNA, 45 bp, Artificial
  Primer
agaagctgcgcaggacagacggagagagccaccagcctgaaccag

SEQ ID NO: 442 - DNA, 35 bp, Artificial
  Primer
tgaggagacggtgacctgggtcccctgaccccaat

SEQ ID NO: 443 - DNA, 45 bp, Artificial
  Primer
gaggtgcaattggtggagtctgggggtggtctggttcagccaggt

SEQ ID NO: 444 - DNA, 32 bp, Artificial
  Primer
tgaggagacggtgacctgggtcccctgacccc

SEQ ID NO: 445 - DNA, 45 bp, Artificial
  Primer
gtgcagcttccggctttacgwtcggctcctacgacatgtcttggg

SEQ ID NO: 446 - DNA, 49 bp, Artificial
  Primer
acgcaccccagtattcaccctgacgcgcccaaatgtagcgatctgcagc

SEQ ID NO: 447 - DNA, 45 bp, Artificial
  Primer
aggtccggaatgggtgtcckctatcaactctggtggtggtagcac

SEQ ID NO: 448 - DNA, 45 bp, Artificial
  Primer
tcttccggtttcaggctgttcatctgcaggtacagcgtgtttttg

SEQ ID NO: 449 - DNA, 45 bp, Artificial
  Primer
aaaggtcgtttcaccatctctcgtgacaacgccaaaaacacgctg

SEQ ID NO: 450 - DNA, 45 bp, Artificial
  Primer
tgaaacgaccttttwcgwagtcggygtagwaggtgctaccaccac

SEQ ID NO: 451 - DNA, 45 bp, Artificial
  Primer
tgaaaccggaagataccgcggtatactactgcgctgcagatcgct

SEQ ID NO: 452 - DNA, 45 bp, Artificial
  Primer
ccattccggacctttacccggagaacgacgaacccaagacatgtc

SEQ ID NO: 453 - DNA, 41 bp, Artificial
  Primer
tactggggtgcgtacghatacgactactggggtcagggtac

SEQ ID NO: 454 - DNA, 41 bp, Artificial
  Primer
tactggggtgcgtaccagtacgactactggggtcagggtac

SEQ ID NO: 455 - DNA, 45 bp, Artificial
  Primer
ccggaagctgcacagctcagacgcagagaaccacctggctgaacc

SEQ ID NO: 456 - DNA, 49 bp, Artificial
  Primer
acgcaccccagtagtaaccctgacgcgcccraatgtagcgatctgcagc

SEQ ID NO: 457 - DNA, 49 bp, Artificial
  Primer
acgcaccccagtaktcaccctgacgcgcccraatgtagcgatctgcagc

SEQ ID NO: 458 - protein, 11 aa, Lama glama
PGIAACRGIHY

SEQ ID NO: 459 - protein, 120 aa, Lama glama; identical to SEQ ID NO: 298

SEQ ID NO: 460 - protein, 513 aa, Artificial
  Mutated Lama sequence
DVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFDDYALGWFRQAPGKEREGVSCIRCSDGSTYYADSVKGRFTISSDNSKNTVYLQMNSLRPEDTAVYYCAASIVPRSKLEPYEYDAWGQGTLVTVSSGGGGSGGGSEVQLVESGGGLVQPGGSLRLSCAASGFTFDDYALGWFRQAPGKEREGVSCIRCSDGSTYYADSVKGRFTISSDNSKNTVYLQMNSLRPEDTAVYYCAASIVPRSKLEPYEYDAWGQGTLVTVSS

SEQ ID NO: 461 - protein, 513 aa, Artificial
  Mutated Lama sequence
DVQLVESGGGLVQPGGSLRLSCAASGFTFDDYALGWFRQAPGKEREGVSCIRCSDGSTYYADSVKGRFTISSDNSKNTVYLQMNSLRPEDTAVYYCAASIVPRSKLEPYEYDAWGQGTLVTVSSGGGGSGGGSEVQLVESGGGLVQPGGSLRLSCAASGFTFDDYALGWFRQAPGKEREGVSCIRCSDGSTYYADSVKGRFTISSDNSKNTVYLQMNSLRPEDTAVYYCAASIVPRSKLEPYEYDAWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSS

SEQ ID NO: 462 - protein, 385 aa, Artificial
  Mutated Lama sequence
DVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTLDDYAIGWFRQAPGKEREGVSSIRDNDGSTYYADSVKGRFTISSDNSKNTVYLQMNSLRPEDTAVYYCAAVPAGRLRFGEQWYPLYEYDAWGQGTLVTVSS

SEQ ID NO: 463 - protein, 385 aa, Artificial
  Mutated Lama sequence
DVQLVESGGGLVQPGGSLRLSCAASGFTLDDYAIGWFRQAPGKEREGVSSIRDNDGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSS

SEQ ID NO: 464 - protein, 523 aa, Artificial
  Mutated Lama sequence
DVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTLDDYAIGWFRQAPGKEREGVSSIRDNDGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGFTLDDYAIGWFRQAPGKEREGVSSIRDNDGSTYYADSVKGRFTISSDNSKNTVYLQMNSLRPEDTAVYYCAAVPAGRLRFGEQWYPLYEYDAWGQGTLVTVSS

SEQ ID NO: 465 - protein, 382 aa, Artificial
  Mutated Lama sequence
DVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFALDYYAIGWFRQAPGKEREGVSCISSSDGITYYADSVKGRFTISRDNSKNTVYLQMNSLRPEDTAVYYCATDSGGYIDYDCMGLGYDYWGQGTLVTVSS

SEQ ID NO: 466 - protein, 382 aa, Artificial
  Mutated Lama sequence
DVQLVESGGGLVQPGGSLRLSCAASGFALDYYAIGWFRQAPGKEREGVSCISSSDGITYYADSVKGRFTISRDNSKNTVYLQMNSLRPEDTAVYYCATDSGGYIDYDCMGLGYDYWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSS

SEQ ID NO: 467 - protein, 523 aa, Artificial
  Mutated Lama sequence
DVQLVESGGGLVQPGGSLRLSCAASGFTLDDYAIGWFRQAPGKEREGVSSIRDNDGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGFTLDDYAIGWFRQAPGKEREGVSSIRDNDGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSS

SEQ ID NO: 468 - protein, 513 aa, Artificial
  Mutated Lama sequence
DVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGGSLRLSCAASGFTFDDYALGWFRQAPGKEREGVSCIRCSDGSTYYADSVKGRFTISSDNSKNTVYLQMNSLRPEDTAVYYCAASIVPRSKLEPYEYDAWGQGTLVTVSSGGGGSGGGSEVQLVESGGGLVQPGGSLRLSCAASGFTFDDYALGWFRQAPGKEREGVSCIRCSDGSTYYADSVKGRFTISSDNSKNTVYLQMNSLRPEDTAVYYCAASIVPRSKLEPYEYDAWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 469 - protein, 513 aa, Artificial
  Mutated Lama sequence
DVQLVESGGGLVQPGGSLRLSCAASGFTFDDYALGWFRQAPGKEREGVSCIRCSDGSTYYADSVKGRFTISSDNSKNTVYLQMNSLRPEDTAVYYCAASIVPRSKLEPYEYDAWGQGTLVTVSSGGGGSGGGSEVQLVESGGGLVQPGGSLRLSCAASGFTFDDYALGWFRQAPGKEREGVSCIRCSDGSTYYADSVKGRFTISSDNSKNTVYLQMNSLRPEDTAVYYCAASIVPRSKLEPYEYDAWGQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 470 - protein, 385 aa, Artificial
  Mutated Lama sequence
DVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGGSLRLSCAASGFTLDDYAIGWFRQAPGKEREGVSSIRDNDGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 471 - protein, 385 aa, Artificial
  Mutated Lama sequence
DVQLVESGGGLVQPGGSLRLSCAASGFTLDDYAIGWFRQAPGKEREGVSSIRDNDGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 472 - protein, 523 aa, Artificial
  Mutated Lama sequence
DVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGGSLRLSCAASGFTLDDYAIGWFRQAPGKEREGVSSIRDNDGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGFTLDDYAIGWFRQAPGKEREGVSSIRDNDGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 473 - protein, 523 aa, Artificial
  Mutated Lama sequence
DVQLVESGGGLVQPGGSLRLSCAASGFTLDDYAIGWFRQAPGKEREGVSSIRDNDGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGFTLDDYAIGWFRQAPGKEREGVSSIRDNDGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 474 - protein, 382 aa, Artificial
  Mutated Lama sequence
DVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGGSLRLSCAASGFALDYYAIGWFRQAPGKEREGVSCISSSDGITYYADSVKGRFTISRDNSKNTVYLQMNSLRPEDTAVYYCATDSGGYIDYDCMGLGYDYWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 475 - protein, 382 aa, Artificial
  Mutated Lama sequence
DVQLVESGGGLVQPGGSLRLSCAASGFALDYYAIGWFRQAPGKEREGVSCISSSDGITYYADSVKGRFTISRDNSKNTVYLQMNSLRPEDTAVYYCATDSGGYIDYDCMGLGYDYWGQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 476 - protein, 513 aa, Artificial
  Mutated Lama sequence
DVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFDDYALGWFRQAPGKEREGVSCIRCSGGSTYYADSVKGRFTISSDNSKNTVYLQMNSLRPEDTAVYYCAASIVPRSKLEPYEYDAWGQGTLVTVSSGGGGSGGGSEVQLVESGGGLVQPGGSLRLSCAASGFTFDDYALGWFRQAPGKEREGVSCIRCSGGSTYYADSVKGRFTISSDNSKNTVYLQMNSLRPEDTAVYYCAASIVPRSKLEPYEYDAWGQGTLVTVSS

SEQ ID NO: 477 - protein, 385 aa, Artificial
  Mutated Lama sequence
DVQLVESGGGLVQPGGSLRLSCAVSGITLDDYAIGWFRQAPGKEREGVSAIRSSGGSTYYADSVKGRFTISSDNSKNTVYLQMNSLRPEDTAVYYCAAVPAGRLRYGEQWYPIYEYDAWGQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 478 - protein, 523 aa, Artificial
  Mutated Lama sequence
DVQLVESGGGLVQPGGSLRLSCAASGFTLDDYAIGWFRQAPGKEREGVSAIRSSGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGFTLDDYAIGWFRQAPGKEREGVSAIRSSGGSTYYADSVKGRFTISSDNSKNTVYLQMNSLRPEDTAVYYCAAVPAGRLRFGEQWYPLYEYDAWGQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 479 - protein, 124 aa, Lama glama
EVQLVESGGGLVQAGGSLRLSCAASGFTFDDYALGWFRQAAGKEREGVSCIRCSDGSTYYADSVKGRFTISSDNAKNTVYLQMNSLKPEDTAVYYCAASIVPRSKLEPYEYDAWGQGTQVTVSS

SEQ ID NO: 480 - protein, 126 aa, Lama glama
EVQLVESGGGLVQPGGSLRLSCAASGFALDYYAIGWFRQVPGKEREGVSCISSSDGITYYVDSVKGRFTISRDNAKNTVYLQMNSLKPEDTAVYYCATDSGGYIDYDCMGLGYDYWGQGTQVTVSS

SEQ ID NO: 481 - protein, 129 aa, Lama glama
EVQLVESGGGLVQAGGSLRLSCAASGFTLDDYAIGWFRQAPGKEREGVSCIRDSDGSTYYADSVKGRFTISSDNDKNTVYLQMNSLKPEDTAVYYCAAVPAGRLRFGEQWYPLYEYDAWGQGTQVTVSS

SEQ ID NO: 482 - protein, 129 aa, Artificial
  Mutated lama sequence
EVQLVESGGGLVQPGGSLRLSCAASGFTLDDYAIGWFRQAPGKEREGVSSIRDNDGSTYYADSVKGRFTISSDNSKNTVYLQMNSLRPEDTAVYYCAAVPAGRLRFGEQWYPLYEYDAWGQGTLVTVSS

SEQ ID NO: 483 - protein, 124 aa, Artificial
  Mutated lama sequence
EVQLVESGGGLVQPGGSLRLSCAASGFTFDDYALGWFRQAPGKEREGVSCIRCSDGSTYYADSVKGRFTISSDNSKNTVYLQMNSLRPEDTAVYYCAASIVPRSKLEPYEYDAWGQGTLVTVSS

SEQ ID NO: 484 - protein, 126 aa, Artificial
  Mutated lama sequence
EVQLVESGGGLVQPGGSLRLSCAASGFALDYYAIGWFRQAPGKEREGVSCISSSDGITYYADSVKGRFTISRDNSKNTVYLQMNSLRPEDTAVYYCATDSGGYIDYDCMGLGYDYWGQGTLVTVSS

SEQ ID NO: 485 - protein, 124 aa, Artificial
  Mutated lama sequence
EVQLVESGGGLVQPGGSLRLSCAASGFTFDDYALGWFRQAPGKEREGVSCIRCSGGSTYYADSVKGRFTISSDNSKNTVYLQMNSLRPEDTAVYYCAASIVPRSKLEPYEYDAWGQGTLVTVSS

SEQ ID NO: 486 - protein, 126 aa, Artificial
  Mutated lama sequence
EVQLVESGGGLVQPGGSLRLSCAASGFALDYYAIGWFRQAPGKEREGVSCISSSGGITYYADSVKGRFTISRDNSKNTVYLQMNSLRPEDTAVYYCATDSGGYIDYDCSGLGYDYWGQGTLVTVSS

SEQ ID NO: 487 - protein, 129 aa, Artificial
  Mutated lama sequence
EVQLVESGGGLVQPGGSLRLSCAVSGITLDDYAIGWFRQAPGKEREGVSAIRSSGGSTYYADSVKGRFTISSDNSKNTVYLQMNSLRPEDTAVYYCAAVPAGRLRYGEQWYPIYEYDAWGQGTLVTVSS

SEQ ID NO: 488 - protein, 129 aa, Artificial
  Mutated lama sequence
EVQLVESGGGLVQPGGSLRLSCAASGFTLDDYAIGWFRQAPGKEREGVSAIRSSGGSTYYADSVKGRFTISSDNSKNTVYLQMNSLRPEDTAVYYCAAVPAGRLRFGEQWYPLYEYDAWGQGTLVTVSS

SEQ ID NO: 489 - protein, 5 aa, Lama glama
DYALG

SEQ ID NO: 490 - protein, 17 aa, Lama glama
CIRCSDGSTYYADSVKG

SEQ ID NO: 491 - protein, 15 aa, Lama glama
SIVPRSKLEPYEYDA

SEQ ID NO: 492 - protein, 5 aa, Lama glama
YYAIG

SEQ ID NO: 493 - protein, 17 aa, Lama glama
CISSSDGITYYVDSVKG

SEQ ID NO: 494 - protein, 17 aa, Lama glama
DSGGYIDYDCMGLGYDY

SEQ ID NO: 495 - protein, 5 aa, Lama glama
DYAIG

SEQ ID NO: 496 - protein, 17 aa, Lama glama
CIRDSDGSTYYADSVKG

SEQ ID NO: 497 - protein, 20 aa, Lama glama
VPAGRLRFGEQWYPLYEYDA

SEQ ID NO: 498 - protein, 5 aa, Lama glama
DYAIG

SEQ ID NO: 499 - protein, 17 aa, Lama glama
SIRDNDGSTYYADSVKG

SEQ ID NO: 500 - protein, 20 aa, Lama glama
VPAGRLRFGEQWYPLYEYDA

SEQ ID NO: 501 - protein, 5 aa, Lama glama
DYALG

SEQ ID NO: 502 - protein, 17 aa, Lama glama
CIRCSDGSTYYADSVKG

SEQ ID NO: 503 - protein, 15 aa, Lama glama
SIVPRSKLEPYEYDA

SEQ ID NO: 504 - protein, 5 aa, Lama glama
YYAIG

SEQ ID NO: 505 - protein, 17 aa, Lama glama
CISSSDGITYYADSVKG

SEQ ID NO: 506 - protein, 17 aa, Lama glama
DSGGYIDYDCMGLGYDY

SEQ ID NO: 507 - protein, 5 aa, Lama glama
DYALG

SEQ ID NO: 508 - protein, 17 aa, Lama glama
CIRCSGGSTYYADSVKG

SEQ ID NO: 509 - protein, 15 aa, Lama glama
SIVPRSKLEPYEYDA

SEQ ID NO: 510 - protein, 5 aa, Lama glama
YYAIG

SEQ ID NO: 511 - protein, 17 aa, Lama glama
CISSSGGITYYADSVKG

SEQ ID NO: 512 - protein, 17 aa, Artificial
  Mutated lama sequence
DSGGYIDYDCSGLGYDY

SEQ ID NO: 513 - protein, 5 aa, Lama glama
DYAIG

SEQ ID NO: 514 - protein, 17 aa, Artificial
  Mutated lama sequence
AIRSSGGSTYYADSVKG

SEQ ID NO: 515 - protein, 20 aa, Artificial
  Mutated lama sequence
VPAGRLRYGEQWYPIYEYDA

SEQ ID NO: 516 - protein, 5 aa, Lama glama
DYAIG

SEQ ID NO: 517 - protein, 17 aa, Artificial
  Mutated lama sequence
AIRSSGGSTYYADSVKG

SEQ ID NO: 518 - protein, 20 aa, Artificial
  Mutated lama sequence
VPAGRLRFGEQWYPLYEYDA

SEQ ID NO: 519 - protein, 115 aa, Artificial [sequence omitted]

SEQ ID NO: 520 - protein, 5 aa, Lama glama
SFGMS

SEQ ID NO: 521 - protein, 17 aa, Lama glama
SISGSGSDTLYADSVKG

SEQ ID NO: 522 - protein, 6 aa, Lama glama
GGSLSR

SEQ ID NO: 523 - protein, 5 aa, Lama glama
LNLMG

SEQ ID NO: 524 - protein, 16 aa, Lama glama
TITVGDSTNYADSVKG

SEQ ID NO: 525 - protein, 8 aa, Lama glama
RRTWHSEL

SEQ ID NO: 526 - protein, 5 aa, Lama glama
INLLG

SEQ ID NO: 527 - protein, 16 aa, Lama glama
TITVGDSTSYADSVKG

SEQ ID NO: 528 - protein, 8 aa, Lama glama
RRTWHSEL

SEQ ID NO: 529 - protein, 5 aa, Lama glama
SFGMS

SEQ ID NO: 530 - protein, 17 aa, Lama glama
SINGRGDDTRYADSVKG

SEQ ID NO: 531 - protein, 7 aa, Lama glama
GRSVSRS

SEQ ID NO: 532 - protein, 5 aa, Lama glama
SFGMS

SEQ ID NO: 533 - protein, 17 aa, Lama glama
AISADSSDKRYADSVKG

SEQ ID NO: 534 - protein, 5 aa, Lama glama
GRGSP

SEQ ID NO: 535 - protein, 5 aa, Lama glama
SFGMS

SEQ ID NO: 536 - protein, 17 aa, Lama glama
AISADSSDKRYADSVKG

SEQ ID NO: 537 - protein, 5 aa, Lama glama
GRGSP

SEQ ID NO: 538 - protein, 5 aa, Lama glama
NYWMY

SEQ ID NO: 539 - protein, 17 aa, Lama glama
RISTGGGYSYYADSVKG

SEQ ID NO: 540 - protein, 13 aa, Lama glama
DREAQVDTLDFDY

(19) United States
(12) Patent Application Publication
(10) Pub. No.: US 2013/0078247 A1
(43) Pub. Date: Mar. 28, 2013

(54) BISPECIFIC BINDING MOLECULES BINDING TO DII4 AND ANG2

(71) Applicants: Andreas GSCHWIND, Vienna (AT); Rene Georg OTT, Vienna (AT); Joachim BOUCNEAU, De Pinte (BE); Marie-Ange BUYSE, Merelbeke (BE); Erik DEPLA, Destelbergen (BE)

(72) Inventors: Andreas GSCHWIND, Vienna (AT); Rene Georg OTT, Vienna (AT); Joachim BOUCNEAU, De Pinte (BE); Marie-Ange BUYSE, Merelbeke (BE); Erik DEPLA, Destelbergen (BE)

(73) Assignee: BOEHRINGER INGELHEIM INTERNATIONAL GMBH, Ingelheim am Rhein (DE)

(21) Appl. No.: 13/433,354

(22) Filed: Mar. 29, 2012

(30) Foreign Application Priority Data
Apr. 1, 2011 (EP) 11160920.2

(51) Int. Cl. C07K 16/46

(52) U.S. Cl. CPC C07K 16/468
USPC 424/136.1; 530/387.3; 536/23.4

(57) ABSTRACT

Bispecific binding molecules binding to both DII4 and Ang2, preferably in the form of immunoglobulin single variable domains like VHHs and domain antibodies, pharmaceutical compositions containing the same and their use in the treatment of diseases that are associated with DII4- and/or Ang2-mediated effects on angiogenesis are disclosed. Further, nucleic acids encoding bispecific binding molecules, host cells and methods for preparing same are also described.

### FIELD OF THE INVENTION

The invention relates to the field of human therapy, in particular cancer therapy and agents and compositions useful in such therapy.

### BACKGROUND OF THE INVENTION

When tumors reach a critical size of approximately 1 mm³ they become dependent on angiogenesis for maintaining blood supply with oxygen and nutrients to allow for further growth. Anti-angiogenesis therapies have become an important treatment option for several types of tumors. These therapies have focused on blocking the VEGF pathway (Ferrara et al., Nat Rev Drug Discov. 2004 May; 3(5):391-400.) by neutralizing VEGF (Avastin) or its receptors (Sutent and Sorafinib). Recent studies in mice have shown, that Angiopoietin2 (Ang2), a ligand of the Tie2 receptor, controls vascular remodeling by enabling the functions of other angiogenic factors, such as VEGF. Ang2 is primarily expressed by endothelial cells, strongly induced by hypoxia and other angiogenic factors and has been demonstrated to regulate tumor vessel plasticity, allowing vessels to respond to VEGF and FGF2 (Augustin et al., Nat Rev Mol Cell Biol. 2009 March; 10(3):165-77.). Consistent with this role, the deletion or inhibition of Ang2 results in reduced angiogenesis (Falcón et al., Am J Pathol. 2009 November; 175(5):2159-70.). Elevated Ang2 serum concentrations have been reported for patients with colorectal cancer, NSCLC and melanoma (Goede et al., Br J Cancer. 2010 Oct. 26; 103(9):1407-14), (Park et al., Chest. 2007 July; 132(1): 200-6.), (Helfrich et al., Clin Cancer Res. 2009 Feb. 15; 15(4):1384-92.). In CRC cancer Ang2 serum levels correlate with therapeutic response to anti-VEGF therapy.

The Ang-Tie system consists of 2 receptors (Tie1 and Tie2) and 3 ligands (Ang1, Ang2 and Ang4) (Augustin et al., Nat Rev Mol Cell Biol. 2009 March; 10(3):165-77.). Tie2, Ang1 and Ang2 are the best studied members of this family, Tie1 is an orphan receptor and the role of Ang4 for vascular remodelling still needs to be defined. Ang2 and Ang1 mediate opposing functions upon Tie2 binding and activation. Ang2-mediated Tie2 activation results in endothelial cell activation, pericyte dissociation, vessel leakage and induction of vessel sprouting. In contrast to Ang2, Ang1 signaling maintains vessel integrity by recruitment of pericytes, thereby maintaining endothelial cell quiescence.

Angiopoietin 2 (Ang2) is a secreted, 66 kDa ligand for the Tie2 receptor tyrosine kinase (Augustin et al., Nat Rev Mol Cell Biol. 2009 March; 10(3):165-77.). Ang2 consists of an N-terminal coiled-coil domain and a C-terminal fibrinogen-like domain, the latter is required for Tie2 interaction. Ang2 is primarily expressed by endothelial cells and strongly induced by hypoxia and other angiogenic factors, including VEGF. Tie2 is found on endothelial cells, haematopoietic stem cells and tumor cells. Ang2-Tie2 has been demonstrated to regulate tumor vessel plasticity, allowing vessels to to respond to VEGF and FGF2.

In vitro Ang2 has been shown to act as a modest mitogen, chemoattractant and inducer of tube formation in human umbilical vein endothelial cells (HUVEC). Ang2 induces tyrosine phosphorylation of ectopically expressed Tie2 in fibroblasts and promotes downstream signaling events, such as phosphorylation of ERK-MAPK, AKT and FAK in HUVEC. An antagonistic role of Ang2 in Ang1-induced endothelial cell responses has been described.

Ang2-deficiency has been shown to result in a profound lymphatic patterning defect in mice. Although the loss of Ang2 is dispensable for embryonic vascular development, Ang2-deficient mice have persistent vascular defects in the retina and kidney. Together with the dynamic pattern of Ang2 expression at sites of angiogenesis (for example ovary), these findings indicate that Ang2 controls vascular remodeling by enabling the functions of other angiogenic factors, such as VEGF.

The Ang2-Tie2 system exerts crucial roles during the angiogenic switch and later stages of tumor angiogenesis. Ang2 expression is strongly up-regulated in the tumor-associated endothelium. Reduced growth of tumors has been observed when implanted into Ang2-deficient mice, especially during early stages of tumor growth. Therapeutic blocking of Ang2 with Ang2 mAbs has shown broad efficacy in a variety of tumor xenograft models.

As summarized in US 2008/0014196, angiogenesis is implicated in the pathogenesis of a number of disorders, including solid tumors and metastasis.

In the case of tumor growth, angiogenesis appears to be crucial for the transition from hyperplasia to neoplasia, and for providing nourishment for the growth and metastasis of the tumor. Folkman et al., Nature 339-58 (1989), which allows the tumor cells to acquire a growth advantage compared to the normal cells. Therefore, anti-angiogenesis therapies have become an important treatment option for several types of tumors. These therapies have focused on blocking the VEGF pathway (Ferrara et al., Nat Rev Drug Discov. 2004 May; 3(5):391-400.

The Notch signaling pathway is important for cell-cell communication, which involves gene regulation mechanisms that control multiple cell differentiation processes during to embryonic development and in adult organisms. Notch signaling is dysregulated in many cancers, e.g. in T-cell acute lymphoblastic leukemia and in solid tumors (Sharma et al. 2007, Cell Cycle 6 (8): 927-30; Shih et al., Cancer Res. 2007 Mar. 1; 67(5): 1879-82).

DII4 (or Delta like 4 or delta-like ligand 4) is a member of the Delta family of Notch ligands. The extracellular domain of DII4 is composed of an N-terminal domain, a Delta/Serrate/Lag-2 (DSL) domain, and a tandem of eight epidermal growth factor (EGF)-like repeats. Generally, the EGF domains are recognized as comprising amino acid residues 218-251 (EGF-1; domain 1), 252-282 (EGF-2; domain 2), 284-322 (EGF-3; domain 3), 324-360 (EGF-4; domain 4), and 362-400 (EGF-5; domain 5), with the DSL domain at about amino acid residues 173-217 and the N-terminal domain at about amino acid residues 27-172 of hDII4 (WO 2008/076379).

It has been reported that DII4 exhibits highly selective expression by vascular endothelium, in particular in arterial endothelium (Shutter et al. (2000) Genes Develop. 14: 1313-1318). Recent studies in mice have shown that DII4 is induced by VEGF and is a negative feedback regulator that restrains vascular sprouting and branching. Consistent with this role, the deletion or inhibition of DII4 results in excessive angiogenesis (Scehnet et al., Blood. 2007 Jun. 1; 109(11):4753-60). This unrestrained angiogenesis paradoxically decreases tumor growth due to the formation of non-productive vasculature, even in tumors resistant to anti-VEGF therapies (Thurston et al., Nat Rev Cancer. 2007 May; 7(5):327-31; WO 2007/070671; Noguera-Troise et al., Nature. 2006 Dec. 21; 444(7122)). Furthermore, the combined inhibition of VEGF and DII4 is shown to provide superior anti-tumor activity compared to anti-VEGF alone in xenograft models of multiple tumor types (Noguera-Troise et al., Nature. 2006 Dec. 21; 444(7122):1032-7; Ridgway et al., Nature. 2006 Dec. 21; 444(7122):1083-7).

Due to these results, DII4 is being considered a promising target for cancer therapy, and several biological compounds that target DII4 are in (pre-)clinical development have been described: REGN-421 (═SAR153192; Regeneron, Sanofi-Aventis; WO2008076379) and OPM-21 M18 (OncoMed) (Hoey et al., Cell Stem Cell. 2009 Aug. 7; 5(2):168-77), both fully human DII4 antibodies; YW152F (Genentech), a humanized DII4 antibody (Ridgway et al., Nature. 2006 Dec. 21; 444(7122):1083-7); DII4-Fc (Regeneron, Sanofi-Aventis), a recombinant fusion protein composed of the to extracellular region of DII4 and the Fc region of human IgG1 (Noguera-Troise et al., Nature. 2006 Dec. 21; 444(7122)).

However, the state-of-the art monoclonal antibodies (MAbs) and fusion proteins have several shortcomings in view of their therapeutic application: To prevent their degradation, they must be stored at near freezing temperatures. Also, since they are quickly digested in the gut, they are not suited for oral administration. Another major restriction of MAbs for cancer therapy is poor transport, which results in low concentrations and a lack of targeting of all cells in a tumor.

It has been an object of the present invention to provide novel anti-angiogenic binding molecules for human therapy.

It has been a further object of the invention to provide methods for the prevention, treatment, alleviation and/or diagnosis of such diseases, disorders or conditions, involving the use and/or administration of such binding molecules and compositions comprising them. In particular, it is has been an object of the invention to provide such pharmacologically active binding molecules, compositions and/or methods that provide advantages compared to the agents, compositions and/or methods currently used and/or known in the art. These advantages include improved therapeutic and/or pharmacological properties and/or other advantageous properties, e.g. for manufacturing purposes, especially as compared to conventional antibodies as those described above, or fragments thereof.

### BRIEF SUMMARY OF THE INVENTION

According to a first aspect, there are provided bispecific binding molecules, preferably bispecific immunoglobulins, preferably immunoglobulin single variable domains like VHHs and domain antibodies, which comprises at least one DLL4 binding component and at least one Ang2-binding component in a single molecule. These bispecific binding molecules may preferably comprise a further binding component, preferably a binding component binding to serum albumin.

More specifically, a bispecific binding molecule of the invention essentially comprises (i) at least one DII4-binding component specifically binding to at least one epitope of DII4 and (ii) at least one Ang2-binding component specifically binding to at least an epitope of Ang2, wherein the components are linked to each other in such a way that they simultaneously bind to DII4 and Ang2 or that they bind to either DII4 or Ang2 at a time.

According to preferred aspects of the invention, the two components comprise one or more immunoglobulin single variable domains that may be, independently of each other, VHHs or domain antibodies, and/or any other sort of immunoglobulin single variable domains, such as VL domains, as defined herein, provided that each of these immunoglobulin single variable domains will bind the antigen, i.e. DII4 or Ang2, respectively.

According to a preferred embodiment, the immunoglobulin single variable domains are of the same type, in particular, all immunoglobulin single variable domains are VHHs or domain antibodies.

According to a particularly preferred embodiment, all immunoglobulin single variable domains are VHHs, preferably humanized (or “sequence-optimized”, as defined herein) VHHs. Accordingly, the invention relates to bispecific binding molecules comprising an (optionally humanized or sequence-optimized) anti-DII4 VHH and an (optionally humanized or sequence-optimized) anti-Ang2 VHH.

However, it will be clear to the skilled person that the teaching herein may be applied analogously to bispecific binding molecules including other anti-DII4 or anti-Ang2 immunoglobulin single variable domains, such as domain antibodies.

In another aspect, the invention relates to nucleic acids encoding the bispecific binding molecules of the invention as well as host cells containing same.

The invention further relates to a product or composition containing or comprising at least one bispecific binding molecule of the invention and optionally one or more further components of such compositions.

The invention further relates to methods for preparing or generating the bispecific binding molecules, nucleic acids, host cells, products and compositions described herein.

The invention further relates to applications and uses of the bispecific binding to molecules, nucleic acids, host cells, products and compositions described herein, as well as to methods for the prevention and/or treatment for diseases and disorders that can be modulated by inhibition of DII4.

It has been found that the Ang2-binding component of the bispecific binding molecules according to the present invention binds to Ang2 with a potency at least 5,000 times higher, preferably 10,000 times higher than to Ang1 or Ang4. This will largely avoid blocking activation of Ang1-mediated signalling, which would counter the intended anti-angiogenetic effect.

It has further been found that the DLL4-binding component of the bi-specific binding molecules according to the present invention binds to DLL4-A with an affinity of at least 1,000 times higher than to DII1, Jagged1 and preferably also against Jagged2. Due to this selectivity unwanted side reactions can be avoided.

In a preferred embodiment the bispecific binding molecules of the present invention are provided as linked VHH domains. Such molecules are significantly smaller than conventional antibodies and have thus the potential for penetrating into a tumor deeper than such conventional antibodies. This benefit is further accentuated by the specific sequences disclosed herein after being free of glycosylation sites.

Further, due to the bispecific nature (DII4- and Ang2-binding components in one molecule) the tumor penetration of both functionalities will be necessarily equal, which will ensure that the beneficial effects of the combined antagonism of DII4 and Ang2 will be provided within the whole depth of penetration of the tumor. This is an advantage over the combination of individual antagonists against these targets, since the depth of penetration of individual antagonists will always vary to some degree. Another advantage of a preferred bispecific binding molecules of the present invention is their increased serum half-like due to a serum albumin binding component such as a serum albumin binding molecule as described herein.

These and other aspects, embodiments, advantages and applications of the invention will become clear from the further description hereinbelow.

### DEFINITIONS

Unless indicated or defined otherwise, all terms used have their usual meaning in the 1 to art, which will be clear to the skilled person. Reference is for example made to the standard handbooks, such as Sambrook et al, “Molecular Cloning: A Laboratory Manual” (2nd Ed.), Vols. 1-3, Cold Spring Harbor Laboratory Press (1989); Lewin, “Genes IV”, Oxford University Press, New York, (1990), and Roitt et al., “Immunology” (2^{nd} Ed.), Gower Medical Publishing, London, New York (1989), as well as to the general background art cited herein; Furthermore, unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

The term “bispecific binding molecule” refers to a molecule comprising at least one Ang2-binding molecule (or “Ang2-binding component”) and at least one DII4-binding molecule (or “DII4-binding component”). A bispecific binding molecule may contain more than one Ang2-binding molecule and/or more than one DII4-binding molecule, i.e. in the case that the bispecific binding molecule contains a biparatopic (as defined below) Ang2-binding molecule and/or a biparatopic DII-4-binding molecule, in the part of the molecule that binds to Ang2 or to DII4, i.e. in its “Ang2-binding component” (or anti-Ang2 component) or “DII4-binding component” (or anti-DII4 component), respectively. The word “bispecific” in this context is however not to be construed as to exclude further binding components with binding specificity to molecules other than DII4 and Ang2 from the bispecific binding molecule. Non-limiting examples of such further binding components are binding components binding to serum albumin.

Unless indicated otherwise, the terms “immunoglobulin” and “immunoglobulin sequence”—whether used herein to refer to a heavy chain antibody or to a conventional 4-chain antibody—are used as general terms to include both the full-size antibody, the individual chains thereof, as well as all parts, domains or fragments thereof (including but not limited to antigen-binding domains or fragments such as VHH domains or VH/VL domains, respectively). In addition, the term “sequence” as used herein (for example in terms like “immunoglobulin sequence”, “antibody sequence”, “(single) variable domain sequence”, “VHH sequence” or “protein sequence”), should generally be understood to include both the relevant amino acid to sequence as well as nucleic acid sequences or nucleotide sequences encoding the same, unless the context requires a more limited interpretation.

The term “domain” (of a polypeptide or protein) as used herein refers to a folded protein structure which has the ability to retain its tertiary structure independently of the rest of the protein. Generally, domains are responsible for discrete functional properties of proteins, and in many cases may be added, removed or transferred to other proteins without loss of function of the remainder of the protein and/or of the domain.

The term “immunoglobulin domain” as used herein refers to a globular region of an antibody chain (such as e.g. a chain of a conventional 4-chain antibody or of a heavy chain antibody), or to a polypeptide that essentially consists of such a globular region. Immunoglobulin domains are characterized in that they retain the immunoglobulin fold characteristic of antibody molecules, which consists of a 2-layer sandwich of about 7 antiparallel beta-strands arranged in two beta-sheets, optionally stabilized by a conserved disulphide bond. An immunoglobulin domain comprises (a) variable domain(s), i.e., one or more immunoglobulin variable domains.

The term “immunoglobulin variable domain” as used herein means an immunoglobulin domain essentially consisting of four “framework regions” which are referred to in the art and hereinbelow as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and hereinbelow as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) that confer specificity to an antibody for the antigen by carrying the antigen-binding site. In the context of the present invention immunoglobulin single variable domains like VHHs and domain antibodies are preferred.

The term “immunoglobulin single variable domain” as used herein means an immunoglobulin variable domain which is capable of specifically binding to an epitope of the antigen without pairing with an additional variable immunoglobulin domain. One example of immunoglobulin single variable domains in the meaning of the present invention are “domain antibodies”, such as the immunoglobulin single variable domains VH and VL (VH domains and VL domains). Another example of immunoglobulin single variable domains are “VHH domains” (or simply “VHHs”) from camelids, as defined hereinafter.

In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e. by a VH-VL pair of immunoglobulin domains, which jointly bind to an epitope of the respective antigen.

“VHH domains”, also known as VHHs, V_{H}H domains, VHH antibody fragments, and VHH antibodies, have originally been described as the antigen binding immunoglobulin (variable) domain of “heavy chain antibodies” (i.e. of “antibodies devoid of light chains”; Hamers-Casterman C, Atarhouch T, Muyldermans S, Robinson G, Hamers C, Songa E B, Bendahman N, Hamers R.: “Naturally occurring antibodies devoid of light chains”; Nature 363, 446-448 (1993)). The term “VHH domain” has been chosen in order to distinguish these variable domains from the heavy chain variable domains that are present in conventional 4-chain antibodies (which are referred to herein as “V_{H} domains” or “VH domains”) and from the light chain variable domains that are present in conventional 4-chain antibodies (which are referred to herein as “V_{L} domains” or “VL domains”). VHH domains can specifically bind to an epitope without an additional antigen binding domain (as opposed to VH or VL domains in a conventional 4-chain antibody, in which case the epitope is recognized by a VL domain together with a VH domain). VHH domains are small, robust and efficient antigen recognition units formed by a single immunoglobulin domain.

In the context of the present invention, the terms VHH domain, VHH, V_{H} H domain, VHH antibody fragment, VHH antibody, as well as “Nanobody®” and “Nanobody® o0 domain” (“Nanobody” being a trademark of the company Ablynx N.V.; Ghent; Belgium) are used interchangeably and are representatives of immunoglobulin single variable domains (having the structure FR1-CDR1-FR2-CDR2-FR3-CDR3-FR4 and specifically binding to an epitope without requiring the presence of a second immunoglobulin variable domain), and which are distinguished from VH domains by the so-called “hallmark residues”, as defined in e.g. WO2009/109635, FIG. 1.

The amino acid residues of a immunoglobulin single variable domain, e.g. a VHH, are numbered according to the general numbering for V_{H} domains given by Kabat et al. (“Sequence of proteins of immunological interest”, US Public Health Services, NIH Bethesda, Md., Publication No. 91), as applied to VHH domains from Camelids, as shown e.g. in FIG. 2 of Riechmann and Muyldermans, J. Immunol. Methods 231, 25-38 (1999). According to this numbering,
FR1 comprises the amino acid residues at positions 1-30,
CDR1 comprises the amino acid residues at positions 31-35,
FR2 comprises the amino acids at positions 36-49,
CDR2 comprises the amino acid residues at positions 50-65,
FR3 comprises the amino acid residues at positions 66-94,
CDR3 comprises the amino acid residues at positions 95-102, and
FR4 comprises the amino acid residues at positions 103-113.

However, it should be noted that—as is well known in the art for V_{H} domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence.

Alternative methods for numbering the amino acid residues of V_{H} domains, which methods can also be applied in an analogous manner to VHH domains, are known in to the art. However, in the present description, claims and figures, the numbering according to Kabat and applied to VHH domains as described above will be followed, unless indicated otherwise.

The total number of amino acid residues in a VHH domain will usually be in the range of from 110 to 120, often between 112 and 115. It should however be noted that smaller and longer sequences may also be suitable for the purposes described herein.

Immunoglobulin single variable domains, e.g. VHHs and domain antibodies, according to the preferred embodiments of the invention, have a number of unique structural characteristics and functional properties which makes them highly advantageous for use in therapy as functional antigen-binding molecules. In particular, and without being limited thereto, VHH domains (which have been “designed” by nature to functionally bind to an antigen without pairing with a light chain variable domain) can function as single, relatively small, functional antigen-binding structural units.

Due to their unique properties, immunoglobulin single variable domains, as defined herein, like VHHs or VHs (or VLs)—either alone or as part of a larger polypeptide, e.g. a biparatopic molecule—offer a number of significant advantages:
only a single domain is required to bind an antigen with high affinity and with high selectivity, so that there is no need to have two separate domains present, nor to assure that these two domains are present in the right spacial conformation and configuration (i.e. through the use of especially designed linkers, as with scFv's);
immunoglobulin single variable domains can be expressed from a single nucleic acid molecule and do not require any post-translational modification (like glycosylation;
immunoglobulin single variable domains can easily be engineered into multivalent and multispecific formats (as further discussed herein);
immunoglobulin single variable domains have high specificity and affinity for their target, low inherent toxicity and can be administered via alternative routes than infusion or injection;
immunoglobulin single variable domains are highly stable to heat, pH, to proteases and other denaturing agents or conditions and, thus, may be prepared, stored or transported without the use of refrigeration equipments;
immunoglobulin single variable domains are easy and relatively inexpensive to prepare, both on small scale and on a manufacturing scale. For example, immunoglobulin single variable domains can be produced using microbial fermentation (e.g. as further described below) and do not require the use of mammalian expression systems, as with for example conventional antibodies;
immunoglobulin single variable domains are relatively small (approximately kDa, or 10 times smaller than a conventional IgG) compared to conventional 4-chain antibodies and antigen-binding fragments thereof, and therefore show high(er) penetration into tissues (including but not limited to solid tumors and other dense tissues) and can be administered in higher doses than such conventional 4-chain antibodies and antigen-binding fragments thereof;
VHHs have specific so-called “cavity-binding properties” (inter alia due to their extended CDR3 loop, compared to VH domains from 4-chain antibodies) and can therefore also access targets and epitopes not accessible to conventional 4-chain antibodies and antigen-binding fragments thereof;
VHHs have the particular advantage that they are highly soluble and very stable and do not have a tendency to aggregate (as with the mouse-derived antigen-binding domains described by Ward et al., Nature 341: 544-546 (1989)).

The immunoglobulin single variable domains of the invention are not limited with respect to a specific biological source from which they have been obtained or to a specific method of preparation. For example, obtaining VHHs may include the following steps:

(1) isolating the VHH domain of a naturally occurring heavy chain antibody; or screening a library comprising heavy chain antibodies or VHHs and isolating VHHs therefrom;

(2) expressing a nucleic acid molecule encoding a VHH with the naturally occurring sequence;

(3) “humanizing” (as described herein) a VHH, optionally after affinity maturation, with a naturally occurring sequence or expressing a nucleic acid encoding such humanized VHH;

(4) “camelizing” (as described below) a immunoglobulin single variable heavy domain from a naturally occurring antibody from an animal species, in particular a species of mammal, such as from a human being, or expressing a nucleic acid molecule encoding such camelized domain;

(5) “camelizing” a VH, or expressing a nucleic acid molecule encoding such a camelized VH;

(6) using techniques for preparing synthetically or semi-synthetically proteins, polypeptides or other amino acid sequences;

(7) preparing a nucleic acid molecule encoding a VHH domain using techniques for nucleic acid synthesis, followed by expression of the nucleic acid thus obtained;

(8) subjecting heavy chain antibodies or VHHs to affinity maturation, to mutagenesis (e.g. random mutagenesis or site-directed mutagenesis) and/or any other technique(s) in order to increase the affinity and/or specificity of the VHH; and/or

(9) combinations or selections of the foregoing steps.

Suitable methods and techniques for performing the above-described steps are known in the art and will be clear to the skilled person. By way of example, methods of obtaining VHH domains binding to a specific antigen or epitope have been described in WO2006/040153 and WO2006/122786.

According to specific embodiments, the immunoglobulin single variable domains of the invention or present in the polypeptides of the invention are VHH domains with an amino acid sequence that essentially corresponds to the amino acid sequence of a naturally occurring VHH domain, but that has been “humanized” or “sequence-optimized” (optionally after affinity-maturation), i.e. by replacing one or more amino acid residues in the amino acid sequence of said naturally occurring VHH sequence by one or more of the amino acid residues that occur at the corresponding position(s) in a variable heavy domain of a conventional 4-chain antibody from a human being. This can be performed using methods known in the art, which can by routinely used to by the skilled person.

A humanized VHH domain may contain one or more fully human framework region sequences, and, in an even more specific embodiment, may contain human framework region sequences derived from the human germline Vh3 sequences DP-29, DP-47, DP-51, or parts thereof, or be highly homologous thereto, optionally combined with JH sequences, such as JH5. Thus, a humanization protocol may comprise the replacement of any of the VHH residues with the corresponding framework 1, 2 and 3 (FRI, FR2 and FR3) residues of germline VH genes such as DP 47, DP 29 and DP 51) either alone or in combination. Suitable framework regions (FR) of the immunoglobulin single variable domains of the invention can be selected from those as set out e.g. in WO 2006/004678 and specifically, include the so-called “KERE” and “GLEW” classes. Examples are immunoglobulin single variable domains having the amino acid sequence G-L-E-W at about positions 44 to 47, and their respective humanized counterparts. A humanized VHH domain may contain one or more fully human framework region sequences.

By way of example, a humanizing substitution for VHHs belonging to the 103 P,R,S-group and/or the GLEW-group (as defined below) is 108Q to 108L. Methods for humanizing immunoglobulin single variable domains are known in the art.

Binding immunoglobulin single variable domains with improved properties in view of therapeutic application, e.g. enhanced affinity or decreased immunogenicity, may be obtained from individual binding molecules by techniques known in the art, such as affinity maturation (for example, starting from synthetic, random or naturally occurring immunoglobulin sequences), CDR grafting, humanizing, combining fragments derived from different immunoglobulin sequences, PCR assembly using overlapping primers, and similar techniques for engineering immunoglobulin sequences well known to the skilled person; or any suitable combination of any of the foregoing, also termed “sequence optimization”, as described herein. Reference is, for example, made to standard handbooks, as well as to the further description and Examples.

If appropriate, a binding molecule with increased affinity may be obtained by affinity-maturation of another binding molecule, the latter representing, with respect to the affinity-matured molecule, the “parent” binding molecule.

Methods of obtaining VHHs that bind to a specific antigen or epitope have been to described earlier, e.g. in WO2006/040153 and WO2006/122786. As also described therein in detail, VHH domains derived from camelids can be “humanized” (also termed “sequence-optimized” herein, “sequence-optimizing” may, in addition to humanization, encompass an additional modification of the sequence by one or more mutations that furnish the VHH with improved properties, such as the removal of potential post translational modification sites) by replacing one or more amino acid residues in the amino acid sequence of the original VHH sequence by one or more of the amino acid residues that occur at the corresponding position(s) in a VH domain from a conventional 4-chain antibody from a human being. A humanized VHH domain can contain one or more fully human framework region sequences, and, in an even more specific embodiment, can contain human framework region sequences derived from DP-29, DP-47, DP-51, or parts thereof, optionally combined with JH sequences, such as JH5.

Domain antibodies, also known as “Dab”s and “dAbs” (the terms “Domain Antibodies” and “dAbs” being used as trademarks by the GlaxoSmithKline group of companies) have been described in e.g. Ward, E. S., et al.: “Binding activities of a repertoire of single immunoglobulin variable domains secreted from Escherichia coli”; Nature 341: 544-546 (1989); Holt, L. J. et al.: “Domain antibodies: proteins for therapy”; TRENDS in Biotechnology 21(11): 484-490 (2003); and WO2003/002609.

Domain antibodies essentially correspond to the VH or VL domains of antibodies from non-camelid mammals, in particular human 4-chain antibodies. In order to bind an epitope as a single antigen binding domain, i.e. without being paired with a VL or VH domain, respectively, specific selection for such antigen binding properties is required, e.g. by using libraries of human single VH or VL domain sequences. Domain antibodies have, like VHHs, a molecular weight of approximately 13 to approximately 16 kDa and, if derived from fully human sequences, do not require humanization for e.g. therapeutical use in humans. As in the case of VHH domains, they are well expressed also in prokaryotic expression systems, providing a significant reduction in overall manufacturing cost.

Furthermore, it will also be clear to the skilled person that it is possible to “graft” one or more of the CDR's mentioned above onto other “scaffolds”, including but not limited to human scaffolds or non-immunoglobulin scaffolds. Suitable scaffolds and techniques for such CDR grafting are known in the art.

The terms “epitope” and “antigenic determinant”, which can be used interchangeably, refer to the part of a macromolecule, such as a polypeptide, that is recognized by antigen-binding molecules, such as conventional antibodies or the polypeptides of the invention, and more particularly by the antigen-binding site of said molecules. Epitopes define the minimum binding site for an immunoglobulin, and thus represent the target of specificity of an immunoglobulin.

A polypeptide (such as an immunoglobulin, an antibody, an immunoglobulin single variable domain of the invention, or generally an antigen-binding molecule or a fragment thereof) that can “bind to” or “specifically bind to”, that “has affinity for” and/or that “has specificity for” a certain epitope, antigen or protein (or for at least one part, fragment or epitope thereof) is said to be “against” or “directed against” said epitope, antigen or protein or is a “binding” molecule with respect to such epitope, antigen or protein. In this context, a DII4-binding component may also be referred to as “DII4-neutralizing”.

Generally, the term “specificity” refers to the number of different types of antigens or epitopes to which a particular antigen-binding molecule or antigen-binding protein (such as an immunoglobulin single variable domain of the invention) molecule can bind. The specificity of an antigen-binding molecule can be determined based on its affinity and/or avidity. The affinity, represented by the equilibrium constant for the dissociation of an antigen with an antigen-binding protein (KD), is a measure for the binding strength between an epitope and an antigen-binding site on the antigen-binding protein: the lesser the value of the KD, the stronger the binding strength between an epitope and the antigen-binding molecule (alternatively, the affinity can also be expressed as the affinity constant (KA), which is 1/KD). As will be clear to the skilled person (for example on the basis of the further disclosure herein), affinity can be determined in a manner known per se, depending on the specific antigen of interest. Avidity is the measure of the strength of binding between an antigen-binding molecule (such as an immunoglobulin, an antibody, an immunoglobulin single variable domain or a polypeptides containing it and the pertinent antigen. Avidity is related to both the affinity between an epitope and its antigen binding site on the antigen-binding molecule and the number of pertinent binding sites present on the antigen-binding molecule.

The part of an antigen-binding molecule that recognizes the epitope is called a paratope.

Unless indicated otherwise, the term “DII4-binding molecule” or “Ang2-binding molecule” includes anti-DII4 or anti-Ang2 antibodies, anti-DII4 antibody or anti-Ang2 antibody fragments, “anti-DII4 antibody-like molecules” or “anti-Ang2 antibody-like molecules”, as defined herein, and conjugates with any of these. Antibodies include, but are not limited to, monoclonal and chimerized monoclonal antibodies. The term “antibody” encompasses complete immunoglobulins, like monoclonal antibodies produced by recombinant expression in host cells, as well as antibody fragments or “antibody-like molecules”, including single-chain antibodies and linear antibodies, so-called “SMIPs” (“Small Modular Immunopharmaceuticals”), as e.g described in WO 02/056910; Antibody-like molecules include immunoglobulin single variable domains, as defined herein. Other examples for antibody-like molecules are immunoglobulin super family antibodies (IgSF), or CDR-grafted molecules.

“Ang2-binding molecule” or “DII4-binding molecule” respectively, refers to both monovalent target-binding molecules (i.e. molecules that bind to one epitope of the respective target) as well as to bi- or multivalent binding molecules (i.e. binding molecules that bind to more than one epitope, e.g. “biparatopic” molecules as defined hereinbelow). Ang2(or DII4)-binding molecules containing more than one Ang2(or DII4)-binding immunoglobulin single variable domain are also termed “formatted” binding molecules, they may, within the target-binding component, in addition to the immunoglobulin single variable domains, comprise linkers and/or moieties with effector functions, e.g. half-life-extending moieties like albumin-binding immunoglobulin single variable domains, and/or a fusion partner like serum albumin and/or an attached polymer like PEG.

The term “biparatopic Ang2(or DII4)-binding molecule” or “biparatopic immunoglobulin single variable domain” as used herein shall mean a binding molecule comprising a first immunoglobulin single variable domain and a second immunoglobulin single variable domain as herein defined, wherein the two molecules bind to two non-overlapping epitopes of the respective antigen. The biparatopic binding molecules are composed of immunoglobulin single variable domains which have different specificities with respect to the epitope. The part of an antigen-binding molecule (such as an antibody or an immunoglobulin single variable domain of the invention) that recognizes the epitope is called a paratope.

A formatted binding molecule may, albeit less preferred, also comprise two identical immunoglobulin single variable domains or two different immunoglobulin single variable domains that recognize the same or overlapping epitopes or their respective antigen. In this case, with respect to VEGF, the two immunoglobulin single variable domains may bind to the same or an overlapping epitope in each of the two monomers that form the VEGF dimer.

Typically, the binding molecules of the invention will bind with a dissociation constant (K_{D}) of 10E-5 to 10E-14 moles/liter (M) or less, and preferably 10E-7 to 10E-14 moles/liter (M) or less, more preferably 10E-8 to 10E-14 moles/liter, and even more preferably 10E-11 to 10E-13, as measured e.g. in a Biacore or in a Kinexa assay), and/or with an association constant (K_{A}) of at least 10E7 ME-1, preferably at least 10E8 ME-1, more preferably at least 10E9 ME-1, such as at least 10E11 ME-1. Any K_{D} value greater than 10E-4 M is generally considered to indicate non-specific binding. Preferably, a polypeptide of the invention will bind to the desired antigen, i.e. VEGF or DII4, respectively, with a K_{D} less than 500 nM, preferably less than 200 nM, more preferably less than 10 nM, such as less than 500 pM. Specific binding of an antigen-binding protein to an antigen or epitope can be determined in any suitable manner known per se, including, for example, the assays described herein, Scatchard analysis and/or competitive binding assays, such as radioimmunoassays (RIA), enzyme immunoassays (EIA) and sandwich competition assays, and the different variants thereof known per se in the art.

Amino acid residues will be indicated according to the standard three-letter or one-letter amino acid code, as generally known and agreed upon in the art. When comparing two amino acid sequences, the term “amino acid difference” refers to insertions, deletions or substitutions of the indicated number of amino acid residues at a position of the reference sequence, compared to a second sequence. In case of substitution(s), such substitution(s) will preferably be conservative amino acid substitution(s), which means that an amino acid residue is replaced with another amino acid residue of similar chemical structure and which has little or essentially no influence on the function, activity or other biological properties of the polypeptide. Such conservative amino acid substitutions are well known in the art, for example from WO 98/49185, wherein conservative amino acid substitutions preferably are substitutions in which one amino acid within the following groups (i)-(v) is substituted by another amino acid residue within the same group: (i) small aliphatic, nonpolar or slightly polar residues: Ala, Ser, Thr, Pro and Gly; (ii) polar, negatively charged residues and their (uncharged) amides: Asp, Asn, Glu and Gln; (iii) polar, positively charged residues: His, Arg and Lys; (iv) large aliphatic, nonpolar residues: Met, Leu, Ile, Val and Cys; and (v) aromatic residues:

Phe, Tyr and Trp. Particularly preferred conservative amino acid substitutions are as follows: Ala into Gly or into Ser; Arg into Lys; Asn into Gin or into His; Asp into Glu; Cys into Ser; Gin into Asn; Glu into Asp; Gly into Ala or into Pro; His into Asn or into Gln; Ile into Leu or into Val; Leu into Ile or into Val; Lys into Arg, into Gin or into Glu; Met into Leu, into Tyr or into Ile; Phe into Met, into Leu or into Tyr; Ser into Thr; Thr into Ser; Trp into Tyr; Tyr into Trp or into Phe; Val into Ile or into Leu.

A polypeptide or nucleic acid molecule is considered to be “(in) essentially isolated (form)”—for example, when compared to its native biological source and/or the reaction medium or cultivation medium from which it has been obtained—when it has been separated from at least one other component with which it is usually associated in said source or medium, such as another protein/polypeptide, another nucleic acid, another biological component or macromolecule or at least one contaminant, impurity or minor component. In particular, a polypeptide or nucleic acid molecule is considered “essentially isolated” when it has been purified at least 2-fold, in particular at least 10-fold, more in particular at least 100-fold, and up to 1000-fold or more. A polypeptide or nucleic acid molecule that is “in essentially isolated form” is preferably essentially homogeneous, as determined using a suitable technique, such as a suitable chromatographical technique, such as polyacrylamide gel electrophoresis.

“Sequence identity” between two DII14-binding molecule sequences or between two Ang2-binding molecule sequences indicates the percentage of amino acids that are identical between the sequences. It may be calculated or determined as described in paragraph f) on pages 49 and 50 of WO 2008/020079. “Sequence similarity” indicates the percentage of amino acids that either are identical or that represent conservative amino acid substitutions.

Alternative methods for numbering the amino acid residues of V_{H} domains, which to methods can also be applied in an analogous manner to VHH domains, are known in the art. However, in the present description, claims and figures, the numbering according to Kabat and applied to VHH domains as described above will be followed, unless indicated otherwise.

An “affinity-matured” DII-4-binding molecule or Ang2-binding molecule, in particular a VHH or a domain antibody, has one or more alterations in one or more CDRs which result in an improved affinity for DII4 or Ang2, as compared to the respective parent DII4-binding molecule or Ang2-binding molecule. Affinity-matured DII4-binding molecules or Ang2-binding molecules of the invention may be prepared by methods known in the art, for example, as described by Marks et al., 1992, Biotechnology 10:779-783, or Barbas, et al., 1994, Proc. Nat. Acad. Sci, USA 91: 3809-3813.; Shier et al., 1995, Gene 169:147-155; Yelton et al., 1995, Immunol. 155: 1994-2004; Jackson et al., 1995, J. Immunol. 154(7):3310-9; and Hawkins et al., 1992, J. Mol. Biol. 226(3): 889 896; KS Johnson and RE Hawkins, “Affinity maturation of antibodies using phage display”, Oxford University Press 1996.

For the present invention, an “amino acid sequences of SEQ ID NO: x”: includes, if not otherwise stated, an amino acid sequence that is 100% identical with the sequence shown in the respective SEQ ID NO: x;
a) amino acid sequences that have at least 80% amino acid identity with the sequence shown in the respective SEQ ID NO: x;
b) amino acid sequences that have 3, 2, or 1 amino acid differences with the sequence shown in the respective SEQ ID NO: x.

The terms “cancer” and “cancerous” refer to or describe the physiological condition in mammals that is typically characterized by unregulated cell growth/proliferation. Examples of cancer to be treated with a bispecific binding molecule of the invention, include but are not limited to carcinoma, lymphoma, blastoma, sarcoma, and leukemia. More particular examples of such cancers, as suggested for treatment with DII4 antagonists in US 2008/0014196, include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian to cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, gastric cancer, melanoma, and various types of head and neck cancer. Dysregulation of angiogenesis can lead to many disorders that can be treated by compositions and methods of the invention. These disorders include both non-neoplastic and neoplastic conditions. Neoplasties include but are not limited those described above.

Non-neoplastic disorders include, but are not limited to, as suggested for treatment with DII4 antagonists in US 2008/0014196, undesired or aberrant hypertrophy, arthritis, rheumatoid arthritis (RA), psoriasis, psoriatic plaques, sarcoidosis, atherosclerosis, atherosclerotic plaques, diabetic and other proliferative retinopathies including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, chronic inflammation, lung inflammation, acute lung injury/ARDS, sepsis, primary pulmonary hypertension, malignant pulmonary effusions, cerebral edema (e.g., associated with acute stroke/closed head injury/trauma), synovial inflammation, pannus formation in RA, myositis ossificans, hypertropic bone formation, osteoarthritis (OA), refractory ascites, polycystic ovarian disease, endometriosis, 3^{rd} spacing of fluid diseases (pancreatitis, compartment syndrome, burns, bowel disease), uterine fibroids, premature labor, chronic inflammation such as IBD (Crohn's disease and ulcerative colitis), renal allograft rejection, inflammatory bowel disease, nephrotic syndrome, undesired or aberrant tissue mass growth (non-cancer), hemophilic joints, hypertrophic scars, inhibition of hair growth, Osier-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

### DETAILED DESCRIPTION OF THE INVENTION

In a first aspect, the present invention relates to a bispecific binding molecule to comprising at least one DII4-binding component and at least one Ang2-binding component.

In a preferred embodiment, the present invention relates to a bispecific binding molecule comprising at least one DII4-binding component and at least one Ang2-binding component which further comprises at least a further binding component, preferably a serum albumin binding component (serum albumin binding molecule).

In a preferred embodiment, the serum albumin binding component of the binding molecule of the present invention is an isolated immunoglobulin single variable domain or a polypeptide containing one or more of said immunoglobulin single variable domains, wherein said immunoglobulin single variable domain consists of four framework regions and three complementarity determining regions CDR1, CDR2 and CDR3, respectively, and wherein said CDR3 has an amino acid sequence selected from amino acid sequences shown in SEQ ID NOs: 522, 525, 528, 531, 534, 537, or 540.

More preferably, said one or more immunoglobulin single variable domain of the serum albumin binding component contain
a. a CDR3 with an amino acid sequence selected from a first group of amino acid sequences shown in SEQ ID NOs: SEQ IDs NOs: 522, 525, 528, 531, 534, 537, or 540;
b. a CDR1 with an amino acid sequences selected from a second group of amino acid sequences shown SEQ ID NOs: 520, 523, 526; 529, 532, 535, or 538;
c. a CDR2 with an amino acid sequences selected from a second group of amino acid sequences shown SEQ ID NOs: 521, 524, 527, 530, 533, 536, or 539.

In a more preferred embodiment, said one or more immunoglobulin single variable domains of the serum albumin binding component are VHHs, preferably having an amino acid sequence shown in SEQ ID NOs: 98 or 519.

According to preferred embodiments, said DII4-binding component and said Ang2-binding component comprise at least one DII4-binding immunoglobulin single variable domain and at least one Ang2-binding immunoglobulin single variable domain, respectively.

In a preferred aspect, said DII4-binding component and said Ang2-binding component each comprise at least one Ang2-binding immunoglobulin single variable domain and at least one DII4-binding immunoglobulin single variable domain, respectively, wherein each of said immunoglobulin single variable domains has four framework regions and three complementarity determining regions CDR1, CDR2 and CDR3, respectively.

Thus, the anti-DII4 and/or the anti-Ang2 component contained in the bispecific binding molecules of the invention may include two (or more) anti-DII4 (or anti-Ang2, respectively) immunoglobulin single variable domains, wherein the immunoglobulin single variable domains are directed against different epitopes within the DII4 (or Ang2) target. Thus, the two immunoglobulin single variable domains in a bispecific binding molecule will have different antigen specificity and therefore different CDR sequences.

Such bivalent binding molecules are also named “biparatopic single domain antibody constructs” (if the immunoglobulin single variable domains consist or essentially consist of single domain antibodies), or “biparatopic VHH constructs” (if the immunoglobulin single variable domains consist or essentially consist of VHHs), respectively, as the two immunoglobulin single variable domains will include two different paratopes.

In the bispecific binding molecule of the invention, one or both of the binding molecules may be bivalent; e.g. the Ang2-binding component may be biparatopic and the DII4-binding component may be one immunoglobulin single variable domain, or the Ang2-binding component may be one immunoglobulin single variable domain and the DII4-binding component may be biparatopic.

In bispecific binding molecules of the invention, it is preferably the Ang2-binding component that contains a bivalent Ang2-binding immunoglobulin single variable domain, e.g. a biparatopic VHH.

The DII4-binding component comprises at least a variable domain with four framework regions and three complementarity determining regions CDR1, CDR2 and CDR3, respectively, wherein said CDR3 has an amino acid sequence selected from amino acid sequences shown in
a) SEQ ID NOs: 1 to 166 and 458,
b) SEQ ID NOs: 333 to 353, or
c) SEQ ID NOs: 375 to 395.

An amino acid sequence a), selected from a first group of SEQ ID NOs: 1 to 166 and 458, is contained as partial sequence in a corresponding amino acid sequence selected from a second group of sequences shown in Table 5 and in SEQ ID NO: 167 to 332 and 459.

An amino acid sequence b), selected from a first group of SEQ ID NOs: 333 to 353, is contained as partial sequence in a corresponding sequence selected from a second group of sequences shown in Table 16-A and in SEQ ID NOs: 354 to 374.

An amino acid sequence c) selected from a first group of SEQ ID NOs: 375 to 395 is contained as partial sequence in a corresponding sequence selected from a second group of sequences shown in Table 16-B and in SEQ ID NOs: 396 to 416.

In a second aspect, said DII4-binding component is an isolated immunoglobulin single variable domain or a polypeptide containing one or more of said immunoglobulin single variable domains, wherein said immunoglobulin single variable domain consists of four framework regions and three complementarity determining regions CDR1, CDR2 and CDR3, respectively, and wherein said CDR3 has an amino acid sequence selected from amino acid sequences shown in
a) SEQ ID NOs: 1 to 166 and 458,
b) SEQ ID NOs: 333 to 353, or
c) SEQ ID NOs: 375 to 395.

In a further aspect, said immunoglobulin single variable domain of the DII4-binding component contains
a) a CDR3 with an amino acid sequence selected from a first group of amino acid sequences shown in SEQ ID NOs: 1 to 166 and 458;
b) a CDR1 and a CDR2 with an amino acid sequence that is contained, as indicated in Table 5, as partial sequence in a sequence selected from a second group of amino acid sequences shown in SEQ ID NOs: 167 to 332 and 459;
wherein a SEQ ID NO: x of said first group, for SEQ ID Nos 1-166: corresponds to SEQ ID NO: y of said second group in that y=x+166.
In a further aspect said immunoglobulin single variable domain contains
a) a CDR3 with an amino acid sequence selected a said first group of amino acid sequences shown in SEQ ID NOs: 333 to 353;
b) a CDR1 and a CDR2 with an amino acid sequence that is contained, as indicated in Table 16-A, as a partial sequence in a sequence selected from a second group of sequences shown in SEQ ID NOs: 354 to 374;

wherein a SEQ ID NO: x of said first group corresponds to SEQ ID NO: y of said second group in that y=x+21.

In a further aspect said immunoglobulin single variable domain has
a) a CDR3 with an amino acid sequence selected a said first group of amino acid sequences shown in SEQ ID NOs: 375 to 395;
b) a CDR1 and a CDR2 with an amino acid sequence that is contained, as indicated in Table 16-B, as a partial sequence in a sequence selected from a second group of sequences shown in SEQ ID NOs: 396 to 416;

wherein a SEQ ID NO: x of said first group corresponds with SEQ ID NO: y of said second group in that y=x+21.

In a preferred embodiment, the immunoglobulin single variable domain is a VHH.

In a further aspect, the VHH has an amino acid sequence selected from amino acid sequences shown in Table 5 and in SEQ ID NOs: 167 to 332 and 459.

The Ang2-binding component comprises at least a variable domain with four framework regions and three complementarity determining regions CDR1, CDR2 and CDR3, respectively, wherein said CDR3 has an amino acid sequence selected from amino acid sequences shown in SEQ ID NOs: 491, 494, 497, 500, 503, 506, 509, 512, 515, or 518.

In a second aspect, said Ang2-binding component is an isolated immunoglobulin single variable domain or a polypeptide containing one or more of said immunoglobulin single variable domains, wherein said immunoglobulin single variable domain consists of four framework regions and three complementarity determining regions CDR1, CDR2 and CDR3, respectively, and wherein said CDR3 has an amino acid sequence selected from amino acid sequences shown in SEQ ID NOs: 491, 494, 497, 500, 503, 506, 509, 512, 515, or 518

In a further aspect, said immunoglobulin single variable domain of the Ang2-binding component contains
a. a CDR3 with an amino acid sequence selected from a first group of amino acid sequences shown in SEQ ID NOs: SEQ IDs NOs: 491, 494, 497, 500, 503, 506, 509, 512, 515, or 518 (see also Table 36);
b. a CDR1 with an amino acid sequences that is contained, as indicated in Table 22-A or 28, as partial sequence in a sequence selected from a second group of amino acid sequences shown SEQ ID NOs: 489, 492, 495, 498, 501, 504, 507, 510, 513, or 516 (see also Table 36);
c. a CDR2 with an amino acid sequences that is contained, as indicated in Table 22-A or 28, as partial sequence in a sequence selected from a second group of amino acid sequences shown SEQ ID NOs: 490, 493, 496, 499, 502, 505, 508, 511, 514, or 517 (see also Table 36).

Preferably; the immunoglobulin single variable domain of the Ang2-binding component is a VHH, preferably having amino acid sequence selected from amino acid sequences shown in SEQ ID NOs: 479, 480, 481, 482, 483, 484, 485, 486, 487, or 488.

In another preferred embodiment, the immunoglobulin single variable domain of the Ang2-binding component has been obtained by affinity maturation or humanization of an immunoglobulin single variable domain as described herein.

Similarly, the present invention also relates to a VHH which has been obtained by affinity maturation or humanization of a VHH of the Ang2-binding component as described herein.

The present invention thus also relates to an Ang2-binding VHH with an amino acid sequence selected from acid sequences shown in SEQ ID NOs: 479, 480, 481, 482, 483, 484, 485, 486, 487, or 488.

DII4- and/or Ang2-binding components with improved properties in view of therapeutic application, e.g. enhanced affinity or decreased immunogenicity, may be obtained from individual DII4- or Ang2-binding components of the invention by techniques such as affinity maturation (for example, starting from synthetic, random or naturally occurring immunoglobulin sequences), CDR grafting, humanizing, combining fragments derived from different immunoglobulin sequences, PCR assembly using overlapping primers, and similar techniques for engineering immunoglobulin sequences well known to the skilled person; or any suitable combination of any of the foregoing. Reference is, for example, made to standard handbooks, as well as to the further description and Examples.

Preferably, a DII4-binding component of the invention with increased affinity is obtained by affinity-maturation of another DII4-binding component, the latter representing, with respect to the affinity-matured molecule, the “parent” DII4-binding component. The same holds true for the Ang2-binding component.

Thus, in yet another preferred embodiment, a DII4- or Ang2-binding molecule of the invention is an immunoglobulin single variable domain that has been obtained by affinity maturation of a parent immunoglobulin single variable domain defined above.

In yet another preferred embodiment, the invention relates to an immunoglobulin single variable domain obtained by affinity-maturation of a VHH.

Suitable parent DII4-binding components for affinity maturation are, by way of example, the above-described VHHs with amino acid sequences shown in SEQ ID NOs: 167 to 332 and 459.

Suitable parent Ang2-binding components for affinity maturation are, by way of example, the above-described VHHs with amino acid sequences shown in SEQ ID NOs: 479, 480, 481, 482, 483, or 484.

Accordingly, the invention also relates to Ang2-binding molecules that have been obtained by affinity maturation and/or sequence optimization of an above-defined VHH, e.g. to a VHH that has been obtained by sequence optimization of a VHH having an amino acid sequence shown as SEQ ID NOs: 482, 483, 484, 485, 486, 487, 488. The “source” amino acid sequences that were used to generate the latter VHHs are shown in SEQ ID NOs: 479, 480, or 481. Also these amino acid sequences are suitable Ang2-binding components that can be applied in the binding molecules of the present invention.

As described herein, the binding molecule of the present invention preferably comprises at least one serum albumin binding component. Particularly preferred binding molecules thus have at least one DII4-binding component, at least one Ang2-binding component and at least one serum albumin binding component. The order of these three binding components could be any possible order such as the order set out in FIG. 16 or 23, e.g., the DII4-, Ang2- or serum albumin binding component can be N-terminal or C-terminal. Notably, “00042”, “00045” or “00050” as referred to in the legend of FIG. 16 stand for Ang2-binding components, while “00018” stands for a DII4-binding component and “ALB11” stands for a serum albumin binding component. None of them is to be construed to a specific sequence, but stands for a Ang2-, DII4- and serum albumin binding component in general when used in the context of possible set-ups of binding molecules of the present invention.

However, it is preferred that the serum albumin binding component is in between the DII4- and Ang2-binding component (or vice versa), while it is particularly preferred that at least one Ang2-binding component is N-terminal, followed by at least one serum albumin binding component, followed by at least one DII4-binding component at the C-Terminus. This set-up is shown to be specifically useful.

The present invention relates thus in a preferred aspect to binding molecules comprising at least one DII4-binding component, at least one Ang2-binding component and at least one serum albumin binding component having an amino acid sequence selected from the amino acid sequences shown in SEQ ID NOs: 460-478.

“At least one” binding component (Ang2, DII4 or serum albumin) when used herein includes that a binding molecule of the present invention may contain one, two, three, four or five Ang2-, DII4, and/or serum albumin binding components (i.e., entities/units) which are preferably represented by an immunoglobulin singly variable domain as described herein.

In yet another preferred embodiment, the invention relates to a DII4 immunoglobulin single variable domain that has been obtained by affinity maturation of a VHH with an amino acid sequence shown in SEQ ID NO: 197.

In yet another embodiment, said immunoglobulin single variable domain that is derived from a VHH with the amino acid sequence shown in SEQ ID NO: 197 is selected from immunoglobulin single variable domains with amino acid sequences shown in SEQ ID NOs: 354 to 374.

In a preferred embodiment, the immunoglobulin single variable domain is a VHH with an amino acid sequence shown in SEQ ID NO: 358.

In an even more preferred embodiment, the immunoglobulin single variable domain has been obtained by humanization of a VHH with an amino acid sequence shown in SEQ ID NO: 358.

In another preferred embodiment, the immunoglobulin single variable domain is a VHH with an amino acid sequence shown in SEQ ID NO: 356.

In an even more preferred embodiment, the invention relates to an immunoglobulin single variable domain that has been obtained by humanization of a VHH with an amino acid sequence shown in SEQ ID NO: 356.

In yet another preferred embodiment, the invention relates to an immunoglobulin single variable domain that has been obtained by affinity maturation of a VHH with an amino acid sequence shown in SEQ ID NO: 224.

In yet another embodiment, said immunoglobulin single variable domain derived from a VHH with the amino acid sequence shown in SEQ ID NO: 224 is selected from immunoglobulin single variable domains with amino acid sequences shown in SEQ ID NOs: 396 to 416.

In another preferred embodiment, the immunoglobulin single variable domain is a VHH with an amino acid sequence shown in SEQ ID NO: 402.

In an even more preferred embodiment, the immunoglobulin single variable domain has been obtained by humanization of the VHH with the amino acid sequence shown in SEQ ID NO: 402.

In another preferred embodiment, the immunoglobulin single variable domain is a VHH with an amino acid sequence shown in SEQ ID NO: 416.

In an even more preferred embodiment, the immunoglobulin single variable domain has been obtained by humanization of the immunoglobulin single variable domain with the amino acid sequence shown in SEQ ID NO: 416

In another preferred embodiment, the immunoglobulin single variable domain is a VHH with an amino acid sequence shown in SEQ ID NO: 407.

In an even more preferred embodiment, the immunoglobulin single variable domain has been obtained by humanization of the immunoglobulin single variable domain with the amino acid sequence shown in SEQ ID NO: 413.

According to another embodiment, the immunoglobulin single variable domain is a VH domain, as defined herein.

In yet another embodiment, the representatives of the class of DII4- and/or Ang2-binding immunoglobulin single variable domains of the invention or present in the polypeptides of the invention have amino acid sequences that correspond to the amino acid sequence of a naturally occurring VH domain that has been “camelized”, i.e. by replacing one or more amino acid residues in the amino acid sequence of a naturally occurring variable heavy chain from a conventional 4-chain antibody by one or more amino acid residues that occur at the corresponding position(s) in a VHH domain of a heavy chain antibody. This can be performed in a manner known per se, which will be clear to the skilled person, and reference is additionally be made to WO 1994/04678. Such camelization may preferentially occur at amino acid positions which are present at the VH-VL interface and at the so-called Camelidae Hallmark residues (see for example also WO 1994/04678). A detailled description of such “humanization” and “camelization” techniques and preferred framework region sequences consistent therewith can additionally be taken from e.g. pp. 46 and pp. 98 of WO 2006/040153 and pp. 107 of WO 2006/122786.

The DII4- or Ang2-binding components of the invention, e.g. immunoglobulin single variable domains and or polypeptides containing them, have specificity for DII4 or Ang2, respectively, in that they comprise one or more immunoglobulin single variable to domains specifically binding to one or more epitopes within the DII4 or Ang2 molecule, respectively.

Specific binding of an DII4- and/or Ang2 binding component to its antigen DII4 or Ang2, respectively, can be determined in any suitable manner known per se, including, for example, the assays described herein, Scatchard analysis and/or competitive binding assays, such as radioimmunoassays (RIA), enzyme immunoassays (EIA and ELISA) and sandwich competition assays, and the different variants thereof known per se in the art.

With regard to the antigen DII4, a DII4-binding component of the invention, e.g. an immunoglobulin single variable domain, is not limited with regard to the species. Thus, the immunoglobulin single variable domains of the invention or polypeptides containing them preferably bind to human DII4, if intended for therapeutic purposes in humans. However, immunoglobulin single variable domains that bind to DII4 from another mammalian species, or polypeptides containing them, are also within the scope of the invention. An immunoglobulin single variable domain of the invention binding to one species form of DII4 may cross-react with DII4 from one or more other species. For example, immunoglobulin single variable domains of the invention binding to human DII4 may exhibit cross reactivity with DII4 from one or more other species of primates and/or with DII4 from one or more species of animals that are used in animal models for diseases, for example monkey (in particular Cynomolgus or Rhesus), mouse, rat, rabbit, pig, dog or) and in particular in animal models for diseases and disorders associated with DII4-mediated effects on angiogenesis (such as the species and animal models mentioned herein). Immunoglobulin single variable domains of the invention that show such cross-reactivity are advantageous in a research and/or drug development, since it allows the immunoglobulin single variable domains of the invention to be tested in acknowledged disease models such as monkeys, in particular Cynomolgus or Rhesus, or mice and rats. The same is true for Ang2.

Also, the DII4-binding components of the invention are not limited to or defined by a specific domain or an antigenic determinant of DII4 against which they are directed. Preferably, in view of cross-reactivity with one or more DII4 molecules from species other than human that is/are intended for use as an animal model during development of a therapeutic DII4 antagonist, a DII4-binding component recognizes to an epitope in a region of the DII4 of interest that has a high degree of identity with human DII4. By way of example, in view of using a mouse model, an immunoglobulin single variable domain of the invention recognizes an epitope which is, totally or in part, located within the EGF-2 domain, which shows a high identity between human and mouse. The same is true for Ang2.

Therefore, according to a preferred embodiment, the invention relates to a DII4-binding component, in particular an immunoglobulin single variable domain or a polypeptide containing same, wherein said immunoglobulin single variable domain is selected from the group that binds to an epitope that is totally or partially contained within the EGF-2 domain that corresponds to amino acid residues 252-282 of SEQ ID NO: 417.

If a polypeptide of the invention is a biparatopic molecule as defined herein, which contains more than one immunoglobulin single variable domain of the invention, at least one of the immunoglobulin single variable domain components binds to the epitope within the EGF-2 domain, as defined above.

Preferably, an immunoglobulin single variable domain of the invention binds to DII4 and/or Ang2 with an affinity less than 500 nM, preferably less than 200 nM, more preferably less than 10 nM, such as less than 500 pM (as determined by Surface Plasmon Resonance analysis, as described in Example 5.7).

Preferably, the immunoglobulin single variable domains of the invention have IC₅₀ values, as measured in a competition ELISA assay as described in Example 5.1. in the range of 10⁻⁶ to 10⁻¹⁰ moles/litre or less, more preferably in the range of 10⁻⁸ to 10⁻¹⁰ moles/litre or less and even more preferably in the range of 10⁻⁹ to 10⁻¹⁰ moles/litre or less.

According to a non-limiting but preferred embodiment of the invention, DII4-binding immunoglobulin single variable domains of the invention or polypeptides containing them bind to DII4 with an dissociation constant (KD) of 10⁻⁸ to 10⁻¹² moles/liter (M) or less, and preferably 10⁻⁷ to 10⁻¹² moles/liter (M) or less and more preferably 10⁻⁸ to 10⁻¹² moles/liter (M), and/or with an association constant (K_{A}) of at least 10⁷ M⁻¹, preferably at least 10⁸ M⁻¹, more preferably at least 10⁹ M⁻¹, such as at least 10¹² M⁻¹; and in particular with a K_{D} less than 500 nM, preferably less than 200 nM, more preferably less than 10 nM, such as less than 500 pM. The K_{D} and K_{A} values of the immunoglobulin single variable domain of the invention against DII4 can be determined. The same is true for Ang2.

In a further embodiment, the invention relates to DII4-binding components comprising two or more immunoglobulin single variable domains that bind to the antigen DII4 or Ang2, respectively, at different non-overlapping epitopes. More specifically, such polypeptide of the invention essentially consists of or comprises (i) a first immunoglobulin single variable domain specifically binding to a first epitope of DII4 or Ang2, respectively, and (ii) a second immunoglobulin single variable domain specifically binding to a second epitope of DII4 or Ang2, respectively, wherein the first epitope of DII4/Ang2 and the second epitope of DII4/Ang2 are not identical epitopes. In other words, such polypeptide of the invention comprises or essentially consists of two or more immunoglobulin single variable domains that are directed against at least two different epitopes present in DII4/Ang2, wherein said immunoglobulin single variable domains are linked to each other in such a way that they are capable of simultaneously binding DII4/Ang2. In this sense, the polypeptide of the invention can also be regarded as a “bivalent” or “multivalent” immunoglobulin construct, and especially as a “multivalent immunoglobulin single variable domain construct”, in that the polypeptide contains at least two binding sites for DII4/Ang2.

Such DII4-binding component of the invention includes (at least) two anti-DII4 immunoglobulin single variable domains, wherein (the) two immunoglobulin single variable domains are directed against different epitopes within the DII4 molecule. Thus, these two immunoglobulin single variable domains will have a different antigen specificity and therefore different CDR sequences. For this reason, such polypeptides of the invention will herein also be named “biparatopic polypeptides”, or “biparatopic single domain antibody constructs” (if the immunoglobulin single variable domains consist or essentially consist of single domain antibodies), or “biparatopic VHH constructs” (if the immunoglobulin single variable domains consist or essentially consist of VHHs), respectively, as the two immunoglobulin single variable domains will include two different paratopes. The same is true for Ang2, mutatis mutandis.

According to a specific embodiment of the invention, in case that the polypeptide of the invention includes more than two anti-DII4 immunoglobulin single variable domains, i.e. three, four or even more anti-DII4 immunoglobulin single variable domains, at least two of the anti-DII4 immunoglobulin single variable domains are directed against different epitopes within the DII4 molecule, wherein any further immunoglobulin single variable domain may bind to any of these two different epitopes and/or a further epitope present in the DII4 molecule. The same is true for Ang2, mutatis mutandis.

According to the invention, the two or more immunoglobulin single variable domains can be, independently of each other, VHs or VHHs, and/or any other sort of immunoglobulin single variable domains, such as VL domains, as defined herein, provided that these immunoglobulin single variable domains will bind the antigen, i.e. DII4 or Ang2, respectively.

The detailed description of the binding components is primarily provided for the DII4-binding component. However, all features and options outlined herein for the DII4-binding component also apply equivalently for the Ang2-binding component, mutatis mutandis.

According to preferred embodiments, the binding molecules present in the bispecific binding molecules (the Ang2-binding molecules within the Ang2-binding component or the DII4-binding molecules within the DII4-binding component or the two adjacent Ang2- and DII4-binding components) may be connected with each other directly (i.e. without use of a linker) or via a linker. The linker is preferably a linker peptide and will be selected so as to allow binding of the two different binding molecules to each of non-overlapping epitopes of the targets, either within one and the same target molecule, or within two different molecules.

In the case of biparatopic binding molecules, selection of linkers within the Ang2- or the DII-4-binding component will inter alia depend on the epitopes and, specifically, the distance between the epitopes on the target to which the immunoglobulin single variable domains bind, and will be clear to the skilled person based on the disclosure herein, optionally after some limited degree of routine experimentation.

Two binding molecules (two VHHs or domain antibodies or VHH and a domain antibody), or two binding components, may be linked to each other via an additional VHH or domain antibody, respectively (in such binding molecules, the two or more immunoglobulin single variable domains may be linked directly to said additional to immunoglobulin single variable domain or via suitable linkers). Such an additional VHH or domain antibody may for example be a VHH or domain antibody that provides for an increased half-life. For example, the latter VHH or domain antibody may be one that is capable of binding to a (human) serum protein such as (human) serum albumin or (human) transferrin.

Alternatively, the two or more immunoglobulin single variable domains that bind to the respective target may be linked in series (either directly or via a suitable linker) and the additional VHH or domain antibody (which may provide for increased half-life) may be connected directly or via a linker to one of these two or more aforementioned immunoglobulin sequences.

Suitable linkers are described herein in connection with specific polypeptides of the invention and may—for example and without limitation—comprise an amino acid sequence, which amino acid sequence preferably has a length of 9 or more amino acids, more preferably at least 17 amino acids, such as about 20 to 40 amino acids. However, the upper limit is not critical but is chosen for reasons of convenience regarding e.g. biopharmaceutical production of such polypeptides.

The linker sequence may be a naturally occurring sequence or a non-naturally occurring sequence. If used for therapeutic purposes, the linker is preferably non-immunogenic in the subject to which the bispecific binding molecule of the invention is administered.

One useful group of linker sequences are linkers derived from the hinge region of heavy chain antibodies as described in WO 1996/34103 and WO 1994/04678.

Other examples are poly-alanine linker sequences such as Ala-Ala-Ala.

Further preferred examples of linker sequences are Gly/Ser linkers of different length such as (glyₓser_{y})_{z} linkers, including (gly₄ser)₃, (gly₄ser)₄, (gly₄ser), (gly₃ser), gly₃, and (gly₃ser₂)₃.

Some non-limiting examples of linkers are shown in FIGS. 40 and 48, e.g. the linkers

(35GS; SEQ ID NO: 90)
GGGGSGGGGSGGGGSGGGGSGGGGSGGGGSGGGGS;
(9GS; SEQ ID NO: 91)
GGGGSGGGS;
(40GS; SEQ ID NO: 92)
GGGGSGGGGSGGGGSGGGGSGGGGSGGGGSGGGGSGGGGS.

If a bispecific binding molecule is modified by the attachment of a polymer, for example of a polyethylene glycol PEG (polyethylene glycol) moiety, the linker sequence preferably includes an amino acid residue, such as a cysteine or a lysine, allowing such modification, e.g. PEGylation, in the linker region.

Examples of linkers useful for PEGylation are:

(“GS9, C5”, SEQ ID NO: 93)
GGGGCGGGS;
(“GS25, C5, SEQ ID NO: 94)
GGGGCGGGGSGGGGSGGGGSGGGGS
(“GS27, C14”, SEQ ID NO: 95)
GGGSGGGGSGGGGCGGGGSGGGGSGGG,
(“GS35,C15”, SEQ ID NO: 96)
GGGGSGGGGSGGGGCGGGGSGGGGSGGGGSGGGGS,
and
(“GS35, C5”, SEQ ID NO: 97)
GGGGCGGGGSGGGGSGGGGSGGGGSGGGGSGGGGS.

Furthermore, the linker may also be a poly(ethylene glycol) moiety, as shown in e.g. WO 2004/081026.

In another embodiment, the immunoglobulin single variable domains are linked to each other via another moiety (optionally via one or two linkers), such as another polypeptide which, in a preferred but non-limiting embodiment, may be a further immunoglobulin single variable domain as described above. Such moiety may either be essentially inactive or may have a biological effect such as improving the desired properties of the polypeptide or may confer one or more additional desired properties to the polypeptide. For example, and without limitation, the moiety may improve the half-life of the protein or polypeptide, and/or may reduce its immunogenicity or improve any other desired property.

According to a preferred embodiment, a bispecific binding molecule of the invention includes, especially when intended for use or used as a therapeutic agent, a moiety to which extends the half-life of the polypeptide of the invention in serum or other body fluids of a patient. The term “half-life” is defined as the time it takes for the serum concentration of the (modified) polypeptide to reduce by 50%, in vivo, for example due to degradation of the polypeptide and/or clearance and/or sequestration by natural mechanisms.

More specifically, such half-life extending moiety can be covalently linked to or fused to an immunoglobulin single variable domain and may be, without limitation, an Fc portion, an albumin moiety, a fragment of an albumin moiety, an albumin binding moiety, such as an anti-albumin immunoglobulin single variable domain, a transferrin binding moiety, such as an anti-transferrin immunoglobulin single variable domain, a polyoxyalkylene molecule, such as a polyethylene glycol molecule, an albumin binding peptide or a hydroxyethyl starch (HES) derivative.

In another embodiment, the bispecific binding molecule of the invention comprises a moiety which binds to an antigen found in blood, such as serum albumin, serum immunoglobulins, thyroxine-binding protein, fibrinogen or transferrin, thereby conferring an increased half-life in vivo to the resulting polypeptide of the invention. According to a specifically preferred embodiment, such moiety is an albumin-binding immunoglobulin and, especially preferred, an albumin-binding immunoglobulin single variable domain such as an albumin-binding VHH domain.

If intended for use in humans, such albumin-binding immunoglobulin single variable domain preferably binds to human serum albumin and preferably is a humanized albumin-binding VHH domain.

Immunoglobulin single variable domains binding to human serum albumin are known in the art and are described in further detail in e.g. WO 2006/122786. Specifically, useful albumin binding VHHs are ALB 1 and its humanized counterpart, ALB 8 (WO 2009/095489). Other albumin binding VHH domains mentioned in the above patent publication may, however, be used as well.

A specifically useful albumin binding VHH domain is ALB8 which consists of or contains the amino acid sequence shown in SEQ ID NO: 98 or 519.

According to a further embodiment of the invention, the two immunoglobulin single variable domains, in preferably VHHs, may be fused to a serum albumin molecule, such as described e.g. in WO01/79271 and WO03/59934. As e.g. described in WO 2001/79271, the fusion protein may be obtained by conventional recombinant technology: a DNA molecule coding for serum albumin, or a fragment thereof, is joined to the DNA coding for the bispecific binding molecule, the obtained construct is inserted into a plasmid suitable for expression in the selected host cell, e.g. a yeast cell like Pichia pastoris or a bacterial cell, and the host cell is then transfected with the fused nucleotide sequence and grown under suitable conditions. The sequence of a useful HSA is shown in SEQ ID NO: 99.

According to another embodiment, a half-life extending modification of a polypeptide of the invention (such modification also reducing immunogenicity of the polypeptide) comprises attachment of a suitable pharmacologically acceptable polymer, such as straight or branched chain poly(ethylene glycol) (PEG) or derivatives thereof (such as methoxypoly(ethylene glycol) or mPEG). Generally, any suitable form of PEGylation can be used, such as the PEGylation used in the art for antibodies and antibody fragments (including but not limited to domain antibodies and scFv's); reference is made, for example, to: Chapman, Nat. Biotechnol., 54, 531-545 (2002); Veronese and Harris, Adv. Drug Deliv. Rev. 54, 453-456 (2003); Harris and Chess, Nat. Rev. Drug. Discov. 2 (2003); and WO 2004/060965.

Various reagents for PEGylation of polypeptides are also commercially available, for example from Nektar Therapeutics, USA, or NOF Corporation, Japan, such as the Sunbright® EA Series, SH Series, MA Series, CA Series, and ME Series, such as Sunbright® ME-100MA, Sunbright® ME-200MA, and Sunbright® ME-400MA.

Preferably, site-directed PEGylation is used, in particular via a cysteine-residue (see for example Yang et al., Protein Engineering 16, 761-770 (2003)). For example, for this purpose, PEG may be attached to a cysteine residue that naturally occurs in a polypeptide of the invention, a polypeptide of the invention may be modified so as to suitably introduce one or more cysteine residues for attachment of PEG, or an amino acid sequence comprising one or more cysteine residues for attachment of PEG may be fused to the N- and/or C-terminus of a polypeptide of the invention, all using techniques of protein engineering known per se to the skilled person.

Preferably, for the polypeptides of the invention, a PEG is used with a molecular to weight of more than 5 kDa, such as more than 10 kDa and less than 200 kDa, such as less than 100 kDa; for example in the range of 20 kDa to 80 kDa.

With regard to PEGylation, its should be noted that generally, the invention also encompasses any bispecific binding molecule that has been PEGylated at one or more amino acid positions, preferably in such a way that said PEGylation either (1) increases the half-life in vivo; (2) reduces immunogenicity; (3) provides one or more further beneficial properties known per se for PEGylation; (4) does not essentially affect the affinity of the polypeptide for its target (e.g. does not reduce said affinity by more than 50%, and more preferably not by more than 10%, as determined by a suitable assay described in the art); and/or (4) does not affect any of the other desired properties of the bispecific binding molecules of the invention. Suitable PEG-groups and methods for attaching them, either specifically or non-specifically, will be clear to the skilled person. Various reagents for PEGylation of polypeptides are also commercially available, for example from Nektar Therapeutics, USA, or NOF Corporation, Japan, such as the Sunbright® EA Series, SH Series, MA Series, CA Series, and ME Series, such as Sunbright® ME-100MA, Sunbright® ME-200MA, and Sunbright® ME-400MA.

According to an especially preferred embodiment of the invention, a PEGylated polypeptide of the invention includes one PEG moiety of linear PEG having a molecular weight of 40 kDa or 60 kDa, wherein the PEG moiety is attached to the polypeptide in a linker region and, specifically, at a Cys residue at position 5 of a GS9-linker peptide as shown in SEQ ID NO: 93, at position 14 of a GS27-linker peptide as shown in SEQ ID NO: 95, or at position 15 of a GS35-linker peptide as shown in SEQ ID NO: 96, or at position 5 of a 35GS-linker peptide as shown in SEQ ID NO: 97.

A bispecific binding molecule of the invention may be PEGylated with one of the PEG reagents as mentioned above, such as “Sunbright®-ME-400MA”, as shown in the following chemical formula:

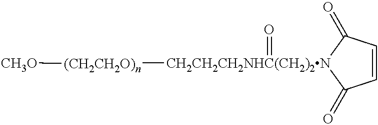

Bispecific binding molecules that contain linkers and/or half-life extending functional groups are shown in SEQ ID NO: 81 and in FIG. 48.

According to another embodiment, the immunoglobulin single variable domains are domain antibodies, as defined herein.

Immunoglobulin single variable domains present in the bispecific binding molecules of the invention may also have sequences that correspond to the amino acid sequence of a naturally occurring VH domain that has been “camelized”, i.e. by replacing one or more amino acid residues in the amino acid sequence of a naturally occurring variable heavy chain from a conventional 4-chain antibody by one or more amino acid residues that occur at the corresponding position(s) in a VHH domain of a heavy chain antibody. This can be performed in a manner known per se, which will be clear to the skilled person, and reference is additionally be made to WO 94/04678. Such camelization may preferentially occur at amino acid positions which are present at the VH-VL interface and at the so-called Camelidae Hallmark residues (see for example also WO-94/04678). A detailled description of such “humanization” and “camelization” techniques and preferred framework region sequences consistent therewith can additionally be taken from e.g. pp. 46 and pp. 98 of WO 2006/040153 and pp. 107 of WO 2006/122786.

The binding components have specificity for Ang2 or DII4, respectively, in that they comprise in a preferred embodiment one or more immunoglobulin single variable domains specifically binding to one or more epitopes within the Ang2 molecule or within the DII4 molecule, respectively.

Specific binding of a binding component to its antigen Ang2 or DII4 can be determined in any suitable manner known per se, including, for example, the assays described herein, Scatchard analysis and/or competitive binding assays, such as radioimmunoassays (RIA), enzyme immunoassays (EIA and ELISA) and sandwich competition assays, and the different variants thereof known per se in the art.

With regard to the antigen Ang2 or DII4, respectively, an immunoglobulin single variable domain is not limited with regard to the species. Thus, the immunoglobulin single variable domains preferably bind to human Ang2 or to human DII4, respectively, if intended for therapeutic purposes in humans. However, immunoglobulin single variable domains that bind to Ang2 or DII4, respectively, from another mammalian species, or polypeptides containing them, are also within the scope of the invention. An immunoglobulin single variable domain binding to one to species form of Ang2 or DII4 may cross-react with the respective antigen from one or more other species. For example, immunoglobulin single variable domains binding to the human antigen may exhibit cross reactivity with the respective antigen from one or more other species of primates and/or with the antigen from one or more species of animals that are used in animal models for diseases, for example monkey (in particular Cynomolgus or Rhesus), mouse, rat, rabbit, pig, dog or) and in particular in animal models for diseases and disorders that can be modulated by inhibition of Ang2 (such as the species and animal models mentioned herein). Immunoglobulin single variable domains of the invention that show such cross-reactivity are advantageous in a research and/or drug development, since it allows the immunoglobulin single variable domains of the invention to be tested in acknowledged disease models such as monkeys, in particular Cynomolgus or Rhesus, or mice and rats.

Also, the binding components are not limited to or defined by a specific domain or an antigenic determinant of the antigen against which they are directed. Preferably, in view of cross-reactivity with one or more antigen molecules from species other than human that is/are intended for use as an animal model during development of a therapeutic Ang2/DII4 antagonist, a binding component recognizes an epitope in a region of the respective antigen that has a high degree of identity with the human antigen. By way of example, in view of using a mouse model, an anti-Ang2 immunoglobulin single variable domain contained in the bispecific binding molecules of the invention recognizes an epitope which is, totally or in part, located within the EGF-2 domain of Ang2, which shows a high identity between human and mouse.

Therefore, according to a preferred embodiment, the bispecific binding molecule of the invention comprises a DII4-binding molecule which is an immunoglobulin single variable domain that is selected from the group that binds to an epitope that is totally or partially contained within the EGF-2 domain that corresponds to amino acid residues 252-282 of SEQ ID NO: 101.

In another aspect, the invention relates to nucleic acid molecules that encode bispecific binding molecules of the invention. Such nucleic acid molecules will also be referred to herein as “nucleic acids of the invention” and may also be in the form of a genetic construct, as defined herein. A nucleic acid of the invention may be genomic DNA, cDNA or synthetic DNA (such as DNA with a codon usage that has been specifically adapted for expression in the intended host cell or host organism). According to one embodiment of the invention, the nucleic acid of the invention is in essentially isolated form, as defined hereabove.

The nucleic acid of the invention may also be in the form of, may be present in and/or may be part of a vector, such as for example a plasmid, cosmid or YAC. The vector may especially be an expression vector, i.e. a vector that can provide for expression of the bispecific binding molecule in vitro and/or in vivo (i.e. in a suitable host cell, host organism and/or expression system). Such expression vector generally comprises at least one nucleic acid of the invention that is operably linked to one or more suitable regulatory elements, such as promoter(s), enhancer(s), terminator(s), and the like. Such elements and their selection in view of expression of a specific sequence in a specific host are common knowledge of the skilled person. Specific examples of regulatory elements and other elements useful or necessary for expressing bispecific binding molecules of the invention, such as promoters, enhancers, terminators, integration factors, selection markers, leader sequences, reporter genes, and the like, are disclosed e.g. on pp. 131 to 133 of WO 2006/040153.

The nucleic acids of the invention may be prepared or obtained in a manner known per se (e.g. by automated DNA synthesis and/or recombinant DNA technology), based on the information on the amino acid sequences for the polypeptides of the invention given herein, and/or can be isolated from a suitable natural source.

In another aspect, the invention relates to host cells that express or that are capable of expressing one or more bispecific binding molecules of the invention; and/or that contain a nucleic acid of the invention. According to a particularly preferred embodiment, said host cells are bacterial cells; other useful cells are yeast cells, fungal cells or mammalian cells.

Suitable bacterial cells include cells from gram-negative bacterial strains such as strains of Escherichia coli, Proteus, and Pseudomonas, and gram-positive bacterial strains such as strains of Bacillus, Streptomyces, Staphylococcus, and Lactococcus. Suitable fungal cell include cells from species of Trichoderma, Neurospora, and Aspergillus. Suitable yeast cells include cells from species of Saccharomyces (for example Saccharomyces cerevisiae), Schizosaccharomyces (for example Schizosaccharomyces pombe), Pichia (for example Pichia pastoris and Pichia methanolica), and Hansenula.

Suitable mammalian cells include for example CHO cells, BHK cells, HeLa cells, COS cells, and the like. However, amphibian cells, insect cells, plant cells, and any other cells used in the art for the expression of heterologous proteins can be used as well.

The invention further provides methods of manufacturing a bispecific binding molecule of the invention, such methods generally comprising the steps of:
culturing host cells comprising a nucleic acid capable of encoding a bispecific binding molecule under conditions that allow expression of the bispecific binding molecule of the invention; and
recovering or isolating the polypeptide expressed by the host cells from the culture; and
optionally further purifying and/or modifying and/or formulating the bispecific binding molecule of the invention.

For production on an industrial scale, preferred host organisms include strains of E. coli, Pichia pastoris, and S. cerevisiae that are suitable for large scale expression, production and fermentation, and in particular for large scale pharmaceutical expression, production and fermentation.

The choice of the specific expression system depends in part on the requirement for certain post-translational modifications, more specifically glycosylation. The production of a bispecific binding molecule of the invention for which glycosylation is desired or required would necessitate the use of mammalian expression hosts that have the ability to glycosylate the expressed protein. In this respect, it will be clear to the skilled person that the glycosylation pattern obtained (i.e. the kind, number and position of residues attached) will depend on the cell or cell line that is used for the expression.

Bispecific binding molecules of the invention may be produced either in a cell as set out above intracellullarly (e.g. in the cytosol, in the periplasma or in inclusion bodies) and then isolated from the host cells and optionally further purified; or they can be produced extracellularly (e.g. in the medium in which the host cells are cultured) and then isolated from the culture medium and optionally further purified.

Methods and reagents used for the recombinant production of polypeptides, such as specific suitable expression vectors, transformation or transfection methods, selection markers, methods of induction of protein expression, culture conditions, and the like, are known in the art. Similarly, protein isolation and purification techniques useful in a method of manufacture of a polypeptide of the invention are well known to the skilled person.

In a further aspect, the invention relates to a peptide having an amino acid sequence of a CDR3 contained in an anti-DII4-VHH having an amino acid sequence selected from sequences shown in SEQ ID NOs: 1 to 166 and 458, SEQ ID NOs: 333 to 353, or SEQ ID NOs: 375 to 395, respectively, and a nucleic acid molecule encoding same.

These peptides correspond to CDR3s derived from the VHHs of the invention. They, in particular the nucleic acid molecules encoding them, are useful for CDR grafting in order to replace a CDR3 in an immunoglobulin chain, or for insertion into a non-immunoglobulin scaffold, e.g. a protease inhibitor, DNA-binding protein, cytochrome b562, a helix-bundle protein, a disulfide-bridged peptide, a lipocalin or an anticalin, thus conferring target-binding properties to such scaffold. The method of CDR-grafting is well known in the art and has been widely used, e.g. for humanizing antibodies (which usually comprises grafting the CDRs from a rodent antibody onto the Fv frameworks of a human antibody).

In order to obtain an immunoglobulin or a non-immunoglobulin scaffold containing a CDR3 of the invention, the DNA encoding such molecule may be obtained according to standard methods of molecular biology, e.g. by gene synthesis, by oligonucleotide annealing or by means of overlapping PCR fragments, as e.g. described by Daugherty et al., 1991, Nucleic Acids Research, Vol. 19, 9, 2471-2476. A method for inserting a VHH CDR3 into a non-immunoglobulin scaffold has been described by Nicaise et al., 2004, Protein Science, 13, 1882-1891.

The invention further relates to a product or composition containing or comprising at least one bispecific binding molecule of the invention and optionally one or more further components of such compositions known per se, i.e. depending on the intended use of the composition.

For pharmaceutical use, a bispecific binding molecule of the invention or a polypeptide containing same may be formulated as a pharmaceutical preparation or composition comprising at least one bispecific binding molecule of the invention and at least one pharmaceutically acceptable carrier, diluent or excipient and/or adjuvant, and optionally one or more further pharmaceutically active polypeptides and/or compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers for use in the preparation thereof, will be clear to the skilled person, and are further described herein.

Thus, in a further aspect, the invention relates to a pharmaceutical composition that contains at least one bispecific binding molecule, in particular one immunoglobulin single variable domain of the invention or a polypeptide containing same and at least one suitable carrier, diluent or excipient (i.e. suitable for pharmaceutical use), and optionally one or more further active substances.

The bispecific binding molecules of the invention may be formulated and administered in any suitable manner known per se: Reference, in particular for the immunoglobulin single variable domains, is for example made to WO 2004/041862, WO 2004/041863, WO 2004/041865, WO 2004/041867 and WO 2008/020079, as well as to the standard handbooks, such as Remington's Pharmaceutical Sciences, 18^{th} Ed., Mack Publishing Company, USA (1990), Remington, the Science and Practice of Pharmacy, 21^{th} Edition, Lippincott Williams and Wilkins (2005); or the Handbook of Therapeutic Antibodies (S. Dubel, Ed.), Wiley, Weinheim, 2007 (see for example pages 252-255).

For example, an immunoglobulin single variable domain of the invention may be formulated and administered in any manner known per se for conventional antibodies and antibody fragments (including ScFv's and diabodies) and other pharmaceutically active proteins. Such formulations and methods for preparing the same will be clear to the skilled person, and for example include preparations suitable for parenteral administration (for example intravenous, intraperitoneal, subcutaneous, intramuscular, intraluminal, intra-arterial or intrathecal administration) or for topical (i.e. transdermal or intradermal) administration.

Preparations for parenteral administration may for example be sterile solutions, suspensions, dispersions or emulsions that are suitable for infusion or injection. Suitable carriers or diluents for such preparations for example include, without limitation, sterile water and pharmaceutically acceptable aqueous buffers and solutions such as physiological phosphate-buffered saline, Ringer's solutions, dextrose solution, and Hank's solution; water oils; glycerol; ethanol; glycols such as propylene glycol or as well as mineral oils, animal oils and vegetable oils, for example peanut oil, soybean oil, as well as suitable mixtures thereof. Usually, aqueous solutions or suspensions will be preferred.

Thus, the bispecific binding molecule of the invention may be systemically administered, e.g., orally, in combination with a pharmaceutically acceptable vehicle such as an inert diluent or an assimilable edible carrier. For oral therapeutic administration, the bispecific binding molecule of the invention may be combined with one or more excipients and used in the form of ingestible tablets, buccal tablets, troches, capsules, elixirs, suspensions, syrups, wafers, and the like. Such compositions and preparations should contain at least 0.1% of the DII4-binding molecule of the invention. Their percentage in the compositions and preparations may, of course, be varied and may conveniently be between about 2 to about 60% of the weight of a given unit dosage form. The amount of the bispecific binding molecule of the invention in such therapeutically useful compositions is such that an effective dosage level will be obtained.

The tablets, pills, capsules, and the like may also contain binders, excipients, disintegrating agents, lubricants and sweetening or flavouring agents, for example those mentioned on pages 143-144 of WO 08/020,079. When the unit dosage form is a capsule, it may contain, in addition to materials of the above type, a liquid carrier, to such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the bispecific binding molecules of the invention, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the bispecific binding molecules of the invention may be incorporated into sustained-release preparations and devices.

Preparations and formulations for oral administration may also be provided with an enteric coating that will allow the constructs of the invention to resist the gastric environment and pass into the intestines. More generally, preparations and formulations for oral administration may be suitably formulated for delivery into any desired part of the gastrointestinal tract. In addition, suitable suppositories may be used for delivery into the gastrointestinal tract.

The bispecific binding molecules of the invention may also be administered intravenously or intraperitoneally by infusion or injection, as further described on pages 144 and 145 of WO 2008/020079.

For topical administration of the bispecific binding molecules of the invention, it will generally be desirable to administer them to the skin as compositions or formulations, in combination with a dermatologically acceptable carrier, which may be a solid or a liquid, as further described on page 145 of WO 2008/020079.

Generally, the concentration of the bispecific binding molecules of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the bispecific binding molecules of the invention required for use in treatment will vary not only with the particular bispecific binding molecule selected, but also with the route of administration, the nature of the condition being treated and the age and condition of the patient and will be ultimately at the discretion of the attendant physician or clinician. Also, the dosage of the bispecific binding molecules to of the invention varies depending on the target cell, tumor, tissue, graft, or organ. The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

An administration regimen may include long-term, daily treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication.

According to a further embodiment, the invention relates to the use of bispecific binding molecules of the invention, e.g. immunoglobulin single variable domains or polypeptides containing them, for therapeutic purposes, such as
for the prevention, treatment and/or alleviation of a disorder, disease or condition, especially in a human being, that is associated with DII4-mediated and/or Ang2 related effects on angiogenesis or that can be prevented, treated or alleviated by modulating the Notch signaling pathway and/or the Tie2 signalling pathway with a bispecific binding molecule according to the invention,
in a method of treatment of a patient in need of such therapy, such method comprising administering, to a subject in need thereof, a pharmaceutically active amount of at least one bispecific binding molecule of the invention, e.g. an immunoglobulin single variable domain, or a pharmaceutical composition containing same;
for the preparation of a medicament for the prevention, treatment or alleviation of disorders, diseases or conditions associated with DII4-mediated and/or Ang2-mediated effects on angiogenesis;
as an active ingredient in a pharmaceutical composition or medicament used for the above purposes.

According to a specific aspect, said disorder, disease or condition is a cancer or cancerous disease, as defined herein.

According to another aspect, the disease is an eye disease associated with associated with DII4-mediated and/or Ang2-mediated effects on angiogenesis or which can be treated or alleviated by modulating the Notch signaling pathway and/or the Tie2 signalling pathway with a bispecific binding molecule.

Depending on the cancerous disease to be treated, a bispecific binding molecule of the invention may be used on its own or in combination with one or more additional therapeutic agents, in particular selected from chemotherapeutic agents like DNA damaging agents or therapeutically active compounds that inhibit angiogenesis, signal transduction pathways or mitotic checkpoints in cancer cells.

The additional therapeutic agent may be administered simultaneously with, optionally as a component of the same pharmaceutical preparation, or before or after administration of the bispecific binding molecule.

In certain embodiments, the additional therapeutic agent may be, without limitation, one or more inhibitors selected from the group of inhibitors of EGFR, VEGFR, HER2-neu, Her3, AuroraA, AuroraB, PLK and PI3 kinase, FGFR, PDGFR, Raf, Ras, KSP, PDK1, PTK2, IGF-R or IR.

Further examples of additional therapeutic agents are inhibitors of CDK, Akt, src/bcr abl, cKit, cMet/HGF, c-Myc, Flt3, HSP90, hedgehog antagonists, inhibitors of JAK/STAT, Mek, mTor, NFkappaB, the proteasome, Rho, an inhibitor of wnt signaling or an inhibitor of the ubiquitination pathway or another inhibitor of the Notch signaling pathway.

Examples for Aurora inhibitors are, without limitation, PHA-739358, AZD-1152, AT 9283, CYC-116, R-763, VX-680, VX-667, MLN-8045, PF-3814735.

An example for a PLK inhibitor is GSK-461364.

Examples for raf inhibitors are BAY-73-4506 (also a VEGFR inhibitor), PLX 4032, RAF-265 (also in addition a VEGFR inhibitor), sorafenib (also in addition a VEGFR inhibitor), and XL 281.

Examples for KSP inhibitors are ispinesib, ARRY-520, AZD-4877, CK-1122697, GSK 246053A, GSK-923295, MK-0731, and SB-743921.

Examples for a src and/or bcr-abl inhibitors are dasatinib, AZD-0530, bosutinib, XL 228 (also an IGF-1R inhibitor), nilotinib (also a PDGFR and cKit inhibitor), imatinib (also a cKit inhibitor), and NS-187.

An example for a PDK1 inhibitor is BX-517.

An example for a Rho inhibitor is BA-210.

Examples for PI3 kinase inhibitors are PX-866, BEZ-235 (also an mTor inhibitor), XL 418 (also an Akt inhibitor), XL-147, and XL 765 (also an mTor inhibitor).

Examples for inhibitors of cMet or HGF are XL-184 (also an inhibitor of VEGFR, cKit, FIt3), PF-2341066, MK-2461, XL-880 (also an inhibitor of VEGFR), MGCD-265 (also an inhibitor of VEGFR, Ron, Tie2), SU-11274, PHA-665752, AMG-102, and AV-299.

An example for a c-Myc inhibitor is CX-3543.

Examples for Flt3 inhibitors are AC-220 (also an inhibitor of cKit and PDGFR), KW 2449, lestaurtinib (also an inhibitor of VEGFR, PDGFR, PKC), TG-101348 (also an inhibitor of JAK2), XL-999 (also an inhibitor of cKit, FGFR, PDGFR and VEGFR), sunitinib (also an inhibitor of PDGFR, VEGFR and cKit), and tandutinib (also an inhibitor of PDGFR, and cKit).

Examples for HSP90 inhibitors are tanespimycin, alvespimycin, IPI-504 and CNF 2024.

Examples for JAK/STAT inhibitors are CYT-997 (also interacting with tubulin), TG 101348 (also an inhibitor of Flt3), and XL-019.

Examples for Mek inhibitors are ARRY-142886, PD-325901, AZD-8330, and XL 518.

Examples for mTor inhibitors are temsirolimus, AP-23573 (which also acts as a VEGF inhibitor), everolimus (a VEGF inhibitor in addition). XL-765 (also a PI3 kinase inhibitor), and BEZ-235 (also a PI3 kinase inhibitor).

Examples for Akt inhibitors are perifosine, GSK-690693, RX-0201, and triciribine.

Examples for cKit inhibitors are AB-1010, OSI-930 (also acts as a VEGFR inhibitor), AC-220 (also an inhibitor of Flt3 and PDGFR), tandutinib (also an inhibitor of Flt3 and to PDGFR), axitinib (also an inhibitor of VEGFR and PDGFR), XL-999 (also an inhibitor of Flt3, PDGFR, VEGFR, FGFR), sunitinib (also an inhibitor of Flt3, PDGFR, VEGFR), and XL-820 (also acts as a VEGFR- and PDGFR inhibitor), imatinib (also a bcr-abl inhibitor), nilotinib (also an inhibitor of bcr-abl and PDGFR).

Examples for hedgehog antagonists are IPI-609 and CUR-61414.

Examples for CDK inhibitors are seliciclib, AT-7519, P-276, ZK-CDK (also inhibiting VEGFR2 and PDGFR), PD-332991, R-547, SNS-032, PHA-690509, and AG 024322.

Examples for proteasome inhibitors are bortezomib, carfilzomib, and NPI-0052 (also an inhibitor of NFkappaB).

An example for an NFkappaB pathway inhibitor is NPI-0052.

An example for an ubiquitination pathway inhibitor is HBX-41108.

In preferred embodiments, the additional therapeutic agent is an anti-angiogenic agent.

Examples for anti-angiogenic agents are inhibitors of the FGFR, PDGFR and VEGFR or the respective ligands (e.g VEGF inhibitors like pegaptanib or the anti-VEGF antibody bevacizumab), and thalidomides, such agents being selected from, without limitation, bevacizumab, motesanib, CDP-791, SU-14813, telatinib, KRN-951, ZK-CDK (also an inhibitor of CDK), ABT-869, BMS-690514, RAF-265, IMC-KDR, IMC-18F1, IMiDs (immunomodulatory drugs), thalidomide derivative CC-4047, lenalidomide, ENMD 0995, IMC-D11, Ki 23057, brivanib, cediranib, XL-999 (also an inhibitor of cKit and Flt3), 1B3, CP 868596, IMC 3G3, R-1530 (also an inhibitor of Flt3), sunitinib (also an inhibitor of cKit and Flt3), axitinib (also an inhibitor of cKit), lestaurtinib (also an inhibitor of Flt3 and PKC), vatalanib, tandutinib (also an inhibitor of Flt3 and cKit), pazopanib, GW 786034, PF-337210, IMC-1121B, AVE-0005, AG-13736, E-7080, CHIR 258, sorafenib tosylate (also an inhibitor of Raf), RAF-265 (also an inhibitor of Raf), vandetanib, CP-547632, OSI-930, AEE-788 (also an inhibitor of EGFR and Her2), BAY-57-9352 (also an inhibitor of Raf), BAY-73-4506 (also an inhibitor of Raf), XL 880 (also an inhibitor of cMet), XL-647 (also an inhibitor of EGFR and EphB4), XL 820 (also an inhibitor of cKit), and nilotinib (also an inhibitor of cKit and brc-abl).

The additional therapeutic agent may also be selected from EGFR inhibitors, it may be a small molecule EGFR inhibitor or an anti-EGFR antibody. Examples for anti-EGFR antibodies, without limitation, are cetuximab, panitumumab, matuzumab; an example for a small molecule EGFR inhibitor is gefitinib. Another example for an EGFR modulator is the EGF fusion toxin.

Among the EGFR and Her2 inhibitors useful for combination with the bispecific binding molecule of the invention are lapatinib, gefitinib, erlotinib, cetuximab, trastuzumab, nimotuzumab, zalutumumab, vandetanib (also an inhibitor of VEGFR), pertuzumab, XL-647, HKI-272, BMS-599626 ARRY-334543, AV 412, mAB-806, BMS-690514, JNJ-26483327, AEE-788 (also an inhibitor of VEGFR), ARRY-333786, IMC-11F8, Zemab.

Other agents that may be advantageously combined in a therapy with the bispecific binding molecule of the invention are tositumumab and ibritumomab tiuxetan (two radiolabelled anti-CD20 antibodies), alemtuzumab (an anti-CD52 antibody), denosumab, (an osteoclast differentiation factor ligand inhibitor), galiximab (a CD80 antagonist), ofatumumab (a CD20 inhibitor), zanolimumab (a CD4 antagonist), SGN40 (a CD40 ligand receptor modulator), rituximab (a CD20 inhibitor) or mapatumumab (a TRAIL-1 receptor agonist).

Other chemotherapeutic drugs that may be used in combination with the bispecific binding molecule s of the present invention are selected from, but not limited to hormones, hormonal-analogues and antihormonals (e.g. tamoxifen, toremifene, raloxifene, fulvestrant, megestrol acetate, flutamide, nilutamide, bicalutamide, cyproterone acetate, finasteride, buserelin acetate, fludrocortisone, fluoxymesterone, medroxyprogesterone, octreotide, arzoxifene, pasireotide, vapreotide), aromatase inhibitors (e.g. anastrozole, letrozole, liarozole, exemestane, atamestane, formestane), LHRH agonists and antagonists (e.g. goserelin acetate, leuprolide, abarelix, cetrorelix, deslorelin, histrelin, triptorelin), antimetabolites (e.g. antifolates like methotrexate, pemetrexed, pyrimidine analogues like 5 fluorouracil, capecitabine, decitabine, nelarabine, and gemcitabine, purine and adenosine analogues such as mercaptopurine thioguanine, cladribine and pentostatin, cytarabine, fludarabine); antitumor antibiotics (e.g. anthracyclines like doxorubicin, daunorubicin, epirubicin to and idarubicin, mitomycin-C, bleomycin dactinomycin, plicamycin, mitoxantrone, pixantrone, streptozocin); platinum derivatives (e.g. cisplatin, oxaliplatin, carboplatin, lobaplatin, satraplatin); alkylating agents (e.g. estramustine, meclorethamine, melphalan, chlorambucil, busulphan, dacarbazine, cyclophosphamide, ifosfamide, hydroxyurea, temozolomide, nitrosoureas such as carmustine and lomustine, thiotepa); antimitotic agents (e.g. vinca alkaloids like vinblastine, vindesine, vinorelbine, vinflunine and vincristine; and taxanes like paclitaxel, docetaxel and their formulations, larotaxel; simotaxel, and epothilones like ixabepilone, patupilone, ZK-EPO); topoisomerase inhibitors (e.g. epipodophyllotoxins like etoposide and etopophos, teniposide, amsacrine, topotecan, irinotecan) and miscellaneous chemotherapeutics such as amifostine, anagrelide, interferone alpha, procarbazine, mitotane, and porfimer, bexarotene, celecoxib.

Particularly preferred combination partners of the bispecific binding molecules of the present invention are VEGF antagonists, like bevacizumab (Avastin®), Vargatef®, Sorafenib and Sunitinib.

The efficacy of bispecific binding molecules of the invention or polypeptides containing them, and of compositions comprising the same, can be tested using any suitable in vitro assay, cell-based assay, in vivo assay and/or animal model known per se, or any combination thereof, depending on the specific disease or disorder of interest. Suitable assays and animal models will be clear to the skilled person, and for example include the assays described herein and used in the Examples below, e.g. a proliferation assay.

The data obtained in the experiments of the invention confirm that DII4-binding components of the invention have properties that are superior to those of DII4-binding molecules of the prior art, as can e.g. be taken from the ELISA data of FIG. 10, showing that affinity-matured VHHs block hDLL4/hNotch1-Fc interaction in a complete manner, as well as the IC₅₀ (nM) values for affinity matured VHHs in hDLL4/hNotch1-Fc competition ELISA; and the affinity KD(nM) of purified affinity matured VHHs on recombinant human DLL4 and mouse DLL4. This indicates that DII4-binding components of the invention are promising candidates to have therapeutic efficacy in diseases and disorders associated with DII4-mediated effects on angiogenesis, such as cancer.

According to another embodiment of the invention, there is provided a method of diagnosing a disease by

a) contacting a sample with a DII4- and/or Ang2 binding component of the invention as defined above, and
b) detecting binding of said DII4- and/or Ang2-binding component to said sample, and
c) comparing the binding detected in step (b) with a standard, wherein a difference in binding relative to said sample is diagnostic of a disease or disorder associated with DII4-mediated effects on angiogenesis.

For this and other uses, it may be useful to further modify a bispecific binding component of the invention, such as by introduction of a functional group that is one part of a specific binding pair, such as the biotin-(strept)avidin binding pair. Such a functional group may be used to link the bispecific binding molecule of the invention to another protein, polypeptide or chemical compound that is bound to the other half of the binding pair, i.e. through formation of the binding pair. For example, a bispecific binding molecule of the invention may be conjugated to biotin, and linked to another protein, polypeptide, compound or carrier conjugated to avidin or streptavidin. For example, such a conjugated bispecific binding molecule of the invention may be used as a reporter, for example in a diagnostic system where a detectable signal-producing agent is conjugated to avidin or streptavidin.

### BRIEF DESCRIPTION OF THE FIGURES

FIG. 1: Amino acid sequence alignment of human, rhesus and cynomolgus DLL4.

FIG. 2: Human and mouse DLL4 deletion mutants (amino acid domain boundaries in superscript).

FIG. 3: Purified VHHs blocking hDLL4/hNotch1-Fc interaction (ELISA).

FIG. 4: Purified VHHs blocking hDLL4/hNotch1-Fc interaction (AlphaScreen).

FIG. 5: Purified VHHs blocking CHO-hDLL4/hNotch1-Fc and CHO-mDLL4/hNotch1-Fc interaction (FMAT).

FIG. 6: Purified VHHs blocking DLL4 mediated Notch1 cleavage (reporter).

FIG. 7: Binding of purified VHHs to recombinant human and mouse DLL4 (ELISA).

FIG. 8: Binding of purified VHHs to recombinant human DLL1 and human Jagged-1 (ELISA).

FIG. 9: Binding of purified VHHs to human/mouse/cynomolgus DLL4 (FACS).

FIG. 10: Affinity matured VHHs blocking hDLL4/hNotch1-Fc interaction (ELISA).

FIG. 11: Purified affinity matured VHHs blocking CHO-hDLL4/hNotch1-Fc and CHO-mDLL4/hNotch1-Fc interaction (FMAT).

FIG. 12: Binding of purified VHHs to human/mouse DLL4 (ELISA).

FIG. 13: Binding of purified affinity matured VHHs to recombinant human DLL1 and human Jagged-1 (ELISA).

FIG. 14: Binding of purified VHHs to human/mouse/cynomolgus DLL4 (FACS).

FIG. 15: Evaluation of VHH effects on DII4-mediated inhibition of HUVEC proliferation.

FIG. 16: Description cycle 1 DLL4xAng2 VHHs

FIG. 17: Purified cycle 1 DLL4xAng2 VHHs blocking hDLL4-hNotch1 interaction (ELISA)

FIG. 18: Purified cycle 1 DLL4xAng2 VHHs blocking CHO-hDLL4/Notch1 (44-1) and CHO-mDLL4/Notch1 (44-2) interaction (FMAT)

FIG. 19: Purified cycle 1 DLL4xAng2 VHHs binding to human, mouse and cynomolgus DLL4 overexpressing CHO cells (FACS)

FIG. 20: Purified cycle 1 DLL4xAng2 VHHs binding to human, mouse and rat

### DLL4 (ELISA)

FIG. 21: Purified cycle 1 DLL4xAng2 VHHs binding to human DLL1 and Jagged-1 (ELISA)

FIG. 22: Purified cycle 1 DLL4xAng2 VHHs blocking hAng2-hTie2 (48-1), mAng2-mTie2 (48-2) and cAng2/cTie2 (48-3) interaction (ELISA)

FIG. 23: Description cycle 2 DLL4xAng2 bispecific VHHs

FIG. 24: Purified cycle 2 DLL4xAng2 VHHs blocking hDLL4-hNotch1 interaction (ELISA)

FIG. 25: Purified cycle 2 DLL4xAng2 VHHs blocking CHO-hDLL4/Notch1 (51-1) and CHO-mDLL4/Notch1 (51-2) interaction (FMAT)

FIG. 26: Purified cycle 2 DLL4xAng2 VHHs blocking hDLL4 mediated Notch1 activation (reporter gene assay)

FIG. 27: Purified cycle 2 DLL4xAng2 VHHs binding to human, mouse and cynomolgus DLL4 overexpressing CHO cells (FACS)

FIG. 28: Purified cycle 2 DLL4xAng2 VHHs binding to human, mouse and rat DLL4 (ELISA)

FIG. 29: Purified cycle 2 DLL4xAng2 VHHs binding to human DLL1 and Jagged-1 (ELISA)

FIG. 30: Purified cycle 2 DLL4xAng2 VHHs blocking hAng2-hTie2 (56-1), mAng2-mTie2 (56-2) and cAng2/cTie2 (56-3) interaction (ELISA)

FIG. 31: Purified cycle 2 DLL4xAng2 VHHs blocking hAng1-hTie2 interaction (ELISA)

FIG. 32: Purified cycle 2 DLL4xAng2 VHHs blocking hAng2 mediated HUVEC survival.

### MATERIALS AND METHODS

### a) Generation CHO and HEK293 Cell Lines Overexpressing Human, Mouse and Cynomolgus DII4

The cDNAs encoding human (SEQ ID NO: 417; NM_{—}019074.2) and mouse DII4 (NM_{—}019454.3) are amplified from a Human Adult Normal Tissue Heart cDNA library (BioChain, Hayward, Calif., USA) and a Mouse Heart Tissue cDNA library (isolated from C57/BI6 strain), respectively, using oligonucleotides designed in the 5′ and 3′ UTR of the corresponding sequence (see Table 1; SEQ ID NO:421 to 426). Amplicons are cloned into the mammalian expression vector pcDNA3.1(+)-neo (Invitrogen, Carlsbad, Calif., USA).

TABLE 1
Oligonucleotide sequences used for amplification
of DLL4 gene full length orthologues.

| Human DLL4 | Mouse DLL4 | Cynomolgus DLLR |
|---|---|---|
| >Fwd_hDLL4 | >Fwd_mDLL4 | >Fwd_cDLLR |
| GCGAACAGAGCCAGATTGAGG | GAGCGACATCCCTAACAAGC | GCGAACAGAGCCAGATTCAGG |
| (SEQ ID NO: 421) | (SEQ ID NO: 423) | (SEQ ID NO: 425) |
| >Rev_hDLL4 | >Rev_mDLL4 | >Rev_cDLL4 |
| GGATGTCCAGGTAGGCTCCTG | CCTCAACTCTGTTCCCTTGG | CCAGACAGACACCCAAAGGT |
| (SEQ ID NO: 422) | (SEQ ID NO: 424) | (SEQ ID NO: 426) |

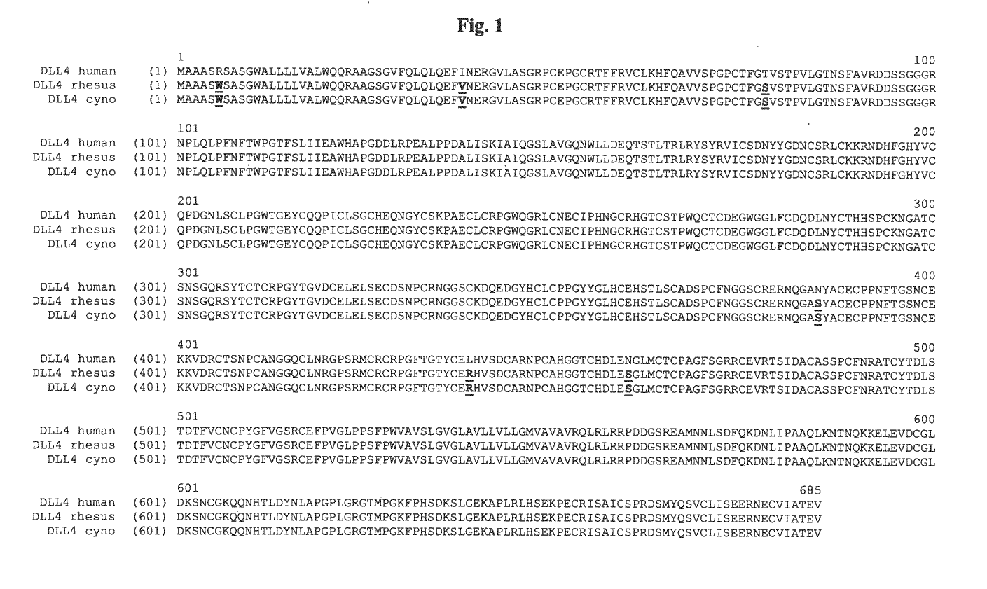

Cynomolgus DII4 cDNA is amplified from a Cynomolgus Normal Tissue Heart cDNA library (BioChain, Hayward, Calif., USA), using primers designed on the 5′ and 3′ UTR of the DII4 encoding sequence of the closely related species rhesus (Macaca mulatta DII4, SEQ ID NO:418; XM_{—}001099250.1) (see Table 1). The final amplicon is cloned in the mammalian expression vector pcDNA3.1(+)-neo (Invitrogen, Carlsbad, Calif., USA). The amino acid sequence of cynomolgus DII4 was shown to be 100% identical to rhesus, and 99% identical to human (see FIG. 1; differences from the human sequence are indicated as bold-underlined).

To establish Chinese Hamster Ovary (CHO) cells overexpressing human DII4, mouse to DII4 or cynomolgus DII4, parental CHO cells are electroporated with pcDNA3.1(+)-neo-hDII4, pcDNA3.1 (+)-neo-mDII4 or pcDNA3.1 (+)-neo-cDII4, respectively. Human Embyonic Kidney (HEK293) cells overexpressing human DII4 and mouse DII4 are generated by lipid-mediated transfection with Fugene (Roche) of pcDNA3.1(+)-neo-hDII4 or mDII4 plasmids, respectively, in the HEK293 parental cell line. For all conditions, transfectants are selected by adding 1 mg/mL geneticin (Invitrogen, Carlsbad, Calif., USA).

### b) Generation of Monoclonal Anti-D/14 IgG and Fab Fragment

In US 2008/0014196 (Genentech) a human/mouse cross-reactive DII4 mAb is described that was used by Ridgway et al. (2006) to show additive effects of VEGF mAb and DII4 mAb on tumor growth in a number of xenograft models. This anti-DII4 mAb and its corresponding Fab are purified to assess the properties of this antibody (fragment) in biochemical/cellular assays and xenograft models and for specific elutions during phage selections. The published variable heavy and light chain sequences of DII4 mAb are cloned into a hIgG2aκ framework, transiently expressed in HEK293 cells and purified from supernatants using protein A chromatography. Purified DII4 mAb shows binding to human DII4 and mouse DII4 in ELISA and FACS (using CHO-mDII4 and CHO-hDII4 cells), sub-nanomolar affinities to both growth factor orthologues in Biacore.

The corresponding DII4 Fab fragment is constructed via gene assembly based on back-translation and codon optimization for expression in E. coli using Leto's Gene Optimization software (www.entechelon.com). Oligonucleotide primers for the assembly of the variable light chain (V_{L}), variable heavy chain (V_{H}), constant light chain (C_{L}) and constant domain 1 of the heavy chain (C_{H}1) are designed and an assembly PCR is performed. The cDNA segments encoding V_{L}+C_{L} and V_{H}+C_{H}1 are cloned into a pUC19-derived vector, which contains the LacZ promotor, a resistance gene for kanamycin, a multiple cloning site and a hybrid gill-pelB leader sequence, using the restriction sites Sfil and Ascl and the restriction sites KpnI and NotI, respectively. In frame with the Fab coding sequence, the expression vector encodes a C-terminal HA and His6-tag. The Fab fragment is expressed in E. coli as His6-tagged protein and subsequently purified from the culture medium by immobilized metal affinity chromatography (IMAC) and size exclusion chromatography (SEC). Relevant amino acid sequences of the variable heavy and variable light chain are depicted (SEQ ID NO: 1 and SEQ ID NO: 2; respectively, of US 2008/0014196); the amino acid sequences of the complete heavy and light chain are shown in SEQ ID NOs: 419 and 420, respectively.

### c) Generation of DII4 Mutants for Epitope Mapping

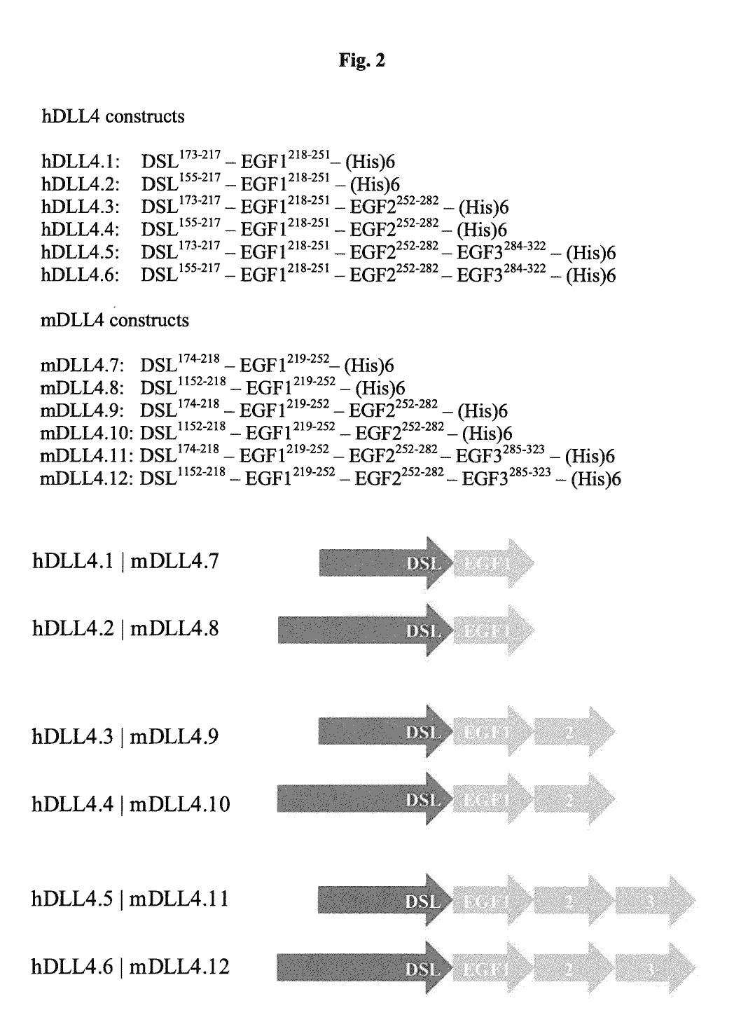

To identify the region in the extracellular domain (ECD) of DII4 that comprises the epitope recognized by the anti-DII4 VHHs, progressive deletion mutants of the DII4 ECD are generated. The mammalian expression vector pSecTag2/Hygro (Invitrogen, Carlsbad, Calif., USA) comprising a CMV promotor upstream of polynucleotides encoding a nested series of deletion fragments of the DII4 ECD fused to a polyHis-tag are generated using standard recombinant DNA technology (see FIG. 2; amino acid domain boundaries in superscript).). These recombinant proteins are expressed transiently transfected HEK293 cells using the Freestyle 293 Expression System (Invitrogen, Carlsbad, Calif., USA) from which conditioned medium is collected and purified via IMAC. Only DII4 mutants lacking the EGF2-like domain showed impaired binding to the humanized human/mouse cross-reactive anti-DII4 mAb described above (immobilized via a capturing anti-human IgG coated Biacore sensor chip). This IgG is known to have a specific binding epitope in this DII4 domain (patent application Genentech, US 2008/0014196A1).

### d) Generation of DII4 Reporter Assay Plasmids

A reporter assay is developed based on the γ-secretase mediated cleavage of Notch1 and nuclear translocation of the intracellular domain of Notch1 (NICD) upon stimulation with DII4, essentially as described (Struhl and Adachi, Cell. 1998 May 15; 93(4):649-60). Gal4NP16 coding sequences are inserted into the NICD-coding sequence. The potent hybrid transcriptional activator GAL4-VP16, which consists of a DNA binding fragment of yeast GAL4 fused to a Herpes simplex viral transcriptional activator domain VP16, is inserted carboxy-terminal to the transmembrane domain of Notch1. Cleavage of this construct by γ-secretase results in the release of the Gal4NP16 NICD fusion protein which will translocate to the nucleus where it will bind to and transcriptionally activate a co-transfected luciferase reporter plasmid, containing a strong GAL4-UAS promoter sequence (Struhl, G. and Adachi, A., Cell, vol. 93, 649-660, 1998). The human Notch1-Gal4NP16 expression cassette is to cloned in pcDNA3.1(+)-neo (Invitrogen, Carlsbad, Calif., USA). The pGL4.31-[Luc2P/Gal4UAS/Hygro] vector (Promega, Madison, Wis., USA) is used as luciferase reporter plasmid.

### Example 1

### Immunization with DII4 from Different Species Induces a Humoral Immune Response in Llama

### 1.1. Immunizations

After approval of the Ethical Committee of the faculty of Veterinary Medicine (University Ghent, Belgium), 4 llamas (designated No. 208, 209, 230, 231) are immunized with 6 intramuscular injections (100 or 50 μg/dose at weekly intervals) of recombinant human DII4 (R&D Systems, Minneapolis, Minn., US). The DII4 antigen is formulated in Stimune (Cedi Diagnostics BV, Lelystad, The Netherlands). Three additional llamas (designated No. 127b, 260, 261) are immunized according to standard protocols with 4 subcutaneous injections of alternating human DII4 and mouse DII4 overexpressing CHO cells which are established as described above. Cells are re-suspended in D-PBS and kept on ice prior to injection. Furthermore, three additional llamas (designated No. 282, 283, 284) are immunized according to standard protocols with 4 intramuscular injections (100 or 50 μg/dose at biweekly intervals) of alternating recombinant human DII4 and mouse DII4 (R&D Systems, Minneapolis, Minn., US). The first injection at day 0 with human DII4 is formulated in Complete Freund's Adjuvant (Difco, Detroit, Mich., USA), while the subsequent injections with human and mouse DII4 are formulated in Incomplete Freund's Adjuvant (Difco, Detroit, Mich., USA).

### 1.2. Evaluation of Induced Immune Responses in Llama

To evaluate the induction of an immune responses in the animals against human DII4 by ELISA, sera are collected from llamas 208, 209, 230 and 231 at day 0 (pre-immune), day 21 and day 43 (time of peripheral blood lymphocyte [PBL] collection), from llamas 127b, 260 and 261 at day 0 and day 51, and from llamas 282, 283 and 284 at day 0, day 28 and day 50. In short, 2 μg/mL of recombinant human DII4 or mouse DII4 (R&D. Systems, Minneapolis, Minn., USA) are immobilized overnight at 4° C. in a 96-well MaxiSorp plate (Nunc, Wiesbaden, Germany). Wells are blocked with a casein solution (1%). After addition of serum dilutions, specifically bound immunoglobulins are detected using a horseradish peroxidase (HRP)-conjugated goat anti-llama immunoglobulin (Bethyl Laboratories Inc., Montgomery, Tex., USA) and a subsequent enzymatic reaction in the presence of the substrate TMB (3,3′,5,5′-tetramentylbenzidine) (Pierce, Rockford, Ill., USA), showing that a significant antibody-dependend immune response against DII4 is induced. The antibody response is mounted both by conventional and heavy-chain only antibody expressing B-cell repertoires since specifically bound immunoglobulins can be detected with antibodies specifically recognizing the conventional llama IgG1 antibodies or the heavy chain only llama IgG2 or IgG3 antibodies (Table 2-A). In all llamas injected with mouse DII4, an antibody response is mounted by conventional and heavy chain only antibody expressing B-cells specifically against mouse DII4. Additionally, serum titers of cell immunized animals are confirmed by FACS analysis on human and mouse DII4 overexpressing HEK293 cells (Table 2-B). The DII4 serum titer responses for each llama are depicted in Table 2.

TABLE 2
Antibody mediated specific serum response against DLL4.
A) ELISA (recombinant protein solid phase coated)

| | | Recombinant human | | | | Recombinant mouse | | | |
|---|---|---|---|---|---|---|---|---|---|
| | | DLL4 | | | | DLL4 | | | |
| | Immuno- | Total | | | | Total | | | |
| Llama | gen | IgG | IgG1 | IgG2 | IgG3 | IgG | IgG1 | IgG2 | IgG3 |
| 208 | rec. | + | + | +/− | +/− | ND | ND | ND | ND |
| | human | | | | | | | | |
| | DLL4 | | | | | | | | |
| 209 | rec. | + | + | +/− | +/− | ND | ND | ND | ND |
| | human | | | | | | | | |
| | DLL4 | | | | | | | | |
| 230 | rec. | ++ | ++ | +/− | +/− | ND | ND | ND | ND |
| | human | | | | | | | | |
| | DLL4 | | | | | | | | |
| 231 | rec. | ++ | ++ | ++ | ++ | ND | ND | ND | ND |
| | human | | | | | | | | |
| | DLL4 | | | | | | | | |
| 127b | CHO- | ++ | ++ | +/− | +/− | + | ++ | +/− | +/− |
| | hDLL4 + | | | | | | | | |
| | CHO- | | | | | | | | |
| | mDLL4 | | | | | | | | |
| 260 | CHO- | ++ | ++ | + | + | ++ | ++ | + | ++ |
| | hDLL4 + | | | | | | | | |
| | CHO- | | | | | | | | |
| | mDLL4 | | | | | | | | |
| 261 | CHO- | ++ | ++ | +/− | +/− | + | + | +/− | +/− |
| | hDLL4 + | | | | | | | | |
| | CHO- | | | | | | | | |
| | mDLL4 | | | | | | | | |
| 282 | rec. | ++ | ++ | ++ | ++ | ++ | ++ | + | + |
| | human | | | | | | | | |
| | DLL4 + | | | | | | | | |
| | mouse | | | | | | | | |
| | DLL4 | | | | | | | | |
| 283 | rec. | ++ | ++ | ++ | ++ | ++ | ++ | ++ | ++ |
| | human | | | | | | | | |
| | DLL4 + | | | | | | | | |
| | mouse | | | | | | | | |
| | DLL4 | | | | | | | | |
| 284 | rec. | + | + | + | + | + | ++ | + | ++ |
| | human | | | | | | | | |
| | DLL4 + | | | | | | | | |
| | mouse | | | | | | | | |
| | DLL4 | | | | | | | | |

ND: not determined

B) FACS (natively expressed protein on HEK293 cells)

| | | human DLL4 | | | | mouse DLL4 | | | |
|---|---|---|---|---|---|---|---|---|---|
| Llama | Immunogen | Total IgG | IgG1 | IgG2 | IgG3 | Total IgG | IgG1 | IgG2 | IgG3 |
| 208 | rec. | ND | ND | ND | ND | ND | ND | ND | ND |
| | human | | | | | | | | |
| | DLL4 | | | | | | | | |
| 209 | rec. | ND | ND | ND | ND | ND | ND | ND | ND |
| | human | | | | | | | | |
| | DLL4 | | | | | | | | |
| 230 | rec. | ND | ND | ND | ND | ND | ND | ND | ND |
| | human | | | | | | | | |
| | DLL4 | | | | | | | | |
| 231 | rec. | ND | ND | ND | ND | ND | ND | ND | ND |
| | human | | | | | | | | |
| | DLL4 | | | | | | | | |
| 127b | CHO- | + | ND | ND | ND | + | ND | ND | ND |
| | hDLL4 + | | | | | | | | |
| | CHO- | | | | | | | | |
| | mDLL4 | | | | | | | | |
| 260 | CHO- | ++ | ND | ND | ND | ++ | ND | ND | ND |
| | hDLL4 + | | | | | | | | |
| | CHO- | | | | | | | | |
| | mDLL4 | | | | | | | | |
| 261 | CHO- | + | ND | ND | ND | + | ND | ND | ND |
| | hDLL4 + | | | | | | | | |
| | CHO- | | | | | | | | |
| | mDLL4 | | | | | | | | |
| 282 | rec. | ND | ND | ND | ND | ND | ND | ND | ND |
| | human | | | | | | | | |
| | DLL4 + | | | | | | | | |
| | mouse | | | | | | | | |
| | DLL4 | | | | | | | | |
| 283 | rec. | ND | ND | ND | ND | ND | ND | ND | ND |
| | human | | | | | | | | |
| | DLL4 + | | | | | | | | |
| | mouse | | | | | | | | |
| | DLL4 | | | | | | | | |
| 284 | rec. | ND | ND | ND | ND | ND | ND | ND | ND |
| | human | | | | | | | | |
| | DLL4 + | | | | | | | | |
| | mouse | | | | | | | | |
| | DLL4 | | | | | | | | |

ND: not determined

### Example 2

### Cloning of the Heavy-Chain Only Anti-DII4 Antibody Fragment Repertoires and Preparation of Phage

Following the final immunogen injection, immune tissues as the source of B-cells that produce the heavy-chain antibodies are collected from the immunized llamas. Typically, two 150-ml blood samples, collected 4 and 8 days after the last antigen injection, and one lymph node biopsy, collected 4 days after the last antigen injection are collected per animal. From the blood samples, peripheral blood mononuclear cells (PBMCs) are prepared using Ficoll-Hypaque according to the manufacturer's instructions (Amersham Biosciences, Piscataway, N.J., USA). From the PBMCs and the lymph node biopsy, total RNA is extracted, which is used as starting material for RT-PCR to amplify the VHH encoding DNA segments, as described in WO 05/044858. For each immunized llama, a library is constructed by pooling the total RNA isolated from all collected immune tissues of that animal. In short, the PCR-amplified VHH repertoire is cloned via specific restriction sites into a vector designed to facilitate phage display of the VHH library. The vector is derived from pUC119 and contains the LacZ promoter, a M13 phage gill protein coding sequence, a resistance gene for ampicillin or carbenicillin, a multiple cloning site and a hybrid glll-pelB leader sequence (pAX050). In frame with the VHH coding sequence, the vector encodes a C-terminal c-myc tag and a His6 tag. Phage are prepared according to standard protocols and stored after filter sterilization at 4° C. for further use.

### Example 3

### Selection of DII4 Specific VHHs Via Phage Display

VHH repertoires obtained from all llamas and cloned as phage library are used in different selection strategies, applying a multiplicity of selection conditions. Variables include i) the DII4 protein format (C-terminally His-tagged recombinantly expressed extracellular domain of human DII4 (Met1-Pro524) and mouse DII4 (Met1-Pro525) (R&D Systems, Minneapolis, Minn., USA), or full length human DII4 and mouse DII4 present on DII4-overexpressing CHO or HEK293 cells, ii) the antigen presentation method (plates directly coated with DII4 or Neutravidin plates coated with DII4 via a biotin-tag; solution phase: incubation in solution followed by capturing on Neutravidin-coated plates), iii) the antigen concentration and iv) different elution methods (non-specific via trypsin or specific via cognate receptor Notch1/Fc chimera or anti-DII4 IgG/Fab). All selections are done in Maxisorp 96-well plates (Nunc, Wiesbaden, Germany).

Selections are performed as follows: DII4 antigen preparations for solid and solution phase selection formats are presented as described above at multiple concentrations. After 2 h incubation with the phage libraries followed by extensive washing, bound phage are eluted with trypsin (1 mg/mL) for 30 minutes. In case trypsin is used for phage elution, the protease activity is immediately neutralized applying 0.8 mM protease inhibitor ABSF. As control, selections w/o antigen are performed in parallel. Phage outputs that show enrichment over background (non-antigen control) are used to infect E. coli. Infected E. coli cells are either used to prepare phage for the next selection round (phage rescue) or plated on agar plates (LB+amp+glucose^{2%}) for analysis of individual VHH clones. In order to screen a selection output for specific binders, single colonies are picked from the agar plates and grown in 1 mL 96-deep-well plates. LacZ-controlled VHH expression is induced by adding IPTG (0.1-1 mM final) in the absence of glucose. Periplasmic extracts (in a volume of ˜80 uL) are prepared according to standard protocols

### Example 4

### Screening of Periplasmic Extracts in DII4-Notch1 AlphaScreen and FMAT Competition Assay

Periplasmic extracts are screened in a human DII4/human Notch1 AlphaScreen assay to assess the blocking capacity of the expressed VHHs. Human DII4 is biotinylated using biotin (Sigma, St Louis, Mo., USA) and biotinamidohexanoic acid 3-sulfo-N-hydroxysuccinimide ester sodium salt (Sigma, St Louis, Mo., USA). Notch1/Fc chimera (R&D Systems, Minneapolis, Minn., USA) is captured using an anti-Fc VHH which is coupled to acceptor beads according to the manufacturer's instructions (Perkin Elmer, Waltham, Mass., US). To evaluate the neutralizing capacity of the VHHs, dilution series of the periplasmic extracts are pre-incubated with biotinylated human DII4. To this mixture, the acceptor beads and the streptavidin donor beads are added and further incubated for 1 hour at room temperature. Fluorescence is measured by reading plates on the Envision Multilabel Plate reader (Perkin Elmer, Waltham, Mass., USA) using an excitation wavelength of 680 nm and an emission wavelength of 520 nm. Decrease in fluorescence signal indicates that the binding of biotinylated human DII4 to the human Notch1/Fc receptor is blocked by the VHH expressed in the periplasmic extract.

Alternatively, CHO-hDII4 and CHO-mDII4 cells are used in a human Notch1/Fc FMAT (Fluorometric Microvolume Assay Technology) competition assay. Recombinant human Notch1/Fc chimera (R&D Systems, Minneapolis, Minn., USA) is randomly labeled with Alexa-647 (Invitrogen, Carlsbad, Calif., USA). In brief, 5 μL periplasmic material is added to 100 pM or 175 pM labeled human Notch1/Fc together with 7,500 CHO-hDII4 or CHO-mDII4 overexpressing cells, respectively, and readout is performed after 2 hours of incubation. To set the no-competition baseline, at least replicates of cells with human Notch1/Fc-Alexa647 are included and the percentage of inhibition is calculated from this baseline. All calculations are based on the FL1_total signal which comprises the average of the fluorescence per well times the number of counts per well.

From this screening, inhibiting VHHs are selected and sequenced. Sequence analysis revealed 167 unique VHHs belonging to 40 different B-cell lineages. The to total number of variants found for each B-cell lineage is depicted in Table 3. An overview of periplasmic screening data is given in Table 4. The amino acid sequences of all obtained unique VHHs are shown in the Sequence Listing (SEQ ID NO:167-332 and 459) and in Table 5 (CDRs and framework regions are indicated).

TABLE 3
Selection parameters used for the identification of DLL4 specific VHH B-cell lineages.

| B-cell | | # | | selection | phage | selection |
|---|---|---|---|---|---|---|
| lineage | VHH ID | variants | library | format | elution | rounds |
| 1 | DLLBII8A09 | 31 | 231 | rhDLL4 (3 nM) | trypsin | 1 |
| 2 | DLLBII5B11 | 1 | 231 | rhDLL4 (3 nM) | trypsin | 1 |
| 3 | DLLBII7B5 | 21 | 231 | RI: biot- | trypsin | 2 |
| | | | | rhDLL4 | | |
| | | | | (3 nM) | | |
| | | | | RII: biot- | | |
| | | | | rhDLL4 (0.03 nM) | | |
| 4 | DLLBII6B11 | 13 | 231 | biot-rhDLL4 | trypsin | 1 |
| | | | | (3M) | | |
| 5 | DLLBII8C11 | 5 | 231 | RI: biot- | trypsin | 2 |
| | | | | rhDLL4 | | |
| | | | | (3 nM) | | |
| | | | | RII: biot- | | |
| | | | | rhDLL4 | | |
| | | | | (3 nM) | | |
| 6 | DLLBII19D10 | 1 | 231 | biot-rhDLL4 | trypsin | 1 |
| | | | | (3 nM) | | |
| 7 | DLLBII33C5 | 2 | 231 | CHO-hDLL4 | trypsin | 1 |
| | | | | (2E6/mL) | | |
| 8 | DLLBII28B6 | 2 | 231 | rmDLL4 | trypsin | 1 |
| | | | | (0.5 ug/mL) | | |
| 9 | DLLBII17G10 | 1 | 231 | biot-rhDLL4 | trypsin | 1 |
| | | | | (3 nM) | | |
| 10 | DLLBII17C1 | 8 | 231 | biot-rhDLL4 | trypsin | 1 |
| | | | | (3 nM) | | |
| 11 | DLLBII19F4 | 1 | 231 | biot-rhDLL4 | trypsin | 1 |
| | | | | (3 nM) | | |
| 12 | DLLBII17F10 | 1 | 231 | biot-rhDLL4 | trypsin | 1 |
| | | | | (3 nM) | | |
| 13 | DLLBII17B3 | 5 | 231 | biot-rhDLL4 | trypsin | 1 |
| | | | | (3 nM) | | |
| 14 | DLLBII19F12 | 2 | 231 | biot-rhDLL4 | trypsin | 1 |
| | | | | (3 nM) | | |
| 15 | DLLBII42B7 | 1 | 231 | RI: biot- | rhNotch | 2 |
| | | | | rhDLL4 | 1/Fc | |
| | | | | (3 nM) | | |
| | | | | RII: biot- | | |
| | | | | rhDLL4 | | |
| | | | | (3 nM) | | |
| 16 | DLLBII47D1 | 1 | 230 | RI: biot- | rhNotch | 2 |
| | | | | rhDLL4 | 1/Fc | |
| | | | | (3 nM) | | |
| | | | | RII: biot- | | |
| | | | | rhDLL4 | | |
| | | | | (3 nM) | | |
| 17 | DLLBII56A09 | 15 | 230 | RI: CHO- | rhNotch | 2 |
| | | | | mDLL4 | 1/Fc | |
| | | | | (2E6/mL) | | |
| | | | | RII: CHO- | | |
| | | | | mDLL4 | | |
| | | | | (2E6/mL) | | |
| 18 | DLLBII95F2 | 5 | 230 | RI: CHO- | trypsin | 2 |
| | | | | mDLL4 | | |
| | | | | (2E6/mL) | | |
| | | | | RII: CHO- | | |
| | | | | mDLL4 | | |
| | | | | (2E6/mL) | | |
| 19 | DLLBII96C3 | 20 | 230 | RI: CHO- | trypsin | 2 |
| | | | | mDLL4 | | |
| | | | | (2E6/mL) | | |
| | | | | RII: CHO- | | |
| | | | | mDLL4 | | |
| | | | | (2E6/mL) | | |
| 20 | DLLBII104G1 | 1 | 230 | RI: CHO- | rhNotch | 3 |
| | | | | mDLL4 | 1/Fc | |
| | | | | (2E6/mL) | (RI-RII) | |
| | | | | RII: CHO- | trypsin | |
| | | | | mDLL4 | (RIII) | |
| | | | | (2E6/mL) | | |
| | | | | RIII: biot- | | |
| | | | | rhDLL4 | | |
| | | | | (+rhDLL4) | | |
| 21 | DLLBII102F8 | 3 | 230 | RI: CHO- | rhNotch | 3 |
| | | | | mDLL4 | 1/Fc | |
| | | | | (2E6/mL) | (RI-RII) | |
| | | | | RII: CHO- | trypsin | |
| | | | | mDLL4 | (RIII) | |
| | | | | (2E6/mL) | | |
| | | | | RIII: biot- | | |
| | | | | rhDLL4 (0.01 nM) | | |
| 22 | DLLBII112A3 | 1 | 209 | RI: CHO- | trypsin | 2 |
| | | | | mDLL4 | | |
| | | | | (2E6/mL) | | |
| | | | | RII: CHO- | | |
| | | | | mDLL4 | | |
| | | | | (2E6/mL) | | |
| 23 | DLLBII102G4 | 2 | 230 | RI: CHO- | rhNotch | 3 |
| | | | | mDLL4 | 1/Fc | |
| | | | | (2E6/mL) | (RI-RII) | |
| | | | | RII: CHO- | trypsin | |
| | | | | mDLL4 | (RIII) | |
| | | | | (2E6/mL) | | |
| | | | | RIII: biot- | | |
| | | | | rhDLL4 (0.01 nM) | | |
| 24 | DLLBII101G8 | 1 | 230 | RI: CHO- | rhNotch | 3 |
| | | | | mDLL4 | 1/Fc | |
| | | | | (2E6/mL) | (RI-RII) | |
| | | | | RII: CHO- | trypsin | |
| | | | | mDLL4 | (RIII) | |
| | | | | (2E6/mL) | | |
| | | | | RIII: biot- | | |
| | | | | rhDLL4 (0.1 nM) | | |
| 25 | DLLBII112A4 | 1 | 209 | RI: CHO- | trypsin | 2 |
| | | | | mDLL4 | | |
| | | | | (2E6/mL) | | |
| | | | | RII: CHO- | | |
| | | | | mDLL4 | | |
| | | | | (2E6/mL) | | |
| 26 | DLLBII101H9 | 1 | 230 | RI: CHO- | rhNotch | 3 |
| | | | | mDLL4 | 1/Fc | |
| | | | | (2E6/mL) | (RI-RII) | |
| | | | | RII: CHO- | trypsin | |
| | | | | mDLL4 | (RIII) | |
| | | | | (2E6/mL) | | |
| | | | | RIII: biot- | | |
| | | | | rhDLL4 (0.1 nM) | | |
| 27 | DLLBII101H5 | 1 | 230 | RI: CHO- | rhNotch | 3 |
| | | | | mDLL4 | 1/Fc | |
| | | | | (2E6/mL) | (RI-RII) | |
| | | | | RII: CHO- | trypsin | |
| | | | | mDLL4 | (RIII) | |
| | | | | (2E6/mL) | | |
| | | | | RIII: biot- | | |
| | | | | rhDLL4 | | |
| | | | | (1 nM) | | |
| 28 | DLLBII112E7 | 1 | 209 | RI: CHO- | trypsin | 2 |
| | | | | mDLL4 | | |
| | | | | (2E6/mL) | | |
| | | | | RII: CHO- | | |
| | | | | mDLL4 | | |
| | | | | (2E6/mL) | | |
| 29 | DLLBII101F1 | 1 | 230 | RI: CHO- | rhNotch | 3 |
| | | | | mDLL4 | 1/Fc | |
| | | | | (2E6/mL) | (RI-RII) | |
| | | | | RII: CHO- | trypsin | |
| | | | | mDLL4 | (RIII) | |
| | | | | (2E6/mL) | | |
| | | | | RIII: biot- | | |
| | | | | rhDLL4 | | |
| | | | | (1 nM) | | |
| 30 | DLLBII104A3 | 1 | 230 | RI: CHO- | rhNotch | 3 |
| | | | | mDLL4 | 1/Fc | |
| | | | | (2E6/mL) | (RI-RII) | |
| | | | | RII: CHO- | trypsin | |
| | | | | mDLL4 | (RIII) | |
| | | | | (2E6/mL) | | |
| | | | | RIII: biot- | | |
| | | | | rhDLL4 | | |
| | | | | (1 nM) + | | |
| | | | | rhDLL4 | | |
| 31 | DLLBII104C4 | 1 | 230 | RI: CHO- | rhNotch | 3 |
| | | | | mDLL4 | 1/Fc | |
| | | | | (2E6/mL) | (RI-RII) | |
| | | | | RII: CHO- | trypsin | |
| | | | | mDLL4 | (RIII) | |
| | | | | (2E6/mL) | | |
| | | | | RIII: biot- | | |
| | | | | rhDLL4 | | |
| | | | | (1 nM) + | | |
| | | | | rhDLL4 | | |
| 32 | DLLBII104B5 | 1 | 230 | RI: CHO- | rhNotch | 3 |
| | | | | mDLL4 | 1/Fc | |
| | | | | (2E6/mL) | (RI-RII) | |
| | | | | RII: CHO- | trypsin | |
| | | | | mDLL4 | (RIII) | |
| | | | | (2E6/mL) | | |
| | | | | RIII: biot- | | |
| | | | | rhDLL4 | | |
| | | | | (1 nM) + | | |
| | | | | rhDLL4 | | |
| 33 | DLLBII107C3 | 1 | 208 | RI: CHO- | rhNotch | 2 |
| | | | | mDLL4 | 1/Fc | |
| | | | | (2E6/mL) | | |
| | | | | RII: CHO- | | |
| | | | | mDLL4 | | |
| | | | | (2E6/mL) | | |
| 34 | DLLBII58A11 | 4 | 260 | RI: biot- | rhNotch | 2 |
| | | | | rhDLL4 | 1/Fc | |
| | | | | (3 nM) | | |
| | | | | RII: biot- | | |
| | | | | rmDLL4 (3 nM) | | |
| 35 | DLLBII61F5 | 1 | 260 | RI: HEK293H- | trypsin | 2 |
| | | | | hDLL4 | | |
| | | | | (2E6/mL) | | |
| | | | | RII: | | |
| | | | | HEK293H- | | |
| | | | | hDLL4 | | |
| | | | | (2E6/mL) | | |
| 36 | DLLBII61F7 | 1 | 260 | RI: HEK293H- | trypsin | 2 |
| | | | | hDLL4 | | |
| | | | | (2E6/mL) | | |
| | | | | RII: | | |
| | | | | HEK293H- | | |
| | | | | hDLL4 | | |
| | | | | (2E6/mL) | | |
| 37 | DLLBII62C11 | 1 | 260 | RI: HEK293H- | trypsin | 2 |
| | | | | hDLL4 | | |
| | | | | (2E6/mL) | | |
| | | | | RII: | | |
| | | | | HEK293H- | | |
| | | | | mDLL4 | | |
| | | | | (2E6/mL) | | |
| 38 | DLLBII115A5 | 1 | 230 | RI: CHO- | rhNotch | 4 |
| | | | | mDLL4 | 1/Fc | |
| | | | | (2E6/mL) | (RI-RII) | |
| | | | | RII: CHO- | trypsin | |
| | | | | mDLL4 | (RIII) | |
| | | | | (2E6/mL) | trypsin | |
| | | | | RIII: biot- | (RIV) | |
| | | | | rhDLL4 | | |
| | | | | (1 nM) | | |
| | | | | RIV: CHO- | | |
| | | | | mDLL4 | | |
| | | | | (2E6/mL) | | |
| 39 | DLLBII83G1 | 4 | 284 | RI: CHO- | DLL4 | 2 |
| | | | | mDLL4 | IgG | |
| | | | | (2E6/mL) | | |
| | | | | RI: CHO- | | |
| | | | | hDLL4 | | |
| | | | | (2E6/mL) | | |
| 40 | DLLBII80E8 | 1 | 283 | RI: CHO- | DLL4 | 2 |
| | | | | hDLL4 | IgG | |
| | | | | (2E6/mL) | | |
| | | | | RI: CHO- | | |
| | | | | hDLL4 | | |
| | | | | (2E6/mL) | | |

TABLE 4
Screening of periplasmic extracts containing expressed anti-DLL4 VHH

| | | | | Alpha | | | |
|---|---|---|---|---|---|---|---|
| B- | | # | ELISA | Screen | FMAT | FMAT | |
| cell | Representative | unique | hDLL4 % | hDLL4 % | hDLL4 % | mDLL4 % | Biacore^{(a)} |
| lineage | VHH ID | sequences | inhibition | inhibition | inhibittion | inhibittion | k_{d} (s⁻¹) |
| 1 | DLLBII8A09 | 31 | 96 | — | — | — | (1.2^{E-03}-2.4^{E-04}) |
| 2 | DLLBII5B11 | 1 | 98 | — | — | — | — |
| 3 | DLLBII7B05 | 21 | 84 | — | — | — | (2.4^{E-04}) |
| 4 | DLLBII6B11 | 13 | 98 | — | — | — | (9.4^{E-04}-3.7^{E-04}) |
| 5 | DLLBII8C11 | 5 | 57 | — | — | — | (7.3^{E-04}-6.0^{E-04}) |
| 6 | DLLBII19D10 | 1 | 98 | 85 | — | — | 1.3^{E-03} |
| 7 | DLLBII33C05 | 2 | 86 | 75 | — | — | 9.2^{E-04} |
| | | | | | | | (2.1^{E-03}) |
| 8 | DLLBII28B06 | 2 | 23 | 54 | — | — | 7.5^{E-03} |
| | | | | | | | (1.6^{E-04}) |
| 9 | DLLBII17G10 | 1 | 93 | 82 | — | — | 1.5^{E-03} |
| 10 | DLLBII17C01 | 8 | 82 | 84 | — | — | 5.6^{E-04} |
| | | | | | | | (5.6^{E-04}-5.3^{E-04}) |
| 11 | DLLBII19F04 | 1 | 98 | 95 | — | — | 1.1^{E-03} |
| 12 | DLLBII17F10 | 1 | 98 | 88 | — | — | 1.1^{E-03}/ |
| | | | | | | | 3.1^{E-04 (b)} |
| 13 | DLLBII17B03 | 5 | 76 | 77 | — | — | 1.2^{E-03}/ |
| | | | | | | | 2.2^{E-04 (b)} |
| 14 | DLLBII19F12 | 2 | 98 | 98 | — | — | 4.9^{E-04} |
| | | | | | | | (1.0^{E-03}) |
| 15 | DLLBII42B07 | 1 | — | — | — | — | — |
| 16 | DLLBII47D01 | 1 | — | — | 87 | — | — |
| 17 | DLLBII56A09 | 15 | — | — | — | — | 1.1^{E-03} |
| | | | | | | | (9.5^{E-03}-1.1^{E-03}) |
| 18 | DLLBII95F02 | 5 | — | — | 81 | 71 | 6.7^{E-04} |
| 19 | DLLBII96C03 | 20 | — | — | 75 | 83 | — |
| 20 | DLLBII104G01 | 1 | — | — | 94 | 86 | 1.2^{E-03} |
| | | | | | | | (1.4^{E-03}-9.4^{E-04}) |
| 21 | DLLBII102F08 | 3 | — | — | 85 | 75 | — |
| 22 | DLLBII112A03 | 1 | — | — | 72 | 97 | — |
| 23 | DLLBII102G04 | 2 | — | — | 86 | 82 | — |
| 24 | DLLBII101G08 | 1 | — | — | 91 | 92 | 2.1^{E-03} |
| 25 | DLLBII112A04 | 1 | — | — | 75 | 90 | — |
| 26 | DLLBII101H09 | 1 | — | — | 87 | 75 | — |
| 27 | DLLBII101H05 | 1 | — | — | 85 | 83 | — |
| 28 | DLLBII112E07 | 1 | — | — | 80 | 85 | — |
| 29 | DLLBII101F01 | 1 | — | — | 85 | 78 | 2.0^{E-02} |
| 30 | DLLBII104A03 | 1 | — | — | 86 | 83 | — |
| 31 | DLLBII104C04 | 1 | — | — | 87 | 83 | 1.0^{E-03} |
| 32 | DLLBII104B05 | 1 | — | — | 86 | 78 | — |
| 33 | DLLBII107C03 | 1 | — | — | 75 | 80 | — |
| 34 | DLLBII58A11 | 4 | — | — | 95 | 73 | 1.6^{E-03} |
| | | | | | | | (1.7^{E-03}-1.6^{E-03}) |
| 35 | DLLBII61F05 | 1 | — | — | 74 | 76 | — |
| 36 | DLLBII61F07 | 1 | — | — | 79 | 77 | — |
| 37 | DLLBII62C11 | 1 | — | — | 74 | 71 | — |
| 38 | DLLBII115A05 | 1 | — | — | 74 | 84 | 3.1^{E-03} |
| 39 | DLLBII83G01 | 4 | — | — | 87 | 93 | 4.1^{E-04} |
| 40 | DLLBII80E08 | 1 | — | — | 71 | 82 | — |

^{(a)}if multiple unique variants within a B-cell lineage are identified, the range (max-min) in off-rate or the off-rate of a lineage member is given between brackets in italics).
^{(b)}heterogeneous fit: fast and slow off-rate determined.

TABLE 5
Framework and CDR Sequences of anti-DLL4 VHH

| VHH ID | | | | | | | |
|---|---|---|---|---|---|---|---|
| SEQ ID NO | Framework 1 | CDR 1 | Framework 2 | CDR 2 | Framework 3 | CDR 3 | Framework 4 |
| DLLBII0 | EVQLVESGGGL | TYNIG | WFRQAPGKERE | CISSSDGST | RFTISRDNAKN | PFAYYSDL | WGQGTQVTVSS |
| 5B06 | VQPGGSLRLSC | | WVS | NYADSVKG | TVYLQMNNLKP | CGVNGVDY | |
| 167 | AASGFTLD | | | | EDTAVYYCAA | | |
| DLLBII0 | EVQLVESGGGL | YYNIG | WFRQAPGKERE | CINSSDGST | RFTISRDNAKN | PFSYYSHL | WGQGTQVTVSS |
| 5B08 | VQPGGSLRLSC | | WVS | YYTDSVKG | TVYGLMNSLKP | CGVNGYDY | |
| 168 | AASGFTLD | | | | EDTAIYYCAA | | |
| DLLBII0 | EVQLVESGGGL | YYNIG | WFRQAPGKERE | CISSSDGST | RFTISRDNAKN | PFSYYSSL | WGQGTQVTVSS |
| 5B09 | VQPGGSLRLSC | | WVS | AYADSVKG | TVYLQMNSLKP | CGVNEYDY | |
| 169 | AASGFTLD | | | | EDTAVYYCAA | | |
| DLLBII0 | EVQLVESGGGL | LHVIG | WLRQAPGKERE | CISSSDGST | RFTISRDNAKN | PWDSWYCG | WGQGTQVTVSS |
| 5B11 | VQPGGSLRLSC | | WVS | YYADSVKG | TVYLQMNSLKP | IGNDYDY | |
| 170 | AISGFTLD | | | | EDTAVYYCAA | | |
| DLLBII0 | EVQLVESGGGL | YYNIG | WFRQAPGKERE | CIRGSNGST | RFTISRDNAKN | PFIHYSDL | WGQGTQVTVSS |
| 5D11 | VQPGGSLRLSC | | WVS | GYTDSVKG | TVYLQMNSLKP | CGVNGYDY | |
| 171 | AASGFTLD | | | | EDTAVYYCAA | | |
| DLLBII0 | EVQLVESGGGL | KYAIG | WFRQAPGKERE | CISSRGGST | RFITSRDNAKN | DPIHNCYS | WGQGTQVTVSS |
| 6A02 | VQAGGSLRLSC | | GVS | YYVDSVKG | TVYYQMNSLKP | GSSYYYSP | |
| 172 | AASGFTLD | | | | EDTAVYYCAA | EAVYDY | |
| DLLBII0 | EVQLVESGGGL | YYNIG | WFRQAPGKERE | CITSSNGST | RFTISRDNAKN | PFAHYSDL | WGQGTQVTVSS |
| 6A05 | VQPGGSLRLSC | | WVS | YYTDSVKG | TVYLQMNSLKP | CGVNGYDY | |
| 173 | AASGFTLD | | | | EDTAVYYCAA | | |
| DLLBII0 | EVQLVESEGGL | SYAMG | WYRQAPGKQRE | VISNGGITN | RFTISRDNAKN | SGSYYYPT | WGQGTQVTVSS |
| 6B11 | VQAGGSLRLSC | | LVA | YPNSVKG | TVYLQMNSLKP | DVHEYDY | |
| 174 | AASGSTFS | | | | EDTAVYYCFY | | |
| DLLBII0 | EVQLVESGGGL | YYNIG | WFRQAPGKERE | CINSSDGST | RFTISRDNAKN | PFEYYSDL | WGQGTQVTVSS |
| 6E02 | VQPGGSLRLSC | | WVS | YYADSVKG | TVYLQMNSLKP | CGVNGYDY | |
| 175 | AASGFTLD | | | | EDTAVYYCAA | | |
| DLLBII0 | EVQLVESGGGL | YYAIG | WFRQAPGKERE | CISSRGGST | RFTISRDNAKN | DPIHNCYS | WGQGTQVTVSS |
| 6E04 | VQAGGSLRLSC | | GIS | FYVDSVKG | TVYLQMNSLKP | GRYYYSPE | |
| 176 | AASGFTLD | | | | EDTAVYYCAA | AVYEY | |
| DLLBII0 | EVQLVESGGGL | YHNIG | WFRQAPGKERE | CISSSGGST | RFTISRDNAKN | PFSHYSDL | WGQGTQVTVSS |
| 6E12 | VQPGGSLRLSC | | WVS | AYADSVKG | TVYLQMNSLKP | CGVNAIDY | |
| 177 | AASGFTLD | | | | EDTAVYYCAA | | |
| DLLBII0 | EVQLVESGGGL | YYNIG | WFRQAPGKERE | CINSSDGST | RFTVSRDNAKN | PFEYYSDL | WGQGTQVTVSS |
| 6G09 | VQPGGSLRLSC | | WVS | YYADSVKG | TVYLQMNSLKP | CGVNGYDY | |
| 178 | AASGFTLD | | | | EDTAVYYCAA | | |
| DLLBII0 | EVQLVESGGGL | SYAMG | WYRQAPGKQRE | AFSTGGSTN | RFTISRDNAKN | SGSYYYPT | WGQGTQVTVSS |
| 7A02 | VQAGGSLRLSC | | WVA | YADSVKG | TVYLQMNSLKP | DVFEYDY | |
| 179 | AASGSTFN | | | | EDTAVYYCFY | | |
| DLLBII0 | EVQLVESGGGL | YYAVG | WFRQAPGKERE | CISSRGGST | RFTTSRNNAKN | HPLQNCCG | WGQGTQVTVSS |
| 7B05 | VQAGGSLRLSC | | GVS | FYADSVKG | TVYLQMNSLKP | GSAYASPE | |
| 180 | AASGFALD | | | | EDTAVYYCAA | AVYEY | |
| DLLBII0 | EVQLVESGGGL | YYNIG | WFRQAPGKERE | CINSSDGST | RFTISRDNAKN | PFAYYSNL | WGQGTQVTVSS |
| 8A09 | VQPGGSLRLSC | | WVS | YYADSVKG | TVYLQMNSLKP | CGVNGYDY | |
| 181 | AASGFTLD | | | | EDTAVYYCAA | | |
| DLLBII0 | EVQLVESGGGL | YYAVG | WFRQAPGKERE | CISSRGGST | RFTTSRDNAKN | DPIHNCYS | WGQGTQVTVSS |
| 8B05 | VQAGGSLRLSC | | GVS | YYVSDVKG | TVYLQMNSLKP | GNYYASPE | |
| 182 | AASGFALD | | | | EDTAVYYCAA | AVYDY | |
| DLLBII0 | EVQLVESGGGL | DYAIG | WFRQAPGKERE | CISSHDRTT | RFTISSDNAKN | DPLVCGYN | WGQGTQVTVSS |
| 8C11 | VQAGGSLRLSC | | GVS | YYADSVKG | TVYLQMNSLKP | DPRLADY | |
| 183 | AASGFTFD | | | | EDTAVYYCAA | | |
| DLLBII0 | EVQLVESGGGL | YYNIG | WFRQAPGKERE | CITSSYGST | RFTISRDNAKN | PFAHYSDL | WGQGTQVTVSS |
| 8H06 | VQPGGSLRLSC | | WVS | YYTDSVKG | TVYLQMNSLKP | CGVNGYDY | |
| 184 | AASGFTLD | | | | EDTAVYYCAA | | |
| DLLBII0 | EVQLVESGGGL | YHNIG | WFRQAPGKERE | CISSSDGRT | RFTISRDNAKN | PFTHYSDL | WGQGTQVTVSS |
| 9C01 | VQPGGSLRLSC | | WVS | AYADSVKG | TVYLQMNSLKP | CGVNEYDY | |
| 185 | AASGFTLD | | | | EDTAVYYCAA | | |
| DLLBII1 | EVQLVESGGGL | DYAIG | WFRQAPGKEPE | CISSSGGST | RFTISRDNAKN | PGIAACRG | WGQGTQVTVSS |
| 00G01 | VQPGGSLRLSC | | GIG | YYADSVKG | TVYLQMNSLKP | IHY | |
| 186 | AASGFTFD | | | | EDTAVYYCAT | | |
| DLLBII1 | KVQLVESGGGL | DYAIG | WFRQAPGKEPE | CISSSGGIT | RFTISRDNAKN | PGIAACRG | TGQGTQVTVSS |
| 01B12 | VQPGGSLRLSC | | GIS | YYADSVKG | TVYLQMNLKPE | IHY | |
| 187 | AASGFTFD | | | | DTAVYYCAT | | |
| DLLBII1 | EVQLVESGGGL | NYDMS | WVRWAPGKGPE | AINSGGGTT | RFTISRDNAKN | PRGWGPTG | WGQGTQVTVSS |
| 01E04 | VQPGGSLRLSC | | WVS | YYADSVKG | TLYLQMNSLKP | PHEYGY | |
| 188 | AASGFTFG | | | | EDTAVYYCAT | | |
| DLLBII1 | EVQLVESGGGL | NYAMG | WFRQAPGKERE | AISWSGGDT | RFTISRDNAKN | SFQSGAAP | WGQGTQVTVSS |
| 01F01 | VQAGGSLRLSC | | FVA | YYADSVKG | TVCLQMNSLKP | GANFYDY | |
| 189 | AASGRTFS | | | | EDTAVYYCAA | | |
| DLLBII1 | EVQLVESEGGS | SYAMG | WFRQAPGKERE | AINWSGGYT | RFTISRDNAKN | PAPGSSGY | WGQGTQVTVSS |
| 01F03 | VQAGGSLRLSC | | FVA | YYADSVRG | TVYLQMNSLKP | EYDY | |
| 190 | AASGRTFS | | | | EDTAVYYCAA | | |
| DLLBII1 | EVQLVESGGGL | SYAMG | WFRQAPGKERE | AIFWSGGST | RFTISRDIAKN | PSPGSSGY | WGQGTQVTVSS |
| 01F06 | VQAGGSLRLSC | | FVA | YYADSVRG | TVYLQMNSLKP | EYDY | |
| 191 | AASGRTFS | | | | EDTAVYYCAA | | |
| DLLBII1 | EVQLVESGGGL | NYRMG | WFRQGPGKERE | AIGRNGQNT | RFTVTRDNAKN | SLRGWDTT | WGQGTQVTVSS |
| 01F08 | VQTGDSLRLSC | | FVA | YYTDSVKG | MMYLQMNSLKP | RIDYEY | |
| 192 | AASGFTFS | | | | EDSAVYTCAA | | |
| DLLBII1 | EVQLVESGGGL | VYAIG | WFRQAPGKEPE | CISSSGSIT | RFTTSRDSAKN | PGIAACRG | WGQGTQVTVSS |
| 01F10 | VQPGGSLRLSC | | GIS | YYADSVKG | TVYLQMNSLKP | IHY | |
| 193 | TASGFTFD | | | | EDTAVYYCAT | | |
| DLLBII1 | EVQLVESGGGL | NYDMS | WVRQAPGKGPE | AINSGGDTT | RFTISRDNAKN | PRGWGPTG | WGQGTQVTVSS |
| 01G02 | VQPGGSLRLSC | | WVS | YYADSVKG | TLYLQMNSLKP | PHEYGY | |
| 194 | AASGFTFG | | | | EDTAVYYCAT | | |
| DLLBII1 | EVQLVESRGGL | SYAMG | WFRQAPGKERE | TINWSGGST | RFTISRDNAKN | PAPGSSGY | WGQGTQVTVSS |
| 01G03 | VQAGGSLRLSC | | FVA | YYADSVKG | TAYLQMNSLKP | EYDY | |
| 195 | AASGRTFN | | | | EDTAVYYCAA | | |
| DLLBII1 | EVQLVESGGGS | SYAMG | WFRQAPGKERE | AVYWSGGST | RFTISRDNAKN | PSPGSSGY | WGQGTQVTVSS |
| 01G05 | VQAGGSLRLSC | | FVA | YYADSVRG | TVYLQMNSLKP | EYDY | |
| 196 | AASGRTFS | | | | EDTAVYYCAA | | |
| DLLBII1 | EVQLVESGGGL | SYAMA | WFRQAPGKERE | AIRWSGGTA | RFTISRDNAKN | RAADTRLG | WGQGTQVTVSS |
| 01G08 | VQAGGSLRLSC | | FVA | YYADSVQG | TVYLQMNSLKP | PYEYDY | |
| 197 | AASGFTFS | | | | EDTAVYYCAN | | |
| DLLBII1 | EVQLVESGGGL | NYDMS | WVRQAPGKGPE | AINSGGGIT | RFTISRDNAKN | PRGWGPTG | WGQGTQVTVSS |
| 01H02 | VQPGGSLRLSC | | WVS | YYADFVKG | TLYLQMSSLKP | PHEYGY | |
| 198 | AASGFTFG | | | | EDTAVYYCAT | | |
| DLLBII1 | EVQLVESGGGL | DYAIG | WFRQAPGKEPE | CISSSGGIT | RFTISRDNAKN | PGIAACRG | TGQGTQVTVSS |
| 01H03 | VQPGGSLRLSC | | GIS | YYTDSVKG | TVYLQMNSLKP | IHY | |
| 199 | AASGFTFD | | | | EDTAVYYCAT | | |
| DLLBII1 | EVQLVESGGGS | TYAMG | WFRQAPGKEHE | AIGRGTGAT | RFTISRDNAKN | GRGFYHDY | RGQGTQVTVSS |
| 01H05 | AQAGGSLRLSC | | FVS | SYGDSVKG | TVYLQMNSLQL | SYEY | |
| 200 | AASGRTSS | | | | EDTGDYYCVA | | |
| DLL1BII1 | EVQLVESGGGL | YYTIV | WFRQAPGKERK | CISSRDGSR | RFTISRDNAKN | GPDCSSYD | WGQGTQVTVSS |
| 01H09 | VQPGGSLRLSC | | GVS | YYADSVKG | TVYLRMNSLKP | Y | |
| 201 | AASGFTLG | | | | EDTAVYYCAA | | |
| DLLBII1 | EVQLVESGGGL | NYRMG | WFRQGPGKEREF | AIGRNGQNT | RFTVTRDNAKN | SLRGWDTT | WGQGTQVTVSS |
| 02F08 | VQTGDSLRLSC | | VA | YYTDSVKG | MVYLQMNSLKP | RIDYEY | |
| 202 | AASGSTFS | | | | EDSAYVTCAA | | |
| DLLBII1 | EVQLMESGGGL | SYAMS | WVRQAPGKGLEW | RITSGGRTT | RFTISRDNSKN | ARGDIDVY | RGQGTQVTVSS |
| 02G04 | VQPGGSLRLSC | | VS | YRDSVKG | TLYLQMNSLKP | TLSDS | |
| 203 | AASGFTFS | | | | EDTALYYCAK | | |
| DLLBII1 | EVQLVESGGGL | SYAMS | WVRQAPGKGLEW | RITSGGRAT | RFTISRDNSKN | ARGDIDVY | RGQGTQVTVSS |
| 02H07 | VQPGGSLRLSC | | VS | YRDSVKG | TLYLQMNSLKP | TLSDS | |
| 204 | AASGFTFS | | | | EDTALYYCAK | | |
| DLLBII1 | EVQLVESGGGL | NYDMS | WVRRPPGKGPEW | AINSGGGST | RFTISRDNAKN | PRGWGPTG | WGQGTQVTVSS |
| 02H09 | VQPGGSLRLSC | | VS | YYADSVKG | TLYLQMNSLKP | PHEYGY | |
| 205 | AASGFTFG | | | | EDTAVYYCAT | | |
| DLLBII1 | EVQLVESGGGL | DYAIG | WFRQAPGKEREG | CISSSDGST | RFTVSSNNADD | RLFSGGCA | SGQGTQVTVSS |
| 03A04 | VQAGGSLRLSC | | VS | YYADSVKG | TVYLQMNSLKP | VVAGTSWA | |
| 206 | AASGFTFD | | | | EDTAVYYCAV | DFGS | |
| DLLBII1 | AVQLVESGGGL | DYAIG | WFTQAPGKEREG | CISSSDGST | RFTISSDKVKN | RLFKGGCA | TGQGTQVTVSS |
| 03B05 | VQAGGSLRLSC | | VS | HYADSVKG | TVYLQMNSLKP | VVAGTSWA | |
| 207 | AASGFTFD | | | | EDTAVYYCAV | DFGS | |
| DLLBII1 | EVQLVESGGGL | IAAMG | WYRQAPGKQREL | TVSNAATTR | RFTISRDNAKT | LATTVTPS | WGQGTQVTVSS |
| 04A03 | VQAGGSLRLSC | | VA | YADSAKG | VSLQMDNLKPE | WVNY | |
| 208 | AASGDIPR | | | | DTGVYYCYS | | |
| DLLBII1 | EVQLVESGGGL | YYDMS | WVRQAPGKGPEW | AINSGGGST | RFTISRDNAKN | PRGWGPTG | WGQGTQVTVSS |
| 04A05 | VQPGGSLRLSC | | VS | YYADSVKG | MLYLQMSLKPE | PHEYDY | |
| 209 | AASGFAFG | | | | DTAVYYCAT | | |
| DLLBII1 | EVQLVESGGGL | YYRMG | WFRQAPGKEREF | AIGKSGRNT | RFTVSRDNAKN | SLRGWDTT | WGQGTQVTVSS |
| 04B02 | VQAGGSLRLSC | | VA | YYGDYVKG | TVYLQMNTLKP | WIDYEY | |
| 210 | DASGRGFS | | | | EDTAVYYCAA | | |
| DLLBII1 | EVQLVESGGGS | IDMVA | WYRQAPGKEREL | SISSGGSTN | RFTISREYFKN | DSRRGGVG | WGQGTQVTVSS |
| 040B05 | VQAGGSLRLSC | | VA | YADSVKG | MMYLQMNSLKF | NFFRS | |
| 211 | AASGSISR | | | | EDTAVYYCNA | | |
| DLLBII1 | EVQLVESRGGL | SYDMS | SVRQAPGKGPEW | AINSGGGST | RFTISRDNAKS | PRGWGPTG | WGQGTQVTVSS |
| 04B08 | VQPGGSLRLSC | | VS | YYADSVKG | TLYLQMNSLKP | PIEYAY | |
| 212 | AASGFTFG | | | | EDTAVYYCAI | | |
| DLLBII1 | EVQLVESGGGL | SYVMG | WYRQAPGKQREL | HISTRGITY | RFTISRDNAKN | RRNFLSNY | WGQGTQVTVSS |
| 04C04 | VQAGGSLRLSC | | VA | YADSVKG | TMYLQMNSLKP | | |
| 213 | AAAGSTFS | | | | EDTAVYYCNT | | |
| DLLBII1 | EVQLVESGGGL | DYAIG | WFRQAPGKEPEG | CISSSGGIT | RFTISRDNAKS | PGIAACRG | TGQGTQVTVSS |
| 04C12 | VQPGGSLRLSC | | IS | YYADSVKG | TVYLQMNSLKP | IHY | |
| 214 | AASGFTFD | | | | EDTAVYYCAT | | |
| DLLBII1 | EVQLVESGGGL | DYAIG | WFRQAPGKEREG | CISSSDGST | RFTISSDNAKN | AWCDSSWY | WGQGTQVTVSS |
| 04G01 | VQAGGSLRLSC | | VS | YYADSVKG | TVYLQMNSLKP | RSFVGY | |
| 215 | AASGFTFD | | | | EDTAVYYCAT | | |
| DLLBII1 | EVQLVESGGGL | DYAIG | WFRQAPGKEREG | CISSSDDST | RFTISSNNAKN | RLFSGGCA | SGQGTQVTVSS |
| 05G01 | VQAGGSLRLSC | | VS | YYADSVKG | TAYLQMNSLKP | VVARTSWA | |
| 216 | AASGFTFD | | | | EDTAVYYCAV | DFGS | |
| DLLBII1 | EVQLVESGGGF | DYAIG | WFRQAPGKEPEE | CISSSGGIT | RFTISRDNAKN | PGIAACRG | WGQGTQVTVSS |
| 06A01 | VQPGGSLRLSC | | IS | YYADSVKG | TVYLQMNSLKP | IHY | |
| 217 | AASGFTFD | | | | EDTAVYYCAT | | |
| DLLBII1 | EVQLVESGGGL | SYDMS | WVRQAPGKGPEW | AISNGGGIT | RFTISRDNTKN | PRGWGPTG | WGQGTQVTVSS |
| 06F01 | VQSGGSLRLSC | | VS | YYADSVNG | TLYLQMNSLKP | PHEYGY | |
| 218 | AASGFTFG | | | | EDTAVYYCAT | | |
| DLLBII1 | EVQLVESGGGL | NYAMG | WYRQAPGKQREL | GISSDGSTH | RFTISRDDAKN | PVKVAGLE | WGQGTQVTVSS |
| 06H01 | VQAGGSLRLSC | | VV | YADSAKG | TVYLQMNSLKT | YAY | |
| 219 | AASGFTFS | | | | EDTAVYYCYV | | |
| DLLBII1 | EVQLVESGGGL | VSDMR | WYRQAPGLQYEL | RITSGSITD | RFTISRDNAKN | DVQHSAWL | WGQGTQVTVSS |
| 07C03 | VQPGGSLRLSC | | VA | YSDSVKG | TVYLQMNSLKP | KPLTY | |
| 220 | EVSGSIGS | | | | EDTAVYYCNA | | |
| DLLBII1 | EVQLVESGGGL | INGMG | WYRQAPGKQREA | TITRGGIRD | RFTISRDIARN | DIY | WGRGTQVTVSS |
| 12A03 | VQPGGSLRLSC | | VA | YTDSVKG | TVYLQMNNLKP | | |
| 221 | AASGIRFS | | | | EDSAVYYCNI | | |
| DLLBII1 | EVQLVESGGGL | GMG | WFRQAPGKEREF | AITSDGSTN | RFTISRDNAKN | PYYSDFEG | WGQGTQVTVSS |
| 12A04 | VQAGGSLRLSC | | VA | YADWVKG | AVSLQMNSLKP | TTTEYDY | |
| 222 | AAFGRTPY | | | | EDTAVYYCTA | | |
| DLLBII1 | EVQLVESGGGL | SYATG | WFRQAPGKEREF | ALRWSIGSI | RFTISGDNAEN | TTRGRYSA | WGQGTQVTVSS |
| 12E07 | VQAGGSLRLSC | | VA | ASVYYDDSV | TVYLQMNALKP | LSASAYDY | |
| 223 | AASGRTVR | | | KG | EDTAIYYCAS | | |
| DLLBII1 | EVQLVESGGGL | SYDMS | WVRRSPGKGPEW | SINSGGGST | RFTISRDNAKN | DRYIRARQ | WGQGTQVTVSS |
| 15A05 | VQPGGSLRLSC | | VS | YYADFVKG | TLYQLMNSLKP | GDYWGAYE | |
| 224 | AASGFTFG | | | | EDTAVYYCAA | YDY | |
| DLLBII1 | EVQLVESGGGL | YYAIG | WFRQAPGKEREG | CISSRGGST | RFTISRDNAKN | DPIHNCYS | WGQGTQVTVSS |
| 6A03 | VQAGGSLRLSC | | VS | YYEDSVKG | TVYLQMNSLKP | GRYYASPD | |
| 225 | AASGFTLD | | | | EDTAVYYCAA | AVYDY | |
| DLLBII1 | KVQLVESGGGL | DYAIG | WFRQAPGKEREG | CISSHDGTT | RFTISSDNAKN | DPLVCGYN | WGQGTQVTVSS |
| 6A07 | VQAGGSLRLSC | | VS | YYASSVKG | TVYLQMNSLKP | DPRLADY | |
| 226 | AASGFTFD | | | | EDTAVYYCAA | | |
| DLLBII1 | EVQLVESGGGL | YHNIG | WFRQAPGKEREW | CISSSGGST | RFTISRDNAKN | PFNHYSDL | WGQGTQVTVSS |
| 6A09 | VQPGGSLRLSC | | VS | AYADSVKG | TVYLQMNSLKP | CGVNAIDY | |
| 227 | AASGFTLD | | | | EDTAVYYCAA | | |
| DLLBII1 | EVQLVESGGGL | YYNIG | WFRQAPGKEREW | CISSSDGST | RFTISRDNAKN | PFSYYSSL | WGQGTQVTVSS |
| 6C11 | VQPGGSLRLSC | | VS | AYADSVKG | TVYLQMNSLKP | CGVNEYDY | |
| 228 | AASGFTLD | | | | EDTAVYYCAA | | |
| DLLBII1 | EVQLVESGGGL | YYNIG | WFRQAPGKEREW | CISSTNGNT | RFSISRDNARN | PFSYYNNL | WGQGTQVTVSS |
| 6D11 | VQPGGSLRLSC | | VS | YYADSVKG | TVYLQMNSLKP | CGVNGVDY | |
| 229 | AASGFTLD | | | | EDTAVYYCAA | | |
| DLLBII1 | EVQLVESGGGL | DYAIG | WFRQAPGKEREG | CISSSDDST | RFSISRDNARN | PFSYYNNL | WGQGTQVTVSS |
| 6E02 | VQAGGSLRLSC | | VS | YYADSVKG | TVYLQMNSLKP | CGVNGVDY | |
| 230 | AASGFTFD | | | | EDTAVYYCAA | | |
| DLLBII1 | EVQLVESGGGL | YYNIG | WFRQAPGKEREG | CITSSNGST | RFTISRDNAKN | PFAHYSDL | WGQGTQVTVSS |
| 6E08 | VQPGGSLRLSC | | VS | YYTDSVKG | TVYLQMNSLKP | CGVNGYDY | |
| 231 | AASGFTLD | | | | EDTAVYYCAA | | |
| DLLBII1 | EVQLVESGGGL | YYNIG | WFRQAPGKEREW | CISSSDGST | RFTISRDNAKN | PFAYYSDL | WGQGTQVTVSS |
| 6H02 | VQPGGSLRLSC | | VS | GYADSVKG | TVYLQMNSLKP | CGVNEYDY | |
| 232 | AASGFALD | | | | EDTAVYYCAA | | |
| DLLBII1 | EVQLVESGGGL | SYAMG | WYRQAPGKQREL | AISSDDSTY | RFTISRDYAKN | PHSDYEEA | WGQGTQVTVSS |
| 6H09 | VQAGGSLRLSC | | VA | YADCVKG | TVYLQMNSLKP | PSDFGS | |
| 233 | AASGSTFT | | | | EDTAVYYCNA | | |
| DLLBII1 | EVQLVESGGGL | DYAIG | WFRQAPGKEREG | CISSSDDST | RFTISRNNAKN | RLFSGGCA | SGQGTQVTVSS |
| 7A12 | VQAGGSLRLSC | | VS | YYADSVKG | TVYLQMNSLKP | VVVGTSWA | |
| 234 | AASGFTFD | | | | EDTAVYYCAV | DFGS | |
| DLLBII1 | EVQLVESGGGL | NYALG | WRFQAPGKEREW | CISSSDGTT | RFTISRDNVKN | SLGSSWCA | WGQGTQVTVSS |
| 7B03 | VQPGGSLRLS | | VS | YYADSVKG | TVYLQMNRLKP | YDY | |
| 235 | CAASGFTFE | | | | EDTAIYYCAL | | |
| DLLBII1 | EVQLMESGGGL | DYAIG | WFRQAPGKEREG | CISSYDGTT | RFITSSDNAKN | DPLVCGYN | WGQGTQVTVSS |
| 7B09 | VQAGGSLRLSC | | VS | YYADSVKG | TVYLQMNSLKP | DPRLADY | |
| 236 | AASGFTFD | | | | EDTAVYYCAA | | |
| DLLBII1 | EVQLVESGGGL | DYPIG | WLRQAPGKEREG | CISSSDDST | RFTISSNNAKN | RLFSGGCA | SGQGTQVTVSS |
| 7C01 | VQAGGSLRLSC | | VS | YYADSVKG | TVYLQMNSLKP | VVAGTSWA | |
| 237 | AASGFTFD | | | | EDTAVYYCAV | DFGS | |
| DLLBII1 | EVQLVESGGGL | SYAMG | WYRQAPGKQREL | VISSGDRTN | RFTISRDNAKN | SGSYYYPT | WGQGTQVTVSS |
| 7C08 | VQAGGSLRLSC | | VA | YLDSVKG | TVYLQMNSLKP | DVHEYAY | |
| 238 | AASGSTFS | | | | EDTAVYYCFY | | |
| DLLBII1 | EVQLVESGGGL | YYNIG | WFRQAPGKEREW | CISSGDGST | RFTISRDNAKN | PFEYYSAY | WGQGTQVTVSS |
| 7E04 | LVQPGGSLRLS | | VS | YYADSVKG | TVYLQMNSLKP | CGVNRYDY | |
| 239 | CAASGFTLD | | | | EDTAVYYCAA | | |
| DLLBII1 | EVQLVESGGGL | NYAMG | WFRQAPGKEREF | GINWSGGST | RFTISRDNAEN | AHDNYWFT | WGQGTQVTVSS |
| 7F10 | VQAGGSLRLSC | | VS | YYADSVKG | TVYLHMNSLKP | DSLGRGLK | |
| 240 | ASSGRTLL | | | | EDTAVYYCAA | Y | |
| DLLBII1 | EVQLVESGGGL | SYAMG | WYRHQAPGKQRE | AAISSDGST | RFTISRDNAKN | KTFGSNWY | WGQGTQVTVSS |
| 7G10 | VQAGGSLRLSC | | LV | HYADSVKG | TMYLQMNSLKP | DDY | |
| 241 | AASGSTFS | | | | EDTAVYYCNT | | |
| DLLBII1 | EVQLVESGGGL | YYAIG | WFRQAPGKEREG | CISSRGGST | RFTISRDNAKN | DPIHNCYS | WGQGTQVTVSS |
| 8D02 | VQAGGSLRLSC | | VS | YYTDSVKG | TVYLQMNSLKP | GRYYASPE | |
| 242 | AASGFTLD | | | | EDTGVYYCAA | AVYDY | |
| DLLBII1 | EVQLVESGGGL | YYAVG | WFRQAPGKEREG | CISSSGGST | RFTISRDNAKN | DPFHNCYS | WGQGTQVTVSS |
| 8F05 | VQAGGSLRLSC | | VS | YYEDSVKG | TVYLQMNNLKP | GSHYSSPE | |
| 243 | AASAFTLD | | | | EDTAVYYCAA | AVYEY | |
| DLLBII1 | EVQLVESGGGL | YYNIG | WFRQAPGKGREW | CINSSDGST | RFTISRDNAKN | PFEYYSDL | WGQGTQVTVSS |
| 8H08 | VQPGGSLRLSC | | VS | YYADSVKG | TVYLQMNSLKP | CGVNGYDY | |
| 244 | AASGFTLD | | | | EDTAVYYCAA | | |
| DLLBII1 | EVQLVESGGGL | YYNIG | WFRQAPGKEREW | CISSSDGRT | RFTMSRDNAKN | PFNYYSDL | WGQGTQVTVSS |
| 09B09 | VQPGGSLRLSC | | VS | NYVDSVKG | TVYLQMNSLKP | CGVNGVDY | |
| 245 | AASGFTLD | | | | EDTAVYYCAA | | |
| DLLBII1 | EVQLVESGGGL | SYAMG | WYRQAPGNGREL | VISSGGSTN | RFTISRDNAKN | SGSYYYPT | WGQGTQVTVSS |
| 9D04 | VQAGGSLRLSC | | VA | YADSVKG | TVYLQMNSLKP | DVHEYAY | |
| 246 | AASGSTFS | | | | EDTAVYCCFY | | |
| DLLBII1 | EVQLVESGGGL | NYNIG | WFRQAPGKEREW | CITSSNGST | RFTISRDNAKN | PFAHYSDL | WGQGTQVTVSS |
| 9D07 | VQPGGSLRLSC | | VS | YYTDSVKG | TVYLQMNSLKP | CGVNGYDY | |
| 247 | AASGFTLD | | | | EDTAVYYCAA | | |
| DLLBII1 | EVQLVESGGGL | SYDMS | WVRQGPGKEWRE | SINSGVGKT | RFTIFRDNAKN | EMDGSRYV | EGQGTQVTVSS |
| 9D10 | VQAGDSLRLSC | | WVS | YYADSVKG | MVYLQMNNLKP | | |
| 248 | AASGFTVG | | | | EDTAVYYCAT | | |
| DLLBII1 | EVQLVESGGGL | TYAMA | WYRQAPGKQREL | GISFDGSTH | RFTISRDDAKN | VHPSTGFG | WGQGTQVTVSS |
| 9F04 | VQAEGSLRLSC | | VA | YAESVKG | TVSLQMNSLKP | S | |
| 249 | AASGSTFS | | | | EDAAVYYCYS | | |
| DLLBII1 | EVQLVESGGGL | SYAMG | WYRQAPGKQREL | AISSDDSTY | RFTISRDNAKN | PHSDYDEE | WGQGTQVTVSS |
| 9F12 | VQAGGSLRLSC | | VA | YADCVKG | TVYLQMNSLKP | APSDFGS | |
| 250 | TASGSTFT | | | | EDTAVYYCNA | | |
| DLLBII2 | EVQLVESGGGL | DYAIG | WFRQAPGKEREG | CISSSDDST | RFTISSNNAKN | RLFSGGCA | SGQGTQVTVSS |
| 4C07 | VQAGGSLRLSC | | VS | YYADSVKG | TVYLTMNSLKP | VVASTSWA | |
| 251 | AASGFTFD | | | | EDTAVYYCAV | DFGS | |
| DLLBII2 | EVQLVESGGGL | DYAIG | WFRQAPGKEREG | CISSSDGST | RFTISSNNAKN | RLFRGGCA | SGQGTQVTVSS |
| 4D07 | VQAGGSLRLSC | | VS | YYADSVKG | RAYLQMNSLKP | VVAGTSWA | |
| 252 | AASGFTFD | | | | EDTAVYYCAV | DFGS | |
| DLLBII2 | EVQLVESGGGL | DYAIG | WFRQAPGKEREG | CISSYDGTT | RFTISSDNAKN | DPLVCGYN | WGQGTQVTVSS |
| 5G05 | VQAGGSLRLSC | | VS | YYADSVKG | TVYLQMNSLKP | DPRLADY | |
| 253 | AASGFTFD | | | | EDTAVYYCAA | | |
| DLLBII2 | EVQLVESGGGL | YYAIG | WFRQAPGKEREG | CISSRGGST | RFTISRDNAKN | DPIHNCYS | WGQGTQVTVSS |
| 5H07 | VQAGGSLRLSC | | VS | YYEDSVKG | TVYLQMNSLKP | GRYYASPD | |
| 254 | AASGFTLD | | | | EDTAVYYCAA | AVYEY | |
| DLLBII2 | EVQLVESGGGL | YYAIG | WFRQAPGKEREG | CISSRGSST | RFTISRDNAKN | DPIHNCYS | WGQGTQVTVSS |
| 6C02 | VQAGGSLRLSC | | VS | YYASDVKG | TVYLQMNSLKP | GNGYDSPE | |
| 255 | AASGFTLD | | | | EDTAVYYCAA | AVYDY | |
| DLLBII2 | EVQLVESGGGL | YYAIG | WFRQAPGKEREG | CISSSGGST | RFTISRDNAKN | DPFHNCYS | WGQGTQVTVSS |
| 6H11 | VQAGGSLRLSC | | VS | YYADSVKG | TVYLQMNSLKP | GSAYSSPE | |
| 256 | TASGFTLD | | | | EDTAVYYCAA | AVYEY | |
| DLLBII2 | EVQLVESGGGL | TYAMG | WYRQDPGNQREL | AISSDGSTH | RFTISRDNAKN | PVKVAGLE | WGQGTQVTVSS |
| 8B06 | VQAGGSLRLSC | | VA | YADSVKG | TVYLQMNSLKP | YDY | |
| 257 | AASGSTFS | | | | EDTAVYYCYA | | |
| DLLBII3 | EVQLVESGGGL | NYAIG | WFRQAPGKEREW | CISGFDGST | RFTISRDNAKN | SVGSSWCA | WGQGTQVTVSS |
| 3A06 | VQPGGSLRLSC | | VS | YYADSVKG | TVYLQMNSLKP | YDY | |
| 258 | AASGFTLD | | | | EDTAVYYCAA | | |
| DLLBII3 | EVQLVESGGGL | NYALG | WFRQAPGKEREW | CISSSDGTT | RFTISRDNAKN | SLGSSWCA | WGQGTQVTVSS |
| 3A10 | VQPGGSLRLSC | | VS | YYADSVRG | TVYLQMNRLKP | YDY | |
| 259 | AASGFTFE | | | | EDTAIYYCAL | | |
| DLLBII3 | EVQLVESGGGL | NYVIG | WFRQAPGKEREE | CISSSGGST | RFTISRDNAKN | DSLPCYYD | WGQGTQVTVSS |
| 3C05 | VQAGGSLRLSC | | VS | DYLDSVKG | TVYLQMNSLKP | KMVYDY | |
| 260 | AASGFTLD | | | | EDTAVYYCAA | | |
| DLLBII3 | EVQLVESGGGL | YYVIG | WFRQAPGKEREG | CTSSSGGST | RFTISRDNAKN | DSFACDYG | WGQGTQVTVSS |
| 3C09 | VQAGGSLRLSC | | VS | YYADSVKG | TVYLQMHSLKP | KMIYDY | |
| 261 | TASGFKLD | | | | EDTAVYYCAA | | |
| DLLBII3 | EVQLVESGGGL | NYAMG | WFRQAPGKEREW | CISGSDGST | RFTISRDNAKN | SLGSSWCA | WGQGTQVTVSS |
| 3C10 | VQPGGSLRLSC | | VS | YYADSVKG | TVYLQMHSLKP | YDY | |
| 262 | AASGFGFD | | | | EDTAVYYCAA | | |
| DLLBII3 | EVQLVESGGGL | DYAIG | WFRQAPGKEREG | CISSHYGTT | RFTISSDNAKN | DPLCVGYN | WGQGTQVTVSS |
| 3D02 | VQAGGSLRLSC | | VS | YYADSVKG | TVYLQMNSLKP | DPRLADY | |
| 263 | SASGFTFD | | | | EDTAVYYCAA | | |
| DLLBII3 | EVQLVESGGGL | SYAMG | WYRQAPGKQREL | AISNGGSTN | RFTISRDNAKN | SGSYYYPT | WGQGTQVTVSS |
| 3E01 | VQAGGSLRLSC | | VA | YVDSVKG | TVYLQMNSLKP | DVHEYDY | |
| 264 | AASGSTFS | | | | EDTAVYYCFY | | |
| DLLBII3 | EVQLVESGGGL | YYNIG | WFRQAPGKEREW | CISSSDGRT | RFTMSRDNAKN | PFNYYSNL | WGQGTQVTVSS |
| 3E03 | VQPGGSLRLSC | | VS | NYVDSVKG | TVYLQMNSLKP | CGVNGVDY | |
| 265 | AASGFTLD | | | | EDTAVYYCAA | | |
| DLLBII3 | EVQLVESGGGL | YYAIG | WFRQAPGKEREG | CISGRGGST | RFTISRDNAKN | DPIHNCYS | WGQGTQVTVSS |
| 3F01 | VQAGGSLRLSC | | IS | YYIDSVKG | TVYLQMNSLKP | GSHYYSPE | |
| 266 | AASGFSLD | | | | EDTAVYYCAA | AVYEY | |
| DLLBII3 | EVQLVESGGGL | YYAVG | WFRQAPGKEREG | CISSRGGST | RFTTSRDNAKN | DPIHNCYS | WGQGTQVTVSS |
| 3F04 | VQAGDSLRLAC | | VS | FYADSLKG | TVYLQMNSLKP | GSDYASPE | |
| 267 | AASGFALD | | | | EDTAVYYCAA | AVYEY | |
| DLLBII3 | EVQLVESGGGL | YYNIG | WFRQAPGKEREW | CIRSSDGST | RFTISRNNAKN | PFIHSDLC | WGQGTQVTVSS |
| 3H04 | VQPGDSLRLSC | | VA | YYTDSVKG | TVYLQMNSLKP | GVNGNDY | |
| 268 | AASGFTLD | | | | EDTAVYYCAA | | |
| DLLBII4 | EVQLVESGGGL | SYAMG | WYRQAPGKQREL | VISSGSVTN | RFTISRDNAKN | SGSYYYPT | WGQGTQVTVSS |
| 2A08 | VQAGGSLRLSC | | VA | YADSVKG | TVSLQMNSLKP | DVHEYDY | |
| 269 | AASGSTFN | | | | EDTAVYYCFY | | |
| DLLBII4 | EVQLVESGGGL | YYAIG | WFRQAPGKEREW | CMGSSVRST | RFTISRDNAKN | APIFECPS | WGQGTQVTVSS |
| 2B07 | VQPGGSLRLSC | | VS | YYADSVKG | TVYLQMNSLKP | GEIYDY | |
| 270 | AASGFRLD | | | | EDTAVYYCAA | | |
| DLLBII4 | EVQLVESGGGL | YYAIG | WFRQAPGKEREG | CISSRGGST | RFTISRDNAKN | DPIHNCYS | WGQGTQVTVSS |
| 2B10 | VQAGGSLRLSC | | VS | YYTDSVKG | TVYLQMNSLKP | GTYYASPE | |
| 271 | AASGFTLD | | | | EDTGVYYCAA | AVYEY | |
| DLLBII4 | EVQLVESGGGL | YYAVG | WFRQAPGKEREG | CISSRGGST | RFTTSRDNAKN | DPIHNCYS | WGQGTQVTVSS |
| 2F08 | VQAGGSLRLSC | | VS | YYVDSVKG | TVYLEMNSLKP | GSYYASPE | |
| 272 | AASGFALD | | | | EDTAVYYCAA | AVYDY | |
| DLLBII4 | EVQLVESGGGL | SYAMG | WYRQAPGKQREL | VISSGDSTN | RFTISRDNAKN | SGSYYPTD | WGQGTQVTVSS |
| 2G04 | VQAGGSLRLSC | | VA | YSDSVKG | TVYLQMNSLKP | VHEYAY | |
| 273 | AASGSTFS | | | | EDTAVYYCFY | | |
| DLLBII4 | EVQLVESGGGL | WYAMG | WYRQAPGKQREL | VISSGDRTN | RFTISRDNAKN | SGSYYYPT | WGQGTQVTVSS |
| 3A05 | VQAGGSLRLSC | | VA | YLDSVKG | TVYLQMNSLKP | DVHEYAY | |
| 274 | AASGSSFS | | | | EDTAVYYCFY | | |
| DLLBII4 | EVQLVESGGGL | YYNIG | WFRQAPGKEREW | CISGSDGST | RFTISRDNAKN | PFAYYSDL | WGQGTQVTVSS |
| 3A10 | VQPGGSLRLSC | | VS | GYADSVKG | TVYLQMNSLKP | CGVNEYDY | |
| 275 | AASGFALD | | | | EDTAVYYCAA | | |
| DLLBII4 | EVQLVESGGGL | GHNIG | WFRQAPGKEREW | CINSGDGDT | RFTISRDNAKN | PFNHYSFL | WGQGTQVTVSS |
| 3A12 | VQPGGSLRLSC | | VS | GYADSVKG | TVYLQMNRLKP | CGVNEYDY | |
| 276 | AASGFALD | | | | EDTAVYYCAA | | |
| DLLBII4 | EVQLVESGGGL | SYAMG | WYRQAPGKQREL | VISTGDNTN | RFTISRDNAKN | SGSYYYPT | WGQGTQVTVSS |
| 3B04 | VQAGGSLRLSC | | AA | YADSVKG | TVYLQMNSLKP | EVYEYDY | |
| 277 | AASGSTFS | | | | EDTAVYHCFY | | |
| DLLBII4 | EVQLVESGGGL | SYAMG | WYRQVPGNQREL | VISSGDSAN | RFTISRDNAKN | SGSYYYPT | WGQGTQVTVSS |
| 3B11 | VQAGGSLRLSC | | VA | YADSVKG | TVYLQMNSLKP | DNHEYDY | |
| 278 | AASGSTFR | | | | EDTAVYYCFY | | |
| DLLBII4 | EVQLVESGGGL | YYNIG | WFRQAPGKEREW | CINSSDGTT | RFTISRDNAKN | PFEYYSDL | WGQGTQVTVSS |
| 3C12 | VQPGGSLRLSC | | VS | YYADSVKG | TVYLQMNSLKP | CGVNGYDY | |
| 279 | AASGFTLD | | | | EDTAVYYCAA | | |
| DLLBII4 | EVQLVESGGGL | YYAIG | WFRQAPGKEREG | CISGEGSNT | RFTISRDNAKN | DPIHNCYG | WGQGTQVTVSS |
| 7A05 | VQAGGSLRLSC | | VS | YYLDSVKG | TVYLMANSLKP | GSYYASPE | |
| 280 | ASGFSLD | | | | EDTAVYYCAA | AVYEY | |
| DLLBII4 | EVQLVESGGGL | DYAIG | WFRQAPGKEREG | CISSSGSTY | RFTISRDNAKN | AGASSWCF | WGQGTQVTVSS |
| 7D01 | VQPGGSLRLSC | | VS | YADSVKG | TVYLQMNSLKP | PPGY | |
| 281 | AASGFTFD | | | | EDTAVYYCAI | | |
| DLLBII4 | EVQLVESGGGL | YYAVG | WFRQAPGKEREG | CISSRGGST | RFTTSRDNAKN | DPIHNCYS | WGQGTQVTVSS |
| 7E03 | VQAGGSLRLSC | | VS | YYADSVKG | TVYLQMNSLKP | GIYYASPE | |
| 282 | AASGFALD | | | | EDTAVYYCAA | AVYDY | |
| DLLBII4 | EVQLVESGGGL | SYAMG | WYRQAPVKQREL | VISNGGST | RFTISRDNAKN | SGSYYYPTD | WGQGTQVTVSS |
| 7E12 | VQAGGSLRLSC | | VA | NYADSVKG | TVYLQMNSLKP | VHEYDY | |
| 283 | AASGSTFS | | | | EDTAVYYCFY | | |
| DLLBII4 | EVQLVESGGGL | YYAIG | WFRQAPGKEREG | CISSRGGST | RFTISRDNAKN | DPIHNCYS | WGQGTQVTVSS |
| 7F06 | VQPGGSLRLSC | | VS | YYEDSVKG | TVYLQMNSLKP | GRYYADPD | |
| 284 | AASGFTLD | | | | EDTAVYYCAA | AVYDY | |
| DLLBII4 | EVQLVESGGGL | HYNIG | WFRQAPGKEREW | CISSSDGST | RFTISRDKAKN | PFSYYSDL | WGQGTQVTVSS |
| 7G11 | VQPGGSLRLSC | | VS | GYADSVKG | TVYLQMNSLKP | CGVNGYDY | |
| 285 | AASEFTLD | | | | EDTAVYYCAA | | |
| DLLBII4 | EVQLVESGGGL | SYAMG | WYRQAPGKQREL | VISSGDSTN | RFTMSRDNAKN | SGSYYYPS | WGQGTQVTVSS |
| 7H02 | VQAGGSLRLSC | | VA | YADSVKG | TVYLQMNSLRP | DVHEYDY | |
| 286 | AASGSTFS | | | | EDTAVYYCFY | | |
| DLLBII4 | EVQLVESGGGL | YYNIG | WFRQAPGKEREW | CISSSDGST | RFTISRDNAKN | PFSYYSGL | WGQGTQVTVSS |
| 8A01 | VQPGGSLRLSC | | VS | DYADSVKG | TVYLQMNSLKP | CGVNGVDY | |
| 287 | AASGFTLD | | | | EDTAVYYCAA | | |
| DLLBII4 | EVQLVESGGGL | VYATG | WFRQAPGKEREW | CISGSDGST | RFTISRDNAKN | SLGSSWCA | WGQGTQVTVSS |
| 8A08 | VQPGGSLRLSC | | VS | WYADSVKG | TVYLQMNSPKS | YDY | |
| 288 | AASGFTLG | | | | EDTAVYYCAL | | |
| DLLBII4 | EVQLVESGGGL | YYAIG | WFRQAPGKEREG | CISGRGGST | RFTISRDNAKN | DPVHNCYS | WGQGTQVTVSS |
| 8E03 | VQAGGSLRLSC | | VS | YYTDSVKG | TVYLQMNSLKP | GRYYASPD | |
| 289 | AASGFTLD | | | | EDTGVYYCAA | AVYEY | |
| DLLBII4 | EVQLVESGGGL | SYAMG | WYRQAPGKQREL | VISNGGSTN | RFTISRDNAKN | SGSYYYPT | WGQGTQVTVSS |
| 8F05 | VQPGGSLRLSC | | VA | YADSVKG | TVYLQMNSLKP | DVHEYAY | |
| 290 | AASGSTFS | | | | EDTAVYICFY | | |
| DLLBII4 | EVQLVESGGGL | YHNIG | WFRQAPGKEREW | CISSSGGST | RFTISRDNAKN | PFSHYNDL | WGQGTQVTVSS |
| 9A12 | VQPGGSLRLSC | | VS | AYADSVKG | TVYLQMNSLKP | CGVNAIDY | |
| 291 | AASGFTLD | | | | EDTAVYYCAA | | |
| DLLBII4 | EVQLVESGGGL | YYAIG | WFRQAPGKEREG | CISSRGAST | RFTISRDNAKN | DPIHNCYS | WGQGTQVTVSS |
| 9B05 | VQAGGSLRLSC | | VS | YYADSVKG | TVYLQMNSLKP | GNGYDSPE | |
| 292 | AASGFTLD | | | | EDTAVYYCAA | AVYDY | |
| DLLBII4 | EVQLVESGGGL | YYNIG | WFRQAPGKEREW | CINSSDGST | RFTISRDNAKN | PFEYYSDL | WGQGTQVTVSS |
| 9E01 | VQPGGSLRLSC | | VS | HYADSVKG | TVYLQMNSLKP | CGVNGYDY | |
| 293 | AASGFTLH | | | | EDTAVYYCAA | | |
| DLLBII4 | KVQLVESGGGL | YYNIG | WFRQAPGKEREW | CINSSDGST | RFTISRDNAKN | PFEYYSNL | WGQGTQVTVSS |
| 9F05 | VQPGGSLRLSC | | VS | YYADSVKG | TVYLQMNSLKP | CGVNGYDY | |
| 294 | AASGFTLD | | | | EDTAVYYCAA | | |
| DLLBII4 | EVQLVESGGGL | KYSIG | WFRQAPGKEREG | CISSSGGST | RFTISRDNAKN | DPLHNCYS | WGQGTQVTVSS |
| 9G02 | VQAGGSLRLSC | | VS | YYVDSVKG | TVYLQMNNLKP | GRGYYSPE | |
| 295 | AASGFTLD | | | | EDTAVYYCAA | AVYEY | |
| DLLBII4 | EVQLVESGGGL | YYAIG | WFRQAPGKEREG | CISSRGGST | RFTISRDNAKN | DPIHNCYS | WGQGTQVTVSS |
| 9G05 | VQAGGSLRLSC | | VS | YYTDSVKG | TVYLQMNSLKP | GSYYASPE | |
| 296 | AASGFTLD | | | | EDTAVYYCAA | AVYEY | |
| DLLBII4 | EVQLVESGGGL | YYNIG | WFRQAPGKEREW | CINSSDGST | RFTISRDNAKN | PFEYYSNL | WGQGTQVTVSS |
| 9H05 | VQPGGSLRLSC | | VS | YYADSVKG | TVYLQMNSLKP | CGVNGYDY | |
| 297 | AASGFTLD | | | | EDTAVYYCAA | | |
| DLLBII5 | EVQLVESGGGL | DYAIG | WFRQAPGKEPEG | CISSSGSIT | RFTISRDNAKN | PGIAACRG | WGQGTQVTVSS |
| 5A07 | VQPGGSLRLSC | | IS | YDADSVKG | TVYLQMNSLKP | IHY | |
| 298 | TASGFTFD | | | | EDTAVYYCAT | | |
| DLLBII5 | EVQLVESGGGL | DYAIG | WFRQAPGKEPEG | CISSSGGIT | RFTISRDNAKN | PGIAACRG | TGQGTQVTVSS |
| 5D12 | VQPGGSLRLSC | | IS | YYADSVKG | TVYLQMNSLKP | IHY | |
| 299 | AASGFTFD | | | | EDTAVYYCAT | | |
| DLLBII5 | EVQLVESGGGL | DYAIG | WFRQAPGKEPEG | CISSSGGIT | RFTTSRDNAKN | PGIAACRG | TGQGTQVTVSS |
| 6A09 | VQPGGSLRLSC | | IS | YYADSVKG | TVYLQMNSLKP | IHY | |
| 300 | AASGFTFD | | | | EDTAVYYCAT | | |
| DLLBII5 | EVQLVESGGGL | VYAIG | WFRQAPGKEPEG | CISSSGSIT | RFTTSRDSAKN | PGIAACRG | WGQGTQVTVSS |
| 6C04 | VQPGGSLRLSC | | IS | YYADSVKG | TVYLQMNSLKP | IHY | |
| 301 | TASGFTFD | | | | EDTAVYYCAT | | |
| DLLBII5 | EVQLVESGGGL | DYAIG | WFRQAPGKEPEE | CISSSGGIT | RFTISRDNAKN | PGIAACRG | TGQGTQVTVSS |
| 6H08 | VQPGGSLRLSC | | IS | YYADSVKG | TVYLQMNSLKP | IHY | |
| 302 | AASGFTFD | | | | EDTAVYYCAT | | |
| DLLBII5 | EVQLVESGGGL | RYSMG | WFRQAPGKEREA | TISWSGDST | RFTISRDNTKN | KPNLKYGS | WGQGTQVTVSS |
| 8A11 | VQAGGSLRLSC | | VA | YYADSVKG | TLYLQIDSLKP | YWPPRGYD | |
| 303 | TTSERTVS | | | | EDTAVYYCVA | Y | |
| DLLBII5 | EVQLVESGGGL | RYSMG | WFRQAPGKEREA | TISWSGDST | RFTISRDNTKN | KPNLKYGS | WGQGTQVTVSS |
| 8B01 | VQAGGSLRLSC | | VA | YYADSVKG | MLYLQMNSLKP | TWPPRGYD | |
| 304 | TTSERTVS | | | | EDTAVYYCVA | Y | |
| DLLBII5 | EVQLVESGGGL | RYTMG | WLRQPAGKEREA | TISWSGDST | RFTISRDNKNT | KPNLKYGS | WGQGTQVTVSS |
| 9B01 | VQAGGSLRLSC | | VA | YYADSVKG | LYLQMNSLKPE | YWPPRGYD | |
| 305 | TTSERAVS | | | | DTADYYCAA | Y | |
| DLLBII5 | EVQLVKSGGGL | RYGMG | WFRQAPGKEREA | TISWSGDST | RFTISRDNTKN | KPNLKYGS | WGQGTQVTVSS |
| B911 | VQAGGSLRLSC | | VA | YYADSVKG | TLYLQMNSLKP | DWPPRGYD | |
| 306 | TTSERTVS | | | | EDTAVYYCAA | Y | |
| DLLBII6 | EVQLVESGGGL | SYAMS | WVRQAPGKGLEW | FINKDGSDT | RFTISRDNAKN | RTSRSPRP | RGQGTQVTVSS |
| 1F05 | VQPGGSLRLSC | | VS | GYADSVKG | TMYLQMNSLKP | | |
| 307 | TASGFTFS | | | | EDTAVYFCET | | |
| DLLBII6 | EVQLVESGGGL | RYAMG | WFRQAPGKEREF | AINWSGGST | RFTISRDNAKN | SNYYSVYD | WGQGTQVTVSS |
| 1F07 | VQAGGSLRLSC | | VA | YYADSVKG | TVYLQMNSLKP | DRPVMDY | |
| 308 | AASGRTFS | | | | EDTAVYDCAA | | |
| DLLBII6 | EVQLMESGGGL | NYYMS | WVRQAPGKGLEW | VISPDGSNT | RFTISRGNAKN | GSGSWGV | HGQGTQVTVSS |
| 2C11 | VQPGGSLRLSC | | VS | YYADTVKG | TLFLQMTGLKS | | |
| 309 | VAAGFTFS | | | | EDAAVYYCAR | | |
| DLLBII7 | EVQLVESGGGL | NYIMG | WFRQAPGKEREF | GISRYGDYT | RFTISRDNVKN | NEGYCSGY | WGQGTQVTVSS |
| 3B03 | VQAGGSLRLSC | | VA | AYADSVKG | TVYLRMSNSLK | GCYEDSGQ | |
| 310 | AASGRTFS | | | | PDDTAVYYCAA | YDY | |
| DLLBII7 | EVQLVESGGGL | NYIMG | WRFQAPGKEREF | GISRYGDYT | RFTISRDNVKN | NEGYCSGY | WGQGTQVTVSS |
| 8B04 | VQAGGSLRLSC | | VA | YYADSVKG | TVYLRMNSLKP | GCYEDSGQ | |
| 311 | AASGRTFS | | | | DDTAVYYCAA | YDY | |
| DLLBII8 | EVQLVESGGGL | TYAMG | WFRQAPGKEREF | AVSRFGVSW | RFTISRDNTAN | GGRSFLPF | WGQGTQVTVSS |
| 0E08 | VQAGGSLTLSC | | VA | DYADSVKG | TLKLRMNSLKA | VPAY | |
| E12 | AASGGTFT | | | | DDTAVYYCAA | | |
| DLLBII8 | EVQLVESGGGL | NYIMG | WFRQAPGKEREF | GISRYADYT | RFTISRDNVKN | NEGYCSGY | WGQGTQVTVSS |
| 3G01 | VQAGGSLRLSC | | VA | GYADSVKG | TVYLRMNSLKP | GCYEDSGQ | |
| 313 | AASGRTFS | | | | DDTAVYYCAA | YDY | |
| DLLBII8 | EVQLVESGGGL | IMG | WYRQAPGKEREF | GISRYGDIT | RFTISRDSVKN | NGGYCSGY | WGQGTQVTVSS |
| 3G04 | VKPGGSLRLSC | | VA | YAADSVKG | TVYLRMNSLKP | GCYEDSGQ | |
| 314 | AASGFTLY | | | | DDTAVYYCAA | YDY | |
| DLLBII8 | EVQLVESGGGL | DYAIG | WFRQAPGKEPEG | CISSSGGIT | RFTISRDNAKN | PGIAACRG | TGQGTQVTVSS |
| 7B06 | VQAGGSLRLSC | | IS | YYADSVKG | TVYLQMNSLKP | IHY | |
| 315 | AASGFTFD | | | | EDTAVYYCAT | | |
| DLLBII8 | EVQLVESGGGL | DYAIG | WFRQAPGKEPEE | CISSSGGIT | RFTISRDNAKN | PGIAACRG | TGQGTQVTVSS |
| 9B04 | VQPGGSLRLSC | | IS | YYADSVKG | TVYLQMNSLKP | IHY | |
| 316 | AASGFTFD | | | | EDTAVYYCAT | | |
| DLLBII9 | EVQLVESGGGL | DYAIG | WFRQAPGKEPEG | CISSSGGIT | RFTISRDNAKN | PGIAACRG | TGQGTQVTVSS |
| 0E10 | VQAGGSLRLSC | | IS | YYADSVKG | TVYLQMNSLKP | IHY | |
| 317 | AVSGFSFD | | | | EDTAVYYCAT | | |
| DLLBII9 | EVQLVESGGGL | SYDMS | WVRQAPGKGPEW | AINSGGGST | RFTISRDNAKN | PRGWGPTG | WGQGTQVTVSS |
| 5A01 | VQPGGSLRLSC | | VS | YYADSVKG | TLYLQMNSLKP | PIEYAY | |
| 318 | AASGFTFG | | | | EDTAVYYCAI | | |
| DLLBII9 | EVQLVESGGGL | SYDMS | WVRQAPGKGPEW | AINSGGGST | RFTISRDNAKN | PRGWGPTG | WGQGTQVTVSS |
| 5B03 | VQPGGSLRLSC | | VS | YYADSVKG | TLYLQMNSLKP | PIEYGY | |
| 319 | AASGFTFG | | | | EDTAVYYCAI | | |
| DLLBII9 | EVQLVESGGGL | SYDMS | WVRQAPGKGPEW | AINSGGGST | RFTISRDNAKN | PRGWGPTG | WGQGTQVTVSS |
| 5C03 | VQPGGSLRLSC | | VS | YYADSVKG | TLYLQMNSLKP | PHEYGY | |
| 320 | AASGFTFG | | | | EDTGVYSCAI | | |
| DLLBII9 | EVQLVESGGGL | SYDMS | WVRQAPGKGPEW | AINSGGGST | RFTISRDNAKN | PRGWGPTG | WGQGTQVTVSS |
| 5D02 | VQSGGSLRLSC | | VS | YYADSVKG | TLYLQMNSLKP | PHEYAY | |
| 321 | AASGFTFG | | | | EDTAVYYCAI | | |
| DLLBII9 | EVQLVESGGGS | SYAMG | WFRQAPGKEREF | AINWSGGYT | RFTISRDNAKN | PAPGSSGY | WGQGTQVTVSS |
| 5F02 | VQAGGSLRLSC | | VA | YYADSVRG | TVYLQMNSLKP | EYDY | |
| 322 | AASGRTFS | | | | EDTAVYYCAA | | |
| FLLBII9 | EVQLVESGGGL | VYAIG | WFRQAPGKEPEG | CISSSGSIT | RFTISRDSAKN | PGIAACRG | WGQGTQVTVSS |
| 5F03 | VQPGGSLRLSC | | IS | YYADSVKG | TVYLQMNSLKP | IHY | |
| 323 | TASGFTFD | | | | EDTAVYYCAT | | |
| DLLBII9 | EVQLVESGGGL | NYDMS | WVRHAPGKGPEW | AINSGGGST | RFTISRDNAKN | PRGWGPTG | WGQGTQVTVSS |
| 5H02 | VQPGGSLRLSC | | VS | YYTDSVKG | TLYLQMNSLKP | PHEYAY | |
| 324 | AASGFTFG | | | | EDTAVYYCAI | | |
| DLLBII9 | EVQLVESGGGL | SYDMS | WVRQAPGKGPEW | AINSGGGTT | RFTISRDNAKN | PRGWGPTG | WGQGTQVTVSS |
| 6C02 | VQPGGSLRLSC | | VS | YYADSVKG | TLFLQMNSLKP | PLEYGY | |
| 325 | AASGFTFG | | | | EDTAVYYCAI | | |
| DLLBII9 | EVQLVESGGGL | NYDMS | WVRQAPGKGPEW | AINSGGGDT | RFTISRDNAKN | PRGWGPTG | WGQGTQVTVSS |
| 6C03 | VQPGGSLRLSC | | VS | YYADSVKG | TLYLQMNSLKP | PHEYGY | |
| 326 | AASGFTFG | | | | EDTAVYYCAT | | |
| DLLBII9 | EVQLVESGGGL | NYDMS | WVRQAPGKGPEW | AINSGGGIT | RFAISRDNAKT | PRGWGPTG | WGQGTQVTVSS |
| 6F02 | VQPGGSLRLSC | | VS | YYADSVKG | TLYLQMNNLQP | PHEYGY | |
| 327 | AASGFTFG | | | | EDTAVYYCAT | | |
| DLLBII9 | EVQLVESGGGL | SYDMS | WVRQAPGKGPEW | AINSGGGIT | RFTISRDNAKN | PRGWGPTG | WGQGTQVTVSS |
| 6H02 | VQAGGSLRLSC | | VS | YYADLVKG | TLYLQMNSLKP | PHEYGY | |
| 328 | AASGFTFG | | | | EDTAVYYCAI | | |
| DLLBII9 | EVQLVESGGGL | NYDMS | WVRQAPGKGPEW | AINSGGGIT | RFTISRDNAKN | PRGWGPTG | WGQGTQVTVSS |
| 7B02 | VQPGGSLRLSC | | VS | YYADSVKG | TLYLQMNSLKP | PHEYGY | |
| 329 | AASGFTFG | | | | EDTAVYYCAT | | |
| DLLBII9 | EVQLVESGGGL | SYDMS | WVRQAPGKGPEW | AINSGGGST | RFTISRDNAKN | PRGWGPTG | WGQGTQVTVSS |
| 7D01 | VQPGGSLRLSC | | VS | YYADSVKG | TLYLQMNSLTP | PHEYAY | |
| 330 | AASGFTFG | | | | EDTAVYYCAI | | |
| DLLBII9 | EVQLVESGGGL | NYDMS | WVRRPPGKGPEW | AINSGGGST | RFTISRDNAKN | PRGWGPTG | WGQGTQVTVSS |
| 7E01 | VQPGGSLRLSC | | VS | YYADSVKG | TLYLQMNSLKP | PHEYGY | |
| 331 | TASGFTFG | | | | EDTAVYYCAT | | |
| DLLBII9 | EVQLVESGGGL | SYDMS | WVRQAPGKGPEW | AINSGGGST | RFTISRDNAKN | PRGWGPTG | WGQGTQVTVSS |
| 7E02 | VQPGGSLRLSC | | VS | YYADSVKG | TLYLQMNNLKP | PHEYAY | |
| 332 | AASGFTFG | | | | EDTAVYSCAI | | |

### Example 5

### Characterization of Purified Anti-DII4 VHHs

Inhibitory anti-DII4 VHHs selected from the screening described in Example 4 are further purified and characterized. Selected VHHs are expressed in E. coliTG1 as c-myc, His6-tagged proteins. Expression is induced by addition of 1 mM IPTG and allowed to continue for 4 hours at 37° C. After spinning the cell cultures, periplasmic extracts are prepared by freeze-thawing the pellets. These extracts are used as starting material and VHHs are purified via IMAC and size exclusion chromatography (SEC) resulting in 95% purity as assessed via SDS-PAGE.

### 5.1. Evaluation of DII4 Blocking VHHs in ELISA

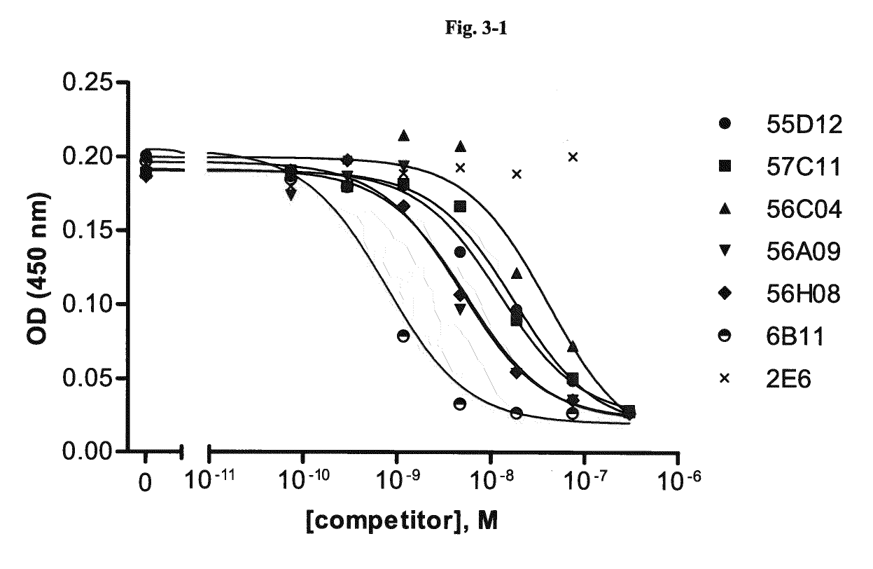

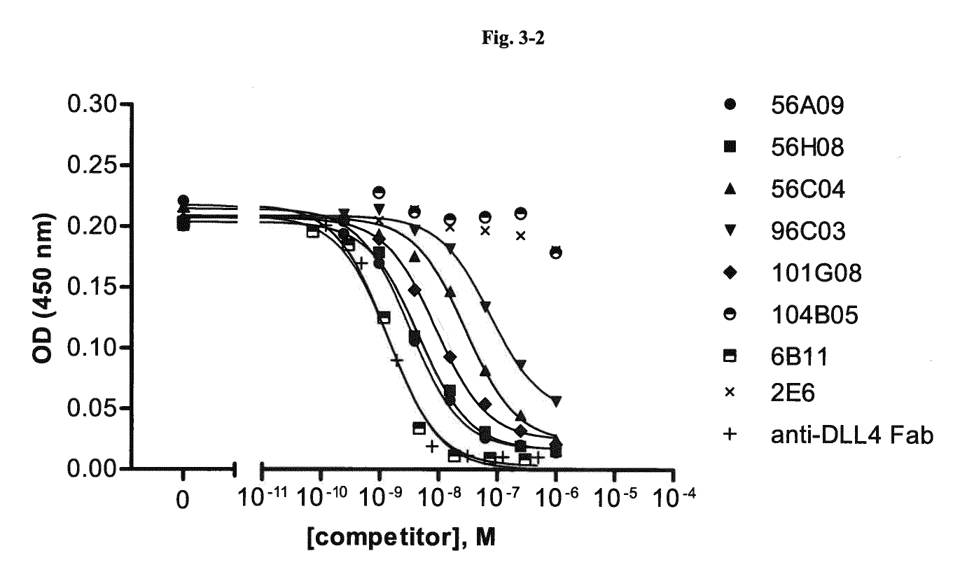

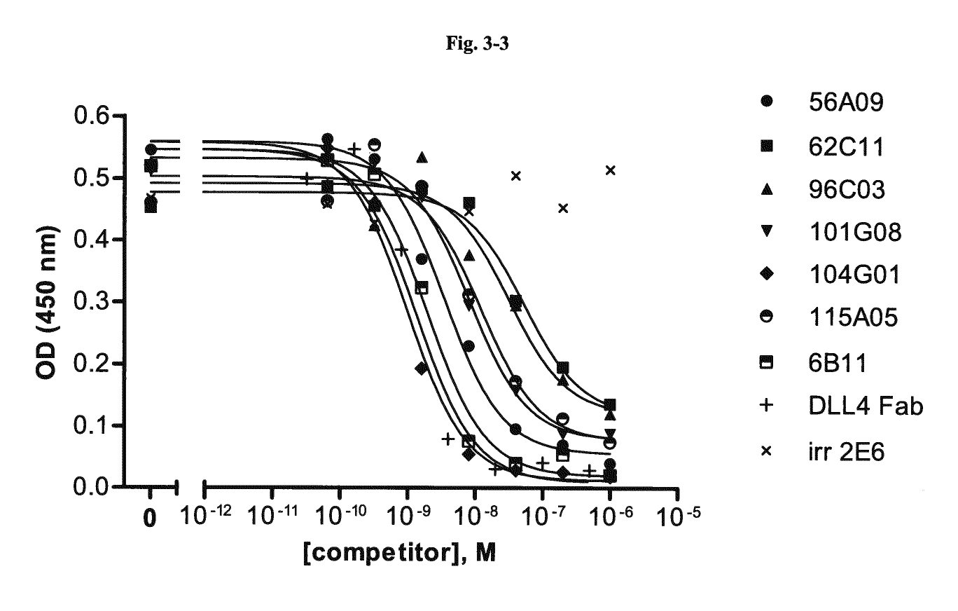

The blocking capacity of the VHHs is evaluated in a human DII4-human Notch1/Fc blocking ELISA. In brief, 1 μg/mL of human Notch1/Fc chimera (R&D Systems, Minneapolis, Minn., USA) is coated in a 96-well MaxiSorp plate (Nunc, Wiesbaden, Germany). A fixed concentration of 15 nM biotinylated human DII4 is preincubated with a dilution series of the VHH for 1 hour, after which the mixture is incubated on the coated Notch1 receptor for an additional hour. Residual binding of biotinylated human DII4 is detected using horseradish peroxidase (HRP) conjugated extravidin (Sigma, St. Louis, Mo., USA) (FIG. 3). Human DII4 is biotinylated as described above. The IC₅₀ values for VHHs blocking the human DII4-human Notch1/Fc interaction are depicted in Table 6.

TABLE 6
IC₅₀ (nM) values for VHHs in hDLL4/hNotch1-Fc competition ELISA

| VHH ID | IC₅₀ (nM) |
|---|---|
| 6B11 | 1.5 |
| 55D12 | 12.3 |
| 56A09 | 4.9 |
| 56C04 | 33.9 |
| 56H08 | 6.9 |
| 57C11 | 17.3 |
| 62C11 | 72.0 |
| 96C03 | 38.4 |
| 101G08 | 9.5 |
| 104G01 | 1.1 |
| 115A05 | 9.1 |
| antiDLL4 Fab | 0.7 |

### 5.2. Evaluation of DII4 Blocking VHHs in AlphaScreen

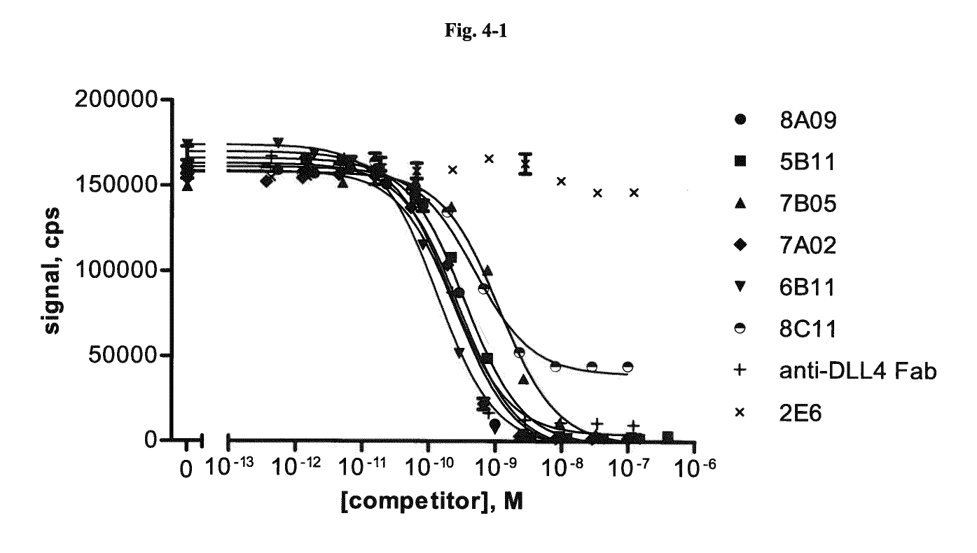

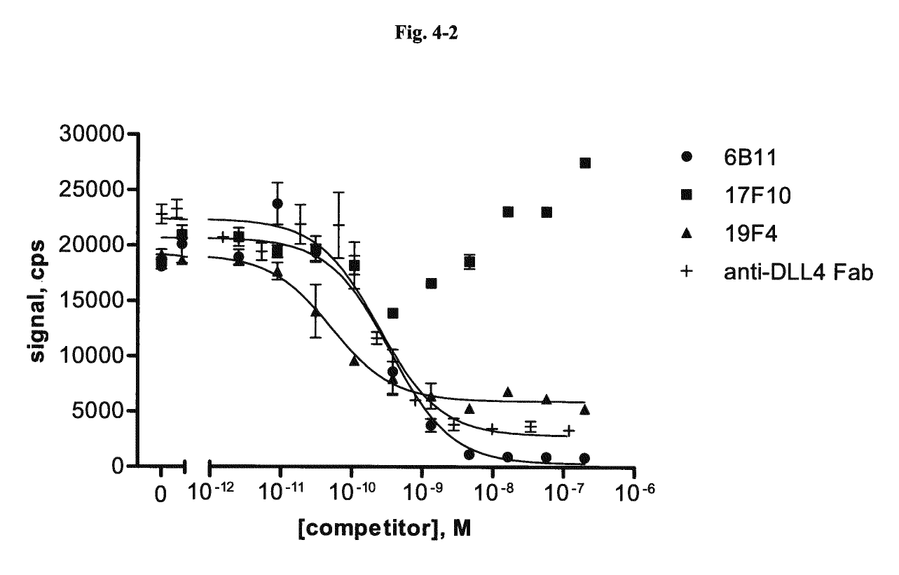

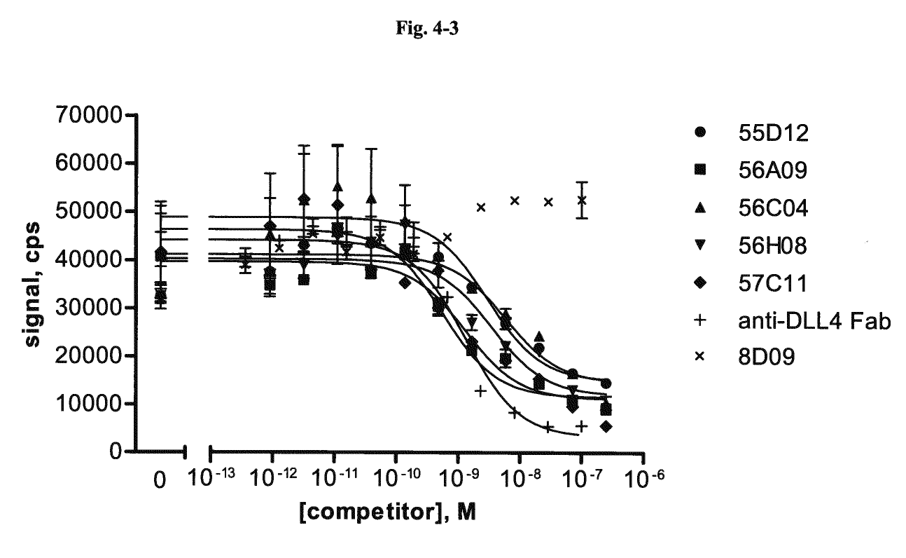

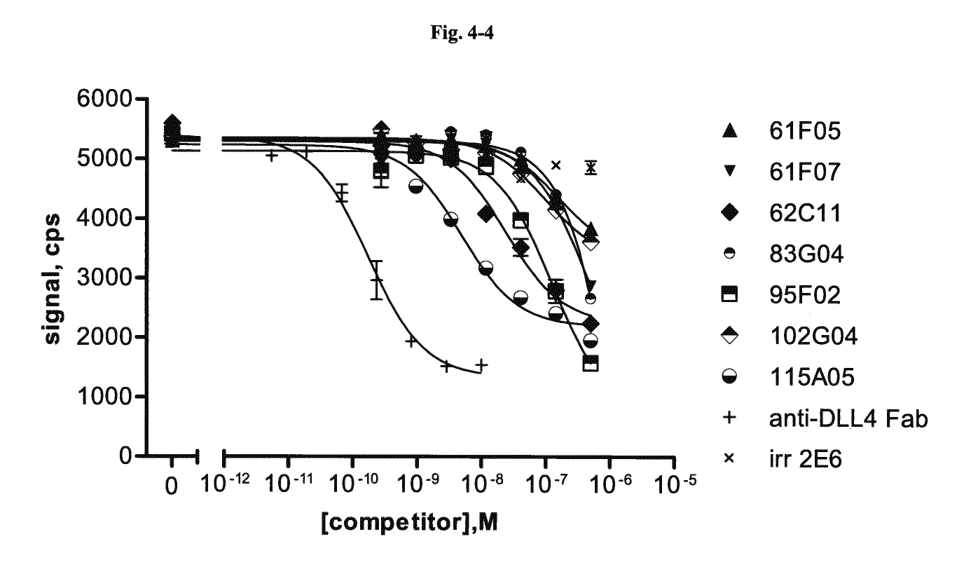

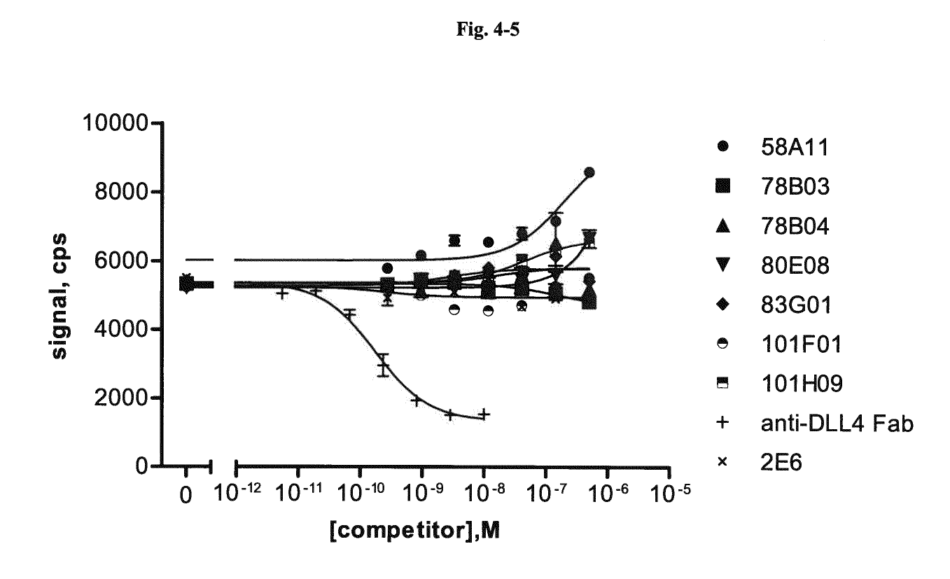

In brief, 1 nM biotinylated human DII4 is captured on streptavidin-coated donor beads (20 μg/mL), while 0.4 nM of the receptor human Notch1 (as a Fc fusion protein) is captured on anti-human Fc VHH-coated acceptor beads (20 μg/mL). Both loaded beads are incubated together with a dilution range of the competing VHH (FIG. 4). The IC₅₀ values for VHHs blocking the human DII4-human Notch1/Fc interaction are depicted in Table 7.

TABLE 7
IC₅₀ (nM) values for VHHs in hDLL4/
hNotch1 competition AlphaScreen

| VHH ID | IC₅₀ (nM) |
|---|---|
| 5B11 | 0.7 |
| 6B11 | 0.3 |
| 7A02 | 0.4 |
| 7B05 | 1.1 |
| 8A09 | 0.4 |
| 8C11 | 0.7 ^{(a)} |
| 19F04 | 0.05 ^{(a)} |
| 55D12 | 2.3 |
| 56A09 | 1.2 |
| 56C04 | 5.4 |
| 56H08 | 1.6 |
| 57C11 | 2.2 |
| 62C11 | 24.1 |
| 115A05 | 5.0 |
| antiDLL4 Fab | 0.3 |

^{(a)} partial inhibitor

### 5.3. Inhibition by Anti-DII4 VHHs of Human Notch 1/Fc Binding to Human or Mouse DII4 Expressed on the CHO Cells

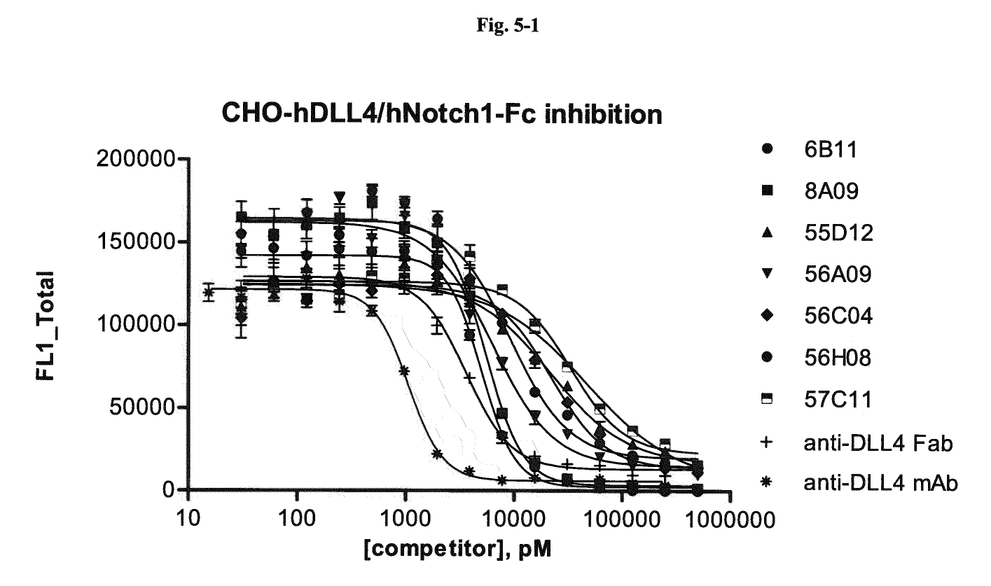

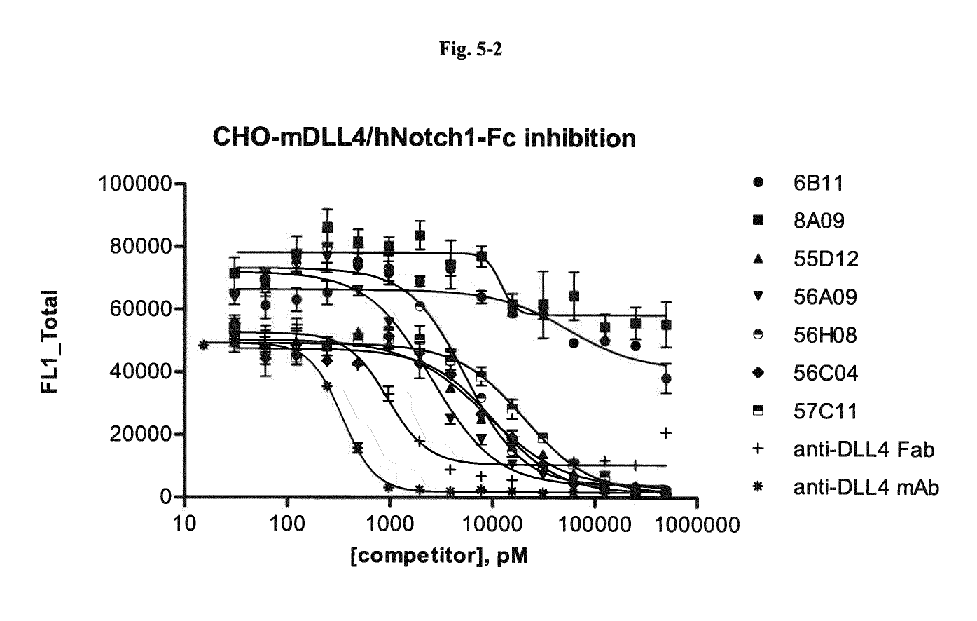

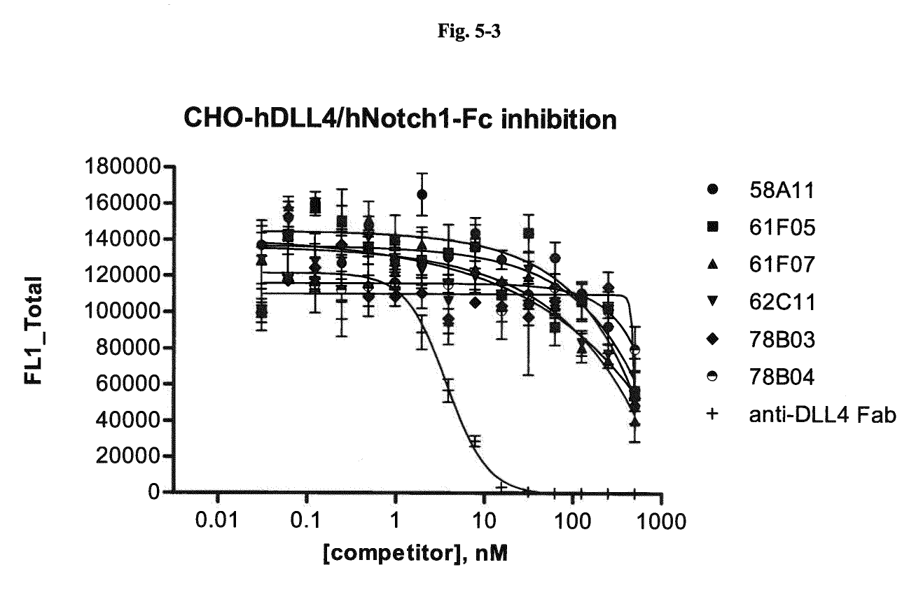

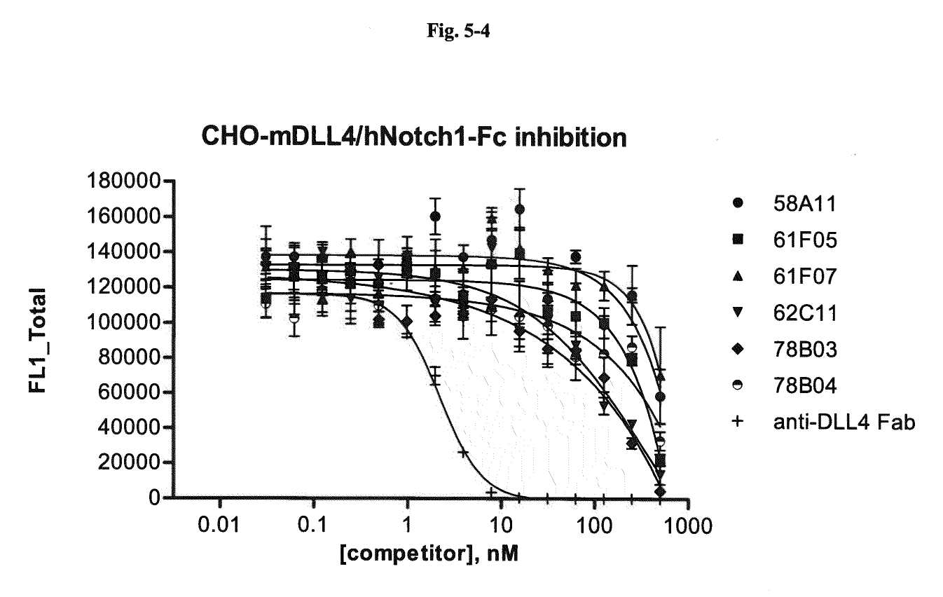

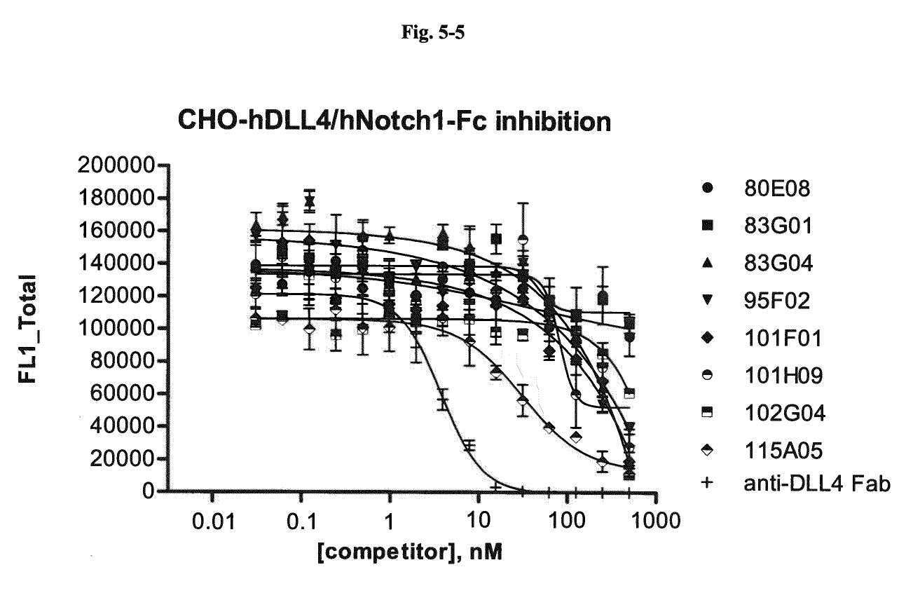

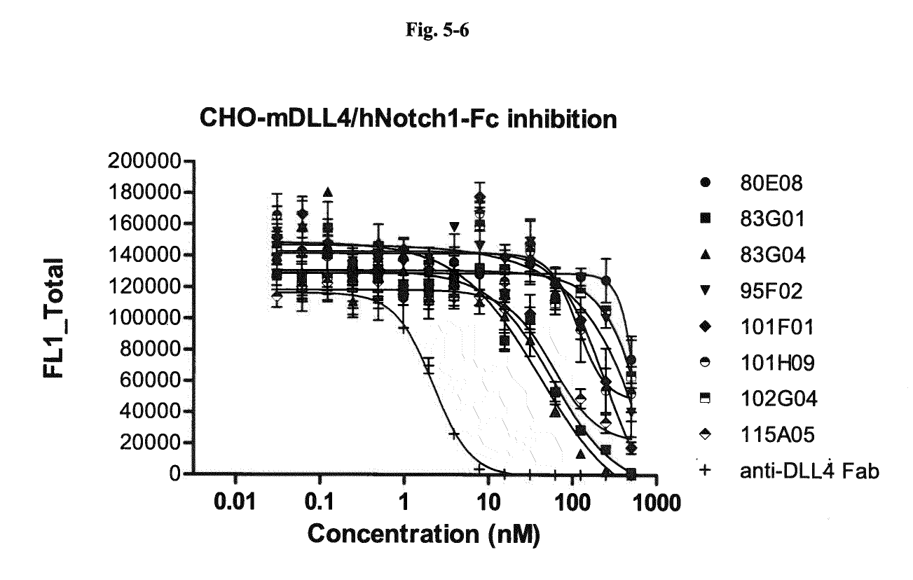

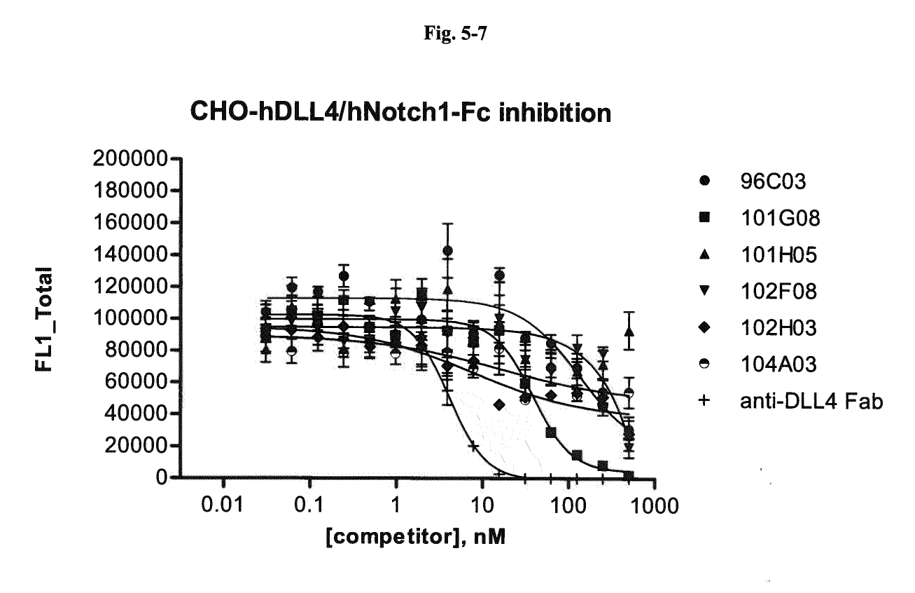

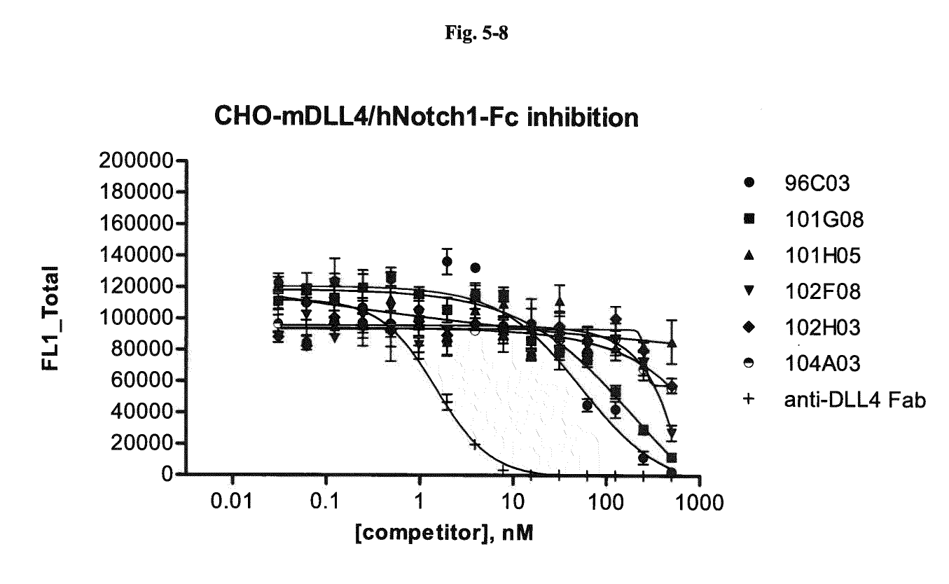

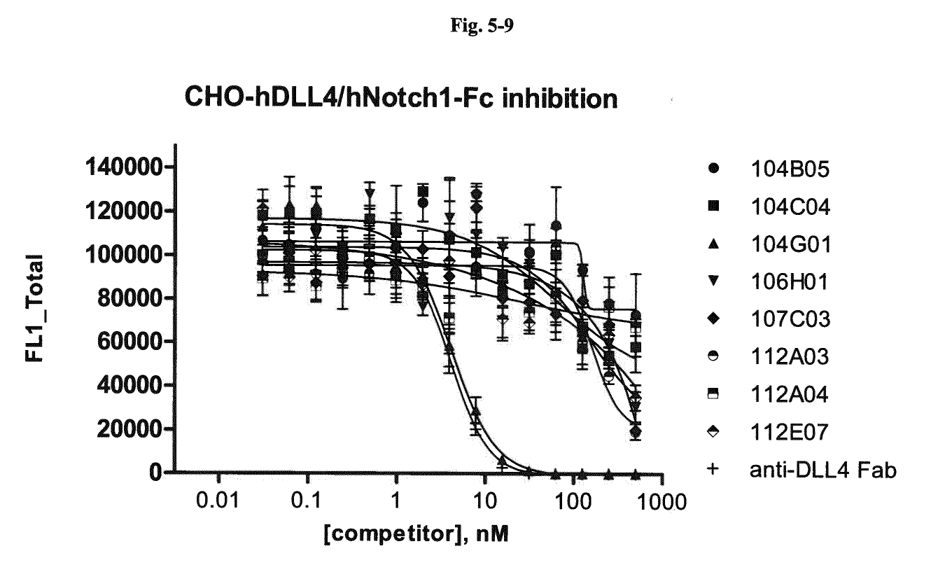

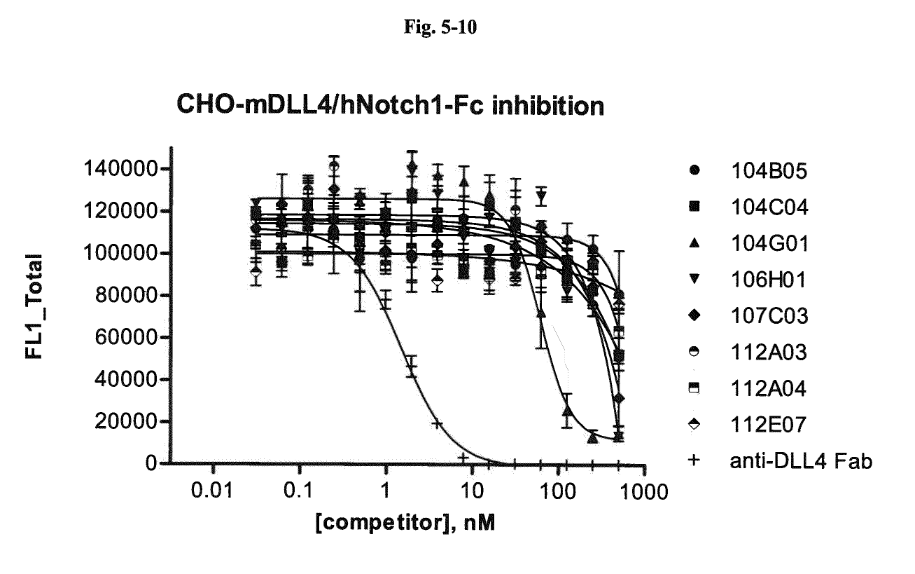

The blocking capacity of the VHHs is evaluated in a human and mouse DII4-human Notch1/Fc competitive FMAT assay (FIG. 5) as outlined in Example 4. The IC₅₀ values for VHHs blocking the interaction of human Notch1/Fc to human or mouse DII4 expressed on CHO cells are depicted in Table 8.

TABLE 8
(Mean) IC₅₀ values (nM) of purified VHHs
blocking the interaction of human Notch1/
Fc to human or mouse DLL4 expressed on
CHO cells (FMAT)

| | hDLL4 | mDLL4 |
|---|---|---|
| VHH ID | IC₅₀ (nM) | IC₅₀ (nM) |
| 6B11 | 8.9 | — |
| 8A09 | 5.5 | — |
| 19F04 | 33.0 | — |
| 55D12 | 39.1 | 41.0 |
| 56A09 | 10.6 | 15.0 |
| 56C04 | 28.7 | 49.6 |
| 56H08 | 22.0 | 33.7 |
| 57C11 | 53.9 | 49.5 |
| 62C11 | 172.2 | 106.3 |
| 96CO3 | 160.8 | 28.8 |
| 101G08 | 24.6 | 92.1 |
| 104G01 | 2.5 | — |
| 115A05 | 22.0 | 43.0 |
| antiDLL4 Fab | 5.4 | 2.3 |

### 5.4. Evaluation of DII4 Blocking VHHs in Reporter Assay

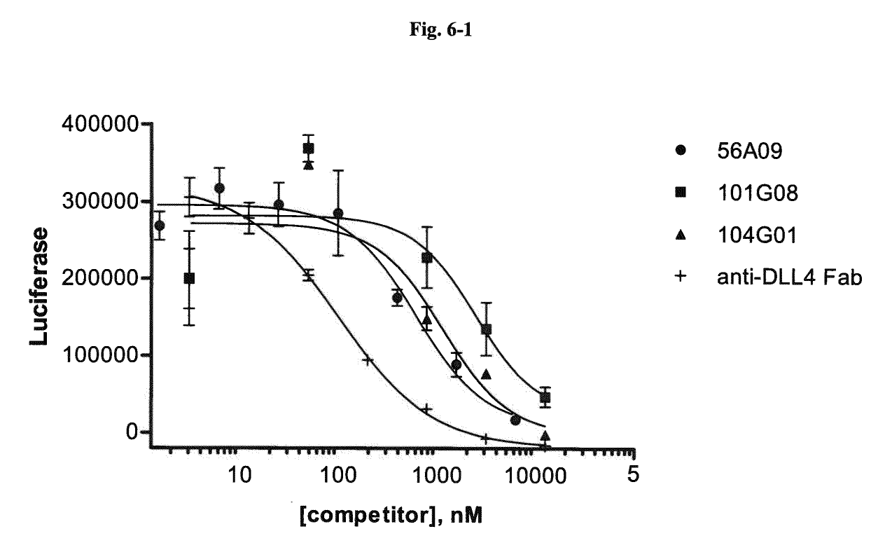

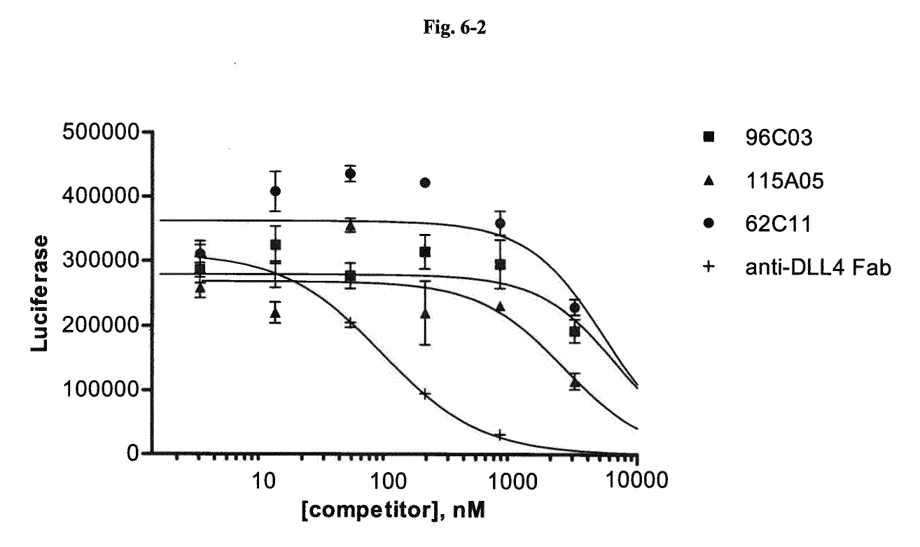

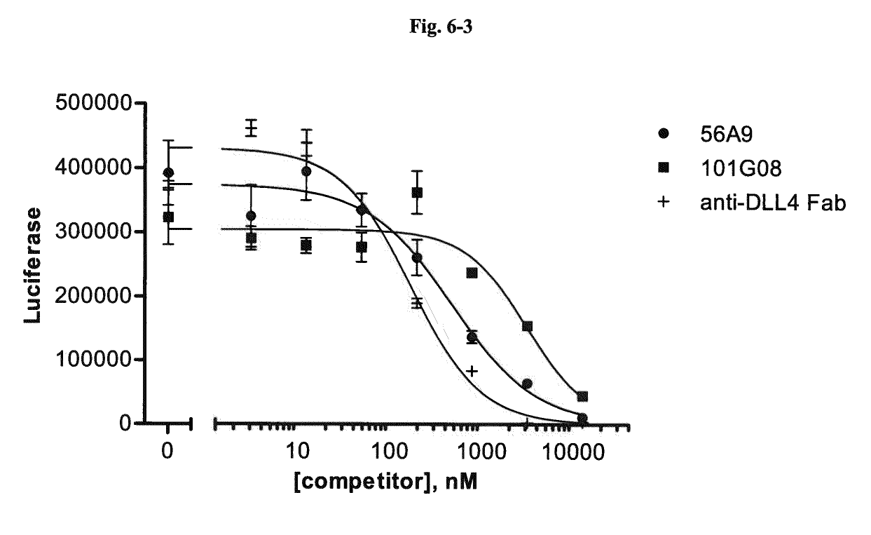

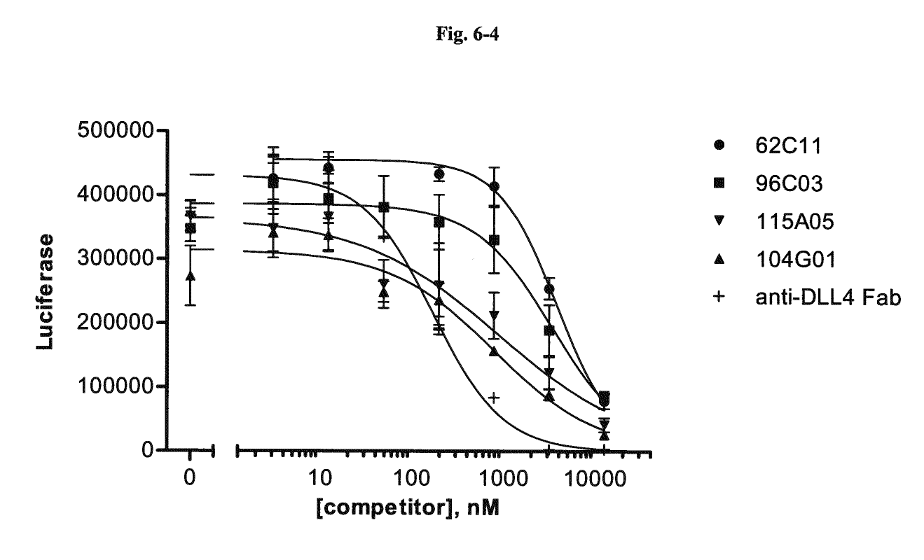

To evaluate the potency of the selected VHHs, a reporter assay is set up which is based on the γ-secretase mediated cleavage of Notch1 and release of the intracellular domain of Notch1 (NICD) upon stimulation with DII4. The Notch1-GAL4NP16 construct is cotransfected with the pGL4.31 [Luc2P/Gal4UAS/Hygro] reporter plasmid in HEK cells resulting in a transient expression of the fusion protein. These transiently transfected cells are stimulated for 24 hours by co-culture with a HEK293-hDII4 stable cell line. Forty-eight hours post-transfection, the readout is performed. The VHHs are preincubated with the HEK293-hDII4 cells to 1 hour before the start of the co-culture and are included during the co-culture (FIG. 6). The IC₅₀ values of the VHHs for blocking the DII4-mediated cleavage of Notch1 and subsequent translocation of its NICD to the nucleus of the receptor cell are depicted in Table 9.

TABLE 9
(Mean) IC₅₀ values (nM) of purified
anti-Dll4VHHs in a DLL4/Notch1
reporter assay

| VHH ID | IC₅₀ |
|---|---|
| 56A09 | 540 |
| 62C11 | 4663 |
| 96C03 | 5156 |
| 101G08 | 2760 |
| 104G01 | 964 |
| 115A05 | 1740 |
| anti-DLL4 Fab | 133 |

### 5.5. Epitope Binning

In order to determine whether VHHs can bind simultaneously to DII4 when e.g. a benchmark antibody is bound, epitope binning experiments are carried out (via Surface Plasmon Resonance (SPR) on a Biacore T100 instrument). Anti-DII4 Fab fragment is irreversibly immobilized on the reference and on the active flow cell of a CM5 sensor chip. For each sample (cycle), human DII4 is injected on the active and reference flow cell and reversibly captured by anti-DII4 Fab. Additional binding of VHHs is evaluated by injection over the immobilized surface. All VHHs and anti-D114 Fab are injected at 100 nM with a surface contact time of 120 seconds and a flow rate of 10 uL/minute. Surface is regenerated using 10 mM glycine (pH1.5). Processed curves are evaluated with Biacore T100 Evaluation software. Table 10-A represents the sequential injection/regeneration path of analysed VHHs and controls. VHHs DLLBII56A09 (SEQ ID NO: 300), DLLBII96C03 (SEQ ID NO: 326), DLLBII101G08 (SEQ ID NO: 197) and DLLBII115A05 (SEQ ID NO: 224) are shown not to additionally bind to human DII4 captured by DII4 Fab. Injection of DII4 Fab also failed to additionally bind human DII4 indicating that all epitopes are saturated. Therefore, it can be concluded that these VHHs recognize an epitope overlapping with DII4 Fab for binding human DII4. Human-only VHHs DLLBII6B11 (SEQ ID NO: 174) and DLLBII104G01 (SEQ ID NO: 215) show additional binding on DII4 Fab captured human DII4, indicating that these VHHs that are specific for human DII4 recognize a different epitope than the human/mouse cross-reactive VHHs.

TABLE 10-A
Epitope binning of anti-DLL4
VHHs - simultaneous binding with
DLL4Fab

| Injection | Binding/ | | | Binding level |
|---|---|---|---|---|
| step | Regeneration | [sample ] | | (RU) |
| 1 | hDLL4 | 100 | nM | 1727 |
| 2 | DLL4 Fab | 100 | nM | no binding |
| 3 | 59A9 | 100 | nM | no binding |
| 4 | 6B11 | 100 | nM | 405 |
| 5 | Glycine pH1.5 | 10 | mM | 90 |
| 6 | hDLL4 | 100 | nM | 1349 |
| 7 | 104G1 | 100 | nM | 276 |
| 8 | Glycine pH1.5 | 10 | mM | 87 |
| 9 | hDLL4 | 100 | nM | 1336 |
| 10 | Glycine pH1.5 | 10 | mM | 70 |
| 11 | hDLL4 | 100 | nM | 1333 |
| 12 | 96C3 | 100 | nM | no binding |
| 13 | 101G8 | 100 | nM | no binding |
| 14 | 115A05 | 100 | nM | no binding |
| 15 | Glycine pH1.5 | 10 | mM | 70 |

### 5.6. Epitope Mapping Using DII4 Deletion Mutants

Binding of the VHHs to these DII4 mutants is assessed in Biacore. In brief, VHHs DLLBII101G08 (SEQ ID NO:197) and DLLBII115A5 (SEQ ID NO: 224) are coated on a CM4 Sensorchip and 200 nM of each deletion mutant is injected across the chip. Binding is qualitatively assessed. No binding of DLLBII56A09 (SEQ ID NO: 300), DLLBII101G08 (SEQ ID NO: 197) and DLLBII115A05 (SEQ ID NO: 224) is observed to human and mouse DII4 mutants hDII4.1 and mDII4.8, respectively, lacking EGF-like 2 domain (Table 10-B). Indirect evidence using a hDII4/DII4 IgG competitive ELISA already pointed to this observation. In brief, 1 μg/mL of DII4 IgG is coated in a 96-well MaxiSorp plate (Nunc, Wiesbaden, Germany). A fixed concentration of 6 nM biotinylated human DII4 is preincubated with a dilution series of the VHH for 1 hour, after which the mixture is incubated on the coated IgG for an additional hour. Residual binding of biotinylated human DII4 is detected using horseradish peroxidase conjugated extravidin (Sigma, St. Louis, Mo., USA) (data not shown). Human DII4 is biotinylated as described above. It is known from patent literature that the monoclonal anti-DII4 IgG (Genentech, US 2008/0014196A1) binds to an epitope within the EGF-like 2 domain of DII4.

TABLE 10-B
Epitope mapping of anti-DLL4 VHHs - binding to DLL4 deletion mutants

| | | | DLLBII101G8 | | DLLBII115A5 | |
|---|---|---|---|---|---|---|
| | DLLBII56A9 | | Bind- | | Bind- | |
| | Binding | | ing | | ing | |
| sample | (RU) | kd (1/s) | (RU) | kd (1/s) | (RU) | kd (1/s) |
| hDLL4 | 281 | 9.5E−04 | 373 | 2.0E−03 | 324 | 3.5E−03 |
| mDLL4 | 389 | 1.9E−03 | 502 | 6.0E−03 | 344 | 6.5E−03 |
| hDLL4.1 | no | | no | | no | |
| | binding | | bind- | | bind- | |
| | | | ing | | ing | |
| hDLL4.3 | 125 | 7.4E−04 | 198 | 4.65E−03 | 137 | 3.5E−03 |
| hDLL4.5 | 143 | 1.2E−03 | 266 | 2.19E−03 | 162 | 4.2E−03 |
| hDLL4.6 | 136 | 1.1E−03 | 229 | 2.20E−03 | 152 | 4.1E−03 |
| mDLL4.8 | no | | no | | no | |
| | binding | | bind- | | bind- | |
| | | | ing | | ing | |
| mDLL4.10 | 141 | 1.1E−03 | 189 | 5.14E−03 | 121 | 3.8E−03 |
| mDLL4.11 | 132 | 1.6E−03 | 210 | 6.16E−03 | 121 | 6.6E−03 |
| mDLL4.12 | 161 | 1.3E−03 | 244 | 4.52E−03 | 152 | 3.1E−03 |

5.7. Determining the Affinity of the hDII4-VHH Interaction

Kinetic analysis to determine the affinity of the DII4-VHH interaction is performed by Surface Plasmon Resonance (SPR) on a Biacore T100 instrument. Recombinant human DII4 is immobilized onto a CM5 chip via amine coupling using EDC and NHS) or biotinylated human DII4 is captured on a SA chip (streptavidin surface). Purified VHHs or Fab fragment are injected for 2 minutes at different concentrations (between 10 and 300 nM) and allowed to dissociate for 20 min at a flow rate of 45 μl/min. Between sample injections, the surfaces are regenerated to with 10 mM glycine pH1.5 and 100 mM HCl. HBS-N (Hepes buffer pH7.4) is used as running buffer. If possible, data are evaluated by fitting a 1:1 interaction model (Langmuir binding) onto the binding curves. The affinity constant K_{D} is calculated from resulting association and dissociation rate constants (kₐ) and (k_{d}). The affinities of the anti-DII4 VHHs are depicted in Table 11.

TABLE 11
Affinity K_{D} (nM) of purified VHHs
for recombinant human DLL4

| | rhDLL4 | | |
|---|---|---|---|
| VHH ID | kₐ (M⁻¹ · s⁻¹) | k_{d} (s⁻¹) | K_{D} (nM) |
| 56A09 | 1.7E+05 | 9.3E−04 | 5.6 |
| 56C04 | 1.1E+05 | 4.9E−03 | 45 |
| 56H08 | 1.2E+05 | 1.1E−03 | 9.4 |
| 62C11 | 1.2E+06 | 1.3E−01 | 120 |
| 96C03 | 1.6E+05 | 4.8E−02 | 310 |
| 101G08 | 4.3E+04 | 2.2E−03 | 52 |
| 104G01 ^{(a)} | 1.2E+05 − | 3E−03 − | 4-24 |
| | 1.5E+05 | 6E−04 | |
| 115A05 | 1.5E+05 | 3.9E−03 | 25 |
| antiDLL4 Fab | 2.3E+05 | 3.4E−04 | 1.5 |

^{(a)} heterogeneous binding curve resulting in no 1:1 fit

5.8. Binding to Orthologues (mDII4, cDII4) and Family Members (hJagged-1,hDLL 1)

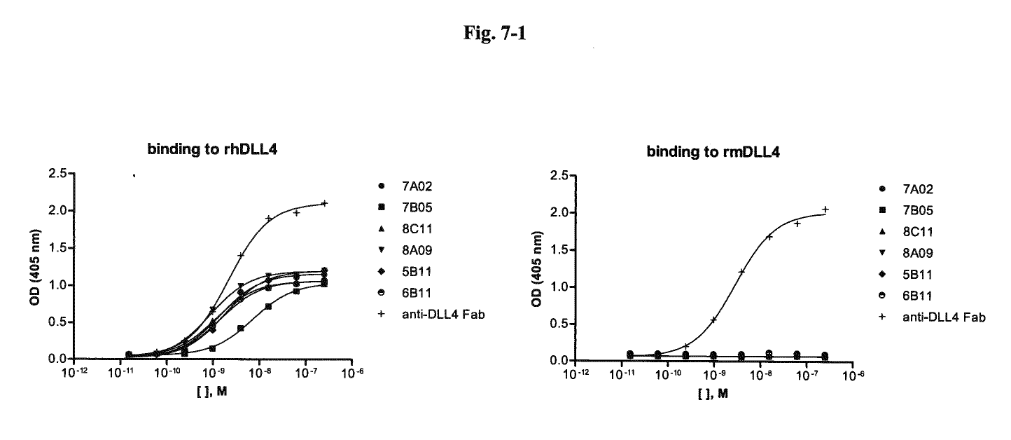

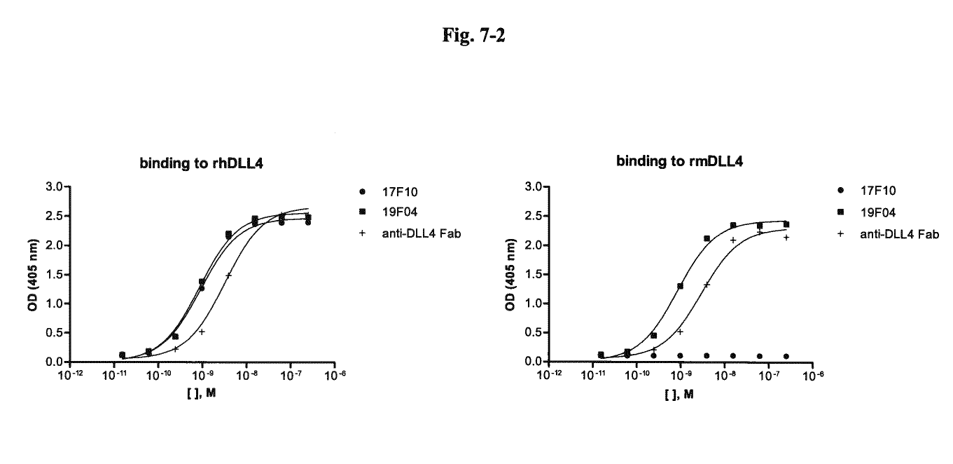

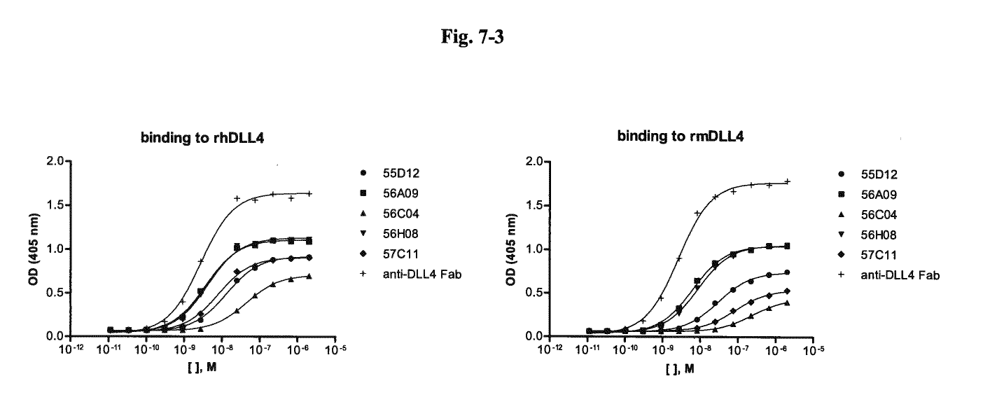

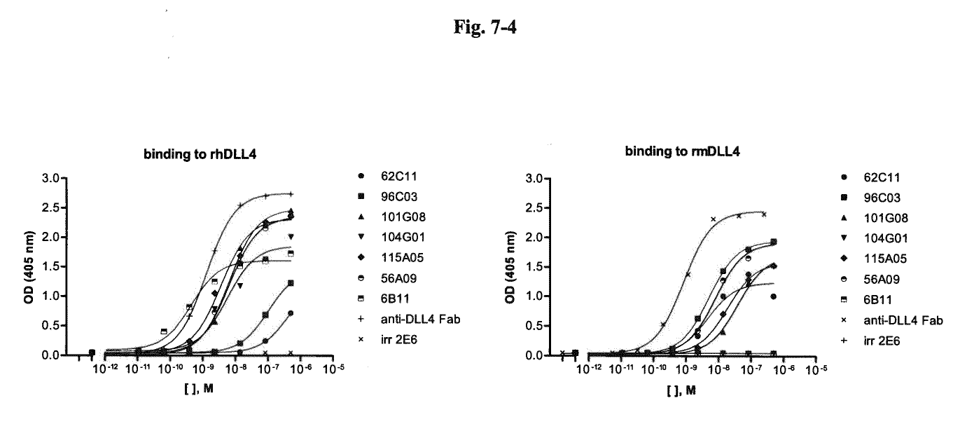

In order to determine cross-reactivity to mouse DII4 a binding ELISA is performed. In brief, recombinant mouse DII4 (R&D Systems, Minneapolis, Miss., USA) is coated overnight at 4° C. at 1 μg/mL in a 96-well MaxiSorp plate (Nunc, Wiesbaden, Germany). Wells are blocked with a casein solution (1% in PBS). VHHs are applied as dilution series and binding is detected using a mouse anti-myc (Roche) and an anti-mouse-AP conjugate (Sigma, St Louis, Mo., USA) (FIG. 7). As reference, binding to human DII4 is measured. EC₅₀ values are summarized in Table 12.

TABLE 12
EC₅₀ (nM) values for VHHs in a recombinant
human DLL4 and mouse DLL4 binding ELISA

| | rhDLL4 | rmDLL4 |
|---|---|---|
| VHH ID | EC₅₀ (nM) | EC₅₀ (nM) |
| 5B11 | 1.8 | — |
| 6B11 | 1.4 | — |
| 7A02 | 1.4 | — |
| 7B05 | 7.2 | — |
| 8A09 | 0.9 | — |
| 8C11 | 1.1 | — |
| 17F10 | 0.9 | — |
| 19F04 | 0.9 | 0.8 |
| 55D12 | 13.1 | 30.0 |
| 56A09 | 3.6 | 6.3 |
| 56C04 | 44.3 | 244.0 |
| 56H08 | 4.1 | 8.7 |
| 57C11 | 7.9 | 83.4 |
| 62C11 | 137.0 | 13.1 |
| 96C03 | 86.5 | 8.7 |
| 101G08 | 8.9 | 53.9 |
| 104G01 | 8.4 | — |
| 115A05 | 5.0 | 33.4 |
| antiDLL4 Fab | 3.0 | 3.0 |

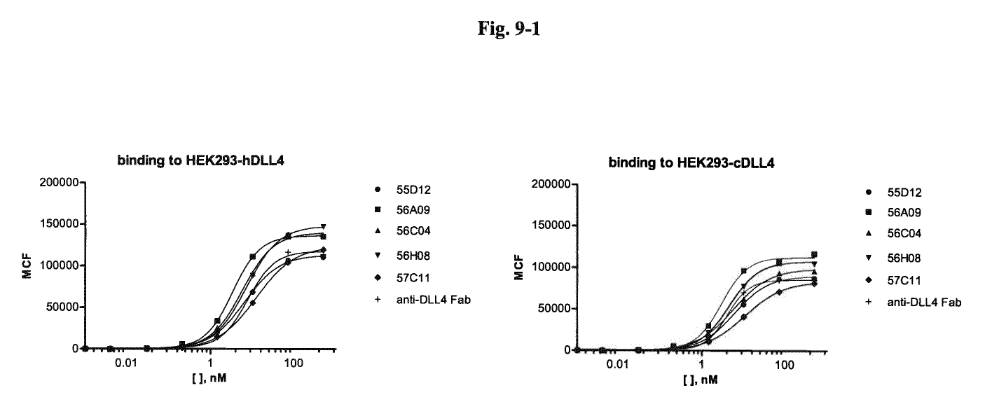

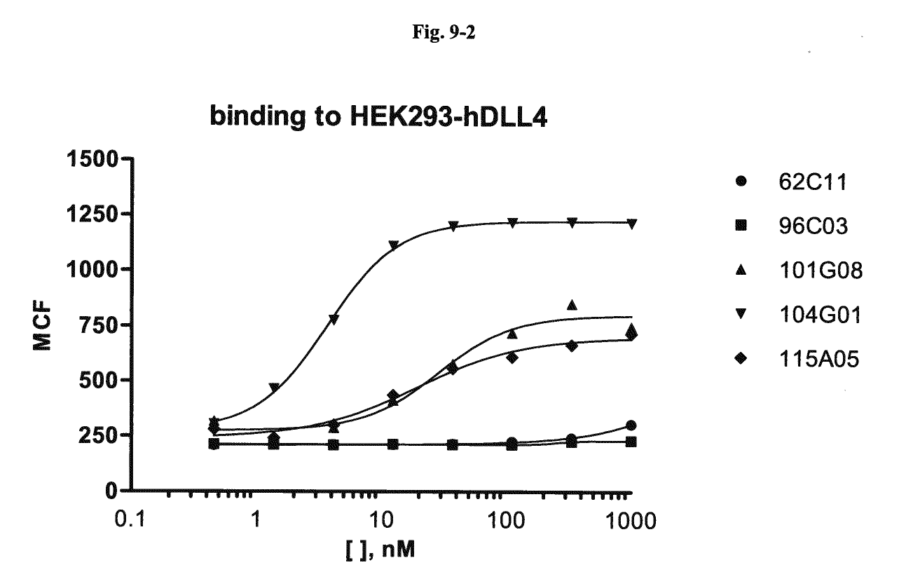

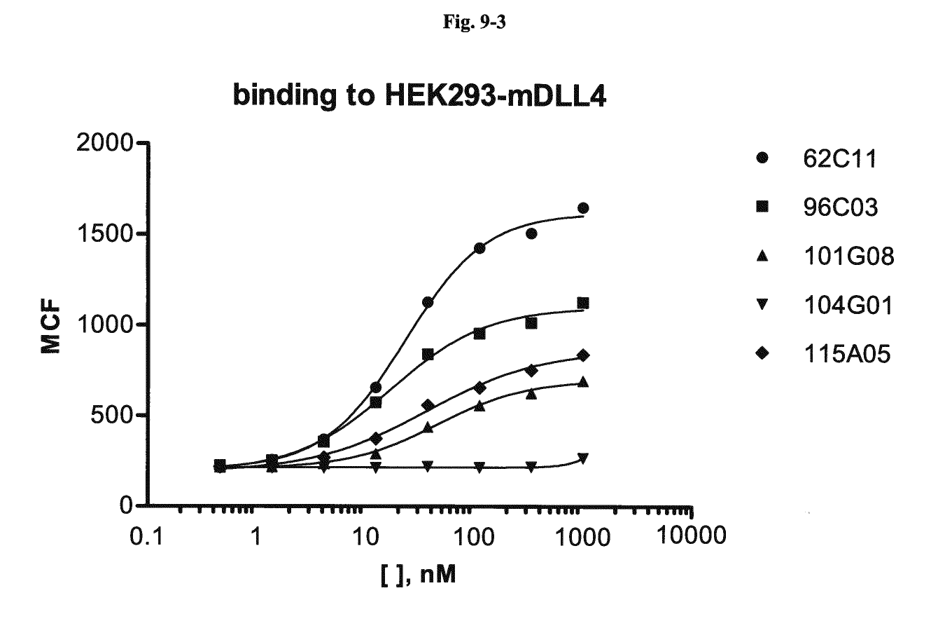

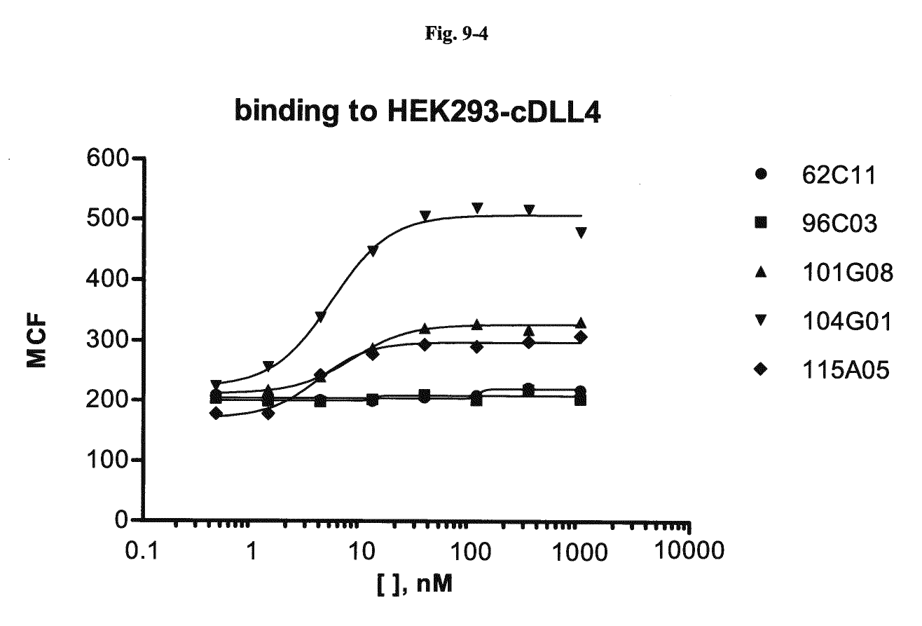

In order to determine the cynomologus cross-reactivity of the VHHs, a FACS binding experiment is performed. Cynomolgus DII4 expressing HEK293 cells (transient or stable transfection) are used for a titration binding experiment of the VHHs. After a 30 minutes incubation on ice, all samples are washed and detection is performed by applying anti-c-myc-Alexa647 (Santa Cruz Biotechnology, Santa Cruz, Calif., USA). Human and mouse DII4 overexpressing HEK293 cells are taken as reference. The mean MCF value is determined on the FACS Array and used for calculation of the EC₅₀ value (see FIG. 9).

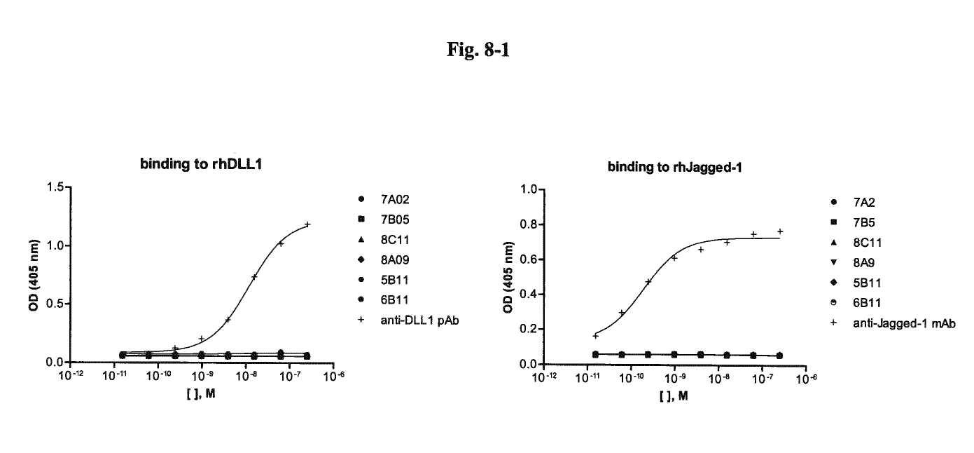

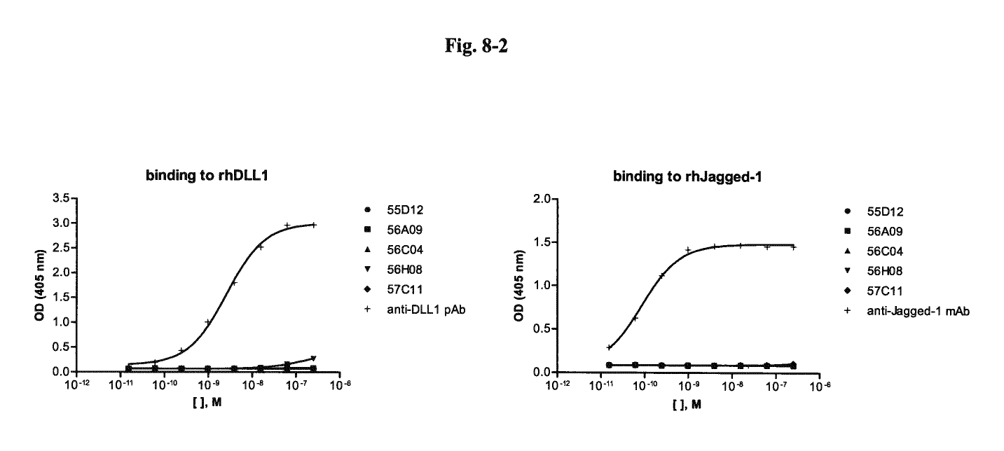

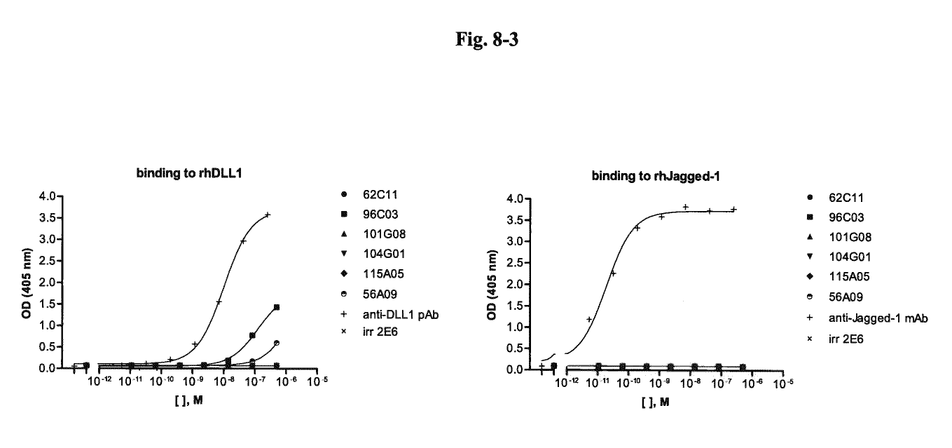

Absence of binding to homologous ligands human DLL1 and human Jagged-1 is assessed via solid phase binding assay (ELISA). In brief, human DLL1 (Alexis, San Diego, Calif., USA) and human Jagged-1 (Alexis, San Diego, Calif., USA) are coated overnight at 4° C. at 1 μg/mL in a 96-well MaxiSorp plate (Nunc, Wiesbaden, Germany). Wells are blocked with a casein solution (1% in PBS). VHHs are applied as dilution series and binding is detected using a mouse anti-myc (Roche) and an anti-mouse-AP conjugate (Sigma, St. Louis, Mo., USA). All anti-DII4 VHHs are considered as being non-cross reactive to these homologous ligands (FIG. 8).

### 5.9. Evaluation of VHHs in Blocking DII4-Mediated HUVEC Proliferation

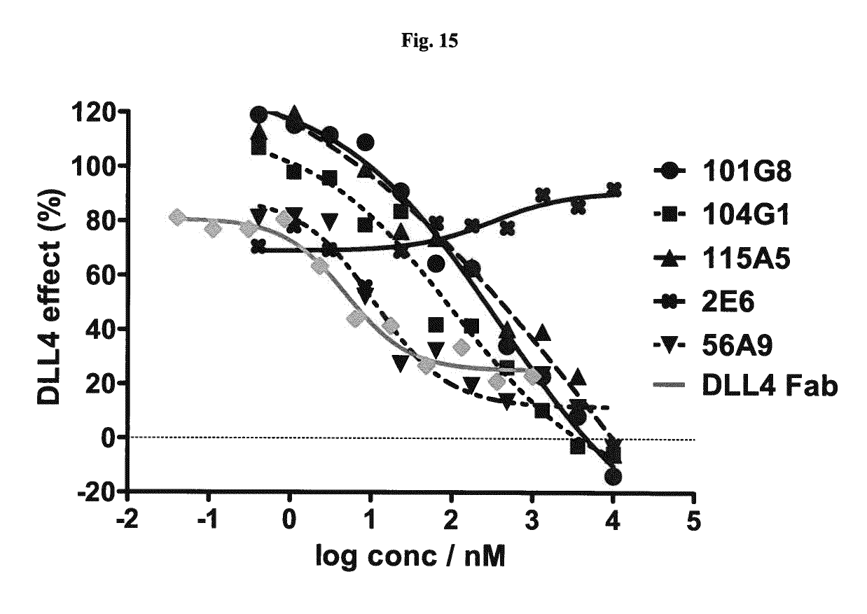

The potency of the selected VHHs is evaluated in a proliferation assay, as described by Ridgway et al., Nature. 2006 Dec. 21; 444(7122):1083-7), in modified form. In brief, 96-well tissue culture plates are coated with purified DII4-His (RnD Systems; C-terminal His-tagged human DII4, amino acid 27-524, 0.75 ml/well, ng/ml) in coating buffer (PBS, 0.1% BSA). Wells are washed in PBS before 4000 HUVE cells/well are seeded in quadruplicate. Cell proliferation is measured by [³H]-Thymidine incorporation on day 4. The results, shown in FIG. 15, demonstrate that the DLL4 VHHs DLLBII101G08, DLLBII104G01, DLLBII115A05, DLLBII56A09 and the DLL4 Fab inhibit the DLL4-dependent effect on HUVEC proliferation in a dose-dependent manner, the IC₅₀ values are summarized in Table 13. The tested VHHs achieve a complete inhibition of the DLL4-dependent effect at 10 μM.

TABLE 13
IC₅₀ values obtained in the DLL4
proliferation assay

| VHH/Fab | Fab | 56A9 | 104G1 | 101G8 | 115A5 |
|---|---|---|---|---|---|
| IC₅₀ (nM) | 4.9 | 11.0 | 103 | 401 | 10002 |
| (experiment 1) | | | | | |
| IC₅₀ (nM) | 5.6 | 6.8 | 32 | 112 | N.D. |
| (experiment 2) | | | | | |
| n | 2 | 2 | 2 | 2 | 1 |

### Example 6

### Affinity Maturation of Selected Anti-DII4 VHHs

VHHs DLLBII101G08 and DLLBII115A05 are subjected to two cycles of affinity maturation.

In a first cycle, amino acid substitutions are introduced randomly in both framework (FW) and complementary determining regions (CDR) using the error-prone PCR method. Mutagenesis is performed in a two-round PCR-based approach (Genemorph II Random Mutagenesis kit obtained from Stratagene, La Jolla, Calif., USA) using 1 ng of the DLLBII101G08 or DLLBII115A05 cDNA template, followed by a second error-prone PCR using 0.1 ng of product of round 1. After a polish step, PCR products are inserted via unique restriction sites into a vector designed to facilitate phage display of the VHH library. Consecutive rounds of in-solution selections are performed using decreasing concentrations of biotinylated recombinant human DLL4 (biot-rhDLL4) and trypsin elutions. Affinity-driven selections in a third round using cold rhDLL4 (at least 100× excess over biot-rhDLL4) are also performed. No selections on murine DLL4 are included as (conservation of) cross-reactivity is assessed at the screening level. Individual mutants are produced as recombinant protein using an expression vector derived from pUC119, which contains the LacZ promoter, a resistance gene for ampicillin, a multiple cloning site and an ompA leader sequence (pAX50). E. coli TG1 cells are transformed with the expression vector library and plated on agar plates (LB+Amp+2% glucose). Single colonies are picked from the agar plates and grown in 1 mL 96-deep-well plates. VHH expression is induced by adding IPTG (1 mM). Periplasmic extracts (in a volume of ˜80 uL) are prepared according to standard methods and screened for binding to recombinant human and mouse DII4 in a ProteOn (BioRad, Hercules, Calif., USA) off-rate assay. In brief, a GLC ProteOn Sensor chip is coated with recombinant human DII4 on the “ligand channels” L2 and L4 (with L1/L3 as reference channel), while “ligand channels” L3 and L6 is coated with mouse DII4. Periplasmic extract of affinity matured clones is diluted 1/10 and injected across the “analyte channels” A1-A6. An average off-rate is calculated of the wild type clones present in the plate and served as a reference to calculate off-rate improvements.

In a second cycle, a combinatorial library is created by simultaneously randomising the susceptible positions identified in cycle one. For this, the full length DLLBII101G8 or DLLBII115A05 cDNA is synthesized by overlap PCR using oligonucleotides degenerated (NNS) at the randomisation positions and a rescue PCR is performed. A list of the primers used for generating the combinatorial library can be found in Table 14 and SEQ ID NOs: 427 to 457. The randomised VHH genes are inserted into a phage display vector (pAX50) using specific restriction sites as described above (Example 2). Preparation of periplasmic extracts of individual VHH clones is performed as described before.

TABLE 14
Oligonucleotides affinity maturation libraries

| 101G08 combinatorial | 115A5 combinatorial |
|---|---|
| library oligonucleotides | library oligonucleotides |
| >101G08CL_fwd1-bis | >115A05CL_fwd_1 |
| gaggtgcaattggtggagtctgggGGTGGTCT | gaggtgcaattggtggagtctgggGGTGGTCTGGT |
| GGTTCAGGCTGGT | TCAGCCAGGT |
| (SEQ ID NO: 427) | (SEQ ID NO: 443) |
| >101G08CL_fwd_2 | >115A5CL_rev1-bis |
| TCCTGCGCAGCTTCTGGTCGTACCTTCTCCAG | TGAGGAGACGGTGACCTGGGTCCCCTGACCCC |
| CTACGCGATGGCT | (SEQ ID NO: 444) |
| (SEQ ID NO: 428) | |
| >101G08CL_fwd_3 | >115A05CL_fwd_2 |
| CCAGGCAAAGAACGCGAGTWCGTAGCCGCAAT | GTGCAGCTTCCGGCTTTACGWTCGGCTCCTACGAC |
| CCGTTGGAGCGGT | ATGTCTTGGG |
| (SEQ ID NO: 429) | (SEQ ID NO: 445) |
| >101G08CL_fwd_4 | >115A05CL1_rev_2 |
| CTGATTCCGTTCAGGGTCGTTTCACCATCTCT | ACGCACCCCAGTATTCACCCTGACGCGCCCAAATG |
| CGTGACAACGCG | TAGCGATCTGCAGC |
| (SEQ ID NO: 430) | (SEQ ID NO: 446) |
| >101G08CL_fwd_5 | >115A05CL_fwd_3 |
| CTGCAGATGAACTCTCTGAAACCGGAAGATAC | AGGTCCGGAATGGGTGTCCKCTATCAACTCTGGTG |
| GGCAGTCTACTAC | GTGGTAGCAC |
| (SEQ ID NO: 431) | (SEQ ID NO: 447) |
| >101G08CL_fwd_6-4 | >115A05CL_rev_3 |
| GACACTCGTCTGcgtCCGTACctgTACGACYA | TCTTCCGGTTTCAGGCTGTTCATCTGCAGGTACAG |
| TTGGGGTCAGGGTA | CGTGTTTTTG |
| (SEQ ID NO: 432) | (SEQ ID NO: 448) |
| >101G08CL_fwd_6-3 | >115A05CL_fwd_4 |
| GACACTCGTCTGGvACCGTACctgTACGACYA | AAAGGTCGTTTCACCATCTCTCGTGACAACGCCAA |
| TTGGGGTCAGGGTA | AAACACGCTG |
| (SEQ ID NO: 433) | (SEQ ID NO: 449) |
| >101G08CL_fwd_6-2 | >115A05CL_rev_4 |
| GACACTCGTCTGcgtCCGTACGAGTACGACYA | TGAAACGACCTTTTWCGWAGTCGGYGTAGWAGGTG |
| TTGGGGTCAGGGTA | CTACCACCAC |
| (SEQ ID NO: 434) | (SEQ ID NO: 450) |
| >101G08CL_fwd_6-1 | >115A05CL_fwd_5 |
| GACACTCGTCTGGVACCGTACGAGTACGACYA | TGAAACCGGAAGATACCGCGGTATACTACTGCGCT |
| TTGGGGTCAGGGTA | GCAGATCGCT |
| (SEQ ID NO: 435) | (SEQ ID NO: 451) |
| >101G08CL_rev_2-2 | >115A05CL_rev_5 |
| CAGACGAGTGTCcggCGCACGGTTTGCACAGT | CCATTCCGGACCTTTACCCGGAGAACGACGAACCC |
| AGTAGACTGCCGT | AAGACATGTC |
| (SEQ ID NO: 436) | (SEQ ID NO: 452) |
| >101G08CL_rev_2-1 | >115A05CL_fwd_6-1 |
| CAGACGAGTGTCTRCCGCACGGTTTGCACAGT | TACTGGGGTGCGTACGHATACGACTACTGGGGTCA |
| AGTAGACTGCCGT | GGGTAC |
| (SEQ ID NO: 437) | (SEQ ID NO: 453) |
| >101G08CL_rev_3 | >115A05CL_fwd_6-2 |
| AGAGTTCATCTGCAGATAGACGGTGTTTTTCG | TACTGGGGTGCGTACcagTACGACTACTGGGGTCA |
| CGTTGTCACGAGA | GGGTAC |
| (SEQ ID NO: 438) | (SEQ ID NO: 454) |
| >101G08CL_rev_4 | >115A05CL_rev_6 |
| CTGAACGGAATCAGSGTAATACGCAGTTYCAC | CCGGAAGCTGCACAGCTCAGACGCAGAGAACCACC |
| CGCTCCAACGGAT | TGGCTGAACC |
| (SEQ ID NO: 439) | (SEQ ID NO: 455) |
| >101G08CL_rev_5 | >115A05CL2_rev_2-2 |
| GCGTTCTTTGCCTGGAGCCTGACGAWACCAAG | ACGCACCCCAGTAGTAACCCTGACGCGCCCRAATG |
| CCATCGCGTAGCT | TAGCGATCTGCAGC |
| (SEQ ID NO: 440) | (SEQ ID NO: 456) |
| >101G08CL_rev_6 | >115A05CL2_rev_2-1 |
| AGAAGCTGCGCAGGACAGACGGAGAGAGCCAC | ACGCACCCCAGTAKTCACCCTGACGCGCCCRAATG |
| CAGCCTGAACCAG | TAGCGATCTGCAGC |
| (SEQ ID NO: 441) | (SEQ ID NO: 457) |
| >101G08CL_rev1-bis | |
| TGAGGAGACGGTGACCTGGGTCCCCTGACCCC | |
| AAT | |
| (SEQ ID NO: 442) | |

Screening for binding to recombinant human DII4 in a ProteOn off-rate assay identifies clones with up to 38-fold (DLLBII101G08) and 11-fold (DLLBII115A05) improved off-rates (Table 15).

TABLE 15
Off-rate screening of DLLBII101G08 and
DLLBII115A05 affinity-matured clones.

| | hDLL4 | | mDLL4 | |
|---|---|---|---|---|
| | k_{d} (s⁻¹) | fold | k_{d} (s⁻¹) | fold |
| DLLBII101G08 | 2.2E−03 | 1 | 6.7E−03 | 1 |
| DLLBII129D08 | 5.9E−05 | 38 | 1.9E−04 | 35 |
| DLLBII129H04 | 6.8E−05 | 33 | 2.5E−04 | 27 |
| DLLBII129G10 | 7.3E−05 | 31 | 2.6E−04 | 26 |
| DLLBII129H07 | 7.4E−05 | 30 | 2.5E−04 | 27 |
| DLLBII129B02 | 7.6E−05 | 30 | 2.6E−04 | 26 |
| DLLBII129E11 | 8.0E−05 | 28 | 2.5E−04 | 26 |
| DLLBII130F06 | 6.5E−05 | 27 | 2.6E−04 | 19 |
| DLLBII130B03 | 6.7E−05 | 27 | 2.4E−04 | 20 |
| DLLBII129D01 | 8.5E−05 | 26 | 2.6E−04 | 26 |
| DLLBII130D06 | 6.9E−05 | 26 | 3.1E−04 | 16 |
| DLLBII129G09 | 8.8E−05 | 26 | 3.4E−04 | 20 |
| DLLBII129B05 | 9.3E−05 | 24 | 3.4E−04 | 20 |
| DLLBII130E03 | 7.5E−05 | 24 | 2.7E−04 | 18 |
| DLLBII129H05 | 9.4E−05 | 24 | 3.5E−04 | 19 |
| DLLBII130A05 | 7.5E−05 | 24 | 3.0E−04 | 17 |
| DLLBII130B02 | 7.8E−05 | 23 | 2.9E−04 | 17 |
| DLLBII129H02 | 9.9E−05 | 23 | 3.4E−04 | 19 |
| DLLBII130B04 | 8.3E−05 | 22 | 2.9E−04 | 17 |
| DLLBII129E07 | 1.1E−04 | 21 | 2.8E−04 | 24 |
| DLLBII129E03 | 1.1E−04 | 20 | 3.6E−04 | 18 |
| DLLBII129A03 | 1.2E−04 | 19 | 3.8E−04 | 18 |

The best top DLLBII101G08 variants and DLLBII115A05 variants are cloned into expression vector pAX100 in frame with a C-terminal c-myc tag and a (His)6 tag. Off-rates on recombinant mouse DII4 are also improved. VHHs are produced in E. coli as His6-tagged proteins and purified by IMAC and SEC. Sequences are represented in Tables 16-A (LLBII101G08) and 16-B (DLLBII115A05), respectively.

TABLE 16-A
Framework and CDR region sequences of DLLBII101G08 variants

| VHH ID | | | | | | | |
|---|---|---|---|---|---|---|---|
| SEQ ID NO | FR1 | CDR1 | FR2 | CDR2 | FR3 | CDR3 | FR4 |
| DLLBII12 | EVQLVESGGGLV | SYAMA | WFRQAPGK | AIRWSGGT | RFTISRDNAKN | RAPDTRLR | WGQGTQV |
| 9A03 | QAGGSLRLSCAA | | EREYVA | AYYADSVQ | TVYLQMNSLRP | PYLYDY | TVSS |
| 354 | SGRTFS | | | G | EDTAVYYCAN | | |
| DLLBII12 | EVQLVESGGGLV | SYAMA | WYRQAPGK | AIRWSGGT | RFTISRDNAKN | RAPDTRLE | WGQGTQV |
| 9B02 | QAGGSLSLSCAA | | EREYVA | AYYADSVQ | TVYLQMNSLKP | PYEYDH | TVSS |
| 355 | SGRTFS | | | G | EDTAVYYCAN | | |
| DLLBII12 | EVQLVESGGGLV | SYAMA | WYRQAPGK | AIRWSGGT | RFTISRDNAKN | RAPDTRLA | WGQGTQV |
| 9B05 | QAGGSLRLSCAA | | EREYVA | AYYADSVQ | TVYLQMNSLKP | PYEYDH | TVSS |
| 356 | SGRTFS | | | G | EDTAVYYCAN | | |
| DLLBII12 | EVQLVESGGGLV | SYAMA | WFRQAPGK | AIRWSGGT | RFTITRDNAKN | RAPDTRLE | WGQGTQV |
| 9D01 | QAGGSLRLSCAA | | EREYVA | AYYADSVQ | TVYLAMNSLKP | PYLYDH | TVSS |
| 357 | SGRTFS | | | S | EDTAVYYCAN | | |
| DLLBII12 | EVQLVESGGGLV | SYAMA | WYRQAPGK | AIRWSGGT | RFTISRDNAKN | RAPDTRLE | WGQGTQV |
| 9D08 | QAGGSLRLSCAA | | EREYVA | AYYADSVQ | TVYLQMNSLKP | PYLYDY | TVSS |
| 358 | SGRTFS | | | G | EDTAVYYCAN | | |
| DLLBII12 | EVQLVESGGGLV | SYAMA | WFRQAPGK | AIRWSGGT | RFTISRDNAKN | RAPDTRLA | WGQGTQV |
| 9E03 | QAGGSLRLSCAA | | DREYVA | AYYADSVQ | TVYLQMNSLKP | PYLYDY | TVSS |
| 359 | SGRTFS | | | G | EDTAVYYCAN | | |
| DLLBII12 | EVQLVESGGGLV | SYAMA | WYRQAPGK | AIRWSGGT | RFTISRDNAKN | RAPDTRLA | WGQGTQV |
| 9E07 | QAGGSLRLSCSA | | EREYVA | AYYPDSVQ | TVYLQMNSLKP | PYEYDH | TVSS |
| 360 | SGRTFS | | | G | EDTAVYYCAN | | |
| DLLBII12 | EVQLVESGGGLV | SYAMA | WYRQAPGK | AIRWSGGT | RFTISRDNAKN | RAPDTRLR | WGQGTQV |
| 9E11 | QAGGSLRLSCAA | | EREYVA | AYYADSVQ | TVYLQMNSLKP | PYLYDY | TVSS |
| 361 | SGRTFS | | | G | EDTAVYYCAN | | |
| DLLBII12 | EVQLVESGGGLV | SYAMA | WYRQAPGK | AIRWSGET | RFTISRDNAKN | RAPDTRLE | WGQGTQV |
| 9G09 | QAGGSLRLSCAA | | EREYVA | AYYADSVQ | TVYLQMNSLKP | PYLYDH | TVSS |
| 362 | SGRTFS | | | G | EDTAVYYCAN | | |
| DLLBII12 | EVQLVESGGGLV | SYAMA | WYRQAPGK | AIRWSGGT | RFTISRDNAKN | RAPDTRLE | WGQGTQV |
| 9G10 | QAGGSLRLSCAA | | EREYVA | AYYADSVQ | TVYLQMNSLKP | PYEYDH | TVSS |
| 363 | SGRTFS | | | S | EDTAVYYCAN | | |
| DLLBII12 | EVQLVESGGGLV | SYAMA | WYRQAPGK | AIRWSGGT | RFTISRDNAKN | RAPDTRLR | WGQGTQV |
| 9H02 | QAGGSLRLSCAA | | EREYVA | AYYADSVQ | TVYLQMNSLKP | PYEYDY | TVSS |
| 364 | SGRTFS | | | G | EDTAVYYCAN | | |
| DLLBII12 | EVQLVESRGGLV | SYAMA | WFRQAPGK | AIRWSGGT | RFTISRDNAKN | RAPDTRLE | WGQGTQV |
| 9H04 | QAGGSLRLSCAA | | EREYVA | AYYADSVQ | TVYLQMNSLKP | PYLYDH | TVSS |
| 365 | SGRTFS | | | G | EDTAVYYCAN | | |
| DLLBII12 | EVQLVESGGGLV | SYAMA | WYRLAPGK | AIRWSGGT | RFTISRDNAKN | RAPDTRLG | WGQGTQV |
| 9H05 | QAGGSLRLSCAA | | EREYVA | AYYADSVQ | TVYLQMNSLKP | PYLYDY | TVSS |
| 366 | SGRTFS | | | G | EDTAVYYCAN | | |
| DLLBII12 | EVQLVESGGGLV | SYAMA | WYRQAPGK | AIRWSGGT | RFTISRDNAKN | RAPDTRLE | WGQGTQV |
| 9H07 | QAGGSLRLSCAA | | EREYVA | AYYADSVQ | TVYLQMNSLKP | PYEYDY | TVSS |
| 367 | SGRTFS | | | G | EDTAVYYCAN | | |
| DLLBII13 | EVQLVESGGGLV | SYAMA | WYRQAPGK | AIRWSGGT | RFTISRDNAKN | RAPDTRLG | WGQGTQV |
| 0A05 | QAGGSLRLSCAA | | EREYVA | AYYADSVQ | TVYLQMNSLKP | PYLYDH | TVSS |
| 368 | SGRTFS | | | G | EDTAVYYCAN | | |
| DLLBII13 | EVQLVESGGGLV | SYAMA | WFRQAPGK | AIRWSGGT | RFTISRDNAKN | RAPDTRLA | WGQGTQV |
| 0B02 | QAGGSLRLSCAA | | EREYVA | AYYADSVQ | TVYLQMYSLKP | PYLYDH | TVSS |
| 369 | SGRTFS | | | G | EDTAVYYCAN | | |
| DLLBII13 | EVQLVESGGGLV | SYAMA | WYRQAPGK | AIRWSGGT | RFTISRDNAKN | RAPDTRLA | WGQGTQV |
| 0B03 | QAGGSLRLSCAA | | EREYVA | AYYADSVQ | TVYLQMNSLKP | PYLYDY | TVSS |
| 370 | SGRTFS | | | G | EDTAVYYCAN | | |
| DLLBII13 | EVQLVESGGGLV | SYAMA | WFRQAPGK | AIRWSGGT | RFTISRDNAKN | RAPDTRLR | WGQGTQV |
| 0B04 | QAGGSLRLSCAA | | EREYVA | AYYADSVQ | TVYLQMNSLKP | PYLYDH | TVSS |
| 371 | SGRTFS | | | G | EDTAVYYCAN | | |
| DLLBII13 | EVQLVESGGGLV | SYAMA | WYRQAPGK | AIRWSGET | RFTISRDNAKN | RAPDTRLE | WGQGTQV |
| 0D06 | QAGGSLRLSCSA | | EREYVA | AYYADSVQ | TVYLQMNSLKP | PYLYDH | TVSS |
| 372 | SGRTFS | | | G | EDTAVYYCAN | | |
| DLLBII13 | EVQLVESGGGLV | SYAMA | WYRQAPGK | AIRWSGGT | RFTISRDNAKN | RAPDTRLE | WGQGTQV |
| 0E03 | QAGGSLRLSCAA | | EREYVA | AYYADSVQ | TVYLQMNSLKP | PYEYDH | TVSS |
| 373 | SGRTFS | | | G | EDTAVYYCAN | | |
| DLLBII13 | EVQLVESGGGLV | SYAMA | WYRQAPGK | AIRWSGGT | RFTISRDNAKN | RAPDTRLA | WGQGTQV |
| 0F06 | QAGGSLRLSCAA | | EREYVA | AYYADSVQ | TVYLQMNSLKP | PYEYDY | TVSS |
| 374 | SGRTFS | | | G | EDTAVYYCAN | | |

TABLE 16-B
Framework and CDR region sequences of DLLBII115A05 variants

| VHH ID | | | | | | | |
|---|---|---|---|---|---|---|---|
| SEQ ID NO | FR1 | CDR1 | FR2 | CDR2 | FR3 | CDR3 | FR4 |
| DLLBII1 | EVQLVESGGGLV | SYDMS | WVRRSPGK | AINSGGGST | RFTISRDNAKN | DRYIWARQ | WGQGTQV |
| 33A05 | QPGGSLRLSCAA | | GPEWVS | FYTDYVKG | TLYLQMNSLKP | GEYWGAYQ | TVSS |
| 396 | SGFTFS | | | | EDTAVYYCAA | YDY | |
| DLLBII1 | EVQLVESGGGLV | SYDMS | WVRRSPGK | AINSGGGST | RFTISRDNAKN | DRYIWARQ | WGQGTQV |
| 33A09 | QPGGSLRLSCAA | | GPEWVS | YYADYVKG | TLYLQMNSLKP | GEYWGAYA | TVSS |
| 397 | SGFTFG | | | | EDTAVYYCAA | YDY | |
| DLLBII1 | EVQLVESGGGLV | SYDMS | WVRRSPGK | AINSGGGST | RFTISRDNAKN | DRYIWARQ | WGQGTQV |
| 33A12 | QPGGSLRLSCAA | | GPEWVS | YYTDYVKG | TLYLQMNSLKP | GEYWGAYV | TVSS |
| 398 | SGFTFG | | | | EDTAVYYCAA | YDY | |
| DLLBII1 | EVQLVESGGGLV | SYDMS | WVRRSPGK | AINSGGGST | RFTISRDNAKN | DRYIWARQ | WGQGTQV |
| 33D06 | QPGGSLRLSCAA | | GPEWVS | YYADYVKG | TLYLQMNSLKP | GEYWGAYQ | TVSS |
| 399 | SGFTIG | | | | EDTAVYYCAA | YDY | |
| DLLBII1 | EVQLVESGGGLV | SYDMS | WVRRSPGK | AINSGGGST | RFTISRDNAKN | DRYIWARQ | WGQGTQV |
| 33F01 | QPGGSLRLSCAA | | GPEWVS | YYTDYVKG | TLYLAMNSLKP | GEYWGAYV | TVSS |
| 400 | SGFTIG | | | | EDTAVYYCAA | YDY | |
| DLLBII1 | EVQLVESGGGLV | SYDMS | WVRRSPGK | AINSGGGST | RFTISRDNAKN | DRYIWARQ | WGQGTQV |
| 33F06 | QPGGSLRLSCAA | | GPEWVS | YYTDYVKG | TLYLQMNSLKP | GEYWGAYQ | TVSS |
| 401 | SGFTFG | | | | EDTAVYYCAA | YDY | |
| DLLBII1 | EVQLVESGGGLV | SYDMS | WVRRSPGK | AINSGGGST | RFTISRDNAKN | DRYIWARQ | WGQGTQV |
| 33G05 | QPGGSLRLSCAA | | GPEWVS | YYTDYVKG | TLYLQMNSLKP | GEYWGAYA | TVSS |
| 402 | SGFTFG | | | | EDTAVYYCAA | YDY | |
| DLLBII1 | EVQLVESGGGLV | SYDMS | WVRRSPGK | AINSGGGST | RFTISRDNAKN | DRYIWARQ | WGQGTQV |
| 33H03 | QPGGSLRLSCAA | | GPEWVS | YYTDYVKG | TLYLQMNSLKP | GEYWGAYA | TVSS |
| 403 | SGFTIG | | | | EDTAVYYCAA | YDY | |
| DLLBII1 | EVQLVESGGGLV | SYDMS | WVRRSPGK | AINSGGGST | RFTISRDNAKN | DRYIWARQ | WGQGTQV |
| 34B11 | QPGGSLRLSCAA | | GPEWVS | YYTDFVKG | TLYLQMNSLKP | GEYWGAYA | TVSS |
| 404 | SGFTIG | | | | EDTAVYYCAA | YDY | |
| DLLBII1 | EVQLVESGGGLV | SYDMS | WVRRSPGK | SINSGGGST | RFTISRDNAKN | DRYIWARQ | WGQGTQV |
| 34D10 | QPGGSLRLSCAA | | GPEWVS | YYTDYVKG | TLYLQMNSLKP | GEYWGAYA | TVSS |
| 405 | SGFTIG | | | | EDTAVYYCAA | YDY | |
| DLLBII1 | EVQLVESGGGLV | SYDMS | WVRRSPGK | AINSGGGST | RFTISRDNAKN | DRYIWARQ | WGQGTQV |
| 35H04 | QPGGSLRLSCAA | | GPEWVS | YYADYVKG | TLYLQMNSLKP | GEYWGAYQ | TVSS |
| 406 | SGFTIG | | | | EDTAVYYCAA | YDY | |
| DLLBII1 | EVQLVESGGGLV | SYDMS | WVRRSPGK | SINSGGGST | RFTISRDNAKN | DRYIWARQ | WGQGTQV |
| 36C07 | QPGGSLRLSCAA | | GPEWVS | YYADYVKG | TLYLQMNSLKP | GEYWGAYE | TVSS |
| 407 | SGFTFG | | | | EDTAVYYCAA | YDY | |
| DLLBII1 | EVQLVESGGGLV | SYDMS | WVRRSPGK | AINSGGDST | RFTISRDNAKN | DRYIWARQ | WGQGTQV |
| 36D01 | QPGGSLRLSCAA | | GPEWVS | FYADYVKG | TLYLQMNSLKP | GEYWGAYA | TVSS |
| 408 | SGFTIG | | | | EDTAVYYCAA | YDY | |
| DLLBII1 | EVQLVESGGGLV | SYDMS | WLRRSPGK | AINSGGGST | RFTISRDNAKN | DRYIWARQ | WGQGTQV |
| 36H03 | QPGGSLRLSCAA | | GPEWVS | YYADYVKG | TLYLQMNSLKP | GDYWGAYV | TVSS |
| 409 | SGFTFG | | | | EDTAVYYCAA | YDY | |
| DLLBII1 | EVQLVESGGGLV | SYDMS | WVRRSPGK | AINSGGGST | RFTISRDNAKN | DRYIWARQ | WGQGTQV |
| 37A04 | QPGGSLRLSCAA | | GPEWVS | YYTDYVKG | TLYLQMNSLKP | GDYWGAYA | TVSS |
| 410 | SGFTFG | | | | EDTAVYYCAA | YDY | |
| DLLBII1 | EVQLVESGGGLV | SYDMS | WVRRSPGK | AINSGGGST | RFTISRDNAKN | DRYIRARQ | WGQGTQV |
| 37A06 | QPGGSLRLSCAA | | GPEWVS | YYTDYVKG | TLYLQMNSLKP | GEYWGAYA | TVSS |
| 411 | SGFTFG | | | | EDTAVYYCAA | YDY | |
| DLLBII1 | EVQLVESGGGLV | SYDMS | WVRRSPGK | SINSGGGST | RFTISRDNAKN | DRYIWARQ | WGQGTQV |
| 37B06 | QPGGSLRLSCAA | | GPEWVS | YYTDFVKG | TLYLQMNSLKP | GEYWGAYA | TVSS |
| 412 | SGFTIG | | | | EDTAVYYCAA | YDY | |
| DLLBII1 | EVQLVESGGGLV | SYDMS | WVRRSPGK | AINSGGGST | RFTISRDNAKN | DRYIWARQ | WGQGTQV |
| 37C04 | QPGGSLRLSCAA | | GPEWVS | YYTDYVKG | TLYLQMNSLKP | GEYWGAYE | TVSS |
| 413 | SGFTIG | | | | EDTAVYYCAA | YDY | |
| DLLBII1 | EVQLVESGGGLV | SYDMS | WVRRSPGK | SINSGGGST | RFTISRDNAKN | DRYIWARQ | WGQGTQV |
| 37F04 | QPGGSLRLSCAA | | GPEWVS | FYTDFVKG | TLYLQMNSLKP | GEYWGAYA | TVSS |
| 414 | SGFTIG | | | | EDTAVYYCAA | YDY | |
| DLLBII1 | EVQLVESGGGLV | SYDMS | WVRRSPGK | AINSGGGST | RFTISRNNAKN | DRYIWARQ | WGQGTQV |
| 38F12 | QPGGSLRLSCAA | | GPEWVS | YYTDYVKG | TLYLQMNSLKP | GEYWGAYQ | TVSS |
| 415 | SGFTFG | | | | EDTAVYYCAA | YDY | |
| DLLBII0 | EVQLVESGGGLV | SYDMS | WVRRSPGK | AINSGGGST | RFTISRDNAKN | DRYIWARQ | WGQGTQV |
| 15 | APGGSLRLSCAA | | GPEWVS | YYADYVKG | TLYLQMNALKP | GEYWGAYA | TVSS |
| 416 | SGFTIG | | | | EDTAVYYCAA | YDY | |

### Example 7

### Characterization of Affinity Matured Purified Anti-DII4 VHHs

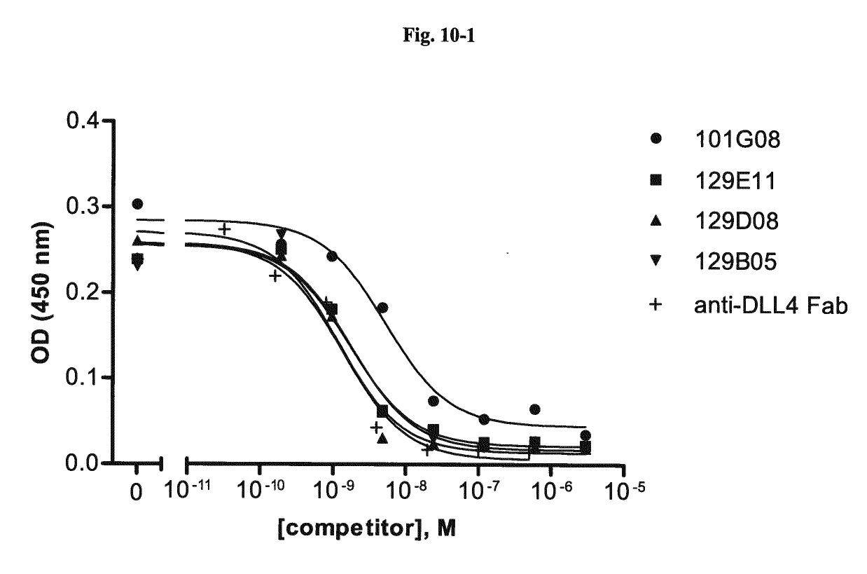

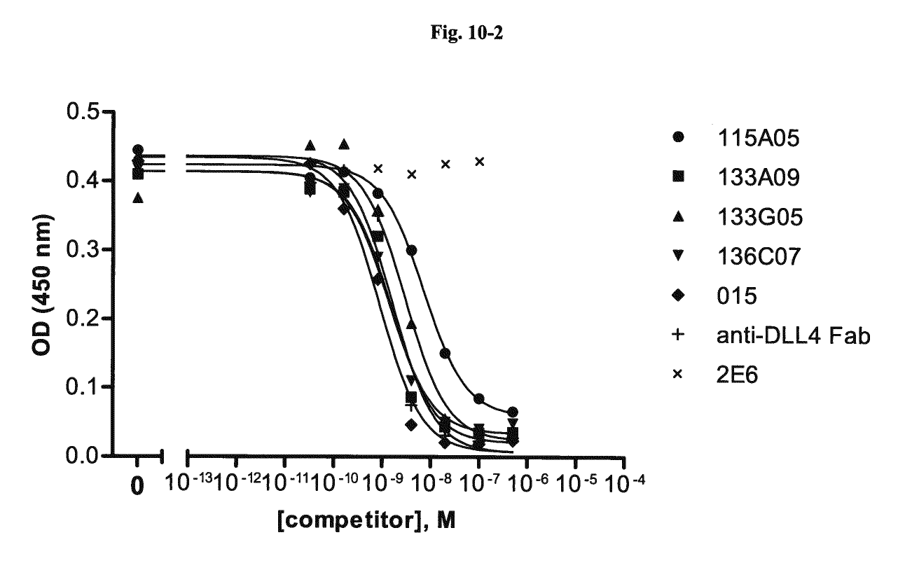

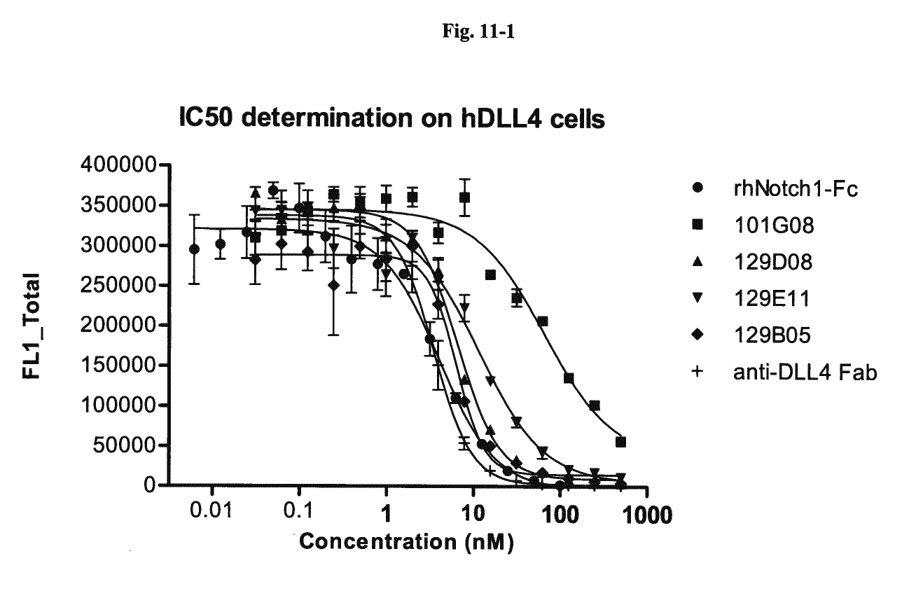

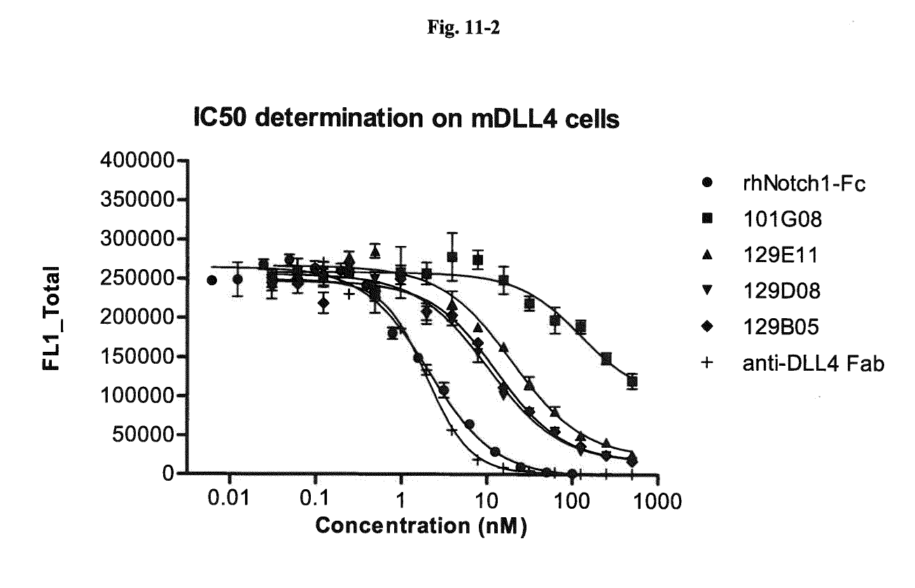

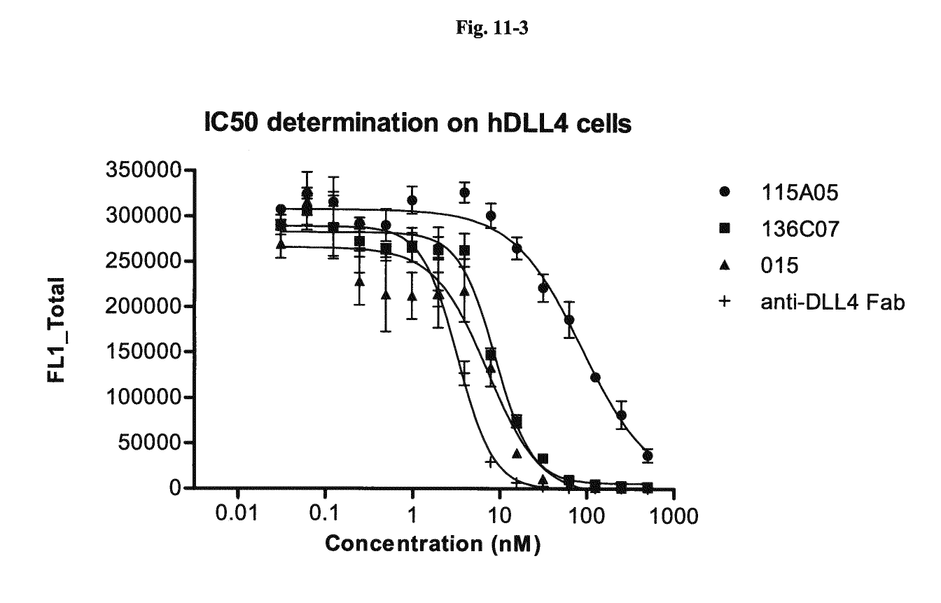

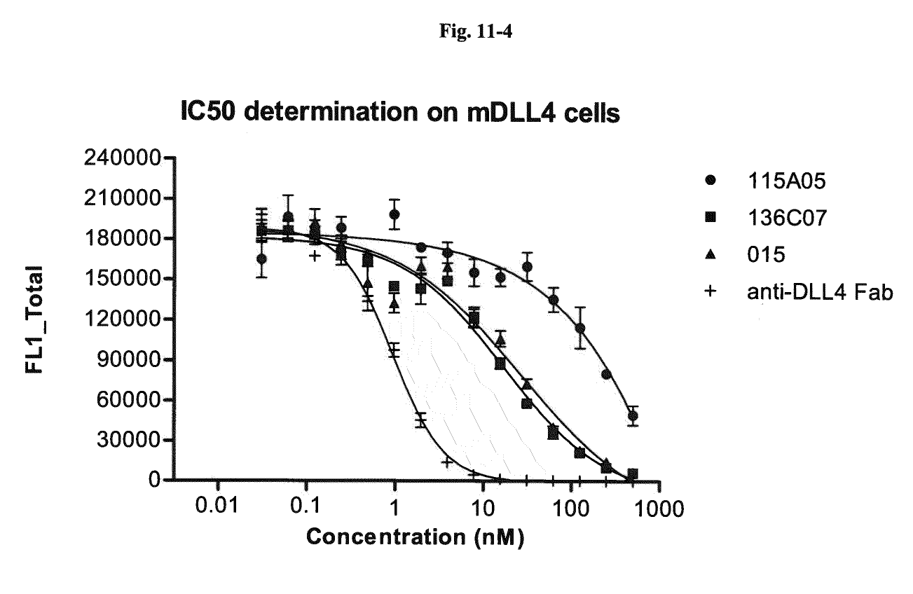

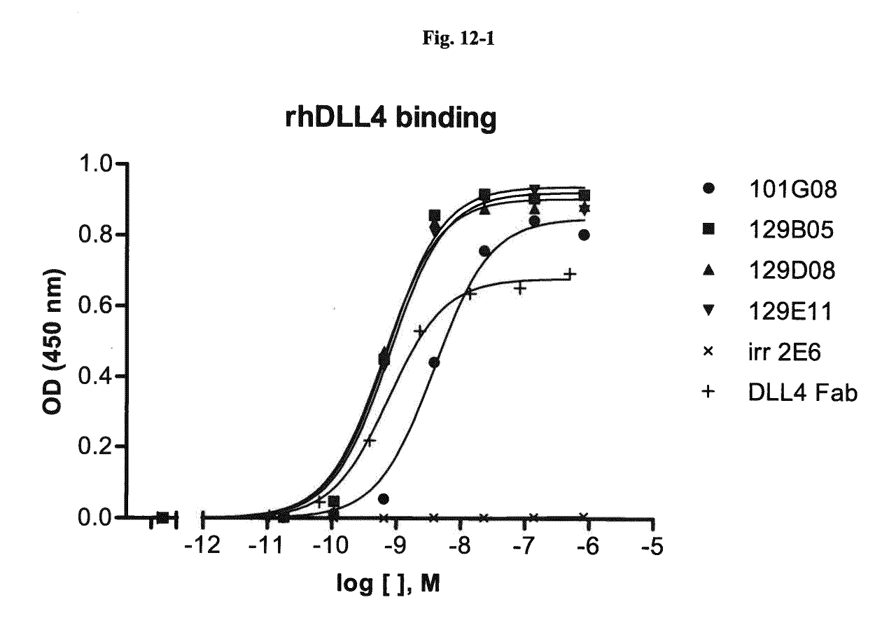

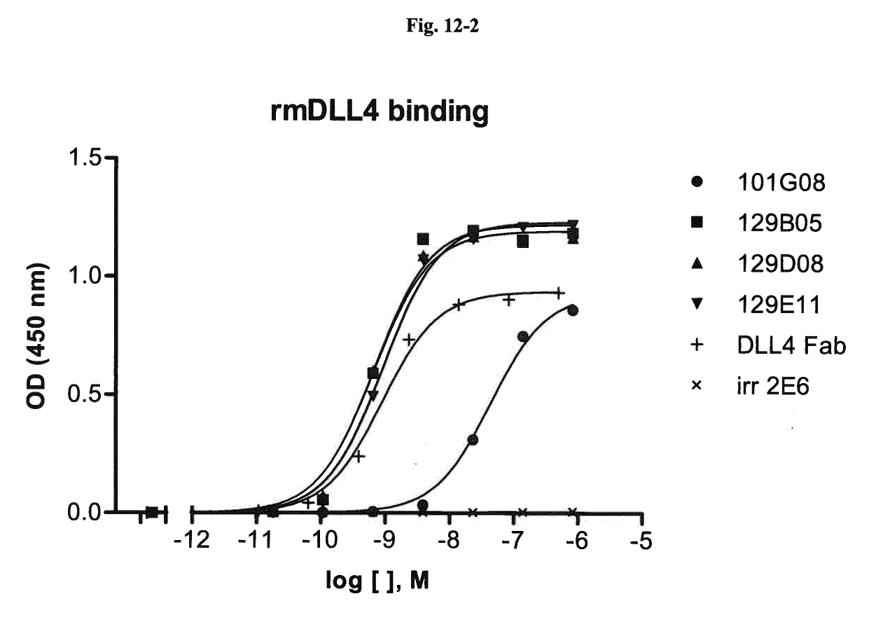

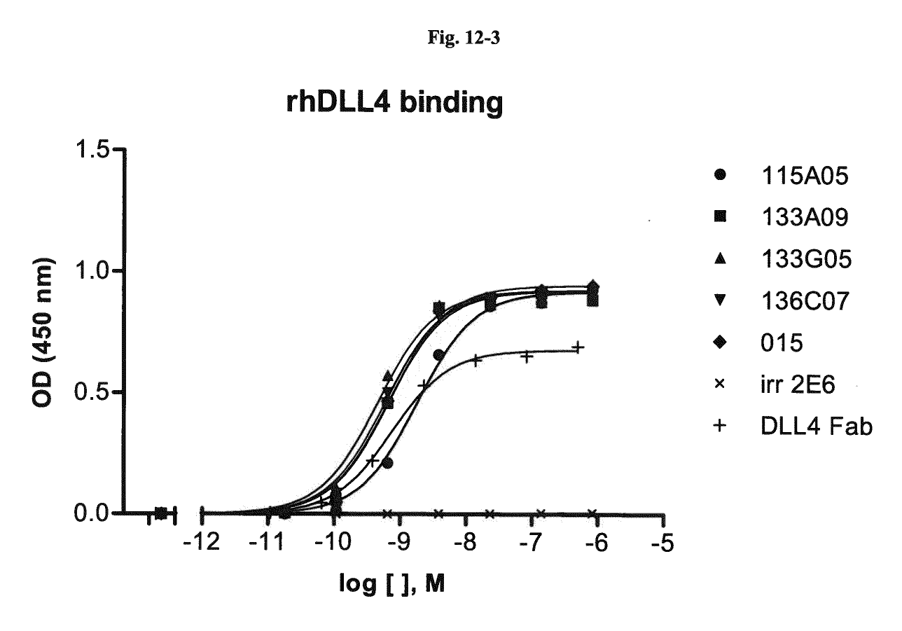

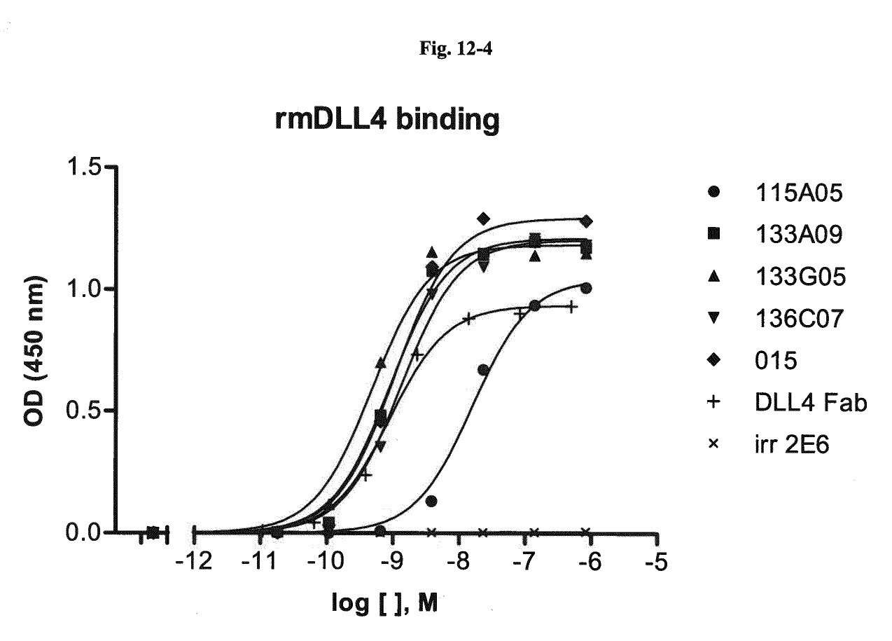

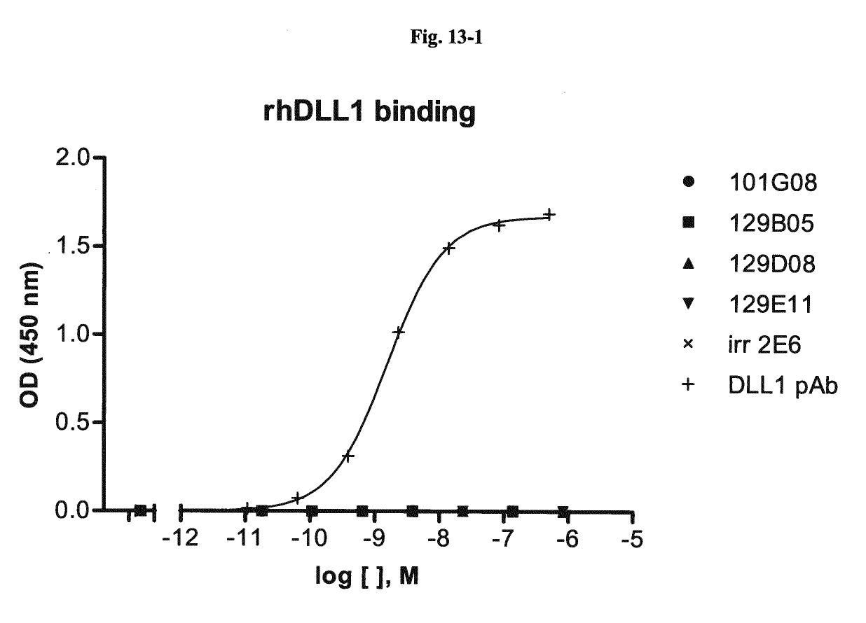

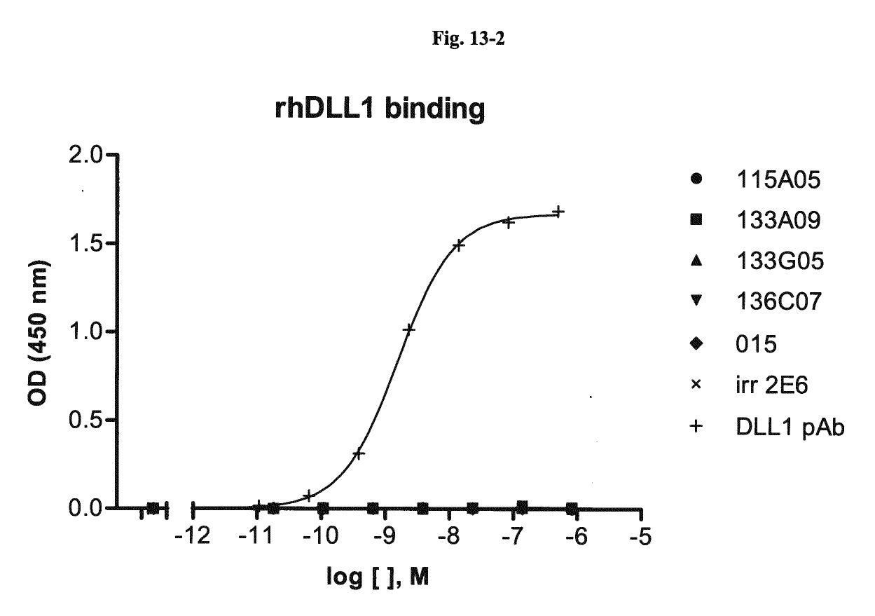

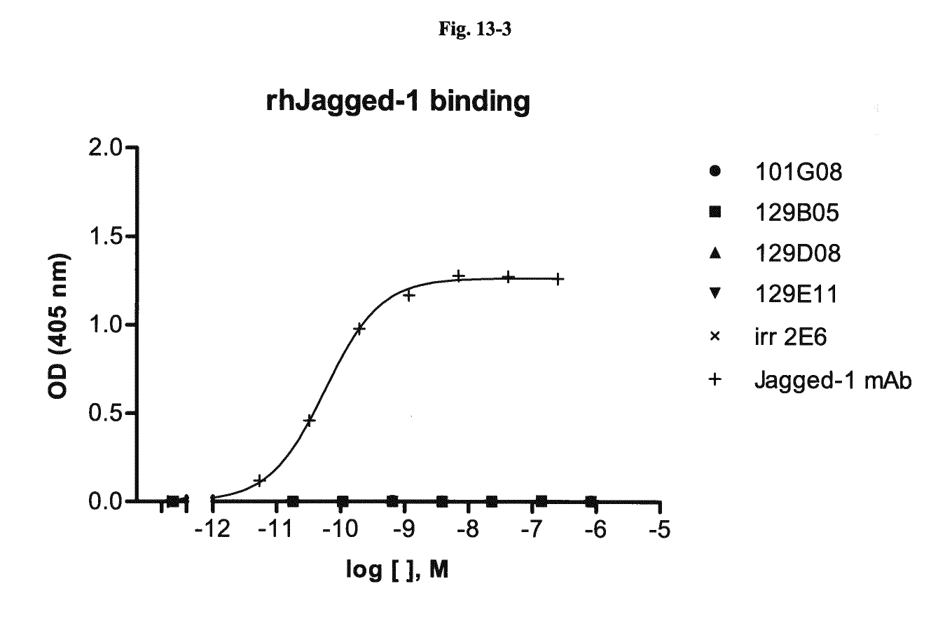

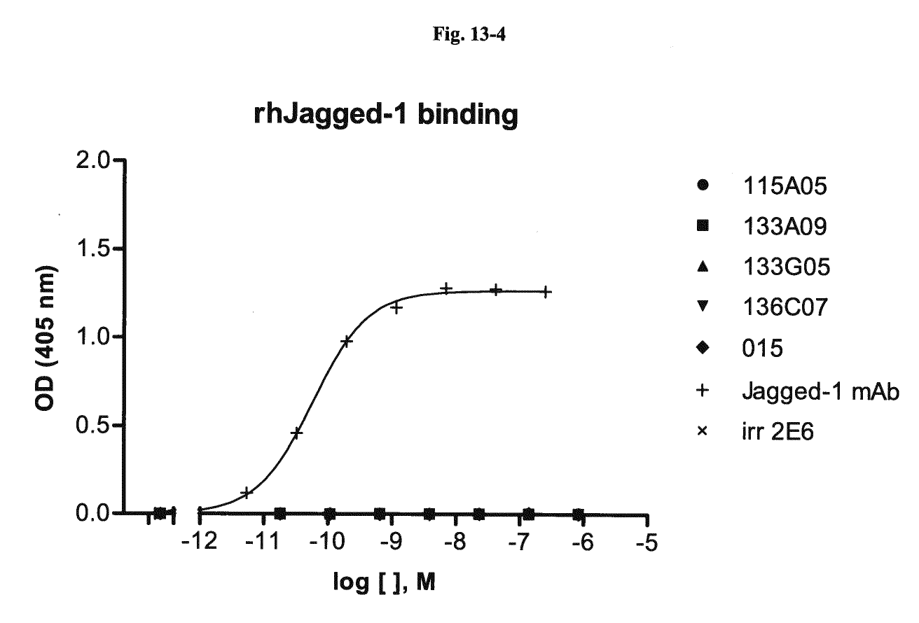

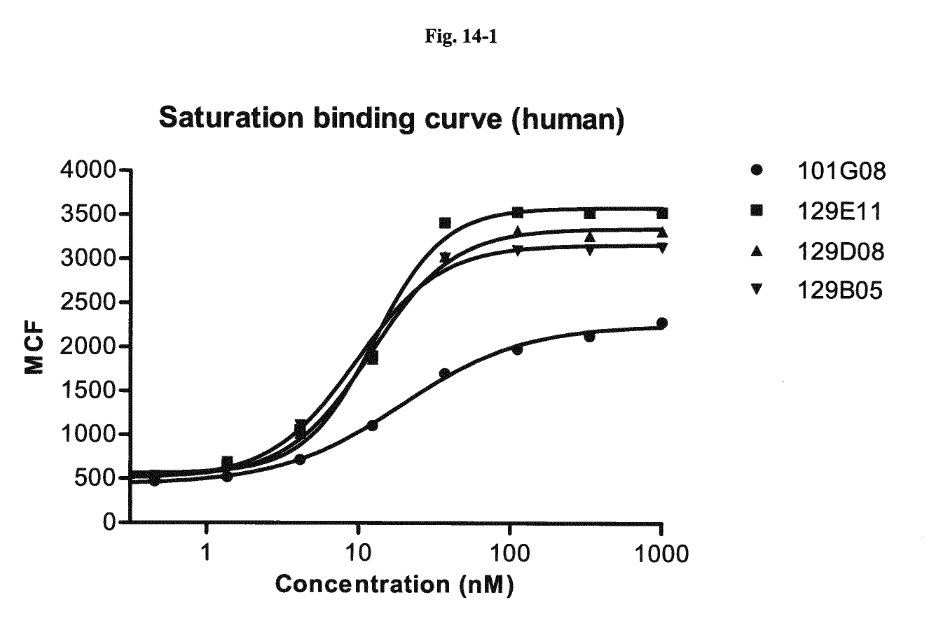

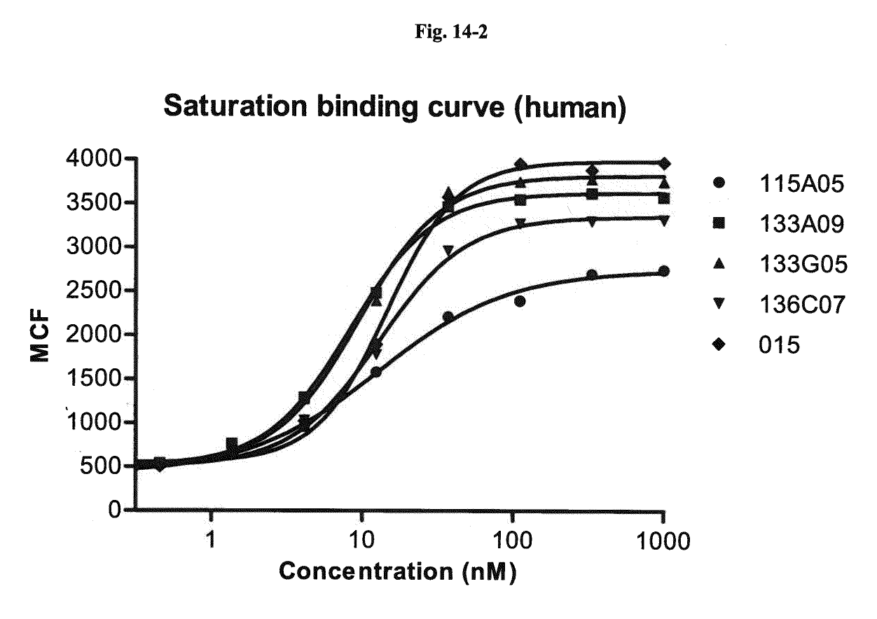

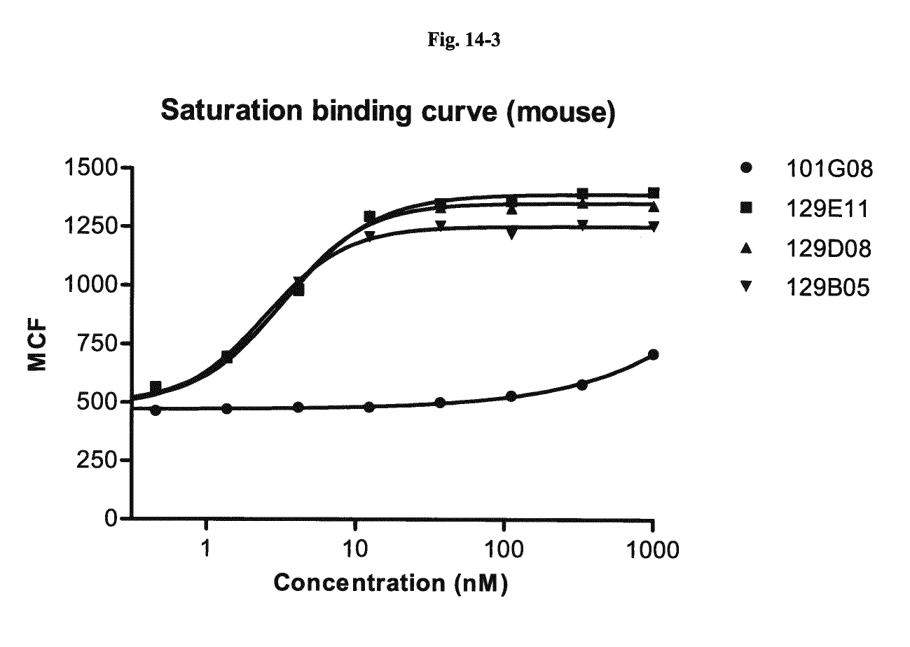

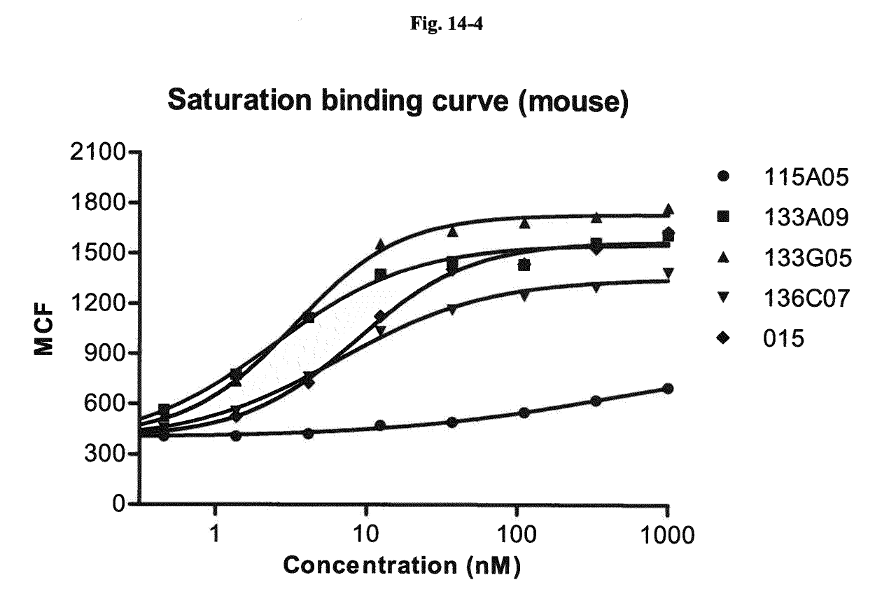

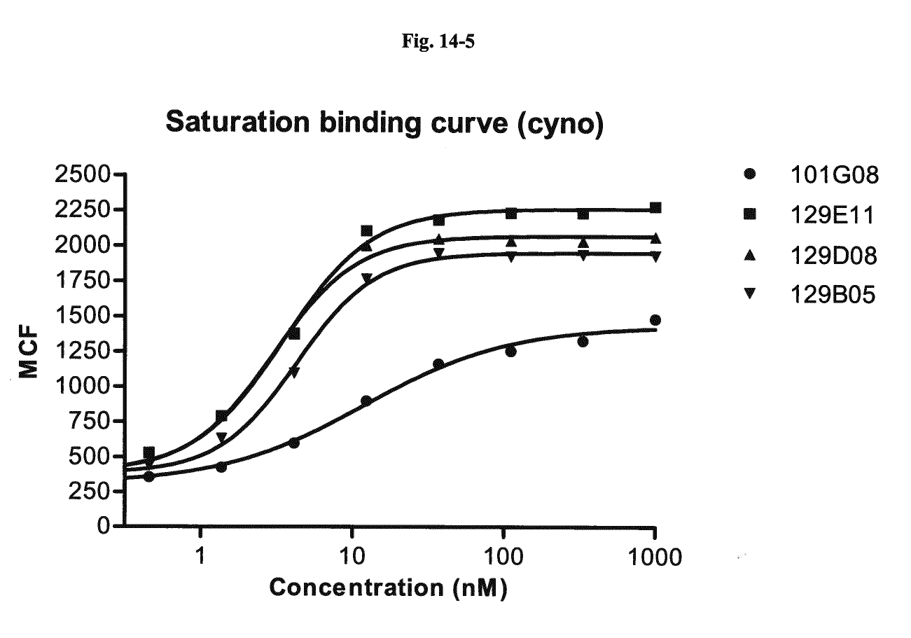

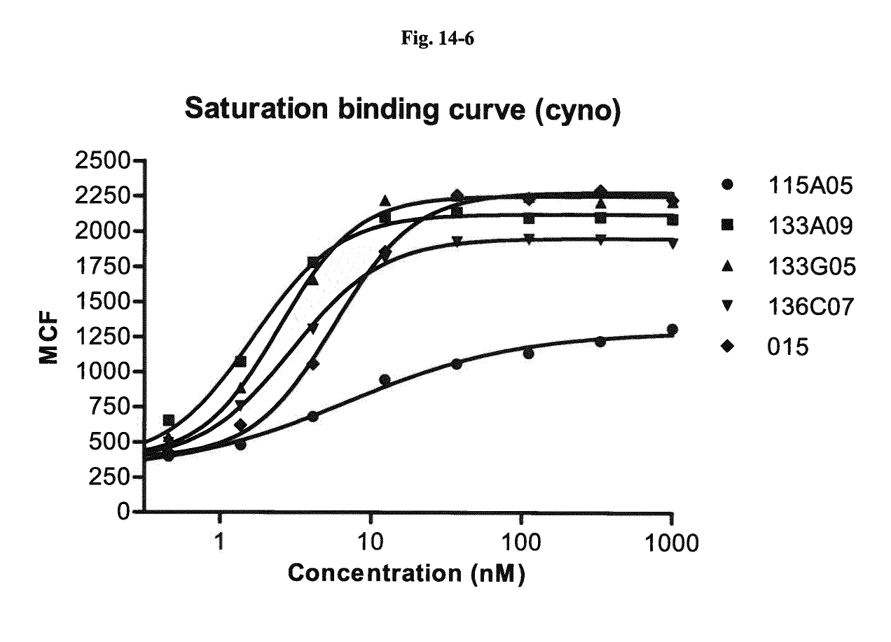

Affinity-matured variants of VHHs DLLBII101GOB and DLLBII115A05 are expressed and purified as described above (Example 6). VHHs are characterized in the rhDLL1/rhJAG1 binding ELISA and hDII4/mDII4/cynoDII4 FACS (Example 5.8; Table 20; FIGS. 12 and 13), the rhDII4-rhNotch1 competition ELISA (Example 5.1; Table 17; FIG. 10), the competition rhNotch1-CHO-hDII4 FMAT (Example 5.3; Table 18; FIG. 11).

Characterization data are summarized in Table 21. Overall, the affinity matured VHHs show clear improvements in affinity and potency, while their binding to mDII4 and cyno DII4 is maintained and no binding to hDLL1 or hJAG1 is observed

TABLE 17
IC₅₀ (nM) values for affinity matured
VHHs in hDLL4 /hNotch1-Fc
competition ELISA

| VHH ID | IC₅₀ (nM) |
|---|---|
| 101G08 | 10.0 |
| 129A03 | 1.8 |
| 129B05 | 0.9 |
| 129D08 | 1.2 |
| 129E11 | 1.3 |
| 129H07 | 1.0 |
| 130B03 | 1.5 |
| 130F06 | 1.3 |
| anti-DLL4 Fab | 1.5 |
| 115A05 | 7.5 |
| 133A05 | 2.1 |
| 133A09 | 1.5 |
| 133G05 | 2.0 |
| 134D10 | 1.3 |
| 136C07 | 1.4 |
| 015 | 0.9 |
| anti-DLL4 Fab | 1.2 |

TABLE 18
IC₅₀ values (nM) of purified affinity matured
VHHs blocking the interaction of human
Notch1/Fc to human or mouse DLL4
expressed on CHO cells (FMAT)

| | hDLL4 | mDLL4 |
|---|---|---|
| VHH ID | IC₅₀ (nM) | IC₅₀ (nM) |
| 101G08 | 69.3 | 140.5 |
| 129B05 | 7.4 | 14.4 |
| 129D08 | 7.8 | 11.0 |
| 129E11 | 8.1 | 12.3 |
| anti-DLL4 Fab | 5.5 | 3.0 |
| 115A05 | 106.7 | 348.9 |
| 133A09 | 6.6 | 18.6 |
| 133G05 | 5.9 | 12.0 |
| 136C07 | 8.0 | 31.2 |
| 015 | 5.7 | 21.2 |
| anti-DLL4 Fab | 3.4 | 1.6 |

TABLE 19
Affinity K_{D} (nM) of purified affinity matured VHHs
on recombinant human DLL4 and mouse DLL4

| | rhDLL4 | | | rmDLL4 | | |
|---|---|---|---|---|---|---|
| | kₐ | | K_{D} | kₐ | | K_{D} |
| VHH ID | (M⁻¹s⁻¹) | k_{d} (s⁻¹) | (nM) | (M⁻¹s⁻¹) | k_{d} (s⁻¹) | (nM) |
| 101G08 | 4.8E+04 | 2.3E−03 | 48.0 | 9.4E+04 | 5.6E−03 | 60.0 |
| (wt) | | | | | | |
| 129A03 | 2.1E+05 | 1.2E−04 | 0.5 | | | |
| 129B05 | 2.3E+05 | 7.9E−05 | 0.3 | 2.7E+05 | 3.1E−04 | 1.1 |
| 129D08 | 1.8E+05 | 6.4E−05 | 0.4 | 2.7E+05 | 2.0E−04 | 0.8 |
| 129E11 | 1.9E+05 | 9.0E−05 | 0.5 | 2.5E+05 | 2.9E−04 | 1.2 |
| 129H07 | 1.6E+05 | 7.3E−05 | 0.5 | | | |
| 130B03 | 2.2E+05 | 6.8E−05 | 0.3 | | | |
| 130F06 | 2.0E+05 | 8.0E−05 | 0.4 | | | |
| anti- | 2.3E+05 | 3.4E−04 | 1.5 | | | |
| DLL4 | | | | | | |
| Fab | | | | | | |
| 115A05 | 2.5E+05 | 4.0E−03 | 16.0 | 1.7E+05 | 9.1E−03 | 53.0 |
| (wt) | | | | | | |
| 133A09 | 4.4E+05 | 9.0E−04 | 2.1 | 3.5E+05 | 2.7E−03 | 7.8 |
| 133G05 | 5.9E+05 | 4.7E−04 | 0.8 | 4.7E+05 | 1.6E−03 | 3.4 |
| 136C07 | 6.2E+05 | 3.9E−04 | 0.6 | 5.0E+05 | 1.3E−03 | 2.6 |
| 015 | 4.5E+05 | 4.7E−04 | 1.0 | 3.5E+05 | 1.5E−03 | 4.3 |
| anti- | 2.3E+05 | 3.4E−04 | 1.5 | | | |
| DLL4 | | | | | | |
| Fab | | | | | | |

TABLE 20
EC₅₀ (nM) values of affinity matured
VHHs for binding on CHO-hDLL4,
CHO-mDLL4 and CHO-cDLL4 (FACS)

| | hDLL4 | mDLL4 | cDLL4 |
|---|---|---|---|
| VHH ID | EC₅₀ (nM) | EC₅₀ (nM) | EC₅₀ (nM) |
| 101G08(wt) | 17.5 | | 11.2 |
| 129B05 | 9.7 | 3.9 | 3.9 |
| 129D08 | 9.6 | 3.7 | 3.8 |
| 129E11 | 1.4 | 4.1 | 4.2 |
| anti-DLL4 Fab | 5.6 | 2.1 | 2.5 |
| 115A05(wt) | 11.3 | | 13.8 |
| 133A09 | 7.2 | 1.7 | 2.3 |
| 133G05 | 8.5 | 2.8 | 2.7 |
| 136C07 | 10.9 | 8.3 | 3.5 |
| 015 | 14.8 | 7.0 | 5.1 |
| anti-DLL4 Fab | 5.6 | 2.1 | 2.5 |

TABLE 21
Characteristics of affinity-matured VHHs derived from
DLLBII101G08 and DLLBII115A05

| | | | | FMAT | FMAT | | | | | |
|---|---|---|---|---|---|---|---|---|---|---|
| | K_{D} | K_{D} | ELISA | hDLL4 | mDLL4 | FACS | FACS | FACS | | ELISA |
| | (nM) | (nM) | IC₅₀ | IC₅₀ | IC₅₀ | EC₅₀ | EC₅₀ | EC₅₀ | ELISA | hJa |
| | hDLL4 | mDLL4 | (nM) | (nM) | (nM) | (nM) | (nM) | (nM) | hDLL1 | g-1 |
| 101G08 | 48.0 | 60.0 | 10.0 | 69.3 | 140.5 | 17.5 | NF | 11.2 | nb | nb |
| 129A03 | 0.5 | | 1.8 | | | | | | | |
| 129B05 | 0.3 | 1.1 | 0.9 | 7.4 | 14.4 | 9.7 | 3.9 | 3.9 | nb | nb |
| 129D08 | 0.4 | 0.8 | 1.2 | 7.8 | 11.0 | 9.6 | 3.7 | 3.8 | nb | nb |
| 129E11 | 0.5 | 1.2 | 1.3 | 8.1 | 12.3 | 10.4 | 4.1 | 4.2 | nb | nb |
| 129H07 | 0.5 | | 1.0 | | | | | | | |
| 130B03 | 0.3 | | 1.5 | | | | | | | |
| 130F06 | 0.4 | | 1.3 | | | | | | | |
| DLL4 | 1.5 | | 1.5 | 5.5 | 3.0 | 5.6 | 2.1 | 2.5 | | |
| Fab | | | | | | | | | | |
| 115A05 | 16.0 | 53.0 | 7.5 | 106.7 | 348.9 | 11.3 | NF | 13.8 | nb | nb |
| 133A05 | | | 2.1 | | | | | | | |
| 133A09 | 2.1 | 7.8 | 1.5 | 6.6 | 18.6 | 7.2 | 1.7 | 2.3 | nb | nb |
| 133G05 | 0.8 | 3.4 | 2.0 | 5.9 | 12.0 | 8.5 | 2.8 | 2.7 | nb | nb |
| 134D10 | | | 1.3 | | | | | | | |
| 136C07 | 0.6 | 2.6 | 1.4 | 8.0 | 31.2 | 10.9 | 8.3 | 3.5 | nb | nb |
| 015 | 1.0 | 4.3 | 0.9 | 5.7 | 21.2 | 14.8 | 7.0 | 5.1 | nb | nb |
| DLL4 | 1.5 | | 1.2 | 3.4 | 1.6 | 5.6 | 2.1 | 2.5 | | |
| Fab | | | | | | | | | | |

nb: no binding

### Example 8

### Construction, Production and Characterization of Bispecific VHHs Targeting DII4 and Ang2 Using Anti-Serum Albumin Binding as Half-Life Extension

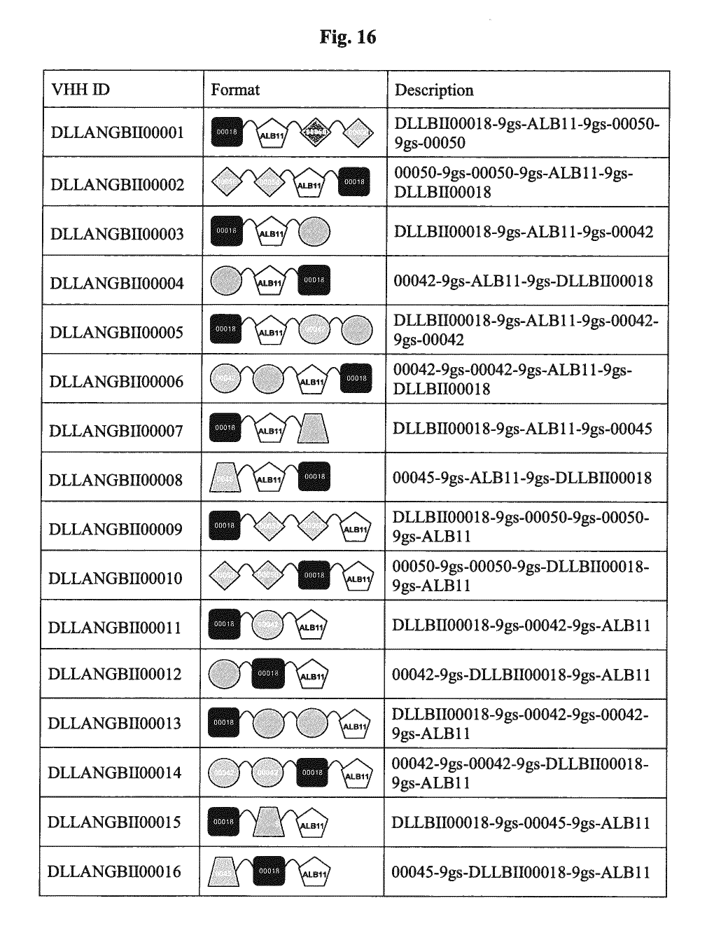

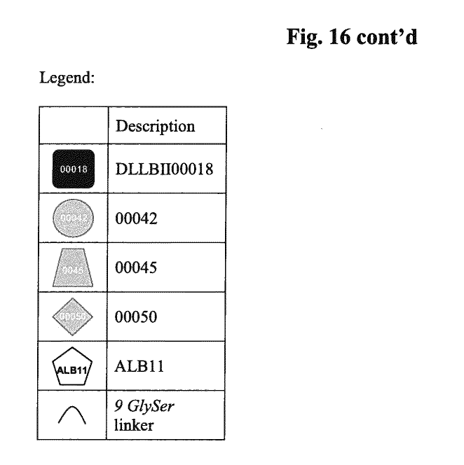

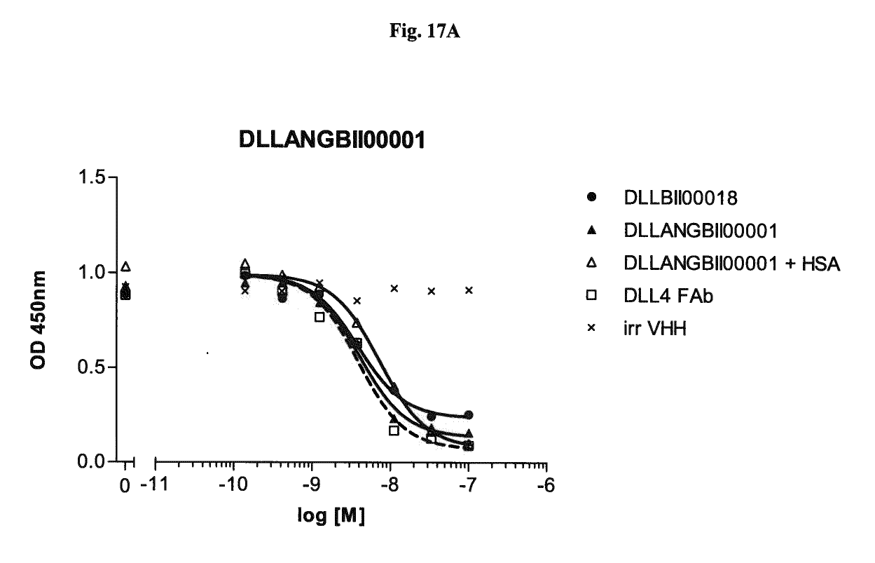

In a first cycle, the anti-DLL4 VHH DLLBII00018 (US 2011/0172398 A1) and the cycle 1 sequence optimized anti-Ang2 VHHs 00042 (SEQ ID NO: 482), 00045 (SEQ ID NO: 484) and 00050 (SEQ ID NO:483) are used as building blocks to generate bispecific VHHs DLLANGBII00001-00016. A genetic fusion to a serum albumin binding VHH is used as half-life extension methodology. Building blocks are linked via a 9 Gly-Ser flexible linker. VHHs are produced and purified as described in Example 5. An overview of the format and sequence of all bispecific VHHs is depicted in FIG. 16 and Table 22-A (linker sequences underlined), SEQ ID Nos 460-475. Expression levels are indicated in Table 22-B.

TABLE 22-A
Sequences of bispecific VHH targeting DLL4 and Ang2

| VHH ID | AA sequence |
|---|---|
| DLLANGBII00001 | DVQLVESGGGLVQPGGSLRLSCAASGRTFSSYAMAWYRQAPGKEREYVAAIRWSGGTAYYADSVKGR |
| | FTISRDNAKNTVYLQMNSLRPEDTAVYYCANRAPDTRLAPYEYDHWGQGTLVTVSSGGGGSGGGSEV |
| | QLVESGGGLVQPGNSLRLSCAASGFTFSSFGMSWVRQAPGKGLEWVSSISGSGSDTLYADSVKGRFT |
| | ISRDNAKTTLYLQMNSLRPEDTAVYYCTIGGSLSRSSQGTLVTVSSGGGGSGGGSEVQLVESGGGLV |
| | QPGGSLRLSCAASGFTFDDYALGWFRQAPGKEREGVSCIRCSDGSTYYADSVKGRFTISSDNSKNTV |
| | YLQMNSLRPEDTAVYYCAASIVPRSKLEPYEYDAWGQGTLVTVSSGGGGSGGGSEVQLVESGGGLVQ |
| | PGGSLRLSCAASGFTFDDYALGWFRQAPGKEREGVSCIRCSDGSTYYADSVKGRFTISSDNSKNTVY |
| | LQMNSLRPEDTAVYYCAASIVPRSKLEPYEYDAWGQGTLVTVSS |
| | (SEQ ID NO: 460) |
| DLLANGBII00002 | DVQLVESGGGLVQPGGSLRLSCAASGFTFDDYALGWFRQAPGKEREGVSCIRCSDGSTYYADSVKGR |
| | FTISSDNSKNTVYLQMNSLRPEDTAVYYCAASIVPRSKLEPYEYDAWGQGTLVTVSSGGGGSGGGSE |
| | VQLVESGGGLVQPGGSLRLSCAASGFTFDDYALGWFRQAPGKEREGVSCIRCSDGSTYYADSVKGRF |
| | TISSDNSKNTVYLQMNSLRPEDTAVYYCAASIVPRSKLEPYEYDAWGQGTLVTVSSGGGGSGGGSEV |
| | QLVESGGGLVQPGNSLRLSCAASGFTFSSFGMSWVRQAPGKGLEWVSSISGSGSDTLYADSVKGRFT |
| | ISRDNAKTTLYLQMNSLRPEDTAVYYCTIGGSLSRSSQGTLVTVSSGGGGSGGGSEVQLVESGGGLV |
| | QPGGSLRLSCAASGRTFSSYAMAWYRQAPGKEREYVAAIRWSGGTAYYADSVKGRFTISRDNAKNTV |
| | YLQMNSLRPEDTAVYYCANRAPDTRLAPYEYDHWGQGTLVTVSS |
| | (SEQ ID NO: 461) |
| DLLANGBII00003 | DVQLVESGGGLVQPGGSLRLSCAASGRTFSSYAMAWYRQAPGKEREYVAAIRWSGGTAYYADSVKGR |
| | FTISRDNAKNTVYLQMNSLRPEDTAVYYCANRAPDTRLAPYEYDHWGQGTLVTVSSGGGGSGGGSEV |
| | QLVESGGGLVQPGNSLRLSCAASGFTFSSFGMSWVRQAPGKGLEWVSSISGSGSDTLYADSVKGRFT |
| | ISRDNAKTTLYLQMNSLRPEDTAVYYCTIGGSLSRSSQGTLVTVSSGGGGSGGGSEVQLVESGGGLV |
| | QPGGSLRLSCAASGFTLDDYAIGWFRQAPGKEREGVSSIRDNDGSTYYADSVKGRFTISSDNSKNTV |
| | YLQMNSLRPEDTAVYYCAAVPAGRLRFGEQWYPLYEYDAWGQGTLVTVSS |
| | (SEQ ID NO: 462) |
| DLLANGBII00004 | DVQLVESGGGLVQPGGSLRLSCAASGFTLDDYAIGWFRQAPGKEREGVSSIRDNDGSTYYADSVKGR |
| | FTISSDNSKNTVYLQMNSLRPEDTAVYYCAAVPAGRLRFGEQWYPLYEYDAWGQGTLVTVSSGGGGS |
| | GGGSEVQLVESGGGLVQPGNSLRLSCAASGFTFSSFGMSWVRQAPGKGLEWVSSISGSGSDTLYADS |
| | VKGRFTISRDNAKTTLYLQMNSLRPEDTAVYYCTIGGSLSRSSQGTLVTVSSGGGGSGGGSEVQLVE |
| | SGGGLVQPGGSLRLSCAASGRTFSSYAMAWYRQAPGKEREYVAAIRWSGGTAYYADSVKGRFTISRD |
| | NAKNTVYLQMNSLRPEDTAVYYCANRAPDTRLAPYEYDHWGQGTLVTVSS |
| | (SEQ ID NO: 463) |
| DLLANGVII00005 | DVQLVESGGGLVQPGGSLRLSCAASGRTFSSYAMAWYRQAPGKEREYVAAIRWSGGTAYYADSVKGR |
| | FTISRDNAKNTVYLQMNSLRPEDTAVYYCANRAPDTRLAPYEYDHWGQGTLVTVSSGGGGSGGGSEV |
| | QLVESGGGLVQPGNSLRLSCAASGFTFSSFGMSWVRQAPGKGLEWVSSISGSGSDTLYADSVKGRFT |
| | ISRDNAKTTLYLQMNSLRPEDTAVYYCTIGGSLSRSSQGTLVTVSSGGGGSGGGSEVQLVESGGGLV |
| | QPGGSLRLSCAASGFTLDDYAIGWFRQAPGKEREGVSSIRDNDGSTYYADSVKGRFTISSDNSKNTV |
| | YLQMNSLRPEDTAVYYCAAVPAGRLRFGEQWYPLYEYDAWGQGTLVTVSSGGGGSGGGSEVQLVESG |
| | GGLVQPGGSLRLSCAASGFTLDDYAIGWFRQAPGKEREGVSSIRDNDGSTYYADSVKGRFTISSDNS |
| | KNTVYLQMNSLRPEDTAVYYCAAVPAGRLRFGEQWYPLYEYDAWGQGTLVTVSS |
| | (SEQ ID NO: 464) |
| DLLANGBII00006 | DVQLVESGGGLVQPGGSLRLSCAASGRTFSSYAMAWYRQAPGKEREYVAAIRWSGGTAYYADSVKGR |
| | FTISRDNAKNTVYLQMNSLRPEDTAVYYCANRAPDTRLAPYEYDHWGQGTLVTVSSGGGGSGGGSEV |
| | QLVESGGGLVQPGNSLRLSCAASGFTFSSFGMSWVRQAPGKGLEWVSSISGSGSDTLYADSVKGRFT |
| | ISRDNAKTTLYLQMNSLRPEDTAVYYCTIGGSLSRSSQGTLVTVSSGGGGSGGGSEVQLVESGGGLV |
| | QPGGSLRLSCAASGFALDYYAIGWFRQAPGKEREGVSCISSSDGITYYADSVKGRFTISRDNSKNTV |
| | YLQMNSLRPEDTAVYYCATDSGGYIDYDCMGLGYDYWGQGTLVTVSS |
| | (SEQ ID NO: 465) |
| DLLANGBII00007 | DVQLVESGGGLVQPGGSLRLSCAASGFALDYYAIGWFRQAPGKEREGVSCISSSDGITYYADSVKGR |
| | FTISRDNSKNTVYLQMNSLRPEDTAVYYCATDSGGYIDYDCMGLGYDYWGQGTLVTVSSGGGGSGGG |
| | SEVQLVESGGGLVQPGNSLRLSCAASGFTFSSFGMSWVRQAPGKGLEWVSSISGSGSDTLYADSVKG |
| | RFTISRDNAKTTLYLQMNSLRPEDTAVYYCTIGGSLSRSSQGTLVTVSSGGGGSGGGSEVQLVESGG |
| | GLVQPGGSLRLSCAASGRTFSSYAMAWYRQAPGKEREYVAAIRWSGGTAYYADSVKGRFTISRDNAK |
| | NTVYLQMNSLRPEDTAVYYCANRAPDTRLAPYEYDHWGQGTLVTVSS |
| | (SEQ ID NO: 466) |
| DLLANGBII00008 | DVQLVESGGGLVQPGGSLRLSCAASGFTLDDYAIGWFRQAPGKEREGVSSIRDNDGSTYYADSVKGR |
| | FTISSDNSKNTVYLQMNSLRPEDTAVYYCAAVPAGRLRFGEQWYPLYEYDAWGQGTLVTVSSGGGGS |
| | GGGSEVQLVESGGGLVQPGGSLRLSCAASGFTLDDYAIGWFRQAPGKEREGVSSIRDNDGSTYYADS |
| | VKGRFTISSDNSKNTVYLQMNSLRPEDTAVYYCAAVPAGRLRFGEQWYPLYEYDAWGQGTLVTVSSG |
| | GGGSGGGSEVQLVESGGGLVQPGNSLRLSCAASGFTFSSFGMSWVRQAPGKGLEWVSSISGSGSDTL |
| | YADSVKGRFTISRDNAKTTLYLQMNSLRPEDTAVYYCTIGGSLSRSSQGTLVTVSSGGGGSGGGSEV |
| | QLVESGGGLVQPGGSLRLSCAASGRTFSSYAMAWYRQAPGKEREYVAAIRWSGGTAYYADSVKGRFT |
| | ISRDNAKNTVYLQMNSLRPEDTAVYYCANRAPDTRLAPYEYDHWGQGTLVTVSS |
| | (SEQ ID NO: 467) |
| DLLANGBII00009 | DVQLVESGGGLVQPGGSLRLSCAASGRTFSSYAMAWYRQAPGKEREYVAAIRWSGGTAYYADSVKGR |
| | FTISRDNAKNTVYLQMNSLRPEDTAVYYCANRAPDTRLAPYEYDHWGQGTLVTVSSGGGGSGGGSEV |
| | QLVESGGGLVQPGGSLRLSCAASGFTFDDYALGWFRQAPGKEREGVSCIRCSDGSTYYADSVKGRFT |
| | ISSDNSKNTVYLQMNSLRPEDTAVYYCAASIVPRSKLEPYEYDAWGQGTLVTVSSGGGGSGGGSEVQ |
| | LVESGGGLVQPGGSLRLSCAASGFTFDDYALGWFRQAPGKEREGVSCIRCSDGSTYYADSVKGRFTI |
| | SSDNSKNTVYLQMNSLRPEDTAVYYCAASIVPRSKLEPYEYDAWGQGTLVTVSSGGGGSGGGSEVQL |
| | VESGGGLVQPGNSLRLSCAASGFTFSSFGMSWVRQAPGKGLEWVSSISGSGSDTLYADSVKGRFTIS |
| | RDNAKTTLYLQMNSLRPEDTAVYYCTIGGSLSRSSQGTLVTVSS |
| | (SEQ ID NO: 468) |
| DLLANGBII00010 | DVQLVESGGGLVQPGGSLRLSCAASGFTFDDYALGWFRQAPGKEREGVSCIRCSDGSTYYADSVKGR |
| | FTISSDNSKNTVYLQMNSLRPEDTAVYYCAASIVPRSKLEPYEYDAWGQGTLVTVSSGGGGSGGGSE |
| | VQLVESGGGLVQPGGSLRLSCAASGFTFDDYALGWFRQAPGKEREGVSCIRCSDGSTYYADSVKGRF |
| | TISSDNSKNTVYLQMNSLRPEDTAVYYCAASIVPRSKLEPYEYDAWGQGTLVTVSSGGGGSGGGSEV |
| | QLVESGGGLVQPGGSLRLSCAASGRTFSSYAMAWYRQAPGKEREYVAAIRWSGGTAYYADSVKGRFT |
| | ISRDNAKNTVYLQMNSLRPEDTAVYYCANRAPDTRLAPYEYDHWGQGTLVTVSSGGGGSGGGSEVQL |
| | VESGGGLVQPGNSLRLSCAASGFTFSSFGMSWVRQAPGKGLEWVSSISGSGSDTLYADSVKGRFTIS |
| | EDNAKTTLYLQMNSLRPEDTAVYYCTIGGSLSRSSQGTLVTVSS |
| | (SEQ ID NO: 469) |
| DLLANGBII00011 | DVQLVESGGGLVQPGGSLRLSCAASGFTLDDYAIGWFRQAPGKEREGVSSIRDNDGSTYYADSVKGR |
| | FTISRDNAKNTVYLQMNSLRPEDTAVYYCANRAPDTRLAPYEYDHWGQGTLVTVSSGGGGSGGGSEV |
| | QLVESGGGLVQPGGSLRLSCAASGFTLDDYAIGWFRQAPGKEREGVSSIRDNDGSTYYADSVKGRFT |
| | ISSDNSKNTVYLQMNSLRPEDTAVYYCAAVPAGRLRFGEQWYPLYEYDAWGQGTLVTVSSGGGGSGG |
| | GSEVQLVESGGGLVQPGNSLRLSCAASGFTFSSFGMSWVRQAPGKGLEWVSSISGSGSDTLYADSVK |
| | GRFTISRDNAKTTLYLQMNSLRPEDTAVYYCTIGGSLSRSSQGTLVTVSS |
| | (SEQ ID NO: 470) |
| DLLANGVII00012 | DVQLVESGGGLVQPGGSLRLSCAASGFTLDDYAIGWFRQAPGKEREGVSSIRDNDGSTYYADSVKGR |
| | FTISSDNSKNTVYLQMNSLRPEDTAVYYCAAVPAGRLRFGEQWYPLYEYDAWGQGTLVTVSSGGGGS |
| | GGGSEVQLVESGGGLVQPGGSLRLSCAASGRTFSSYAMAWYRQAPGKEREYVAAIRWSGGTAYYADS |
| | VKGRFTISRDNAKNTVYLQMNSLRPEDTAVYYCANRAPDTRLAPYEYDHWGQGTLVTVSSGGGGSGG |
| | GSEVQLVESGGGLVQPGNSLRLSCAASGFTFSSFGMSWVRQAPGKGLEWVSSISGSGSDTLYADSVK |
| | GRFTISRDNAKTTLYLQMNSLRPEDTAVYYCTIGGSLSRSSQGTLVTVSS |
| | (SEQ ID NO: 471) |
| DLLANGBII00013 | DVQLVESGGGLVQPGGSLRLSCAASGRTFSSYAMAWYRQAPGKEREYVAAIRWSGGTAYYADSVKGR |
| | FTISRDNAKNTVYLQMNSLRPEDTAVYYCANRAPDTRLAPYEYDHWGQGTLVTVSSGGGGSGGGSEV |
| | QLVESGGGLVQPGGSLRLSCAASGFTLDDYAIGWFRQAPGKEREGVSSIRDNDGSTYYADSVKGRFT |
| | ISSDNSKNTVYLQMNSLRPEDTAVYYCAAVPAGRLRFGEQWYPLYEYDAWGQGTLVTVSSGGGGSGG |
| | GSEVQLVESGGGLVQPGGSLRLSCAASGFTLDDYAIGWFRQAPGKEREGVSSIRDNDGSTYYADSVK |
| | GRFTISSDNSKNTVYLQMNSLRPEDTAVYYCAAVPAGRLRFGEQWYPLYEYDAWGQGTLVTVSSGGG |
| | GSGGGSEVQLVESGGGLVQPGNSLRLSCAASGFTFSSFGMSWVRQAPGKGLEWVSSISGSGSDTLYA |
| | DSVKGRFTISRDNAKTTLYLQMNSLRPEDTAVYYCTIGGSLSRSSQGTLVTVSS |
| | (SEQ ID NO: 472) |
| DLLANGBII00014 | DVQLVESGGGLVQPGGSLRLSCAASGFTLDDYAIGWFRQAPGKEREGVSSIRDNDGSTYYADSVKGR |
| | FTISSDNSKNTVYLQMNSLRPEDTAVYYCAAVPAGRLRFGEQWYPLYEYDAWGQGTLVTVSSGGGGS |
| | GGGSEVQLVESGGGLVQPGGSLRLSCAASGFTLDDYAIGWFRQAPGKEREGVSSIRDNDGSTYYADS |
| | VKGRFTISSDNSKNTVYLQMNSLRPEDTAVYYCAAVPAGRLRFGEQWYPLYEYDAWGQGTLVTVSSG |
| | GGGSGGGSEVQLVESGGGLVQPGGSLRLSCAASGRTFSSYAMAWYRQAPGKEREYVAAIRWSGGTAY |
| | YADSVKGRFTISRDNAKNTVYLQMNSLRPEDTAVYYCANRAPDTRLAPYEYDHWGQGTLVTVSSGGG |
| | GSGGGSEVQLVESGGGLVQPGNSLRLSCAASGFTFSSFGMSWVRQAPGKGLEWVSSISGSGSDTLYA |
| | DSVKGRFTISRDNAKTTLYLQMNSLRPEDTAVYYCTIGGSLSRSSQGTLVTVSS |
| | (SEQ ID NO: 473) |
| DLLANGVII00015 | DVQLVESGGGLVQPGGSLRLSCAASGRTFSSYAMAWYRQAPGKEREYVAAIRWSGGTAYYADSVKGR |
| | FTISRDNAKNTVYLQMNSLRPEDTAVYYCANRAPDTRLAPYEYDHWGQGTLVTVSSGGGGSGGGSEV |
| | QLVESGGGLVQPGGSLRLSCAASGFALDYYAIGWFRQAPGKEREGVSCISSSDGITYYADSVKGRFT |
| | ISRDNSKNTVYLQMNSLRPEDTAVYYCATDSGGYIDYDCMGLGYDYWGQGTLVTVSSGGGGSGGGSE |
| | VQLVESGGGLVQPGNSLRLSCAASGFTFSSFGMSWVRQAPGKGLEWVSSISGSGSDTLYADSVKGRF |
| | TISRDNAKTTLYLQMNSLRPEDTAVYYCTIGGSLSRSSQGTLVTVSS |
| | (SEQ ID NO: 474) |
| DLLANGBII00016 | DVQLVESGGGLVQPGGSLRLSCAASGFALDYYAIGWFRQAPGKEREGVSCISSSDGITYYADSVKGR |
| | FTISRDNSKNTVYLQMNSLRPEDTAVYYCATDSGGYIDYDCMGLGYDYWGQGTLVTVSSGGGGSGGG |
| | SEVQLVESGGGLVQPGGSLRLSCAASGRTFSSYAMAWYRQAPGKEREYVAAIRWSGGTAYYADSVKG |
| | RFTISRDNAKNTVYLQMNSLRPEDTAVYYCANRAPDTRLAPYEYDHWGQGTLVTVSSGGGGSGGGSE |
| | VQLVESGGGLVQPGNSLRLSCAASGFTFSSFGMSWVRQAPGKGLEWVSSISGSGSDTLYADSVKGRF |
| | TISRDNAKTTLYLQMNSLRPEDTAVYYCTIGGSLSRSSQGTLVTVSS |
| | (SEQ ID NO: 475) |

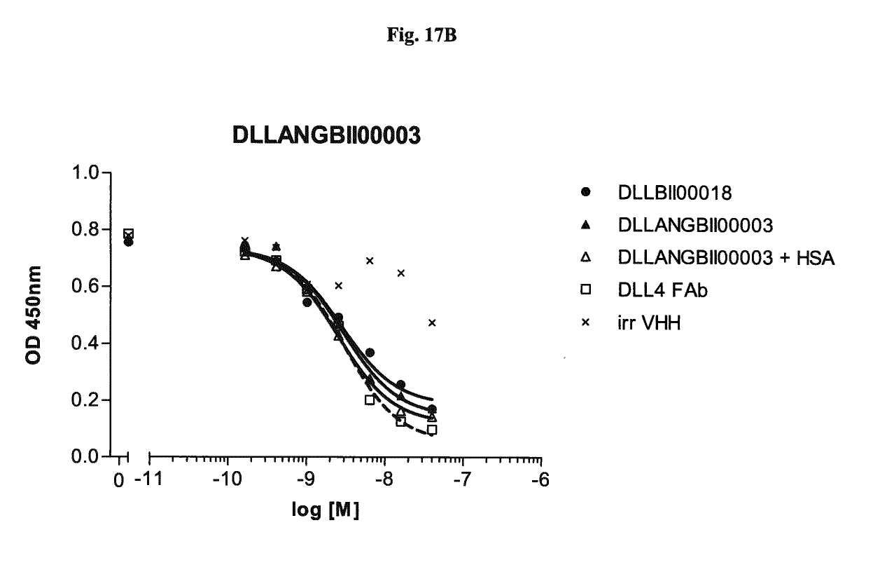

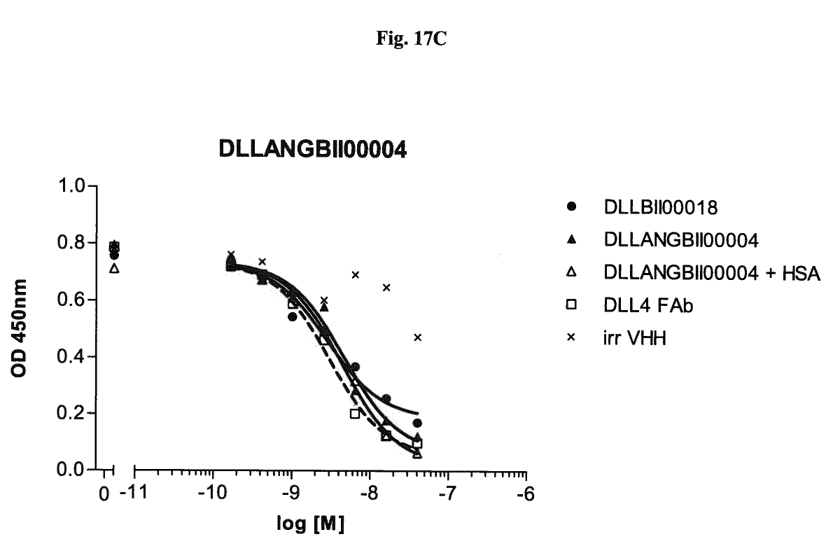

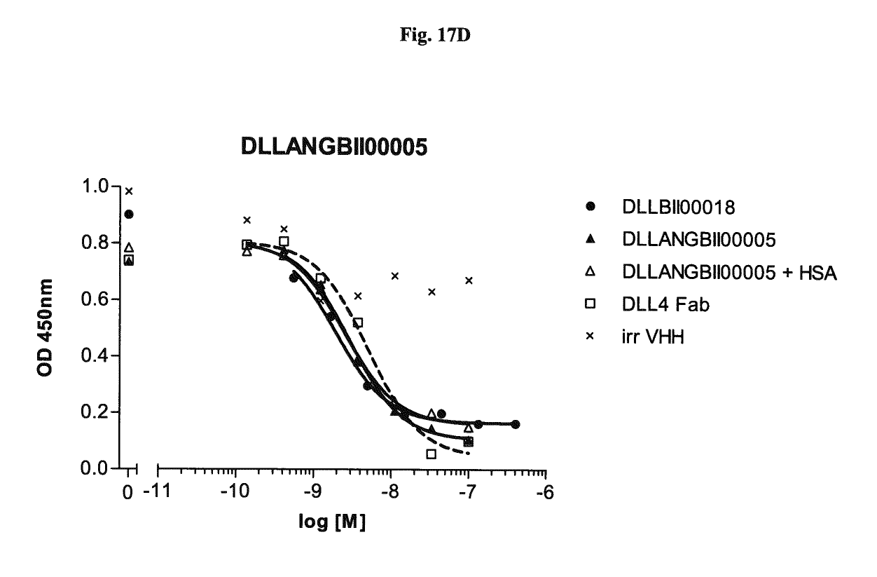

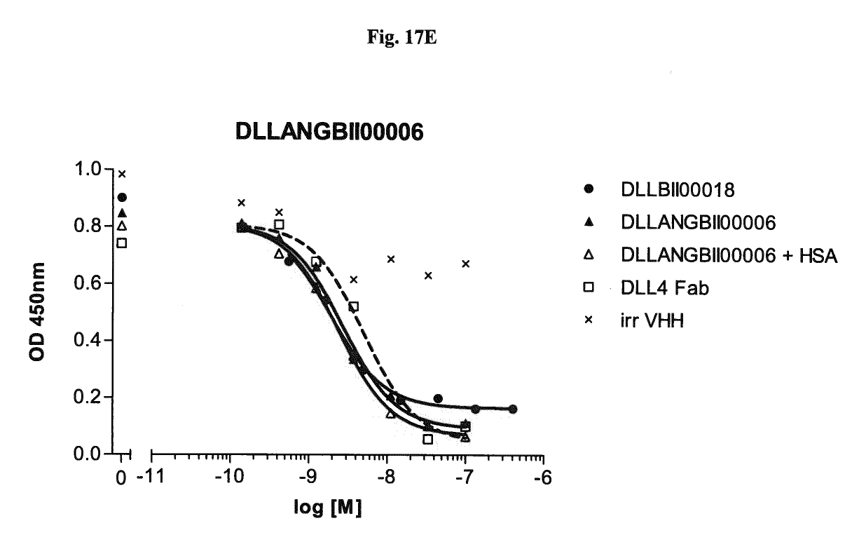

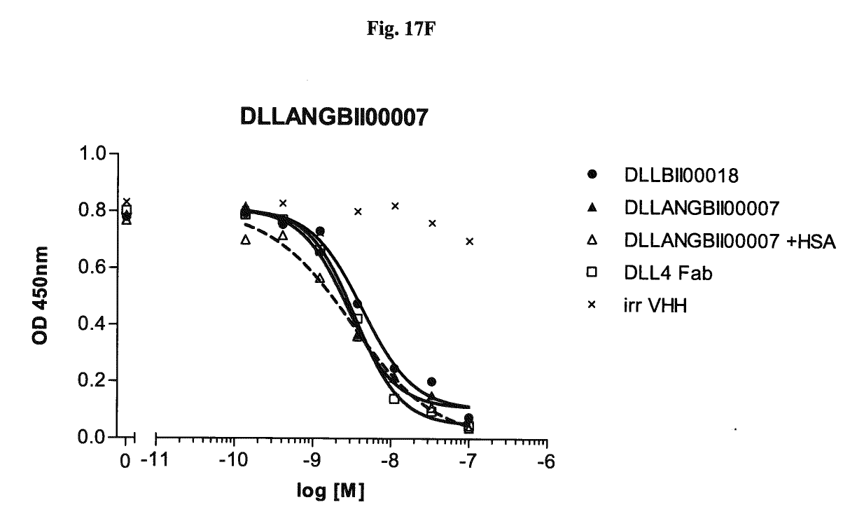

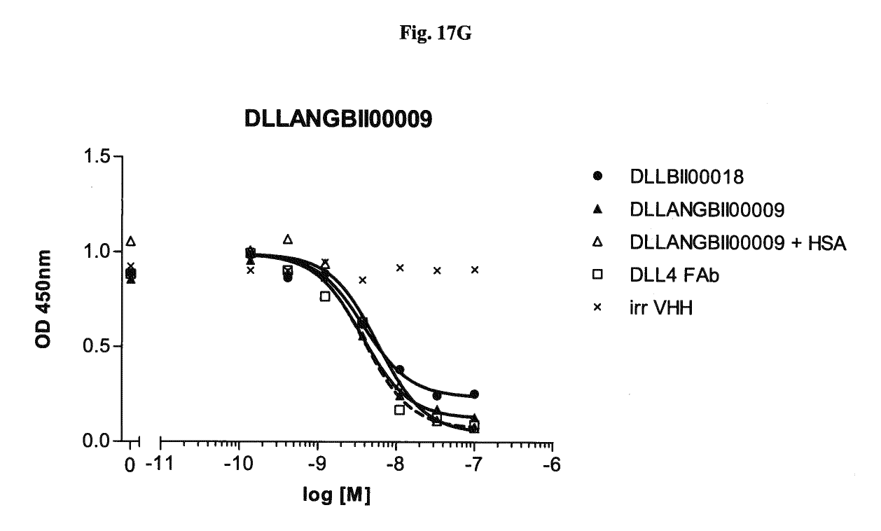

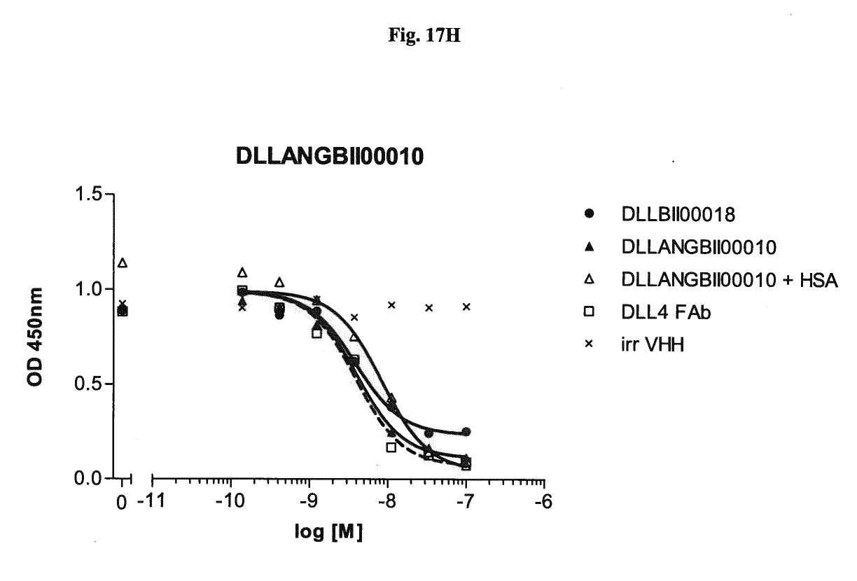

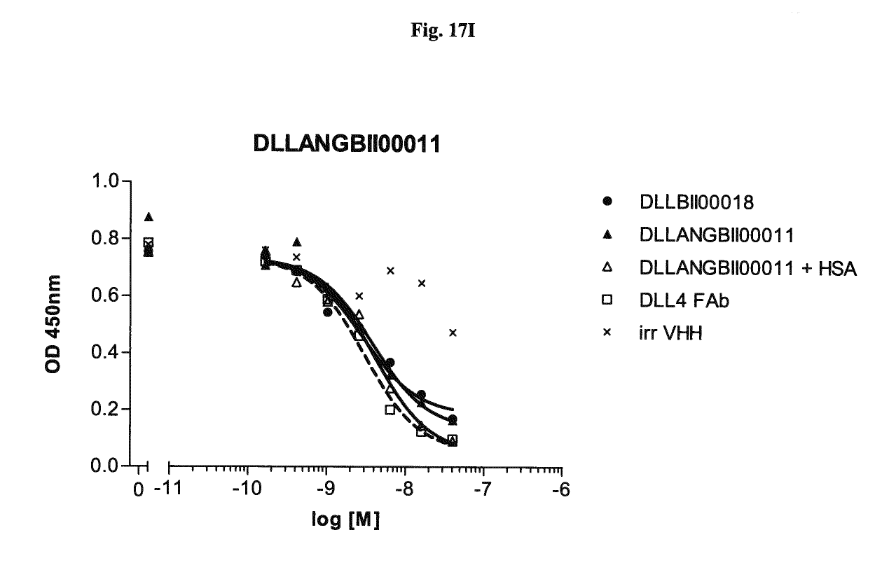

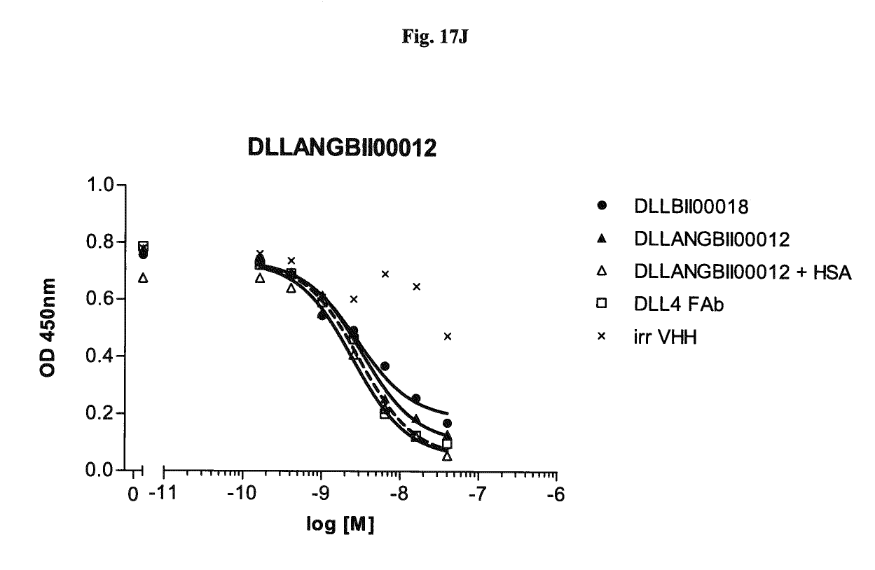

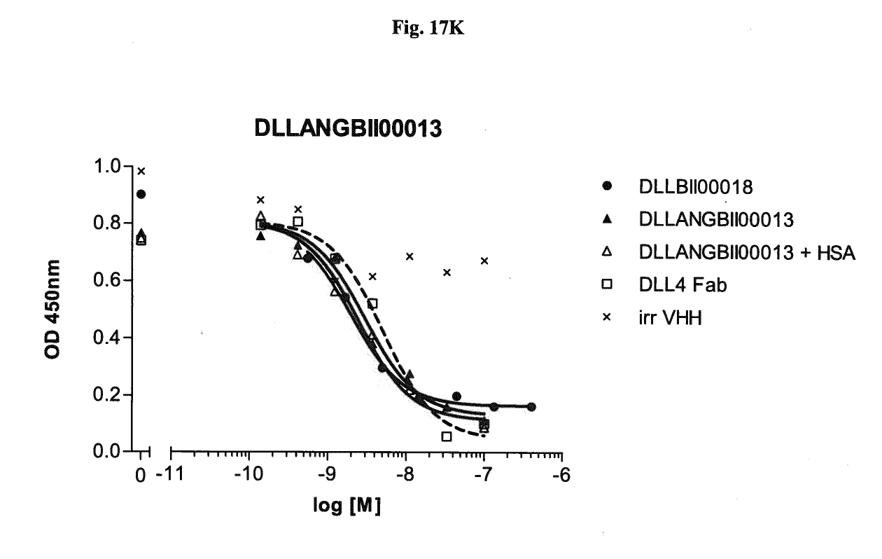

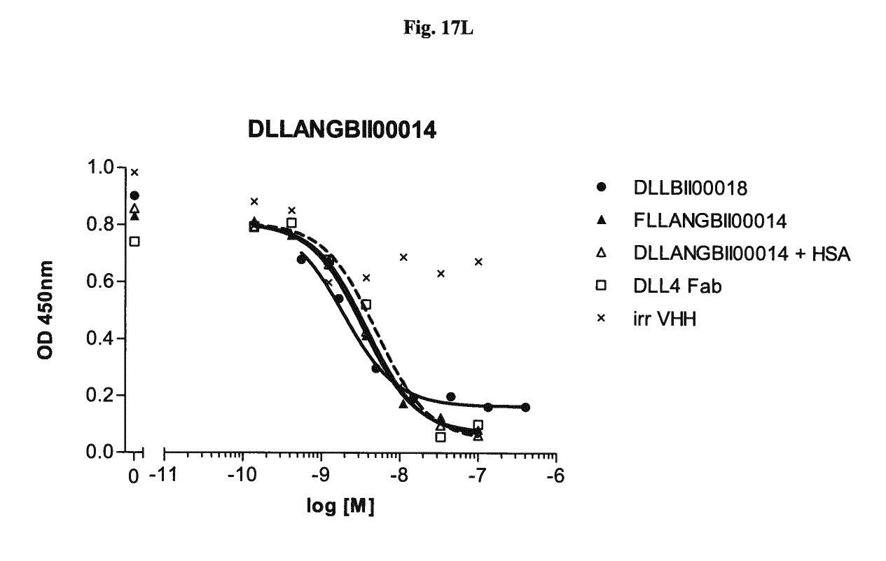

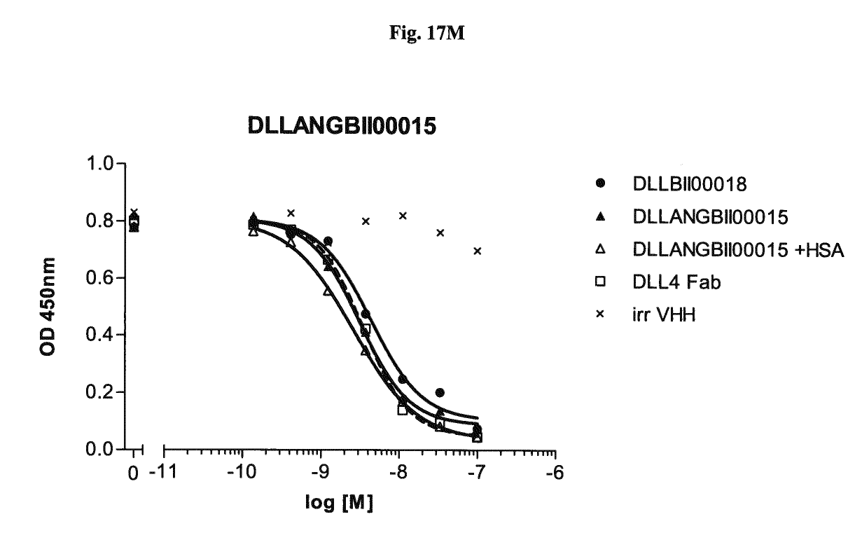

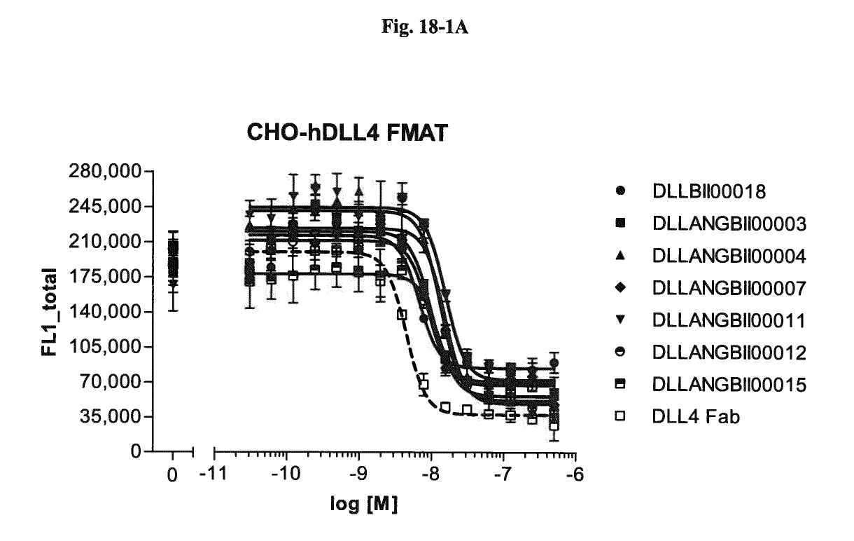

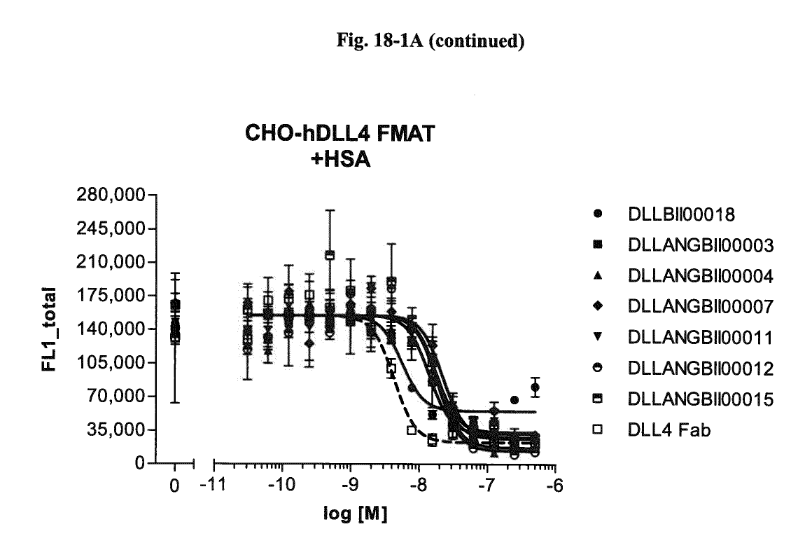

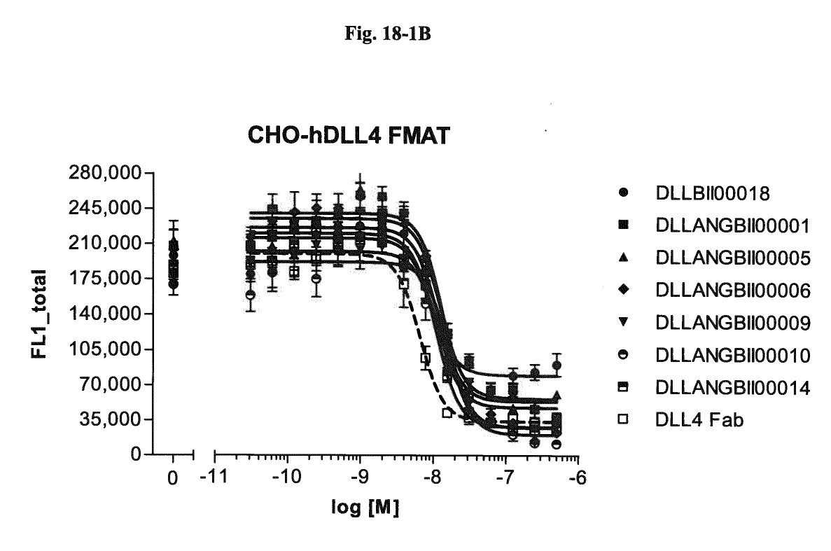

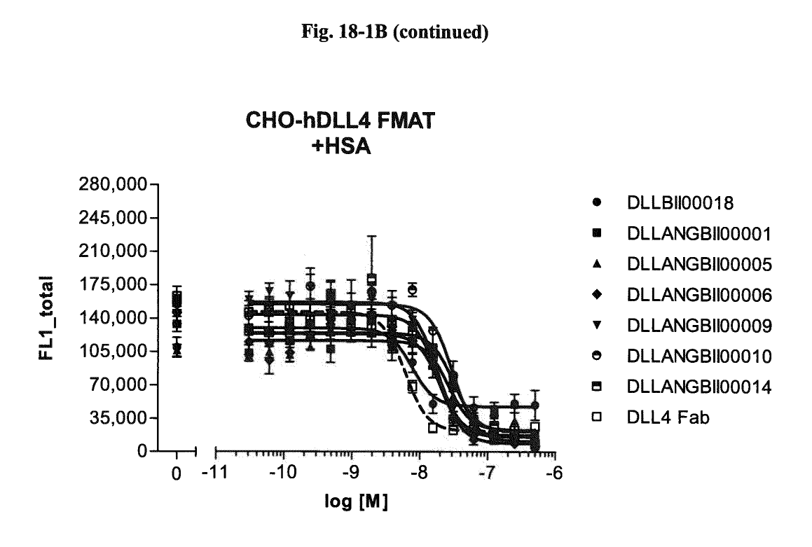

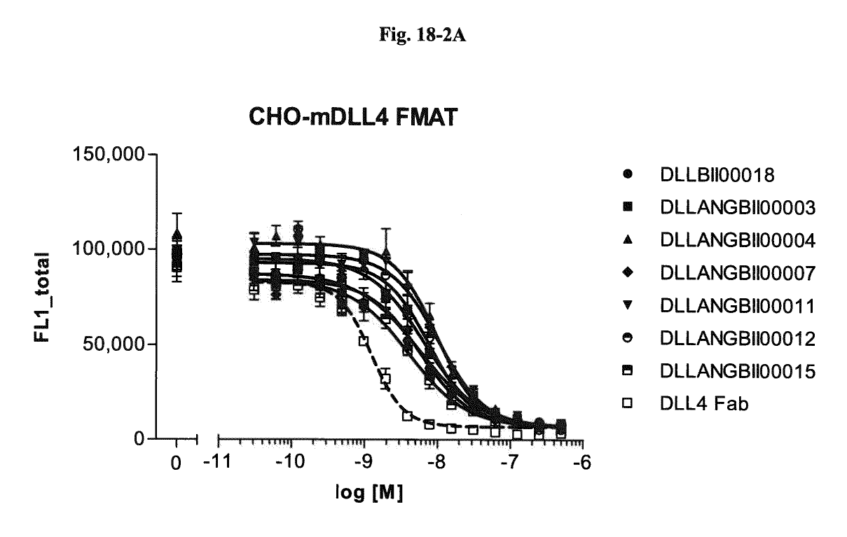

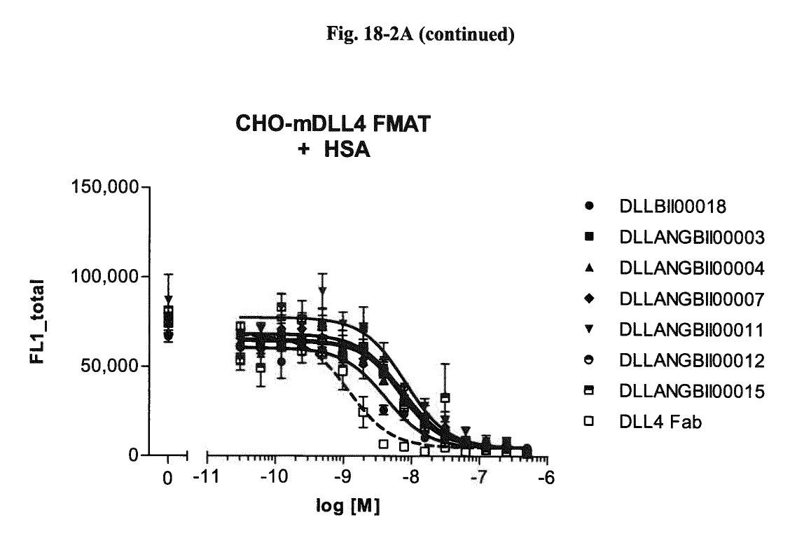

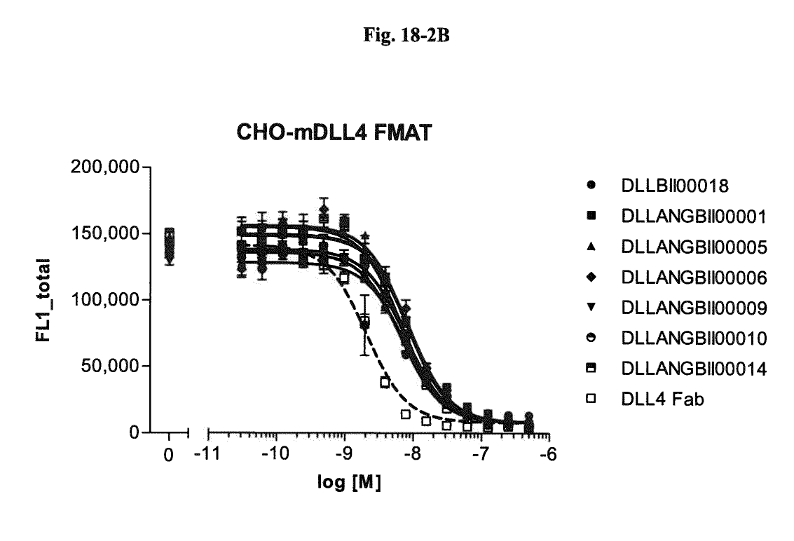

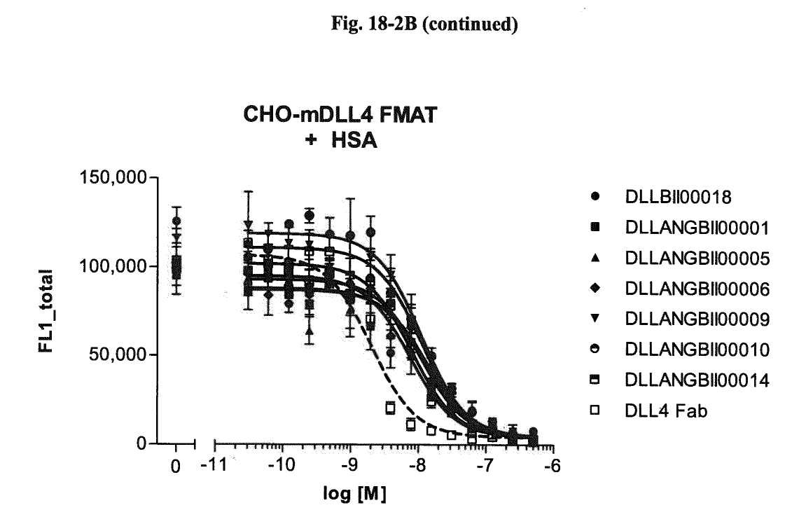

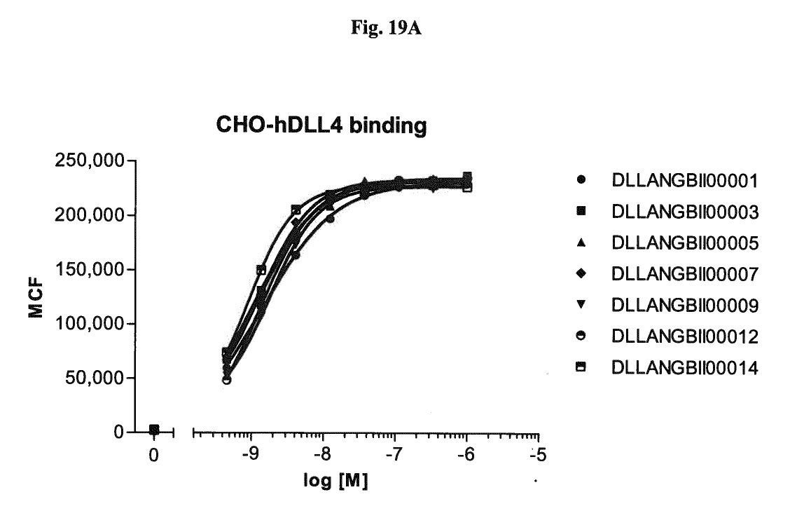

To explore the anti-DLL4 blocking properties in comparison with the monovalent building block DLLBII00018, all purified bispecific VHHs are analyzed in the hDLL4/hNotch1 competition ELISA (see Example 5.1 as described in patent US 2011/0172398 A1) (FIG. 17) and the CHO-hDLL4/CHO-mDLL4 competition FMAT (see Example 5.3 as described in patent US 2011/0172398 A1) (FIG. 18). Here, the ELISA competition assay is performed with a fixed concentration of 8 nM biotinylated hDLL4. Both ELISA and the FMAT competition assay are also performed after preincubation of the VHH with 12.5 μM and 25 μM human serum albumin, respectively. A summary of IC₅₀ values and % inhibition is shown in Table 23.

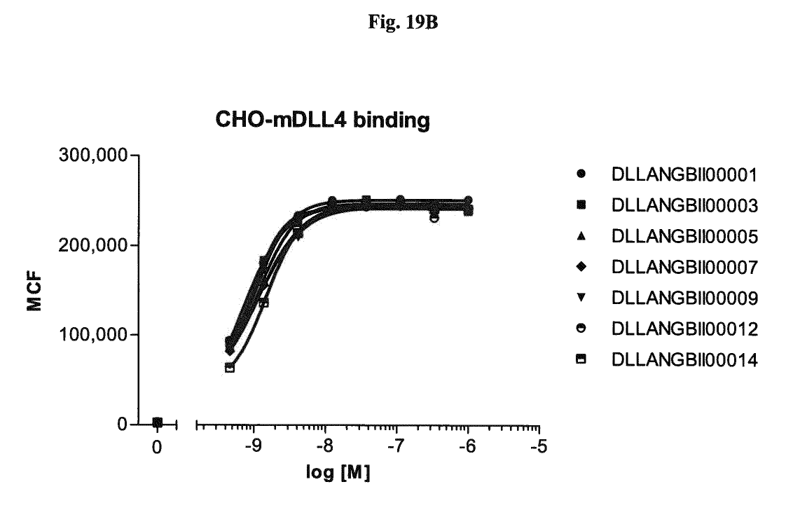

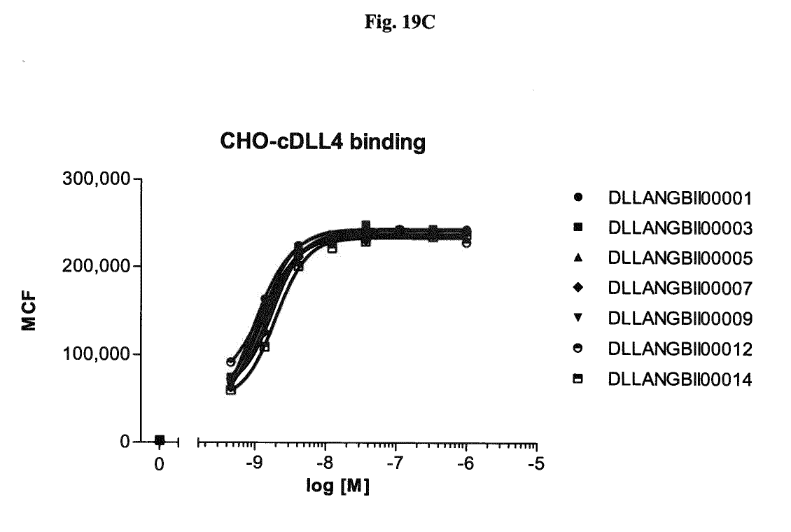

Additionally, in order to determine cross-reactivity of the bispecific VHHs to murine and cynomolgus DLL4, a FACS binding experiment is performed. Briefly, CHO cells overexpressing mouse and cynomolgus DLL4 are used for a titration binding experiment of the VHHs. After a 30 min incubation on ice, all samples are washed and a 2-step detection using anti-c-myc followed by goat-anti mouse IgG-PE labeled is performed. CHO cells overexpressing human DLL4 are taken as reference. The mean MCF value is determined using a FACS Array and used for calculation of the EC₅₀ value (Table 24; FIG. 19).

TABLE 24
EC₅₀ values of bispecific VHHs binding to
human, mouse and cyno DLL4
overexpressed on CHO cells (FACS)

| | CHO-hDLL4 | CHO-mDLL4 | CHO-cDLL4 |
|---|---|---|---|
| | EC₅₀ (nM) | EC₅₀ (nM) | EC₅₀ (nM) |
| DLLBII00018 | 1.5 | 1.2 | 0.8 |
| DLLANGBII00001 | 1.2 | 0.9 | 0.8 |
| DLLANGBII00003 | 1.1 | 0.9 | 0.7 |
| DLLANGBII00005 | 1.1 | 1.1 | 0.8 |
| DLLANGBII00007 | 1.5 | 1.2 | 0.9 |
| DLLANGBII00009 | 1.4 | 1.1 | 0.8 |
| DLLANGBII00012 | 0.8 | 0.8 | 0.7 |
| DLLANGBII00014 | 1.8 | 1.4 | 1.2 |
| DLL4Fab | 7.5 | 2.3 | 1.1 |

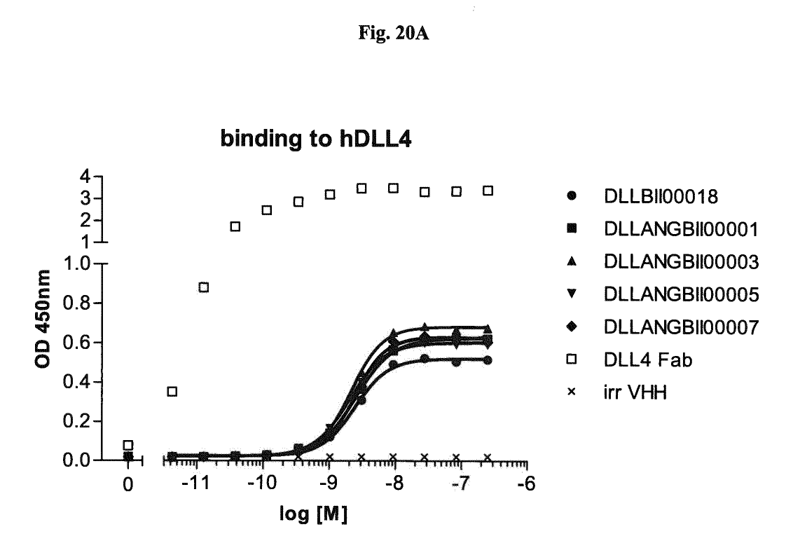

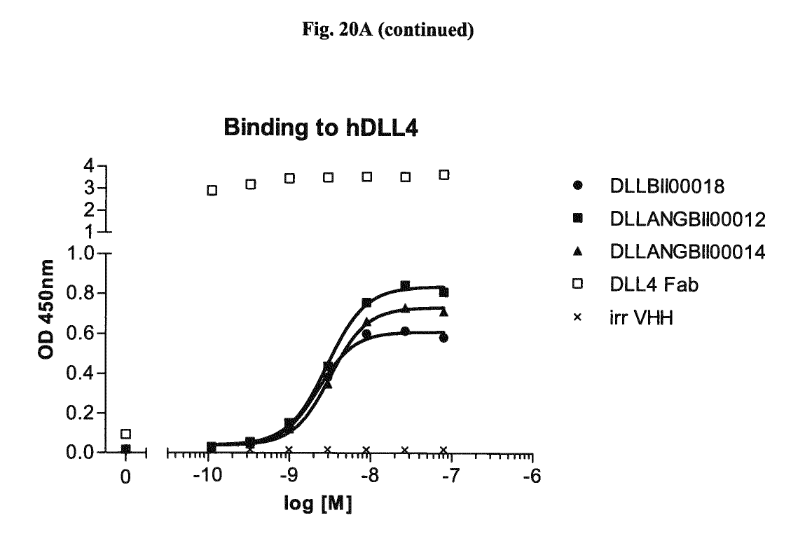

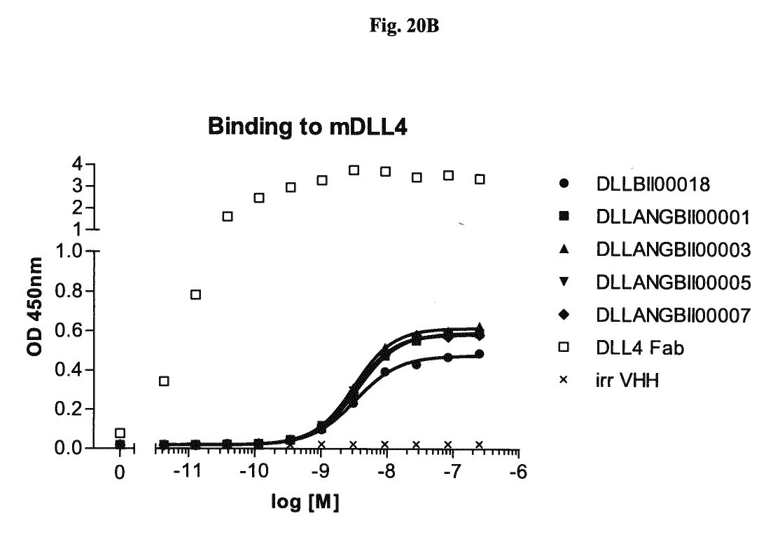

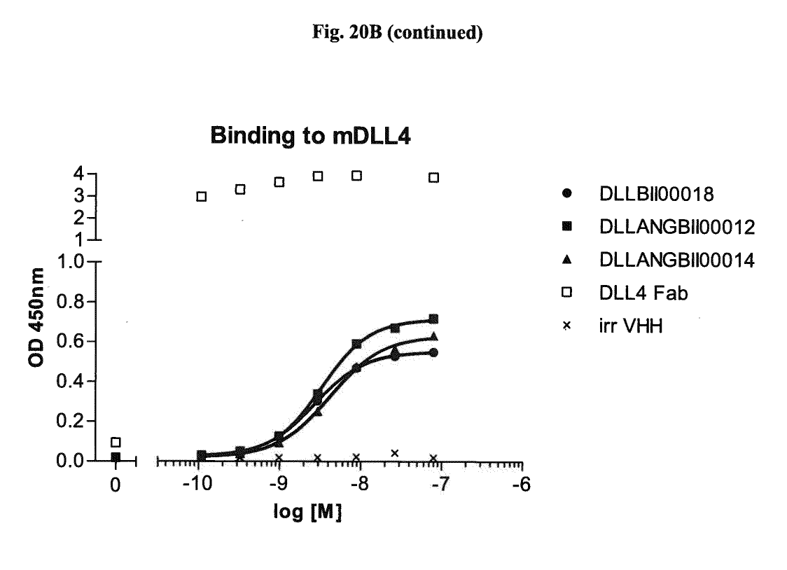

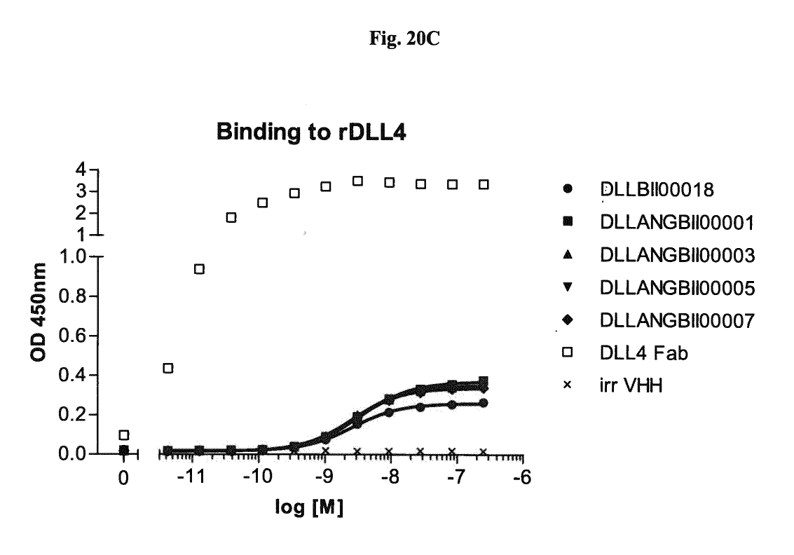

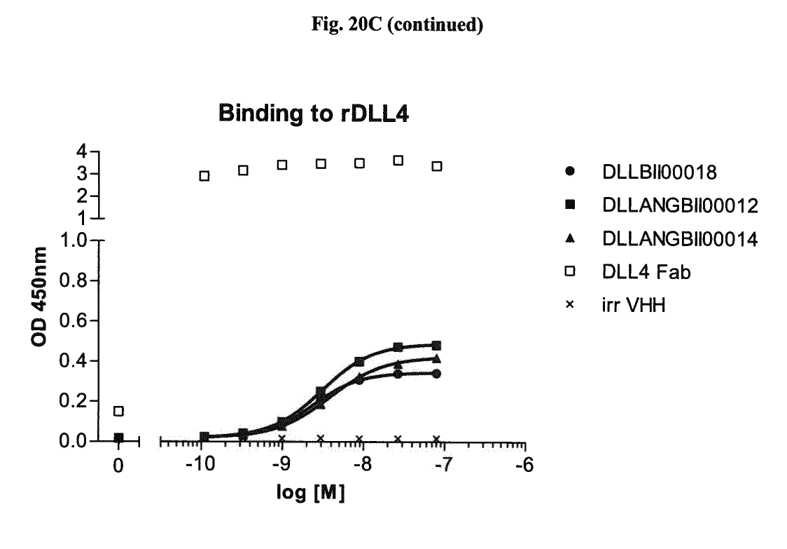

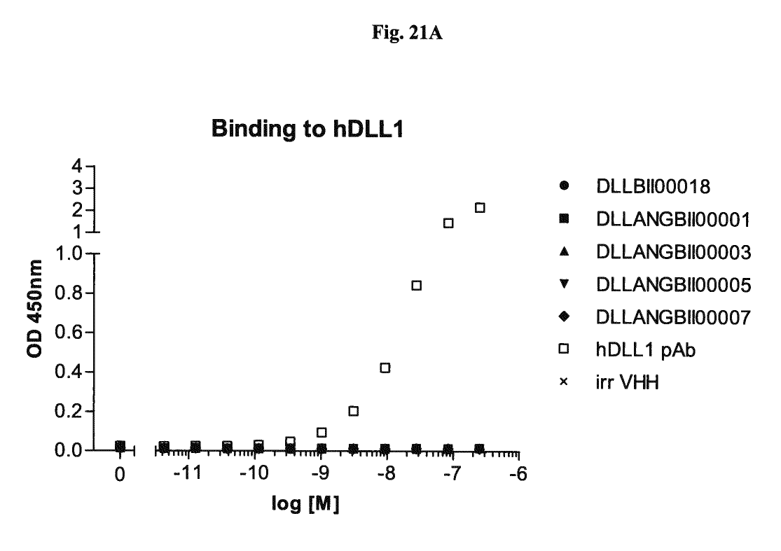

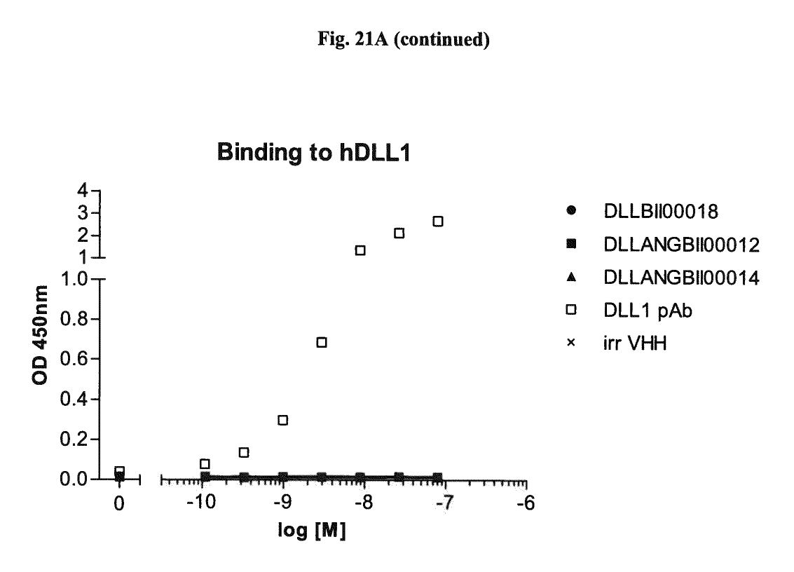

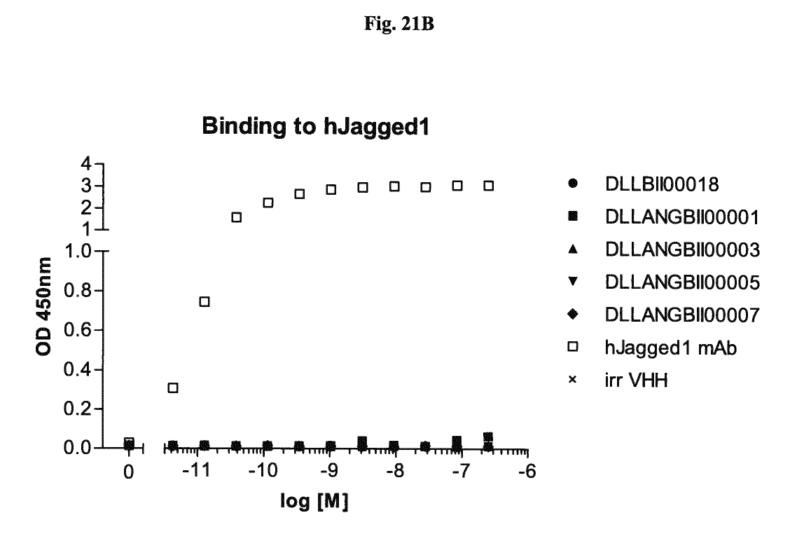

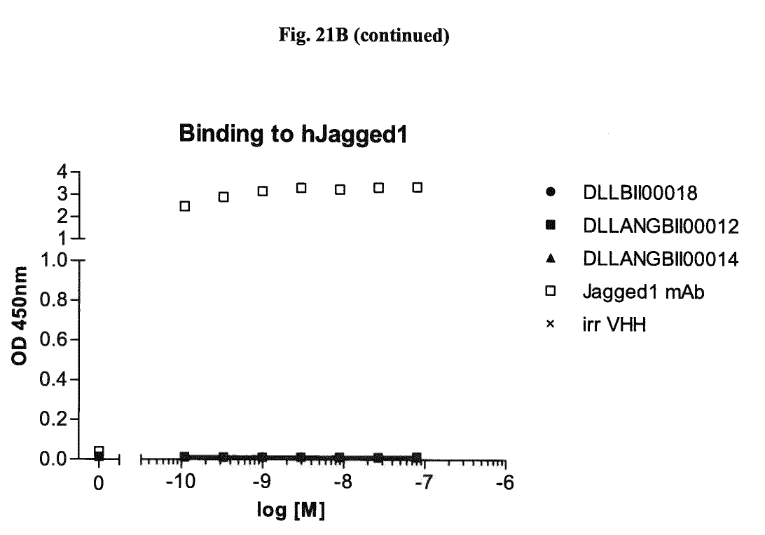

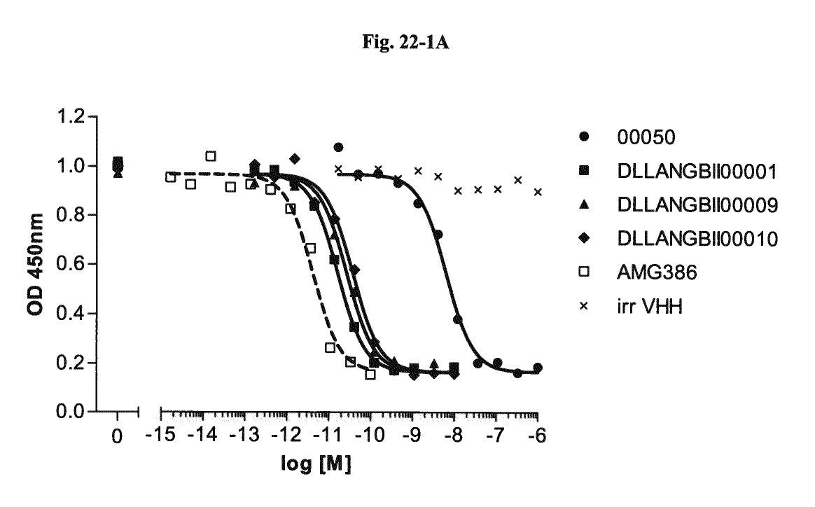

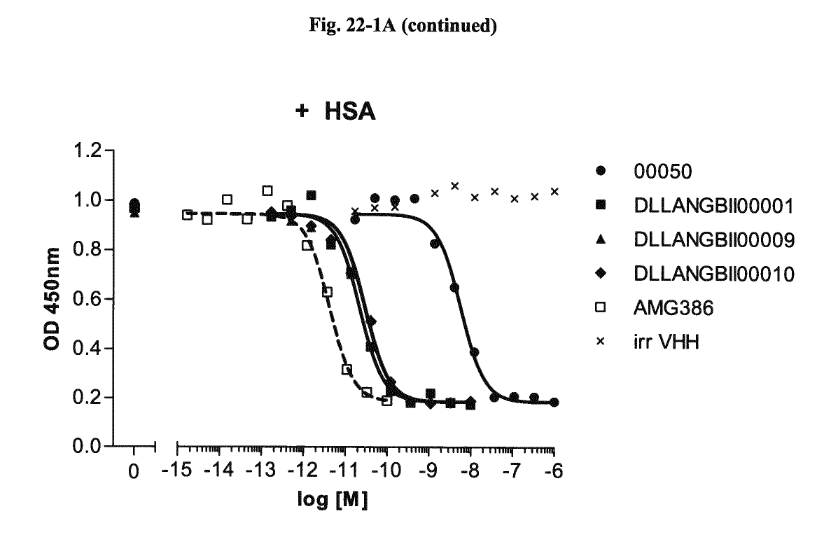

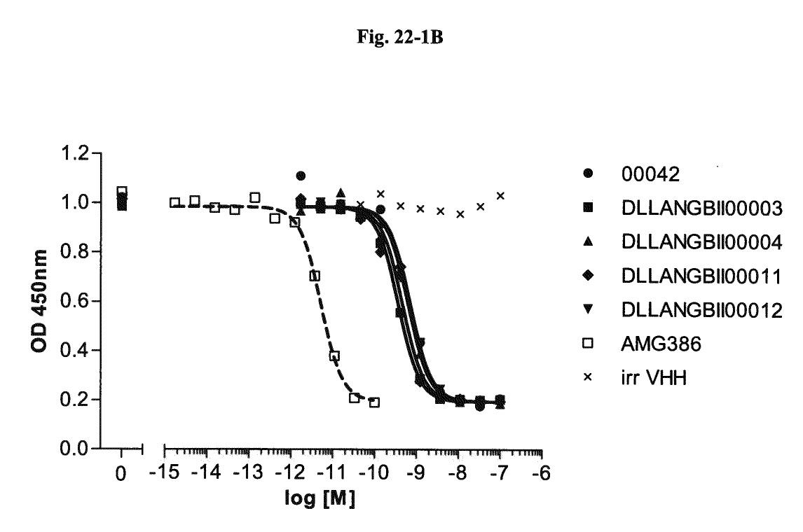

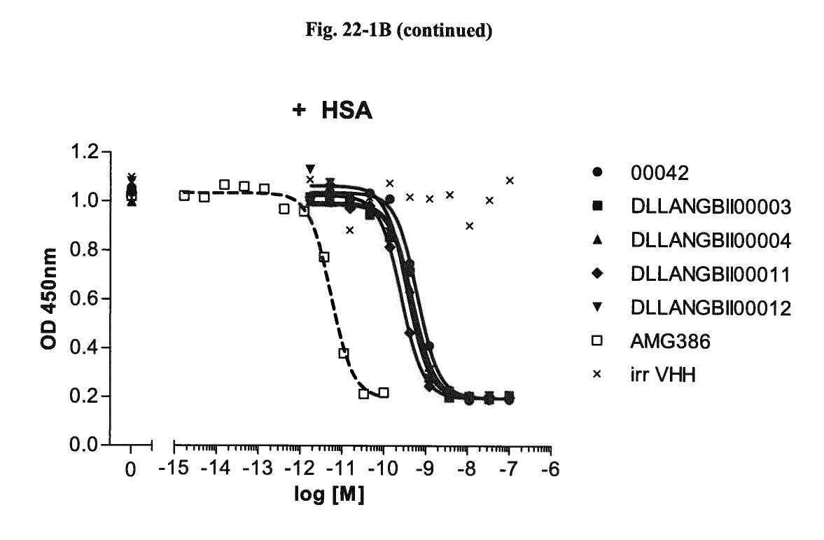

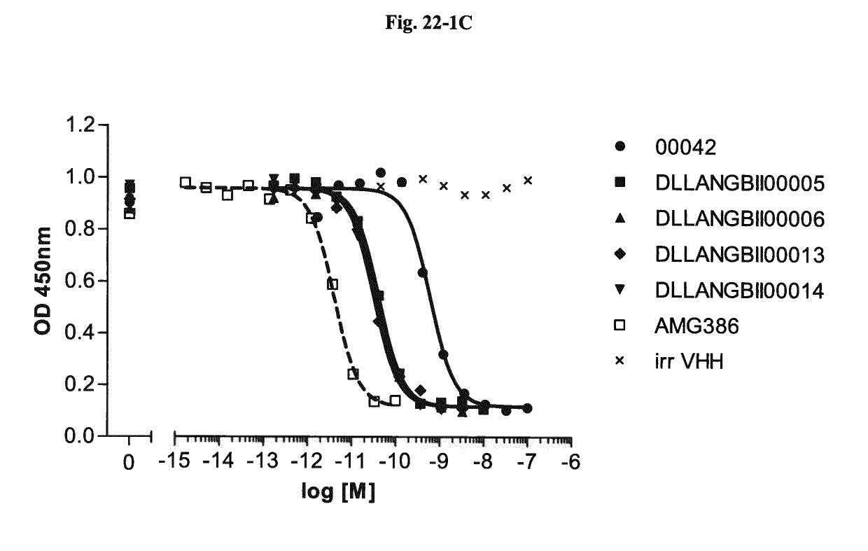

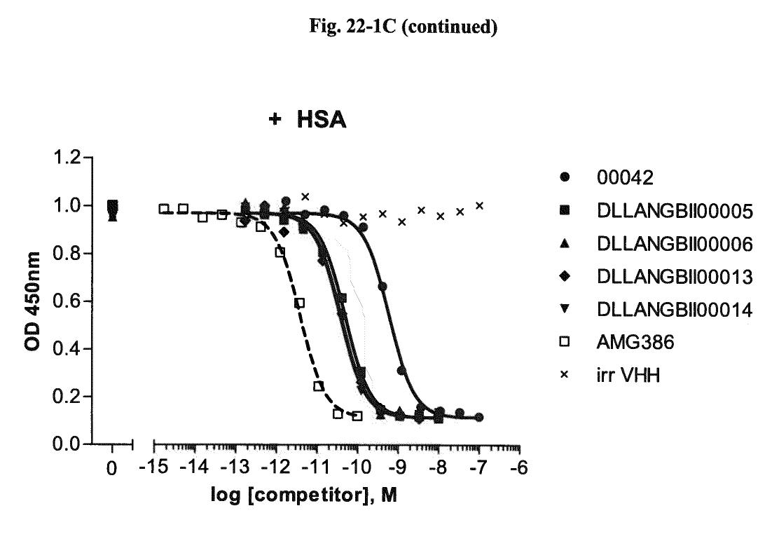

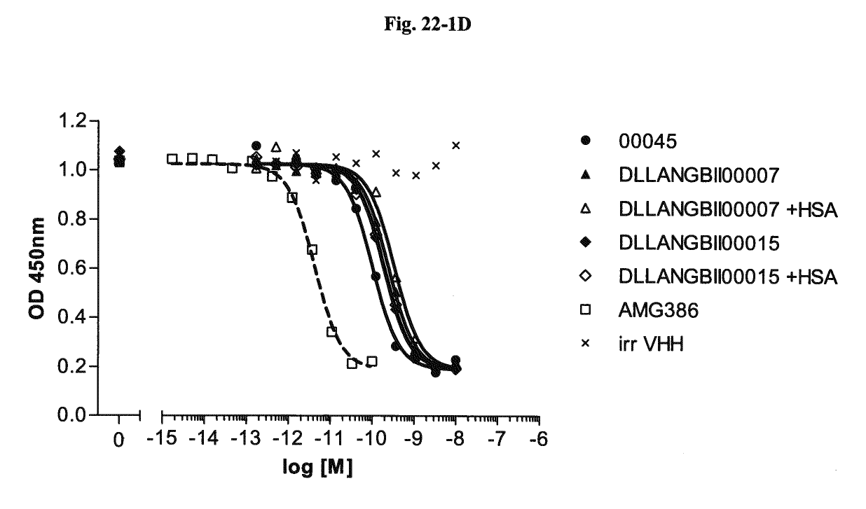

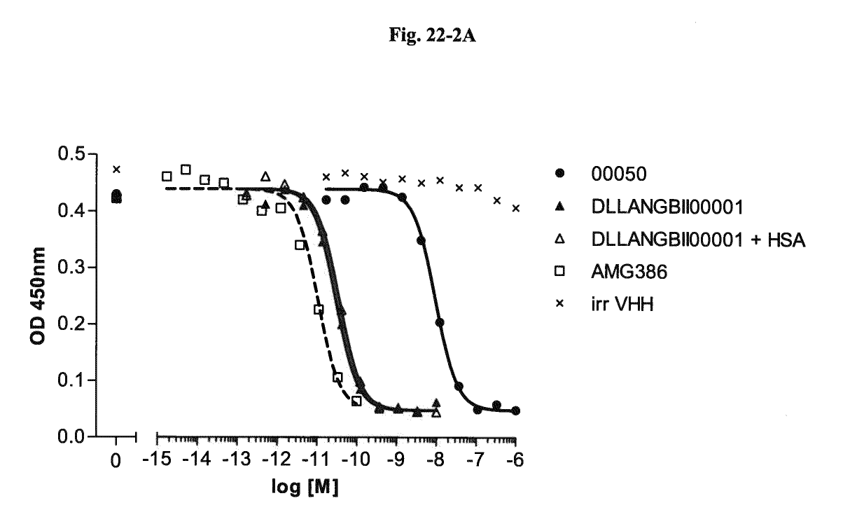

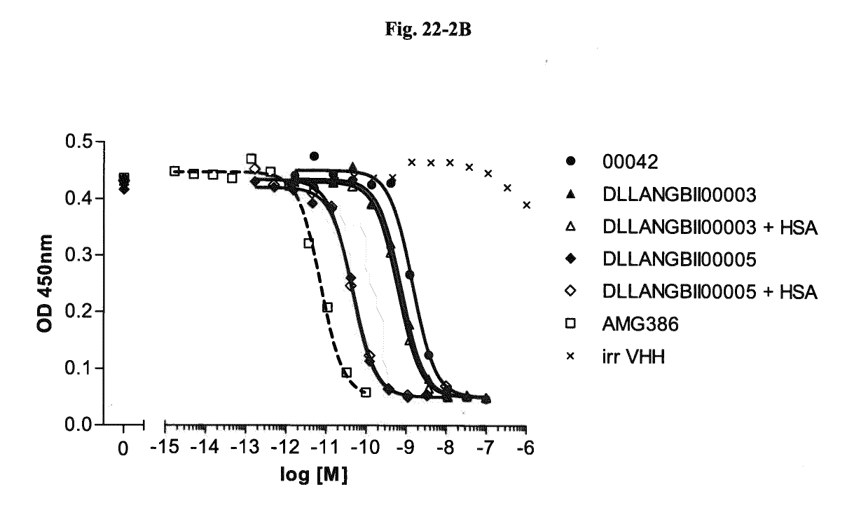

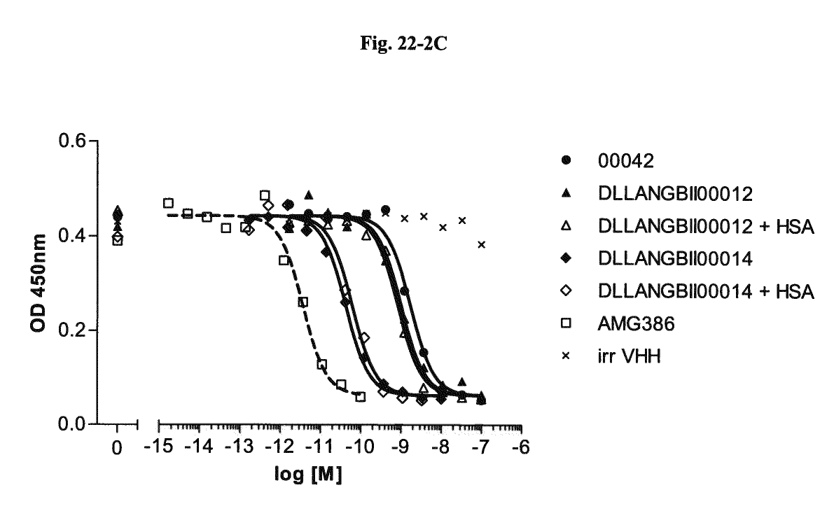

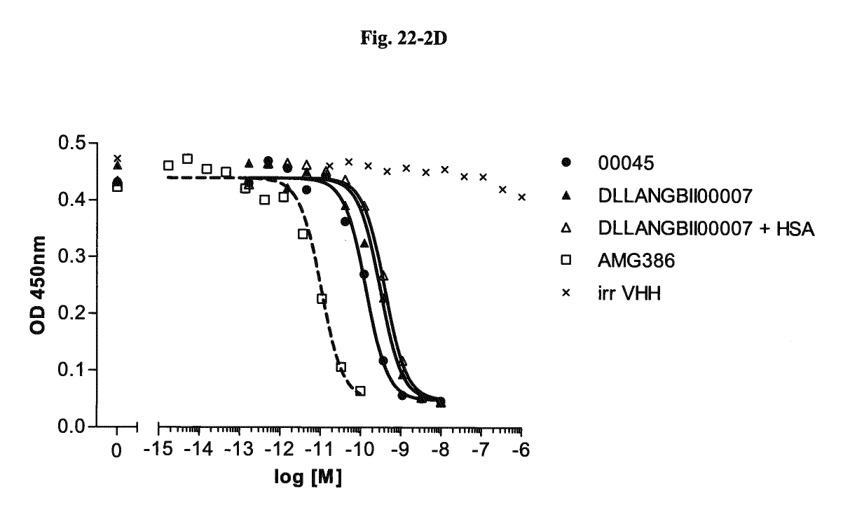

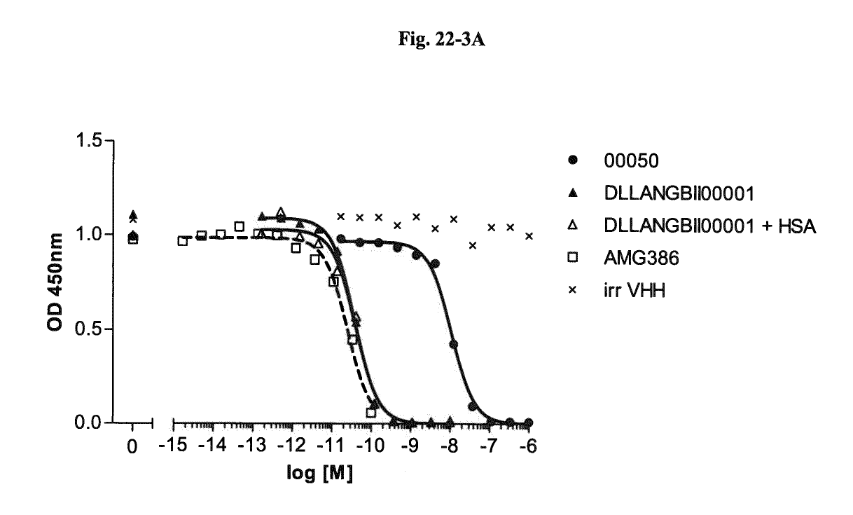

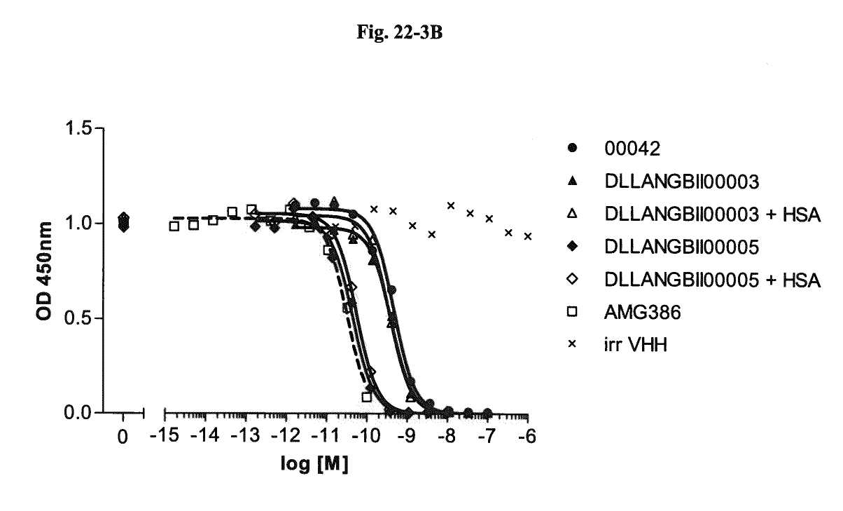

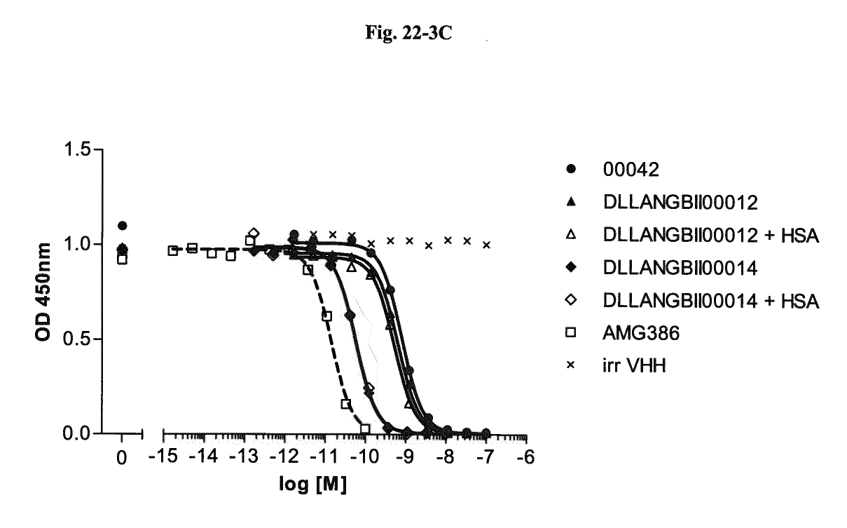

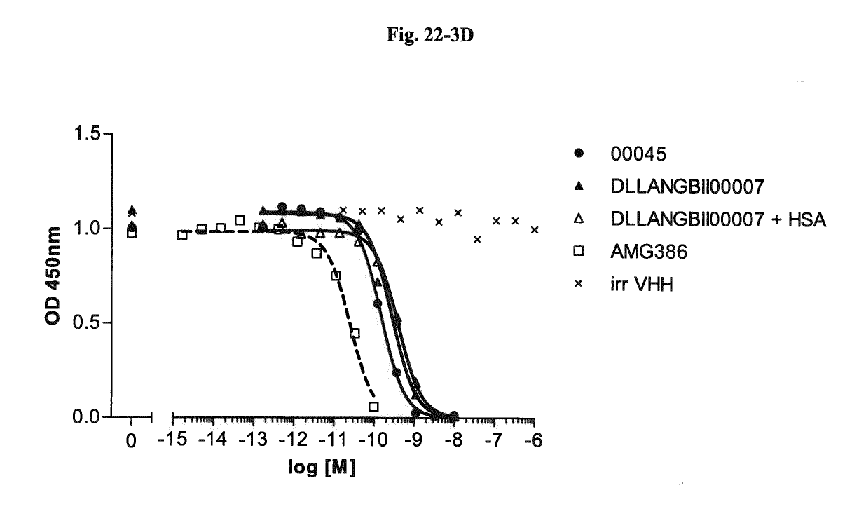

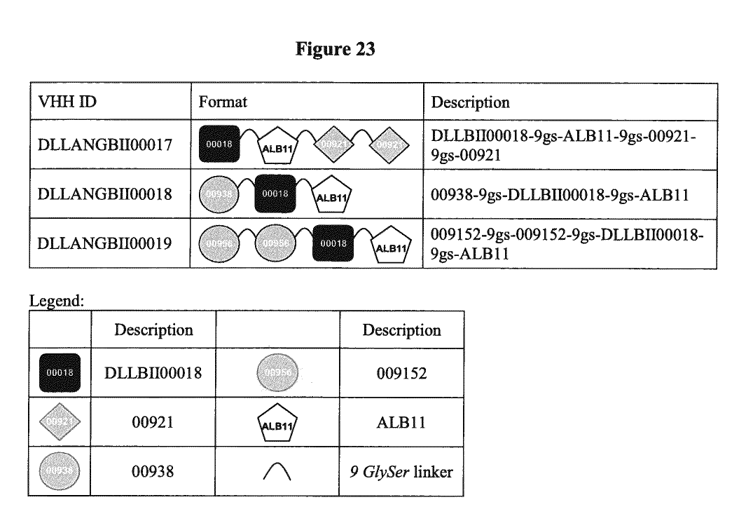

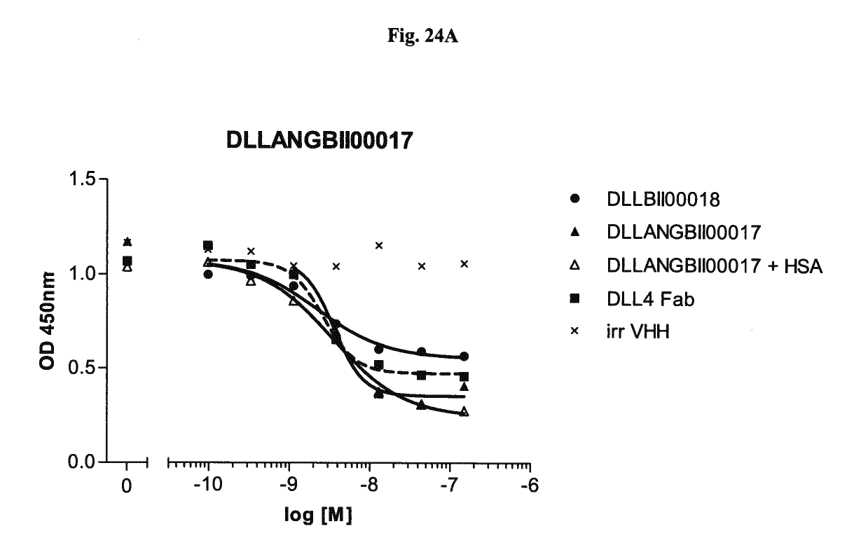

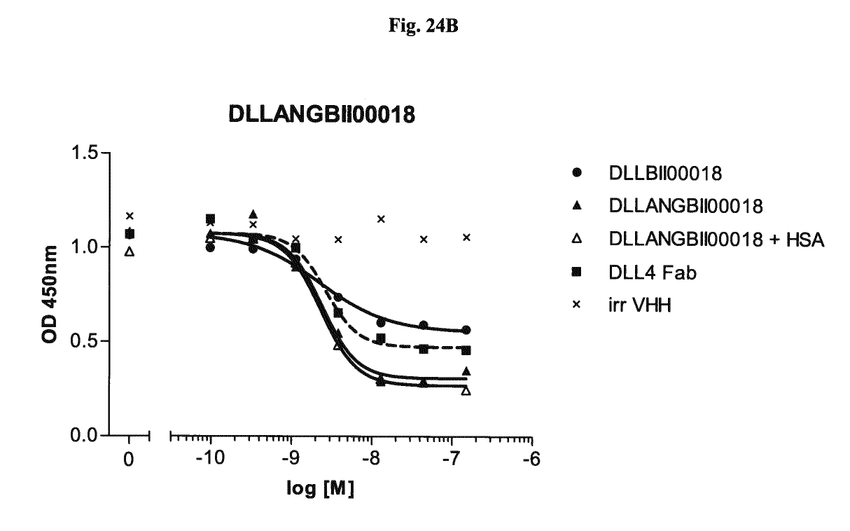

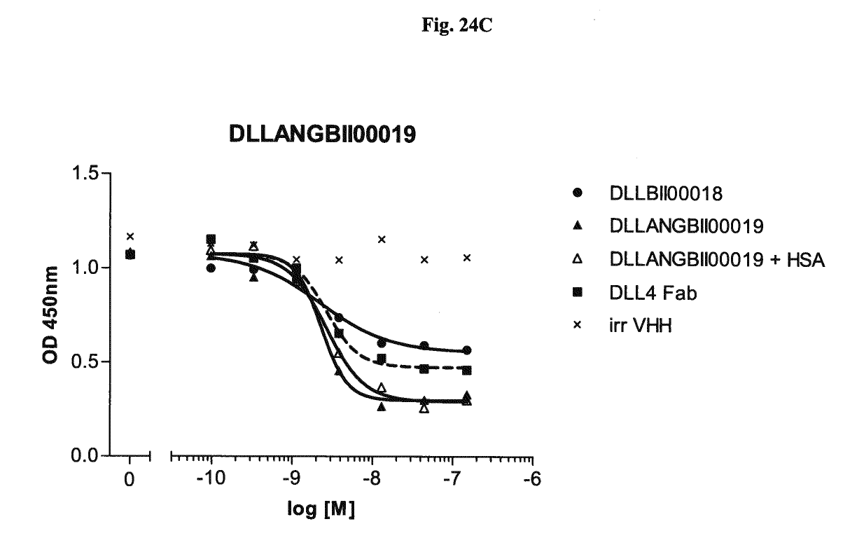

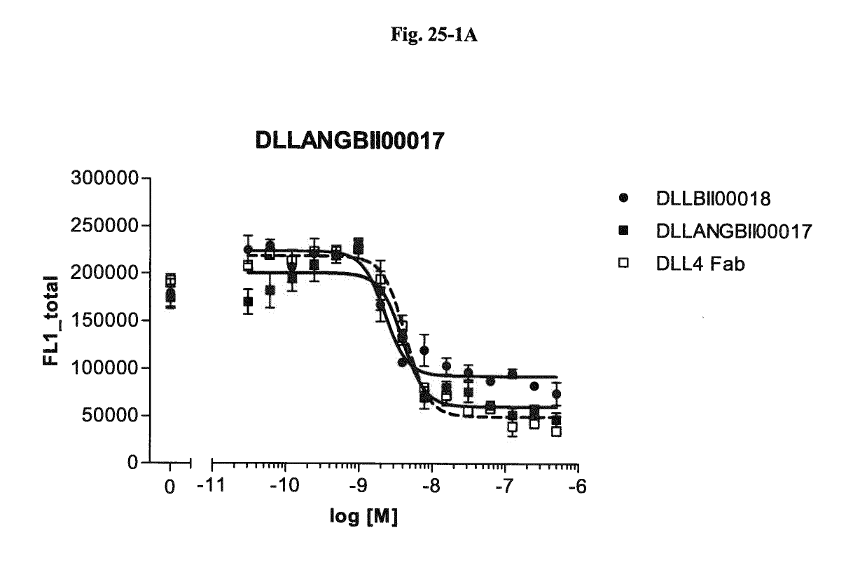

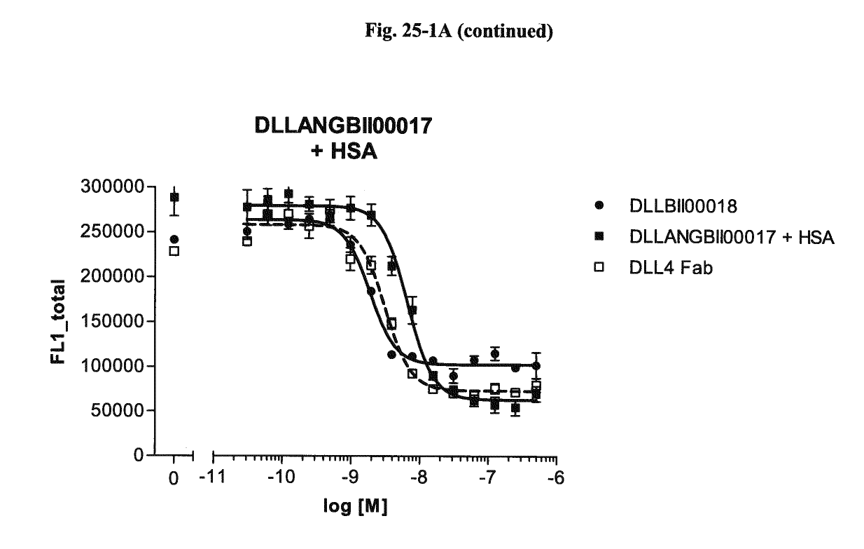

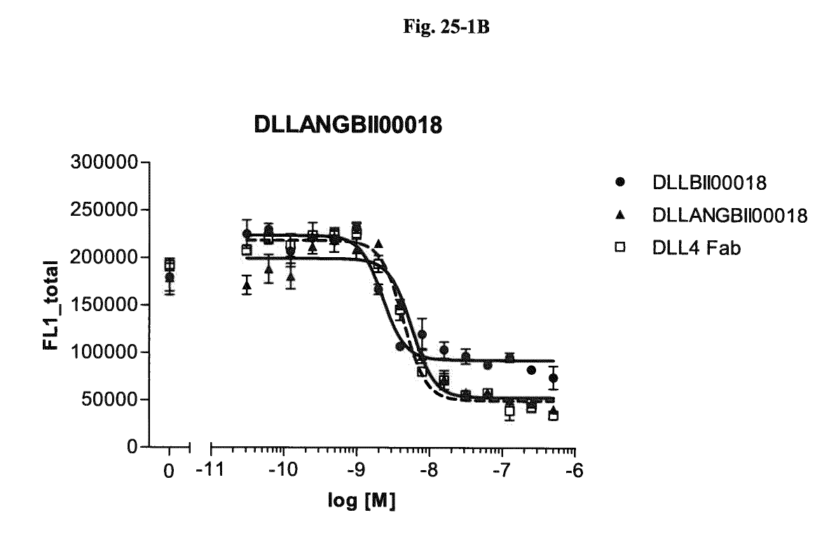

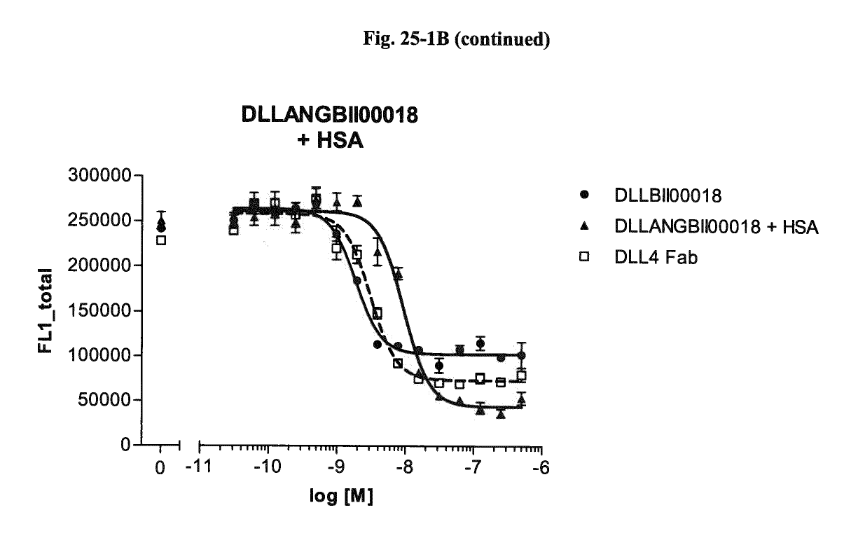

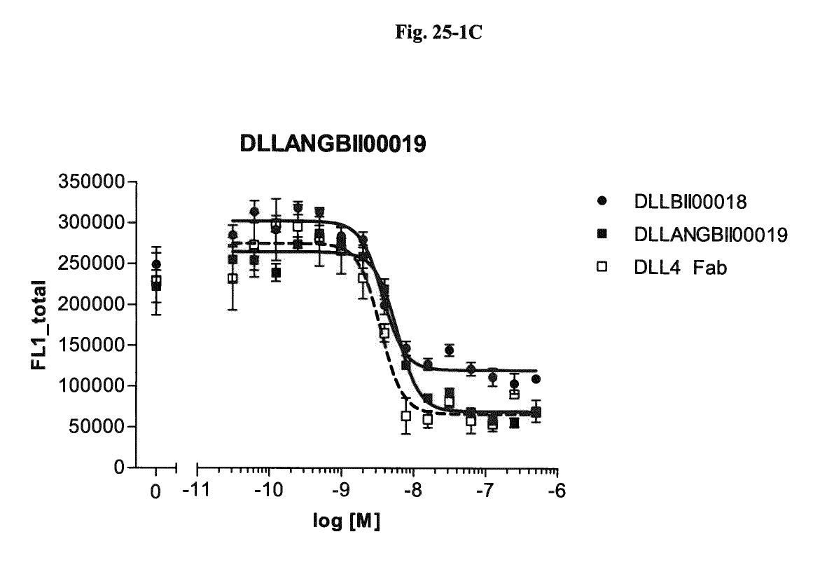

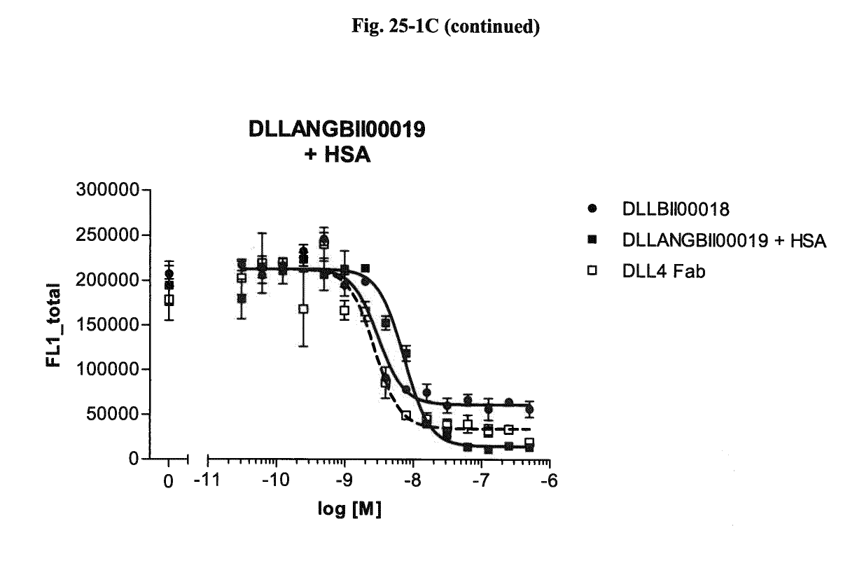

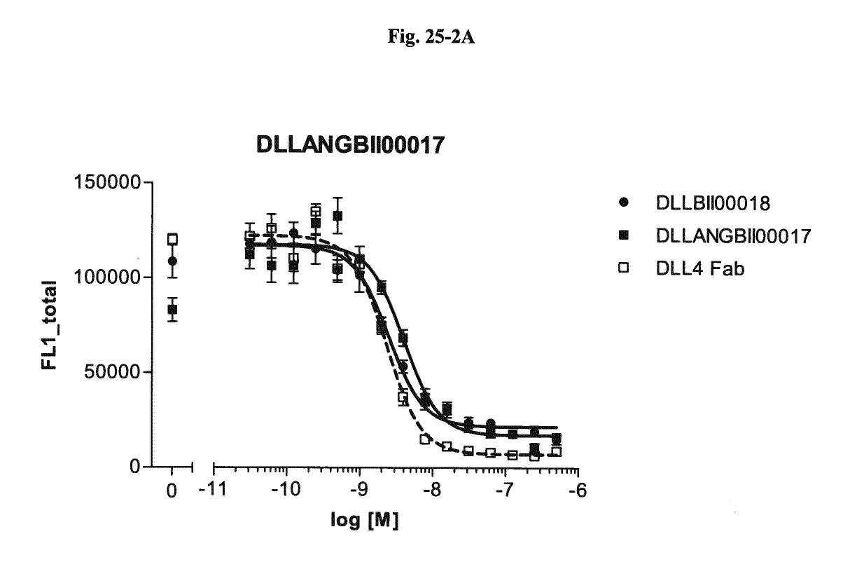

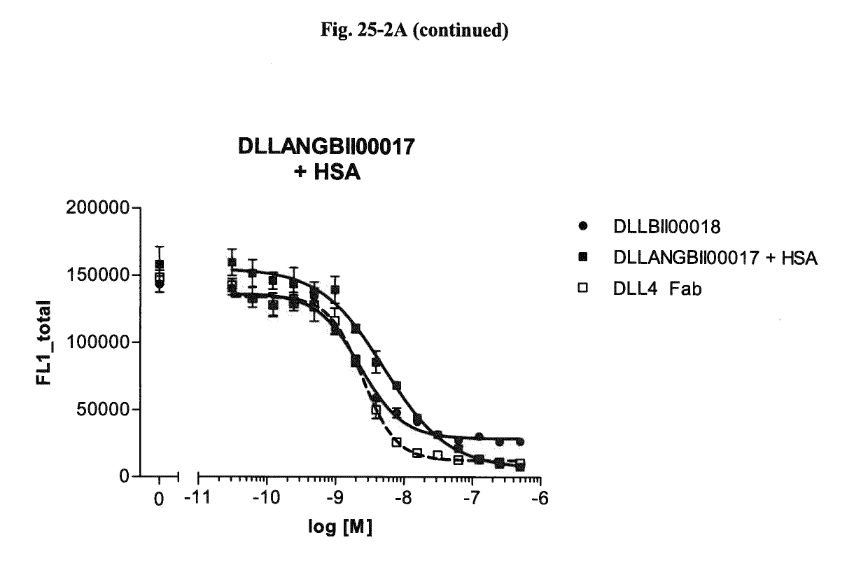

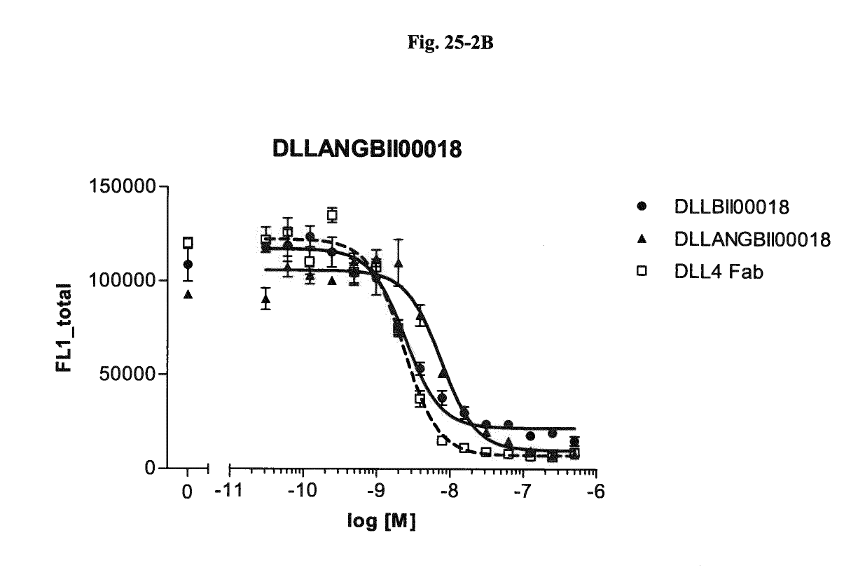

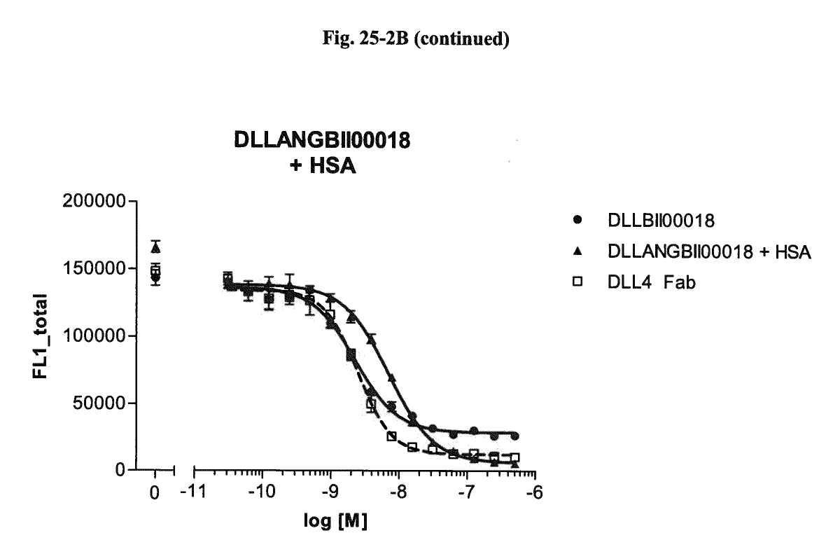

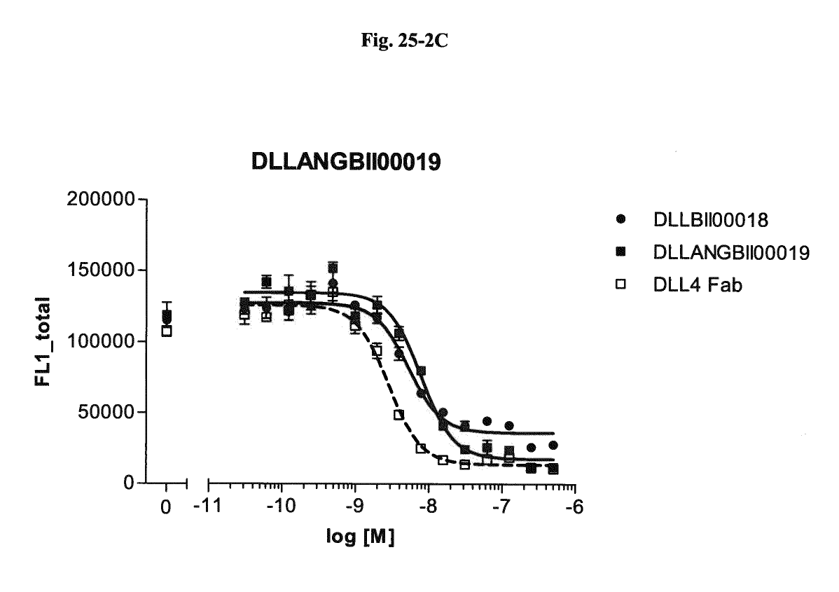

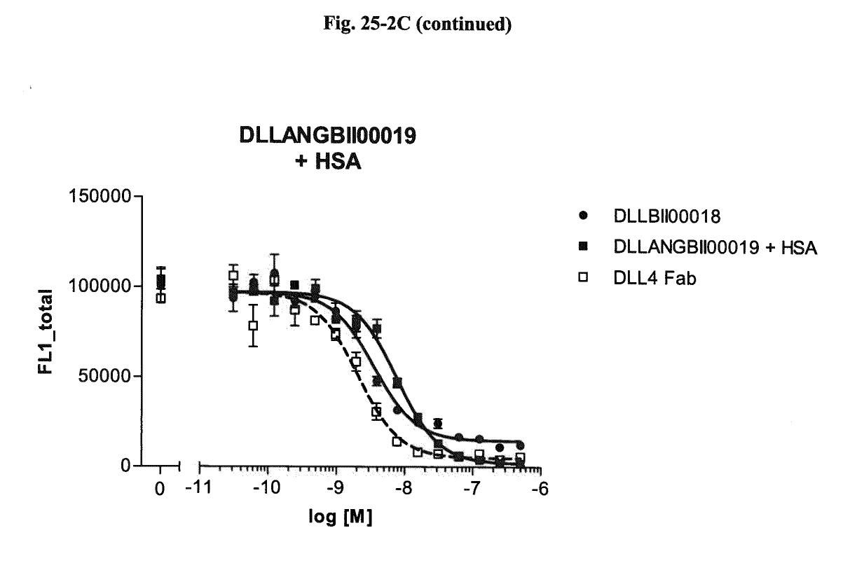

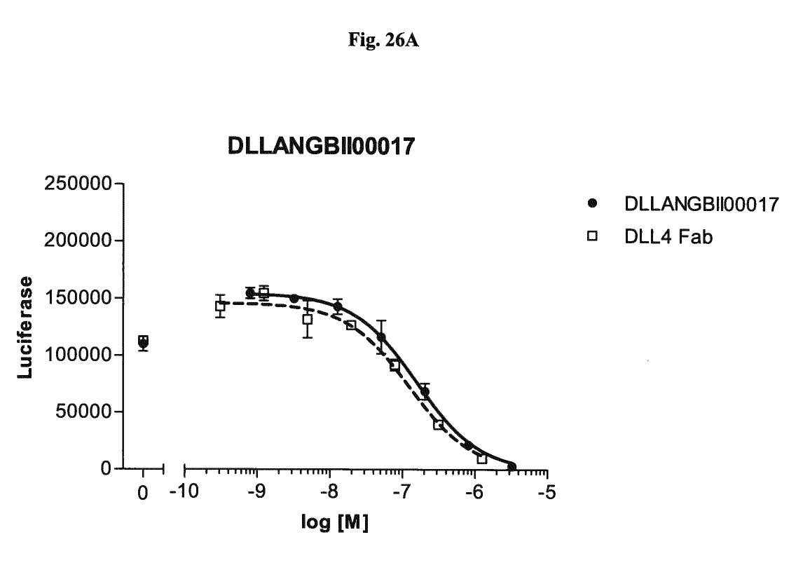

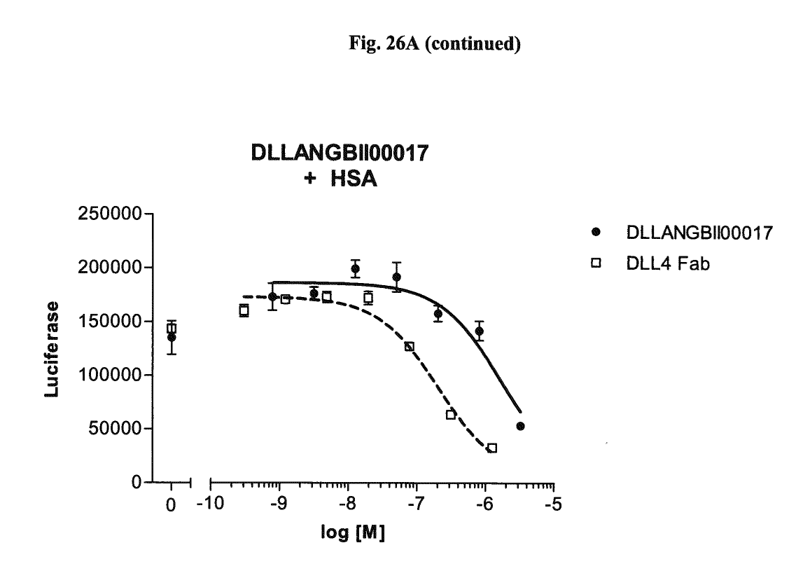

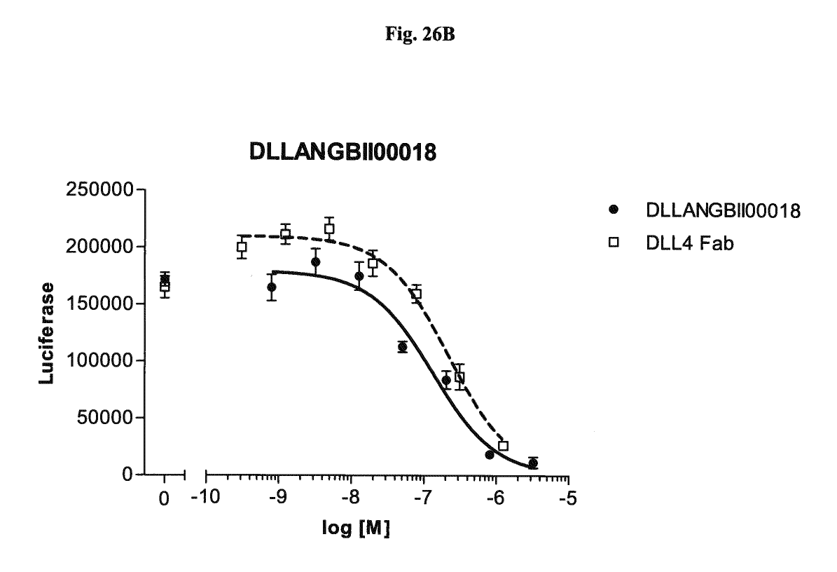

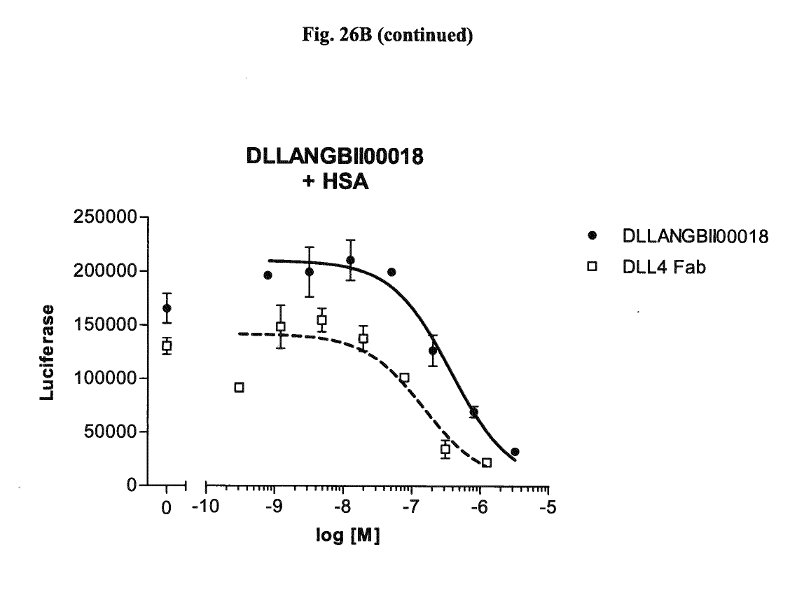

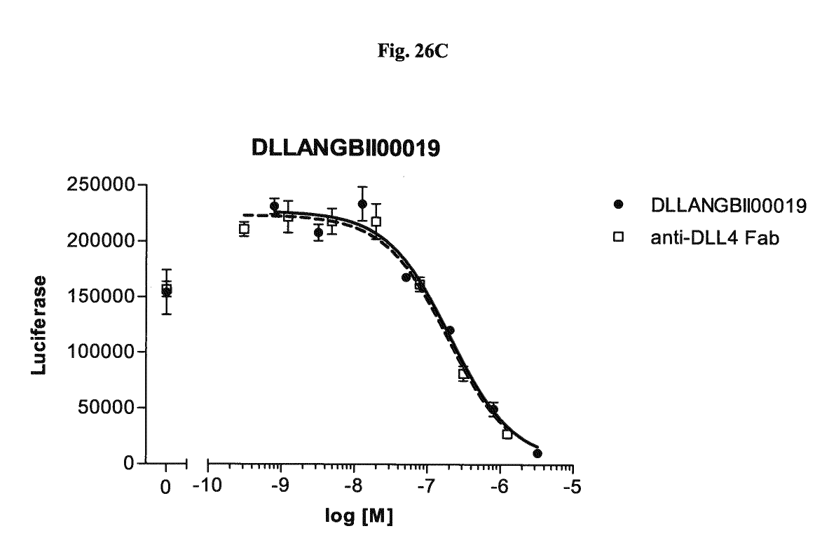

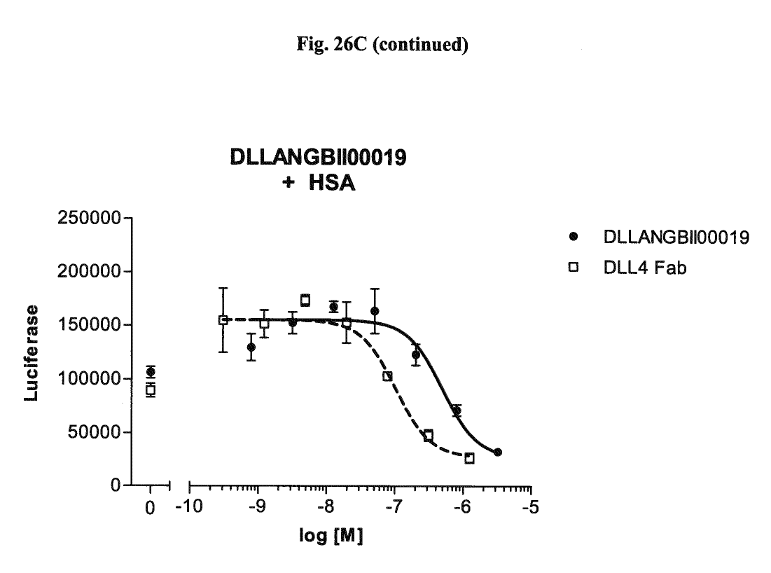

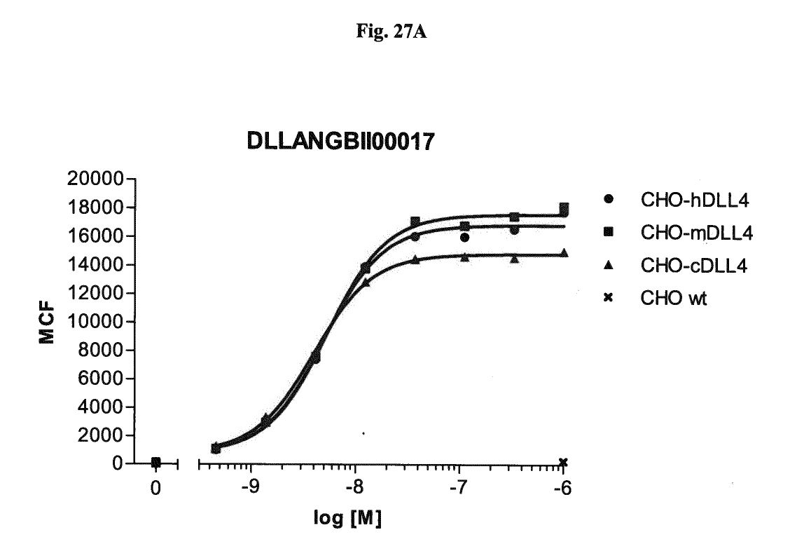

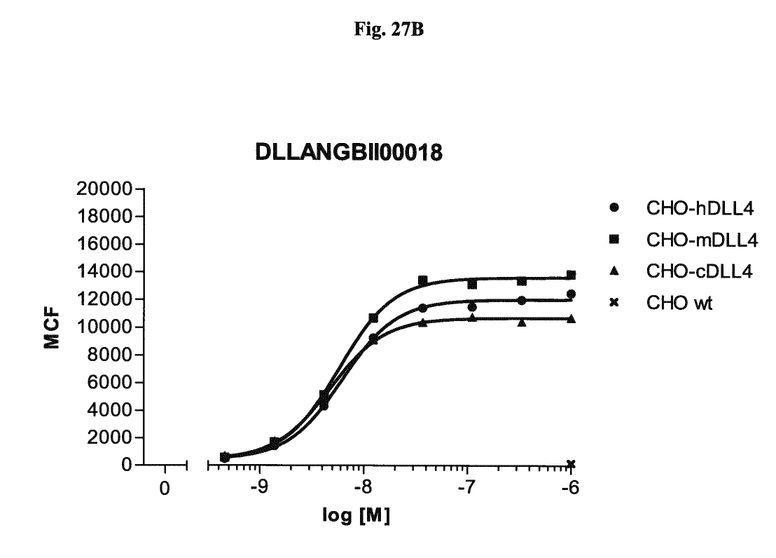

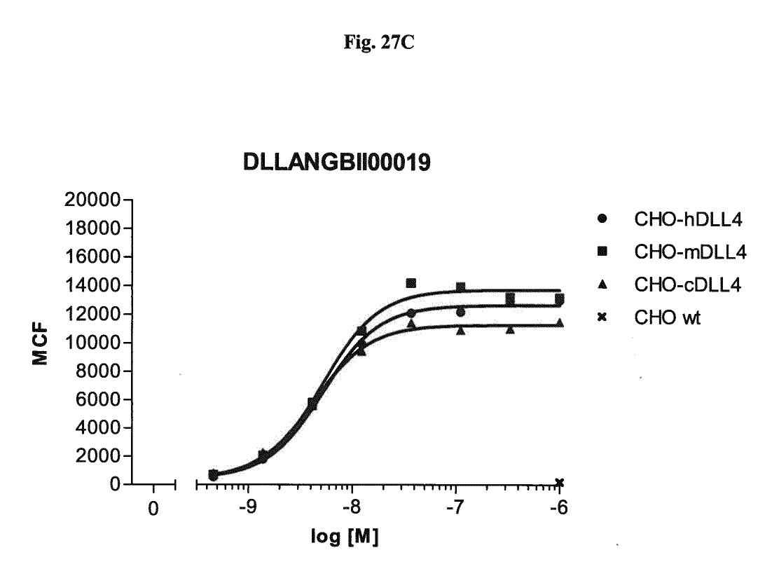

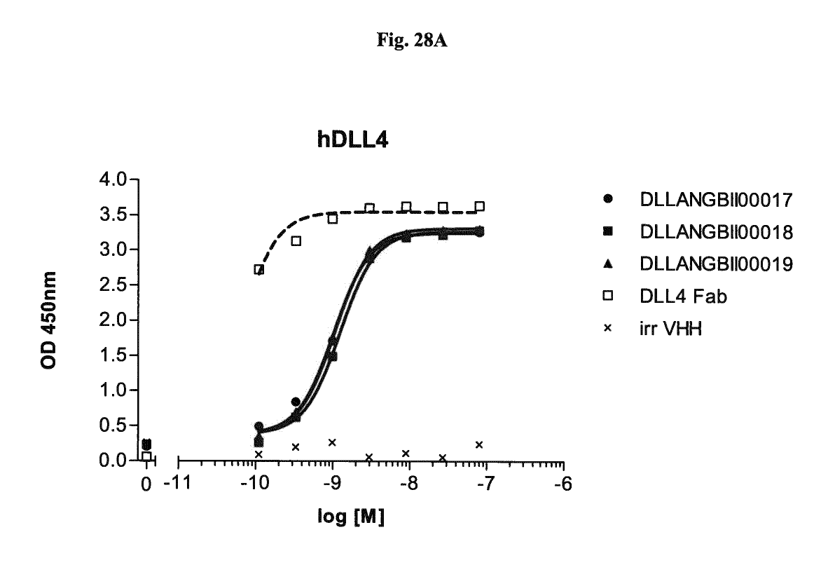

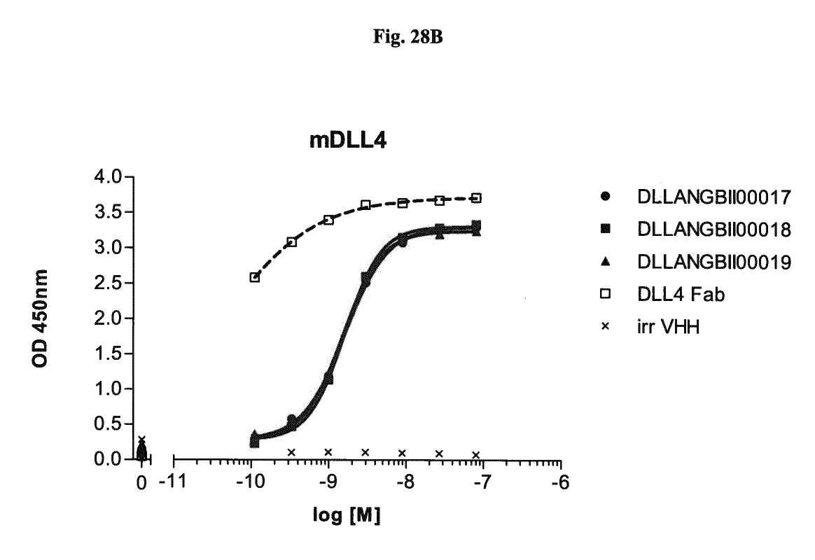

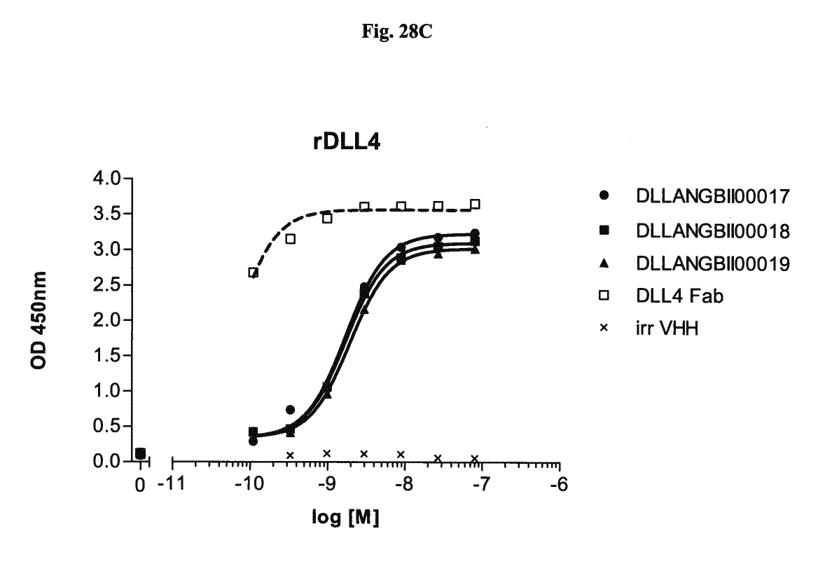

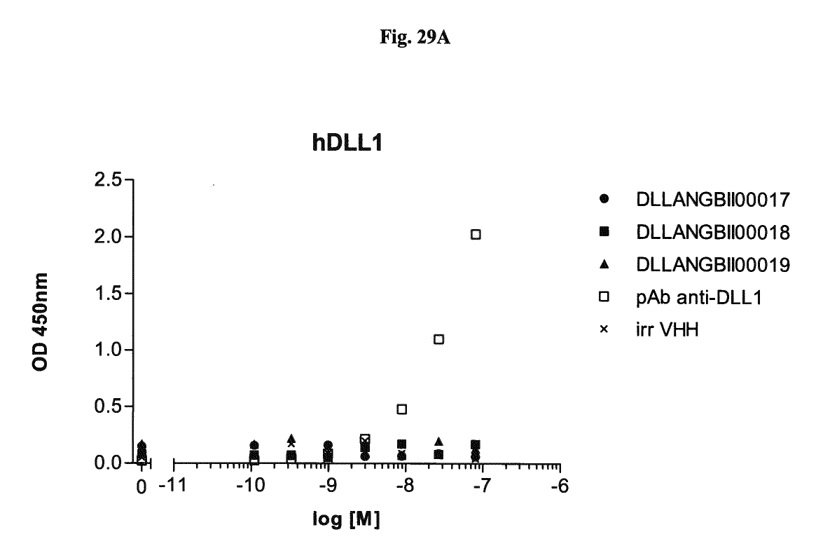

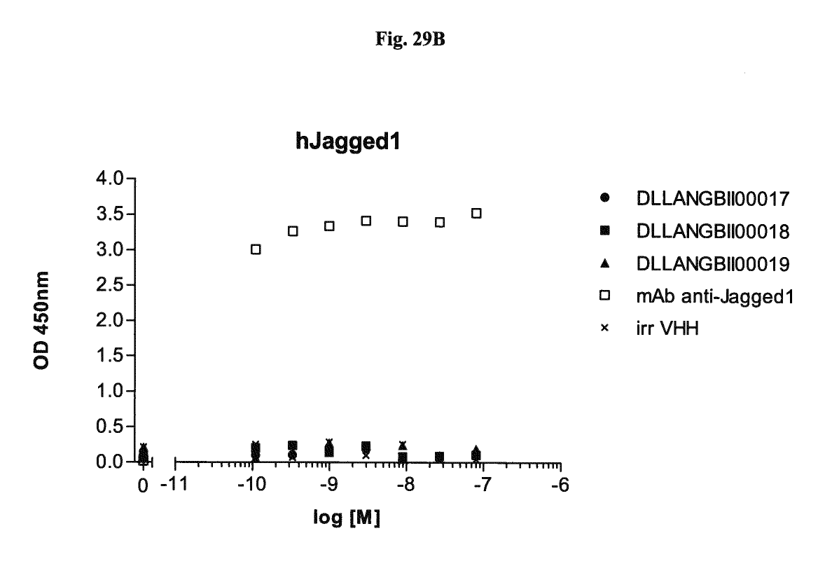

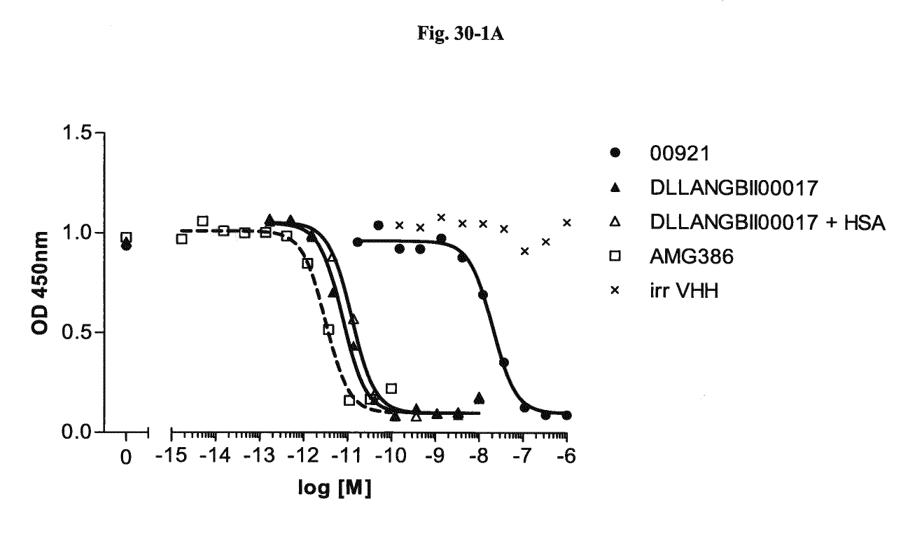

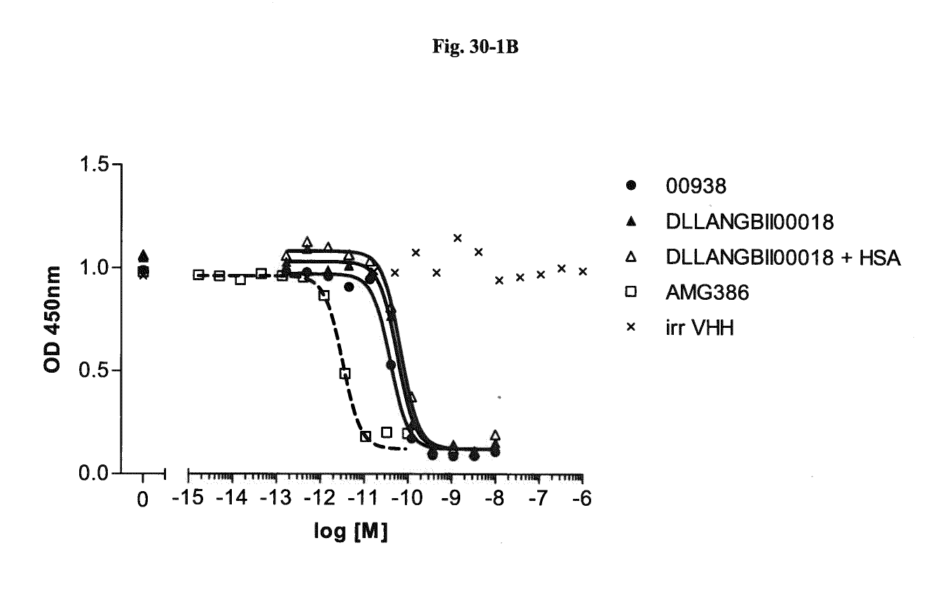

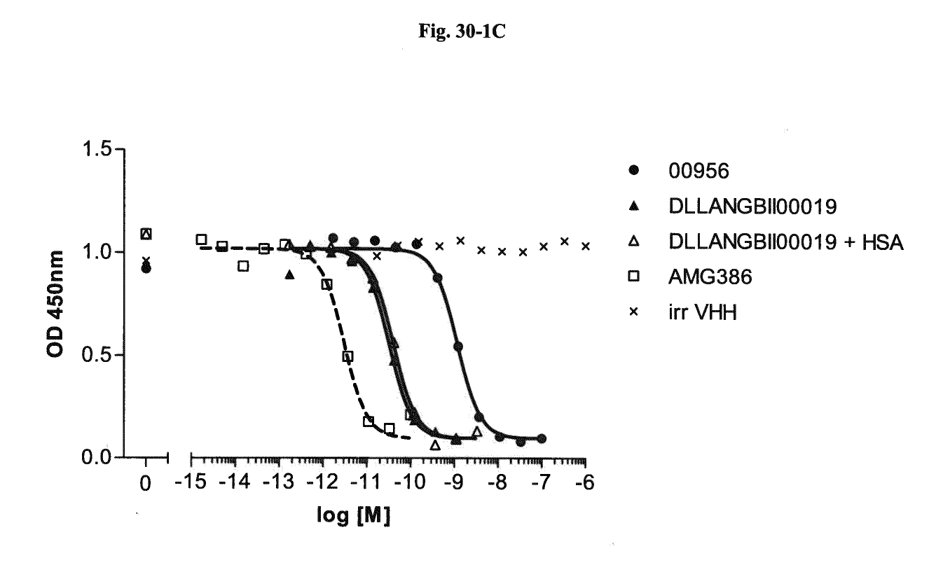

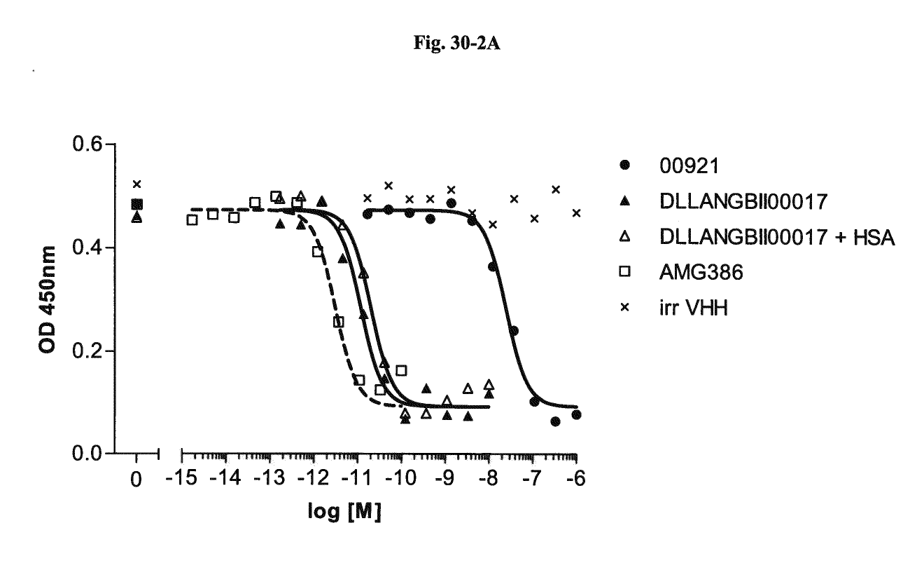

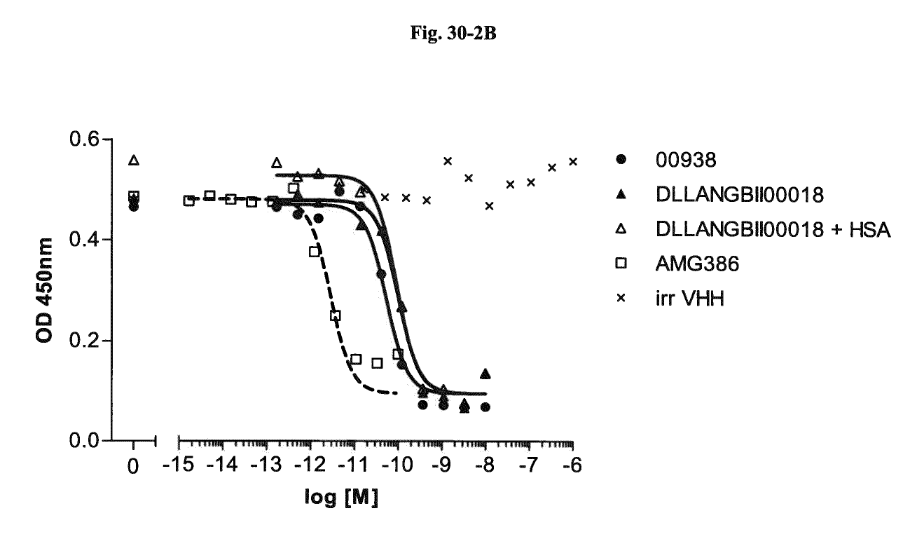

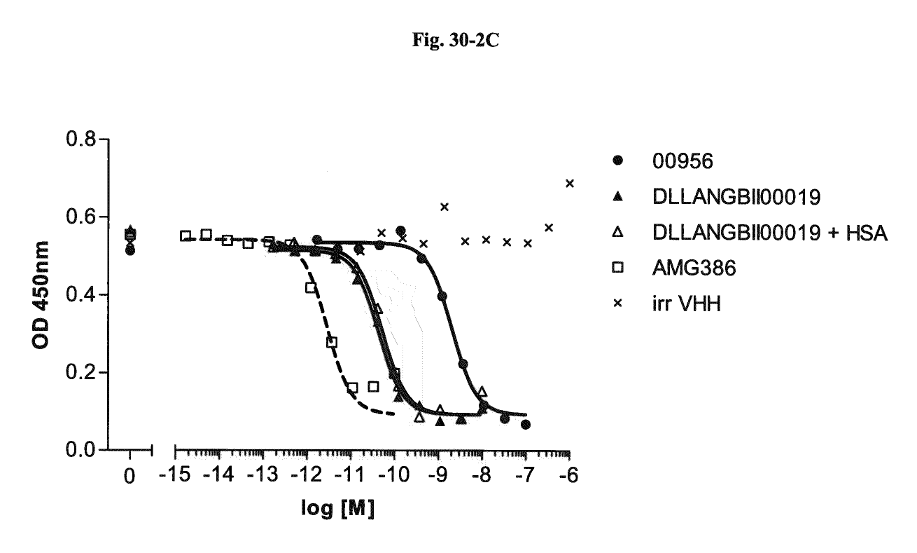

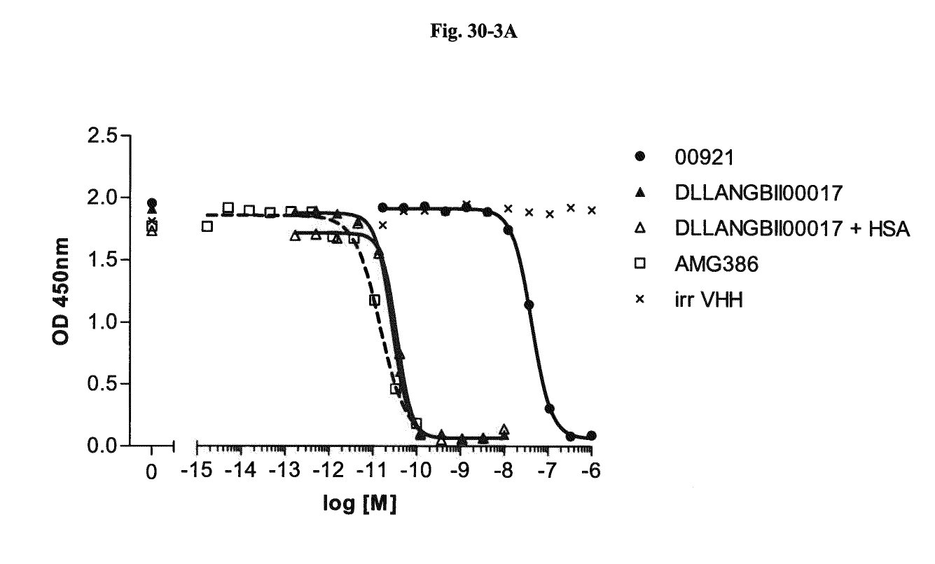

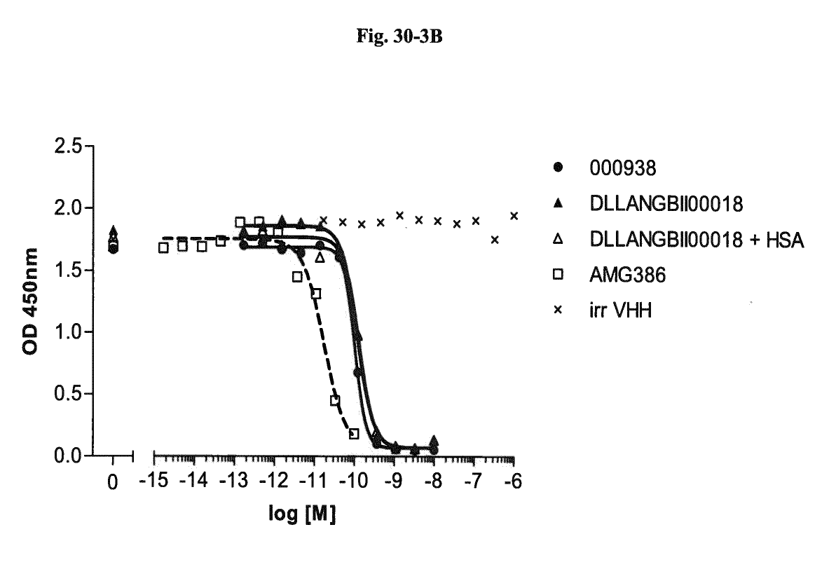

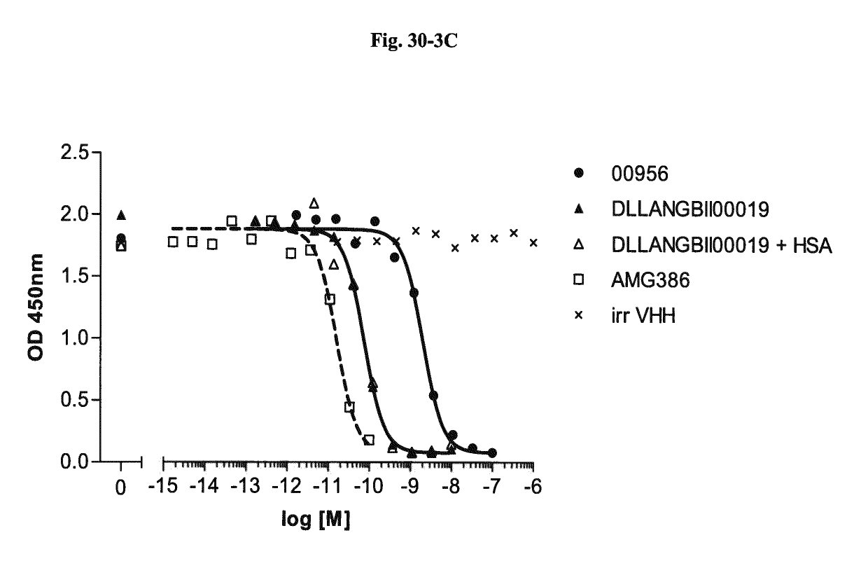

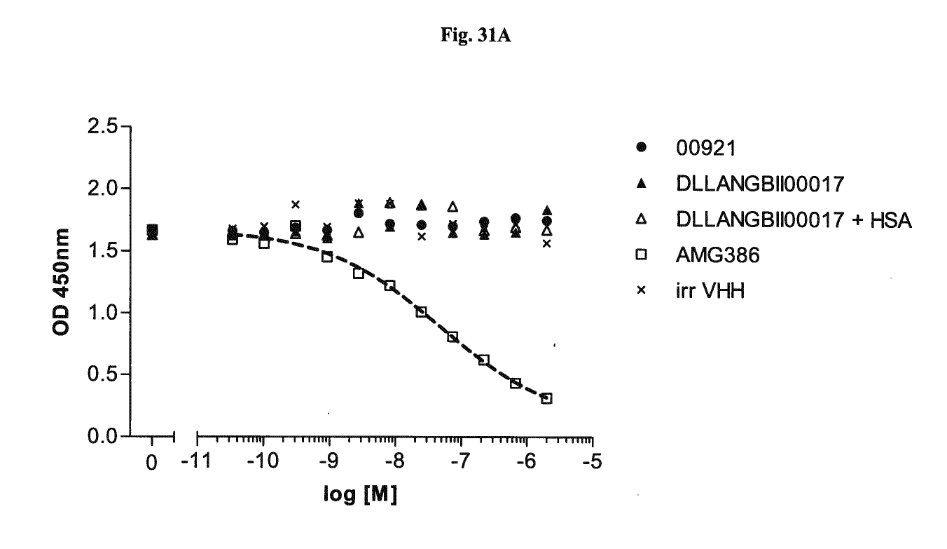

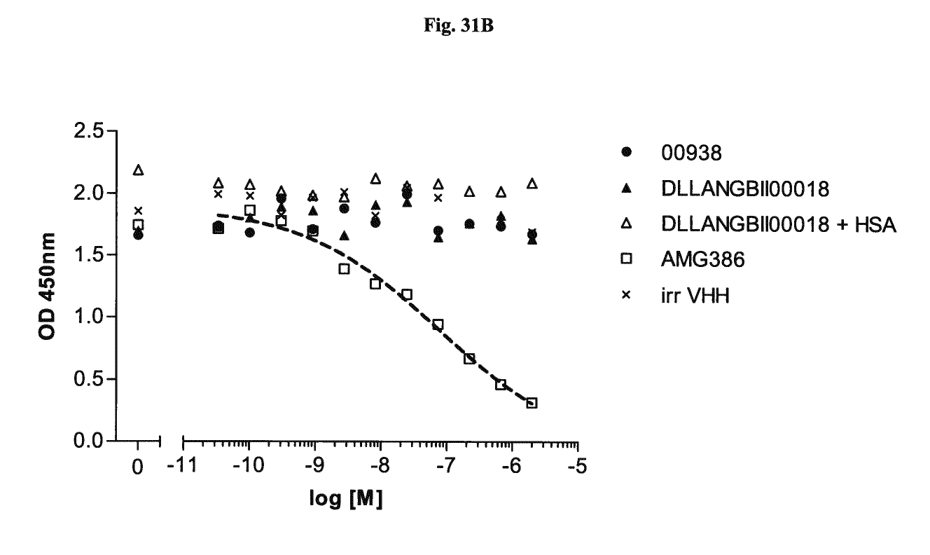

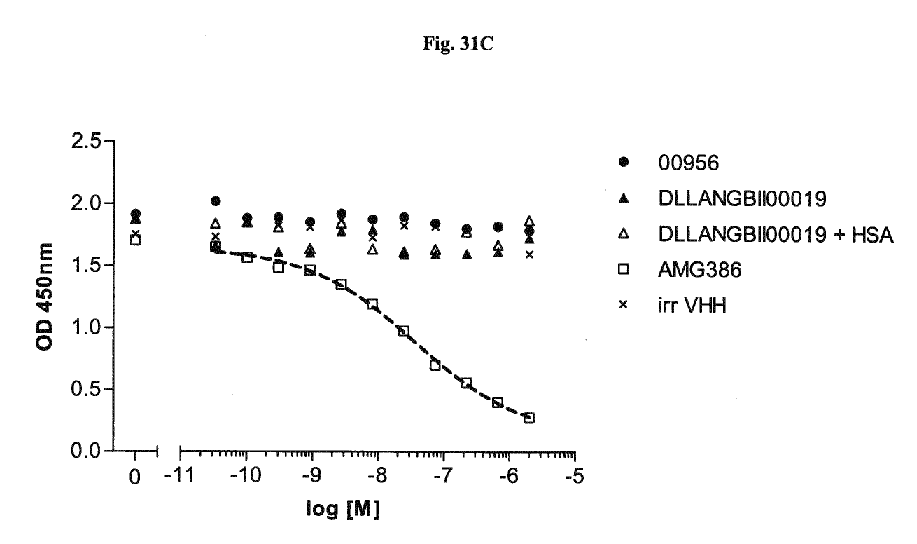

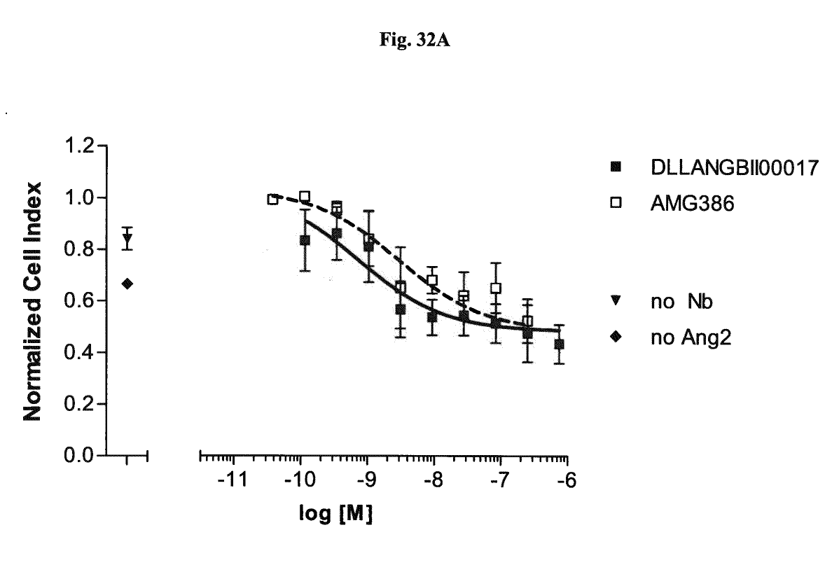

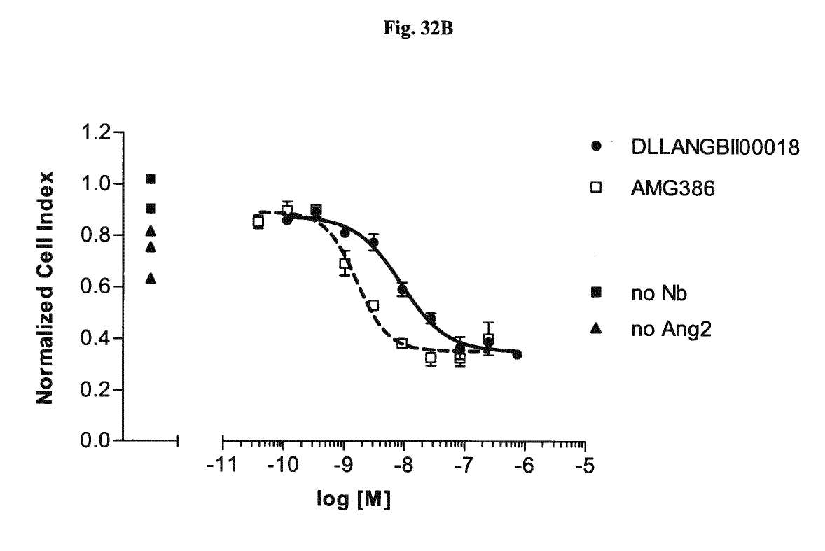

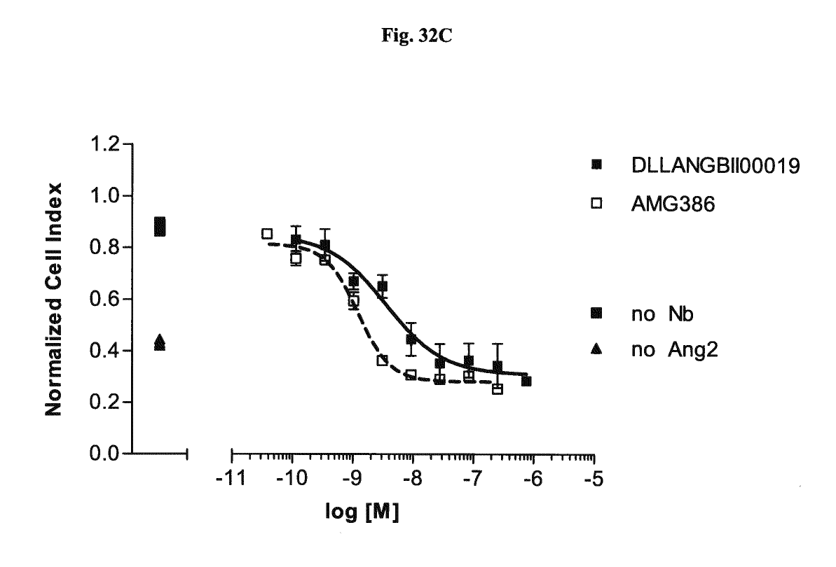

In order to determine cross-reactivity to mouse DLL4 and rat DLL4, a binding ELISA is performed. In brief, recombinant mouse DLL4 (R&D Systems, Minneapolis, Minn., USA) and rat DLL4 is coated overnight at 4° C. in a 96-well MaxiSorp plate (Nunc, Wiesbaden, Germany). Wells are blocked with a 1% casein solution. VHHs are applied as dilution series and binding is detected biotinylated anti-VHH 1A4 followed by extravidin-HRP. 1A4 is an anti-VHH VHH (generated in-house by Ablynx NV). As reference binding to human DLL4 is measured. EC₅₀ values are summarized in Table 25 and FIG. 20.

TABLE 25
EC₅₀ values of bispecific VHHs binding to
human, mouse and rat DLL4 (ELISA)

| | hDLL4 | mDLL4 | rDLLA |
|---|---|---|---|
| | EC₅₀ (nM) | EC₅₀ (nM) | EC₅₀ (nM) |
| DLLBII00018 | 2.5 | 3.3 | 2.6 |
| DLLANGBII00001 | 2.5 | 3.6 | 3.4 |
| DLLANGBII00003 | 2.1 | 3.2 | 2.7 |
| DLLANGBII00005 | 2.0 | 3.1 | 2.9 |
| DLLANGBII00007 | 2.4 | 3.3 | 2.8 |
| DLLANGBII00012 | 2.9 | 3.3 | 3.1 |
| DLLANGBII00014 | 3.2 | 4.2 | 3.8 |

Absence of binding to the homologous human ligands DLL1 and Jagged-1 is assessed via a solid phase binding assay (ELISA). In brief, 1 μg/mL of recombinant human DLL1 (Alexis, San Diego, Calif., USA) or recombinant human Jagged-1 (Alexis, San Diego, Calif., USA) is coated overnight at 4° C. in a 96-well MaxiSorp plate (Nunc, Wiesbaden, Germany). Wells are blocked with a 1% casein solution. VHHs are applied as dilution series and binding is detected biotinylated anti-VHH 1A4 followed by extravidin-HRP. All bispecific VHH are considered as being non-cross reactive to these homologous ligands. Results are shown in FIG. 21.

To explore the anti-Ang2 blocking properties in comparison with the monovalent anti-Ang2 building blocks 00042, 00045 and 00050, all purified bispecific VHHs are analyzed in a human Ang2/hTie2-Fc (FIG. 22-1), mouse Ang2/mTie2 (FIG. 22-2) and cyno Ang2/cTie2 (FIG. 22-3) competition ELISA. This assay is also performed after incubation of the VHH with 0.5 μM human serum albumin. A summary of IC₅₀ and % inhibition values is shown in Table 26.

Affinities of certain DLL4-Ang2 bispecific VHHs for human serum albumin have been determined (see Example 5) and are shown in Table 27. The affinity constant K_{D} is calculated from resulting association and dissociation rate constants kₐ and k_{d}.

In a second cycle, the anti-DLL4 VHH DLLBII00018 (US 2011/0172398 A1) and the final sequence optimized anti-Ang2 VHHs 00921 (SEQ ID NO: 485), 00938 (SEQ ID NO: 486) and 00956 (SEQ ID NO: 488) are used as building blocks to generate bispecific VHHs DLLANGBII00017-00019. A genetic fusion to a serum albumin binding VHH is used as half-life extension methodology. Building blocks are linked via a 9 Gly-Ser flexible linker. An overview of the format and sequence of all bispecific VHHs is depicted in FIG. 23 and Table 28 (linker sequences underlined), SEQ ID Nos 476-478.

TABLE 28
Sequences of bispecific VHH targeting DLL4 and Ang2

| VHH ID | AA sequence |
|---|---|
| DLLANGBII00017 | DVQLVESGGGLVQPGGSLRLSCAASGRTFSSYAMAWYRQAPGKEREYVAAIRWSGGTAYYADSVKGR |
| | FTISRDNAKNTVYLQMNSLRPEDTAVYYCANRAPDTRLAPYEYDHWGQGTLVTVSSGGGGSGGGSEV |
| | QLVESGGGLVQPGNSLRLSCAASGFTFSSFGMSWVRQAPGKGLEWVSSISGSGSDTLYADSVKGRFT |
| | ISRDNAKTTLYLQMNSLRPEDTAVYYCTIGGSLSRSSQGTLVTVSSGGGGSGGGSEVQLVESGGGLV |
| | QPGGSLRLSCAASGFTFDDYALGWFRQAPGKEREGVSCIRCSGGSTYYADSVKGRFTISSDNSKNTV |
| | YLQMNSLRPEDTAVYYCAASIVPRSKLEPYEYDAWGQGTLVTVSSGGGGSGGGSEVQLVESGGGLVQ |
| | PGGSLRLSCAASGFTFDDYALGWFRQAPGKEREGVSCIRCSGGSTYYADSVKGRFTISSDNSKNTVY |
| | LQMNSLRPEDTAVYYCAASIVPRSKLEPYEYDAWGQGTLVTVSS |
| | (SEQ ID NO: 476) |
| DLLANGBII00018 | DVQLVESGGGLVQPGGSLRLSCAVSGITLDDYAIGWFRQAPGKEREGVSAIRSSGGSTYYADSVKGR |
| | FTISSDNSKNTVYLQMNSLRPEDTAVYYCAAVPAGRLRYGEQWYPIYEYDAWGQGTLVTVSSGGGGS |
| | GGGSEVQLVESGGGLVQPGGSLRLSCAASGRTFSSYAMAWYRQAPGKEREYVAAIRWSGGTAYYADS |
| | VKGRFTISRDNAKNTVYLQMNSLRPEDTAVYYCANRAPDTRLAPYEYDHWGQGTLVTVSSGGGGSGG |
| | GSEVQLVESGGGLVQPGNSLRLSCAASGFTFSSFGMSWVRQAPGKGLEWVSSISGSGSDTLYADSVK |
| | GRFTISRDNAKTTLYLQMNSLRPEDTAVYYCTIGGSLSRSSQGTLVTVSS |
| | (SEQ ID NO: 477) |
| DLLANGBII00019 | DVQLVESGGGLVQPGGSLRLSCAASGFTLDDYAIGWFRQAPGKEREGVSAIRSSGGSTYYADSVKGR |
| | FTISSDNSKNTVYLQMNSLRPEDTAVYYCAAVPAGRLRFGEQWYPLYEYDAWGQGTLVTVSSGGGGS |
| | GGGSEVQLVESGGGLVQPGGSLRLSCAASGFTLDDYAIGWFRQAPGKEREGVSAIRSSGGSTYYADS |
| | VKGRFTISSDNSKNTVYLQMNSLRPEDTAVYYCAAVPAGRLRFGEQWYPLYEYDAWGQGTLVTVSSG |
| | GGGSGGGSEVQLVESGGGLVQPGGSLRLSCAASGRTFSSYAMAWYRQAPGKEREYVAAIRWSGGTAY |
| | YADSVKGRFTISRDNAKNTVYLQMNSLRPEDTAVYYCANRAPDTRLAPYEYDHWGQGTLVTVSSGGG |
| | GSGGGSEVQLVESGGGLVQPGNSLRLSCAASGFTFSSFGMSWVRQAPGKGLEWVSSISGSGSDTLYA |
| | DSVKGRFTISRDNAKTTLYLQMNSLRPEDTAVYYCTIGGSLSRSSQGTLVTVSS |
| | (SEQ ID NO: 478) |

To explore the anti-DLL4 blocking properties in comparison with the monovalent building block DLLBII00018, all purified bispecific VHHs are analyzed in the hDLL4/hNotch1 competition ELISA (see Example 5.1 as described in patent US 2011/0172398 A1) (FIG. 24), the CHO-hDLL4/CHO-mDLL4 competition FMAT (see Example 5.3 as described in patent US 2011/0172398 A1) (FIG. 25) and the hDLL4 mediated Notch1 activation (reporter gene) assay (see Example 5.4 as described in patent US 2011/0172398 A1) (FIG. 26). Here, the ELISA competition assay is performed with a fixed concentration of 8 nM biotinylated hDLL4. The ELISA competition assay, the FMAT competition assays and the reporter gene assay are also performed after preincubation of the VHH with 12.5 μM, 25 μM and 162 μM human serum albumin, respectively. A summary of IC₅₀ values and % inhibition is shown in Table 29.

Binding to human DLL4, mouse DLL4 and rat DLL4 is assessed in Biacore. Briefly, kinetic analysis of the bispecific VHHs is performed by SPR on a Biacore T100 instrument. Recombinant human DLL4 (R&D Systems, Minneapolis, Minn., USA) and mouse DLL4 (R&D Systems, Minneapolis, Minn., USA) are immobilized on a CM5 chip via amine coupling. VHHs are injected over these surfaces at different concentrations between 2.5 and 1,800 nM. Samples are injected for 2 min and allowed to dissociate for min at a flow rate of 45 μl/min. Between sample injections, the surfaces were regenerated with a 100 s pulse of 10 mM glycine pH 1.5. Association/dissociation data are evaluated by fitting a 1:1 interaction model (Langmuir binding). The affinity constant K_{D} is calculated from resulting association and dissociation rate constants kₐ and k_{d} (Table 30).

TABLE 30
Binding kinetcs of bispecific VHHs for binding to
human and mouse DLL4 (Biacore)

| | hDLL4 | | | mDLL4 | | |
|---|---|---|---|---|---|---|
| | kₐ | kₐ | K_{D} | kₐ | kₐ | K_{D} |
| | (1/Ms) | (1/s) | (nM) | (1/Ms) | (1/s) | (nM) |
| DLLANGBII00017 | 1.6E+05 | 9.5E−05 | 0.6 | 9.1E+05 | 2.6E−04 | 2.4 |
| DLLANGBII00018 | 2.0E+05 | 9.3E−05 | 0.5 | 1.3E+05 | 2.9E−04 | 1.9 |
| DLLANGBII00019 | 1.1E+05 | 7.8E−05 | 0.7 | 1.5E+05 | 2.8E−04 | 3.0 |

Additionally, in order to determine cross-reactivity of the bispecific VHHs to murine and cynomolgus DLL4, a FACS binding experiment is performed. Briefly, CHO cells overexpressing mouse and cynomolgus DLL4 are used for a titration binding experiment of the VHHs. After a 30 min incubation on ice, all samples are washed and a 2-step detection using biotinylated anti-VHH 1A4 followed by PE labeled streptavidin is performed. CHO cells overexpressing human DLL4 are taken as reference. The mean MCF value is determined using a FACS Array and used for calculation of the EC₅₀ value (Table 31; FIG. 27).

TABLE 31
EC₅₀ values of bispecific VHHs binding to human, mouse
and cyno DLL4 overexpressed on CHO cells (FACS)

| | CHO-hDLL4 | CHO-mDLL4 | CHO-cDLL4 |
|---|---|---|---|
| | EC₅₀ (nM) | EC₅₀ (nM) | EC₅₀ (nM) |
| DLLANGBII00017 | 6.0 | 6.2 | 4.7 |
| DLLANGBII00018 | 7.9 | 6.7 | 5.6 |
| DLLANGBI100019 | 6.7 | 6.3 | 5.3 |
| DLL4Fab | 7.0 | 6.0 | 5.3 |

In order to determine cross-reactivity to mouse DLL4 and rat DLL4, a binding ELISA is performed. In brief, recombinant mouse DLL4 (R&D Systems, Minneapolis, Minn., USA) and rat DLL4 is coated overnight at 4° C. in a 96-well MaxiSorp plate (Nunc, Wiesbaden, Germany). Wells are blocked with a 1% casein solution. VHHs are applied as dilution series and binding is detected biotinylated anti-VHH 1A4 followed by extravidin-HRP. As reference binding to human DLL4 is measured. EC₅₀ values are summarized in Table 32 and FIG. 28.

TABLE 32
EC₅₀ values of bispecific VHHs binding to human,
mouse and rat DLL4 (ELISA)

| | hDLL4 | mDLL4 | cDLL4 |
|---|---|---|---|
| | EC₅₀ (nM) | EC₅₀ (nM) | EC₅₀ (nM) |
| DLLANGBII00017 | 1.0 | 1.6 | 1.7 |
| DLLANGBII00018 | 1.2 | 1.6 | 1.7 |
| DLLANGBII00019 | 1.1 | 1.5 | 1.9 |

Absence of binding to the homologous human ligands DLL1 and Jagged-1 is assessed via a solid phase binding assay (ELISA). In brief, 1 μg/mL of recombinant human DLL1 (Alexis, San Diego, Calif., USA) or recombinant human Jagged-1 (Alexis, San Diego, Calif., USA) is coated overnight at 4° C. in a 96-well MaxiSorp plate (Nunc, Wiesbaden, Germany). Wells are blocked with a 1% casein solution. VHHs are applied as dilution series and binding is detected biotinylated anti-VHH 1A4 followed by extravidin-HRP. All bispecific VHH are considered as being non-cross reactive to these homologous ligands. Results are shown in FIG. 29.

To explore the anti-Ang2 blocking properties in comparison with the final sequence optimized monovalent anti-Ang2 building blocks 00921, 00938 and 00956, all purified bispecific VHHs are analyzed in a human Ang2/hTie2 (FIG. 30-1), mouse Ang2/mTie2 (FIG. 30-2), cyno Ang2/cTie2 (FIG. 30-3), a hAng1/hTie2 (FIG. 31) competition ELISA and the hAng2 mediated HUVEC survival assay (FIG. 32). A summary of IC₅₀ and % inhibition values is shown in Table 33.

Affinities of DLLANGBII00017-18-19 for human, mouse, cyno and rat Ang2 (see Example 5) have been determined and are shown in Table 34.

TABLE 34
Binding kinetics of purified VHHs for recombinant human, cyno, mouse and rat Ang2

| | kₐ | k_{d} | K_{D} | kₐ | k_{d} | K_{D} |
|---|---|---|---|---|---|---|
| | (1/Ms) | (1/s) | (M) | (1/Ms) | (1/s) | (M) |
| | human Ang2-FLD | | | cyno Ang2-FLD | | |
| DLLANGBII00017 | 1.90E+06 | 1.30E−02 | 6.60E−09 | 2.50E+06 | 1.20E−02 | 4.70E−09 |
| DLLANGBII00018 | 8.80E+05 | 3.30E−05 | 3.80E−11 | 1.30E+06 | 3.20E−05 | 2.40E−11 |
| DLLANGBII00019 | 5.10E+05 | 1.60E−03 | 3.10E−09 | 6.30E+05 | 1.30E−03 | 2.10E−09 |
| | mouse Ang2-FLD | | | rat Ang2-FLD | | |
| DLLANGBII00017 | 9.10E+05 | 1.50E−02 | 1.70E−08 | 6.70E+05 | 3.30E−02 | 4.90E−08 |
| DLLANGBII00018 | 4.40E+05 | 6.90E−05 | 1.60E−10 | 3.30E+05 | 9.00E−05 | 2.70E−10 |
| DLLANGBII00019 | 3.80E+05 | 3.80E−03 | 1.00E−08 | 2.80E+05 | 6.00E−03 | 2.10E−08 |

Affinities of DLLANGBII00017-18-19 for human, mouse and cyno serum albumin have been determined (Example 5) and are shown in Table 35. The affinity constant K_{D} is calculated from resulting association and dissociation rate constants kₐ and k_{d}.

TABLE 35
Binding kinetics of purified VHHs for recombinant human, mouse and cyno serum albumin

| | HSA | | | CSA | | | MSA | | |
|---|---|---|---|---|---|---|---|---|---|
| | kₐ | k_{d} | K_{D} | kₐ | k_{d} | K_{D} | kₐ | k_{d} | K_{D} |
| | (1/Ms) | (1/s) | (nM) | (1/Ms) | (1/s) | (nM) | (1/Ms) | (1/s) | (nM) |
| ALB11 | 4.5E+05 | 1.7E−03 | 3.8E−09 | 4.2E+05 | 1.7E−03 | 3.9E−09 | 5.5E+05 | 3.0E−02 | 5.5E−08 |
| DLLANGBII00017 | 1.4E+05 | 4.4E−03 | 3.1E−08 | 1.4E+05 | 4.2E−03 | 2.9E−08 | 1.4E+05 | 1.1E−01 | 7.7E−07 |
| DLLANGBII00018 | 1.6E+05 | 4.7E−03 | 2.9E−08 | 1.6E+05 | 4.6E−03 | 2.9E−08 | * | * | * |
| DLLANGBII00019 | 8.1E+04 | 5.6E−03 | 6.9E−08 | 8.1E+04 | 5.5E−03 | 6.8E−08 | * | * | * |

* could not be properly fitted

TABLE 36
Ang2-binding components
(1D01 (SEQ ID No: 479); 7G08 (SEQ ID No: 480); 027 (SEQ ID No: 481);
00042 (SEQ ID No: 482); 00050 (SEQ ID No: 483); 00045 (SEQ ID No: 484);
00921 (SEQ ID No: 485); 00928 (SEQ ID No: 486); 00938 (SEQ ID No: 487); 00958 (SEQ ID No: 488)

| | FR1 CDR1 FR2 CDR2 |
|---|---|
| 1D01 | EVQLVESGGGLVQAGGSLRLSCAASGFTFD DYALG WFRQAAGKEREGVS CIRCSDGSTYYADSVKG |
| 7G08 | EVQLVESGGGLVQPGGSLRLSCAASGFALD YYAIG WFRQVPGKEREGVS CISSSDGITYYVDSVKG |
| 027 | EVQLVESGGGLVQAGGSLRLSCAASGFTLD DYAIG WFRQAPGKEREGVS CIRDSDGSTYYADSVKG |
| | FR3 CDR3 FR4 |
| 1D01 | RFTISSDNAKNTVYLQMNSLKPEDTAVYYCAA SIVPRSKLEPYEYDA WGQGTQVTVSS |
| 7G08 | RFTISRDNAKNTVYLQMNSLKPEDTAVYYCAT DSGGYIDYDCMGLGYDY SGQGTQVTVSS |
| 027 | RFTISSDNDKNTVYLQMNSLKPEDTAVYYCAA VPAGRLRFGEQWYPLYEYDA WGQGTQVTVSS |
| | FRR1 CDR1 FR2 CDR2 |
| 00042 | EVQLVESGGGLVQPGGSLRLSCAASGFTLD DYAIG WFRQAPGKEREGVS SIRDNDGSTYYADSVKG |
| 00050 | EVQLVESGGGLVQPGGSLRLSCAASGFTFD DYALG WFRQAPGKEREGVS CIRCSDGSTYYADSVKG |
| 00045 | EVQLVESGGGLVQPGGSLRLSCAASGFALD YYAIG YFRQPAGKEREGVS CISSSDGITYYADSVKG |
| | FR3 CDR3 FR4 |
| 00042 | RFTISSDNSKNTVYLQMNSLRPEDTAVYYCAA VPAGRLRFGEQWYPLYEYDA WGQGTLVTVSS |
| 00050 | RFTISSDNSKNTVYLQMNSLRPEDTAVYYCAA SIVPRSKLEPYEYDA WGQGTLVTVSS |
| 00045 | RFTISRDNSKNTVYLQMNSLRPEDTAVYYCAT DSGGYIDYDCMGLGYDY WGQGTLVTVSS |
| | FR1 CDR1 FR2 CDR2 |
| 00921 | EVQLVESGGGLVQPGGSLRLSCAASGFTFD DYALG WFRQAPGKEREGVS CIRCSGGSTYYADSVKG |
| 00928 | EVQLVESGGGLVQPGGSLRLSCAASGFALD YYAIG WFRQAPGKEREGVS CISSSGGITYYADSVKG |
| 00938 | EVQLVESGGGLVQPGGSLRLSCAVSGITLD DYAIG WFRQAPGKEREGVS AIRSSGGSTYYADSVKG |
| 00956 | EVQLVESGGGLVQPGGSLRLSCAASGFTLD DYAIG WFRQAPGKEREGVS AIRSSGGSTYYADSVKG |
| | FR3 CDR3 FR4 |
| 00921 | RFTISSDNSKNTVYLQMNSLRPEDTAVYYCAA SIVPRSKLEPYEYDA WGQGTLVTVSS |
| 00928 | RFTISRDNSKNTVYLQMNSLRPEDTAVYYCAT DSGGYIDYDCSGLGYDY WGQGTLVTVSS |
| 00938 | RFTISSDNSKNTVYLQMNSLRPEDTAVYYCAA VPAGRLRYGEQWYPIYEYDA WGQGTLVTVSS |
| 00956 | RFTISSDNSKNTVYLQMNSLRPEDTAVYYCAA VPAGRLRFGEQWYPLYEYDA WGQGTLVTVSS |

1. A bispecific binding molecule comprising at least one Ang2-binding component and at least one DII4-binding component.

2. The bispecific binding molecule of claim 1, further comprising at least one serum albumin binding component.

3. The bispecific binding molecule of claim 1, comprising a DII4-binding component comprising at least a variable domain with four framework regions and three complementarity determining regions CDR1, CDR2 and CDR3, respectively, wherein said CDR3 has an amino acid sequence selected from amino acid sequences shown in
a. SEQ IDs NOs: 1 to 166 and 458,
b. SEQ ID NOs: 333 to 353, or
c. SEQ ID NOs: 375 to 395.

4. The bispecific binding molecule of claim 3, the DII4-binding component of which is an isolated immunoglobulin single variable domain or a polypeptide containing one or more of said immunoglobulin single variable domains, wherein said immunoglobulin single variable domain consists of four framework regions and three complementarity determining regions CDR1, CDR2 and CDR3, respectively, and wherein said CDR3 has an amino acid sequence selected from amino acid sequences shown in
a. SEQ ID NOs: 1 to 166 and 458,
b. SEQ ID NOs: 333 to 353, or
c. SEQ ID NOs: 375 to 395.

5. The bispecific binding molecule of claim 4, wherein said one or more immunoglobulin single variable domain contain
a. a CDR3 with an amino acid sequence selected from a first group of amino acid sequences shown in SEQ ID NOs: 1 to 166;
b. a CDR1 and a CDR2 with an amino acid sequences that is contained, as indicated in Table 5, as partial sequence in a sequence selected from a second group of amino acid sequences shown SEQ ID NOs: 167 to 332 and 459;
c. wherein a SEQ ID NO: x of said first group, for SEQ ID NOs: 1-166 corresponds to SEQ ID NO: y of said second group in that y=x+166.

6. The bispecific binding molecule of claim 4, wherein said one or more immunoglobulin single variable domains contain
a. a CDR3 with an amino acid sequence selected a said first group of amino acid sequences shown in SEQ ID NOs: 333 to 353;
b. a CDR1 and a CDR2 with an amino acid sequence that is contained, as indicated in Table 16-A, as a partial sequence in a sequence selected from a second group of sequences shown SEQ ID NO: 354 to 374;
c. wherein a SEQ ID NO: x of said first group corresponds with SEQ ID NO: y of said second group in that y=x+21.

7. The bispecific binding molecule of claim 4, wherein said one or more immunoglobulin single variable domains contain
a. a CDR3 with an amino acid sequence selected a said first group of amino acid sequences shown in SEQ ID NOs:375 to 395;
b. a CDR1 and a CDR2 with an amino acid sequence that is contained, as indicated in Table 16-B, as a partial sequence in a sequence selected from a second group of sequences shown in SEQ ID NOs: 396 to 416;
c. wherein a SEQ ID NO: x of said first group corresponds to SEQ ID NO: y of said second group in that y=x+21.

8. The bispecific binding molecule of claim 4, wherein said one or more immunoglobulin single variable domains are VHHs.

9. The bispecific binding molecule of claim 8, wherein said one or more VHHs have an amino acid sequence selected from amino acid sequences shown in SEQ ID NOs: 167 to 332 and 459.

10. The bispecific binding molecule of claim 8, said one or more VHHs have an amino acid sequence selected from amino acid sequences shown in SEQ ID NOs: 354 to 374.

11. The bispecific binding molecule of claim 8, wherein said one or more VHHs have an amino acid sequence selected from amino acid sequences shown in SEQ ID NOs:396 to 416.

12. An immunoglobulin single variable domain which has been obtained by affinity maturation of an immunoglobulin single variable domain as defined in claim 5.

13. A VHH which has been obtained by affinity maturation of a VHH as defined in claim 9.

14. A DII4-binding VHH with an amino acid sequence selected from acid sequences shown in SEQ ID NOs: 356 and 358.

15. An immunoglobulin single variable domain which has been obtained by humanization of a VHH defined in claim 14.

16. A DII4-binding VHH with an amino acid sequence selected from sequences shown in SEQ ID NOs: 402, 407 and 416.

17. An immunoglobulin single variable domain which has been obtained by humanization of a VHH defined in claim 16.

18. An immunoglobulin single variable domain which has been obtained by humanization of an immunoglobulin single variable domain as defined in claim 5.

19. An immunoglobulin single variable domain which has been obtained by humanization of an immunoglobulin single variable domain as defined in claim 12.

20. The bispecific binding molecule of claim 1, which binds to an epitope of DII4 that is totally or partially contained within the EGF-2 domain that corresponds to amino acid residues 252-282 of SEQ ID NO: 417.

21. The bispecific binding molecule of claim 20, which is a immunoglobulin single variable domain or a polypeptide containing same.

22. The bispecific binding molecule of claim 1, comprising an Ang2-binding component comprising at least a variable domain with four framework regions and three complementarity determining regions CDR1, CDR2 and CDR3, respectively, wherein said CDR3 has an amino acid sequence selected from amino acid sequences shown in SEQ IDs NOs: 491, 494, 497, 500, 503, 506, 509, 512, 515, or 518.

23. The bispecific binding molecule of claim 22, the Ang2-binding component of which is an isolated immunoglobulin single variable domain or a polypeptide containing one or more of said immunoglobulin single variable domains, wherein said immunoglobulin single variable domain consists of four framework regions and three complementarity determining regions CDR1, CDR2 and CDR3, respectively, and wherein said CDR3 has an amino acid sequence selected from amino acid sequences shown in SEQ IDs NOs: 491, 494, 497, 500, 503, 506, 509, 512, 515, or 518.

24. The bispecific binding molecule of claim 23, wherein said one or more immunoglobulin single variable domain contain
a. a CDR3 with an amino acid sequence selected from a first group of amino acid sequences shown in SEQ ID NOs: SEQ IDs NOs: 491, 494, 497, 500, 503, 506, 509, 512, 515, or 518 (Table 36);
b. a CDR1 with an amino acid sequences that is contained, as indicated in Table 22-A or 28, as partial sequence in a sequence selected from a second group of amino acid sequences shown SEQ ID NOs: 489, 492, 495, 498, 501, 504, 507, 510, 513, or 516 (Table 36);
c. a CDR2 with an amino acid sequences that is contained, as indicated in Table 22-A or 28, as partial sequence in a sequence selected from a second group of amino acid sequences shown SEQ ID NOs: 490, 493, 496, 499, 502, 505, 508, 511, 514, or 517 (Table 36).

25. The bispecific binding molecule of any one of claim 22, wherein said one or more immunoglobulin single variable domains are VHHs.

26. The bispecific binding molecule of claim 25, wherein said one or more VHHs have an amino acid sequence selected from amino acid sequences shown in SEQ ID NOs: 479, 480, 481, 482, 483, 484, 485, 486, 487, or 488.

27. An immunoglobulin single variable domain which has been obtained by affinity maturation of an immunoglobulin single variable domain as defined in claim 24.

28. A VHH which has been obtained by affinity maturation of a VHH as defined in claim 26.

29. An Ang2-binding VHH with an amino acid sequence selected from acid sequences shown in SEQ ID NOs: 479, 480, 481, 482, 483, 484, 485, 486, 487, or 488.

30. An immunoglobulin single variable domain which has been obtained by humanization of a VHH defined in claim 29.

31. An immunoglobulin single variable domain which has been obtained by humanization of an immunoglobulin single variable domain as defined in claim 24.

32. The binding molecule of any one of claim 2, the serum albumin binding component of which is an isolated immunoglobulin single variable domain or a polypeptide containing one or more of said immunoglobulin single variable domains, wherein said immunoglobulin single variable domain consists of four framework regions and three complementarity determining regions CDR1, CDR2 and CDR3, respectively, and wherein said CDR3 has an amino acid sequence selected from amino acid sequences shown in SEQ ID NOs: 522, 525, 528, 531, 534, 537, or 540.

33. The binding molecule of claim 32, wherein said one or more immunoglobulin single variable domain contain
a. a CDR3 with an amino acid sequence selected from a first group of amino acid sequences shown in SEQ ID NOs: SEQ IDs NOs: 522, 525, 528, 531, 534, 537, or 540;
b. a CDR1 with an amino acid sequences selected from a second group of amino acid sequences shown SEQ ID NOs: 520, 523, 526; 529, 532, 535, or 538;
c. a CDR2 with an amino acid sequences selected from a second group of amino acid sequences shown SEQ ID NOs: 521, 524, 527, 530, 533, 536, or 539.

34. The bispecific binding molecule of claim 32, wherein said one or more immunoglobulin single variable domains are VHHs.

35. The bispecific binding molecule of claim 34, wherein said one or more VHHs have an amino acid sequence shown in SEQ ID NOs: 98 or 519.

36. The bispecific binding molecule of claim 2 having the amino acid sequence selected from amino acid sequences shown in SEQ ID NOs: 460 to 478.

37. A nucleic acid molecule encoding a binding molecule of claim 1 or a vector containing same.

38. A host cell comprising a nucleic acid molecule of claim 37.

39. A pharmaceutical composition comprising at least one bispecific binding molecule of claim 1 as the active ingredient.

40. A method of treating a disease that is associated with DII4-mediated and/or Ang2-mediated effects on angiogenesis comprising administering to a patient an effective amount of a pharmaceutical composition according to claim 39.

41. The method of claim 40 wherein the disease is selected from cancer and cancerous diseases.

42. A method of treating eye diseases comprising administering to a patient an effective amount of a pharmaceutical composition according to claim 39.
